(19) United States
(12) Patent Application Publication
(10) Pub. No.: US 2026/0365635 A1
(43) Pub. Date: Nov. 19, 2026

(54) PYRROLIDINE-INDOLE COMPOUNDS AND COMPOSITIONS COMPRISING THE SAME

(71) Applicant: Compass Pathfinder Limited, Altrincham (GB)

(72) Inventors: John Richard MORPHY, Altrincham (GB); Mark PICHOWICZ, Nottingham (GB)

(73) Assignee: Compass Pathfinder Limited, Altrincham (GB)

(21) Appl. No.: 19/649,356

(22) Filed: Apr. 16, 2026

Related U.S. Application Data
(60) Provisional application No. 63/790,564, filed on Apr. 17, 2025.

(51) Int. Cl. C07D 403/06; A61K 31/404; A61K 31/4155; A61K 31/416; A61K 31/4192; A61K 31/4196; A61K 31/4439; A61K 31/506; C07D 401/14; C07D 403/14; C07D 405/14; C07D 409/14

(52) U.S. Cl. CPC C07D 403/06; A61K 31/404; A61K 31/4155; A61K 31/416; A61K 31/4192; A61K 31/4196; A61K 31/4439; A61K 31/506; C07D 401/14; C07D 403/14; C07D 405/14; C07D 409/14

(57) ABSTRACT

Provided herein are compounds of formula (I), (II), (III), (IV), (V) or pharmaceutically acceptable salts or deuterated forms thereof, wherein R¹, R², R³, R⁴, R⁵, R⁶, R^{7A}, R^{7B}, R⁸, R⁹, R¹⁰, n, and m are defined herein. Also provided herein are pharmaceutical compositions comprising a compound of formula (I), (II), (III), (IV), (V) or pharmaceutically acceptable salt or deuterated form thereof, and methods of using a compound of formula (I), (II), (III), (IV), (V) or pharmaceutically acceptable salt or deuterated form thereof, e.g., in the treatment of the treatment of a serotonin 5-HT2A receptor associated disease/disorder.

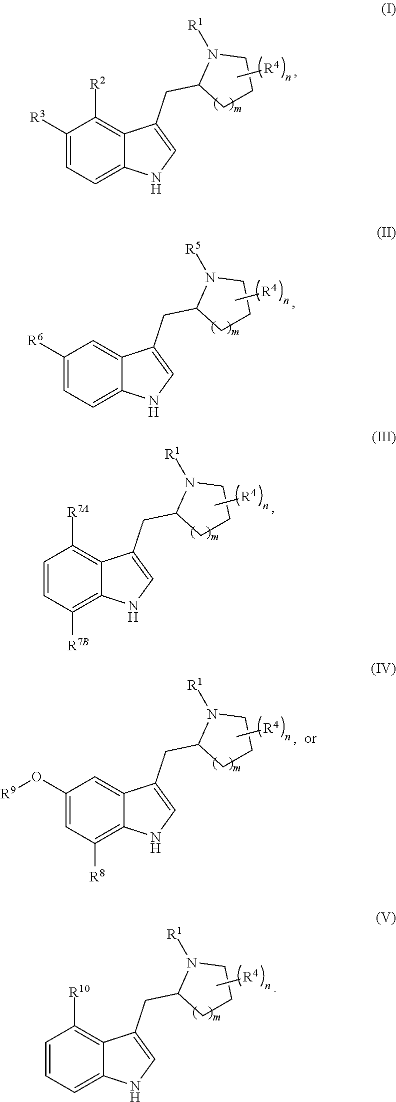

### CROSS-REFERENCE TO RELATED APPLICATIONS

The present application claims priority to U.S. Provisional Patent Application No. 63/790,564 filed Apr. 17, 2025, which is hereby incorporated by reference in its entirety.

### BACKGROUND

Over 50% of adults in the United States will be diagnosed with a psychiatric disorder at some point in their lifetime. Nearly 1 in 5 suffer from mental illness, and nearly 1 in 25 are afflicted with severe mental illness, such as major depression, schizophrenia, or bipolar disorder.

Psychedelics show promising activity in treating mental illness. New psychedelic compounds are needed for treating mental illness.

### SUMMARY

In embodiments, the present disclosure provides a compound of formula (I):

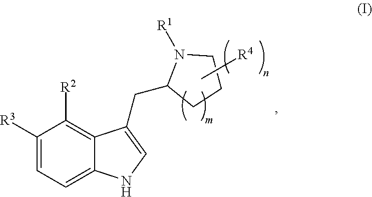

or a pharmaceutically acceptable salt, deuterated form or stereoisomer thereof
wherein:
R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or —C₁₋₆ alkyl;
R² is -halo, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, —O—C₁₋₆ alkyl, —OH, —S—C₁₋₆ alkyl, —S(O)₂—C₁₋₆ alkyl, —SH, —NH—C₁₋₆ alkyl, —N(C₁₋₆ alkyl)₂, —NH₂, —CN, —C(C═O)—C₁₋₆ alkyl, —O(C═O)—C₁₋₆ alkyl, or —NH(C═O)—C₁₋₆ alkyl;
R³ is -halo, —O—C₁₋₆ alkyl, —OH, —S—C₁₋₆ alkyl, —S(O)₂—C₁₋₆ alkyl, —SH, —NH—C₁₋₆ alkyl, —N(C₁₋₆ alkyl)₂, —NH₂, —CN, —C(C═O)—C₁₋₆ alkyl, —O(C═O)—C₁₋₆ alkyl, or —NH(C═O)—C₁₋₆ alkyl;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —OH, —C₁₋₆ alkylene-O—C₁₋₆ alkyl, or —O—C₁₋₆ alkyl;
n is 0, 1, 2, or 3; and
m is 0 or 1,
and wherein, R² and R³ are not both -halo.

In embodiments, the present disclosure provides a compound of formula (II):

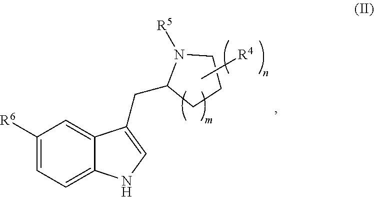

or a pharmaceutically acceptable salt, deuterated form or stereoisomer thereof
wherein:
R⁵ is —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or —C₁₋₆ alkyl;
R⁶ is —O—C₁₋₆ alkyl, —S—C₁₋₆ alkyl, —S(O)₂—C₁₋₆ alkyl, —SH, —N(C₁₋₆ alkyl)₂, —CN, —C(C═O)—C₁₋₆ alkyl, —O(C═O)—C₁₋₆ alkyl, —C₁₋₆ alkylene-OH, or —S(O)—C₁₋₆ alkyl;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —OH, —C₁₋₆ alkylene-O—C₁₋₆ alkyl, or —O—C₁₋₆ alkyl;
n is 0, 1, 2, or 3; and
m is 0 or 1,
and wherein, the compound is not:

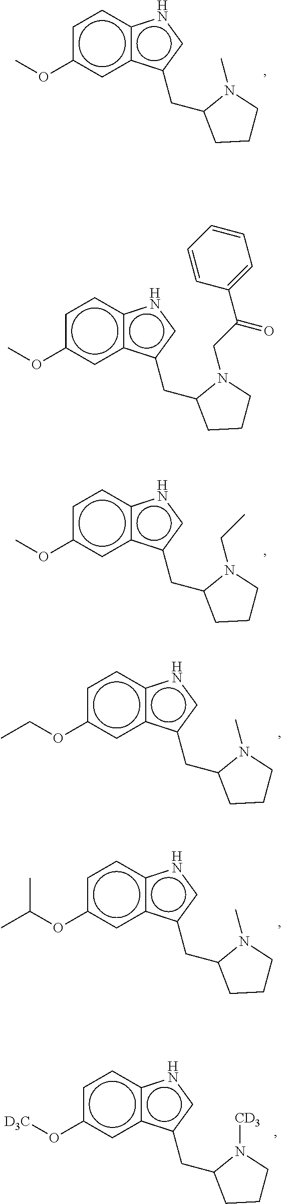

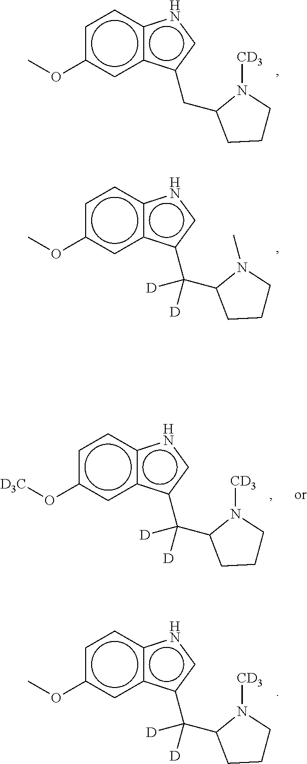

In embodiments, the present disclosure provides a compound of formula (III):

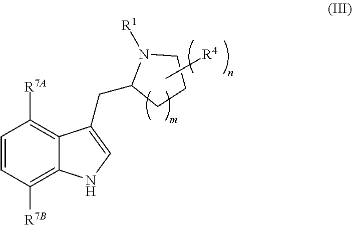

or a pharmaceutically acceptable salt, deuterated form or stereoisomer thereof
wherein:
R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or —C₁₋₆ alkyl;
R^{7A} and R^{7B} are each independently -halo;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —OH, —C₁₋₆ alkylene-O—C₁₋₆ alkyl, or —O—C₁₋₆ alkyl;
n is 0, 1, 2, or 3; and
m is 0 or 1,

In embodiments, the present disclosure provides a compound of formula (IV):

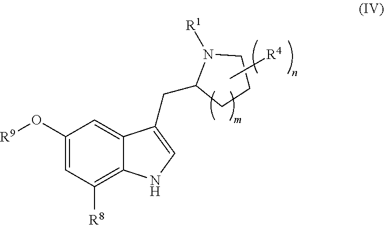

or a pharmaceutically acceptable salt, deuterated form or stereoisomer thereof
wherein:
R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or —C₁₋₆ alkyl;
R⁸ is -halo or -methyl;
R⁹ is —C₁₋₆ alkyl;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —OH, —C₁₋₆ alkylene-O—C₁₋₆ alkyl, or —O—C₁₋₆ alkyl;
n is 0, 1, 2, or 3; and
m is 0 or 1.

In embodiments, the present disclosure provides a compound of formula (V):

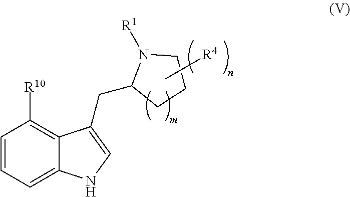

or a pharmaceutically acceptable salt, deuterated form or stereoisomer thereof
wherein:
R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or —C₁₋₆ alkyl;
R¹⁰ is -halo, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, —C₁₋₆ alkylene-O—C₁₋₆ alkyl, —S—C₁₋₆ alkyl, —S(O)₂—C₁₋₆ alkyl, —SH, —NH—C₁₋₆ alkyl, —N(C₁₋₆ alkyl)₂, —NH₂, —CN, —C(C═O)—C₁₋₆ alkyl, —O(C═O)—C₁₋₆ alkyl, or —NH(C═O)—C₁₋₆ alkyl;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —OH, —C₁₋₆ alkylene-O—C₁₋₆ alkyl, or —O—C₁₋₆ alkyl;
n is 0, 1, 2, or 3; and
m is 0 or 1.

In embodiments, the compound is selected from any one of the compounds in Tables A-L.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### DEFINITIONS

Listed below are definitions of various terms used in the specification and claims to describe the present disclosure.

Unless defined otherwise, all technical and scientific terms used in this disclosure have the same meanings as commonly understood by one of ordinary skill in the art to which this disclosure belongs.

The terms below, as used herein, have the following meanings, unless indicated otherwise:

“Alkyl” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl.

“Alkylene” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene is attached to the rest of the molecule through a single bond and to the radical group through a single bond. The points of attachment of the alkylene to the rest of the molecule and to the radical group can be through one carbon or any two carbons within the chain.

“Alkenyl” refers to a straight or branched hydrocarbon chain radical having from two to twelve carbon atoms, and having one or more carbon-carbon double bonds. Each alkenyl is attached to the rest of the molecule by a single bond. Alkenyl comprising any number of carbon atoms from 2 to 12 are included. An alkenyl comprising up to 12 carbon atoms is a C₂-C₁₂ alkenyl, an alkenyl comprising up to 10 carbon atoms is a C₂-C₁₀ alkenyl, an alkenyl comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl.

“Alkynyl” refers to a straight or branched hydrocarbon chain radical having from two to twelve carbon atoms, and having one or more carbon-carbon triple bonds. Each alkynyl is attached to the rest of the molecule by a single bond. Alkynyl comprising any number of carbon atoms from 2 to 12 are included. An alkynyl comprising up to 12 carbon atoms is a C₂-C₁₂ alkynyl, an alkynyl comprising up to 10 carbon atoms is a C₂-C₁₀ alkynyl, an alkynyl comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like.

“Aryl” refers to a hydrocarbon ring system radical comprising hydrogen, 6 to 18 carbon ring atoms and at least one aromatic ring. For purposes of this disclosure, the aryl radical can be a monocyclic, bicyclic, tricyclic or tetracyclic ring system, which can include fused, bridged, or spiro ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene.

“Cycloalkyl” refers to a stable non-aromatic monocyclic or polycyclic fully saturated hydrocarbon radical consisting solely of carbon and hydrogen atoms, which can include fused, bridged, or spiro ring systems, having from three to twenty carbon atoms, e.g., having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like.

“Heteroaryl” refers to a 5- to 20-membered ring system radical comprising one to thirteen carbon ring atoms, one to six heteroatoms as ring atoms selected from nitrogen, oxygen and sulfur, and at least one aromatic ring containing at least one heteroatom as a ring atom. For purposes of this disclosure, the heteroaryl radical can be a monocyclic, bicyclic, tricyclic or tetracyclic ring system, which can include fused, bridged, or spiro ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophene), benzotriazolyl, benzo[4,6]imidazo[1,2-a]pyridinyl, carbazolyl, cinnolinyl, dibenzofuranyl, dibenzothiophene, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophene (i.e. thienyl).

“Heterocyclyl” refers to a stable 3- to 20-membered non-aromatic, saturated or partially unsaturated ring radical which consists of two to twelve carbon ring atoms and from one to six heteroatoms as ring atoms selected from nitrogen, oxygen or sulfur, at least one non-aromatic, saturated or partially unsaturated ring containing at least one heteroatom as a ring atom. Unless stated otherwise specifically in the specification, the heterocyclyl radical can be a monocyclic, bicyclic, tricyclic or tetracyclic ring system, which can include fused, bridged, or spiro ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

As used herein, the symbol

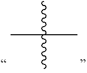

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

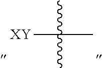

indicates that the chemical entity “XY” is bonded to another chemical entity via the point of attachment bond. Furthermore, the specific point of attachment to the non-depicted chemical entity can be specified by inference.

In this specification, unless stated otherwise, the term “pharmaceutically acceptable” is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have.

Deleterious effects may include, for example, excessive toxicity, irritation, allergic response, and other problems and complications.

The term “pharmaceutically acceptable salt” includes both acid and base addition salts.

Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “substituted” used herein means any of the groups described herein (e.g., alkyl, alkenyl, alkynyl, aryl, carbocyclyl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ, “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heteroaryl, N-heteroaryl and/or heteroarylalkyl group. In embodiments, “substituted” includes any of the above groups in which one or more hydrogen atoms are replaced with halo, nitro, —CN, —C₁-C₆ alkyl, NR^{A}R^{B}, C₁-C₆-haloalkyl, —C₁-C₆-alkyl-NR^{A}R^{B}, —C₁-C₆-alkyl-OH, —C(═O)R^{D}, —C(═O)N(R^{E})R^{F}, —C(═O)—OR^{D}, —N(R^{G})C(═O)R^{D}, —N(R^{G})C(═O)N(R^{E})R^{F}, —N(R^{G})C(═O)OR^{D}, —N(R^{G})S(═O)R^{D}, —N(R^{G})S(═O)₂R^{D}, —N═S(═O)(R^{E})R^{F}, —OR^{D}, —O(C═O)R^{D}, —O(C═O)N(R^{E})R^{F}, —O(C═O)OR^{D}, —C₂-C₆ alkenyl, or —C₂-C₆alkynyl; wherein R^{A} and R^{B} are each independently —H, —C₁-C₆ alkyl, cycloalkyl, aryl, heteroaryl, —C(O)R^{C}, —C(O)OR^{C}, —S(O)R^{C}, or —S(O)₂R^{C}; R^{C} is H, —C₁-C₆ alkyl, aryl, cycloalkyl, or heterocyclyl; and R^{D}, R^{E}, R^{F}, R^{G}, are each independently selected from —H, C₁-C₆-alkyl-, C₁-C₆-haloalkyl-, —C₁-C₆-alkyl-OH, cycloalkyl, C₂-C₆-alkenyl-, heterocyclyl, aryl, or heteroaryl. In addition, each of the foregoing substituents can also be optionally substituted with one or more of the above substituents.

The compounds of the disclosure, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms whether or not they are specifically depicted herein. Optically active (+) and (−), (R)- and (S)-, or (D)- and (L)- isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

### Compounds

The present disclosure provides compounds of formula (I), (II), (III) (IV), (V), (I-E), Table A-L, or a pharmaceutically acceptable salt, a stereoisomer, or a deuterated form thereof.

In embodiments, the present disclosure provides a compound of formula (I):

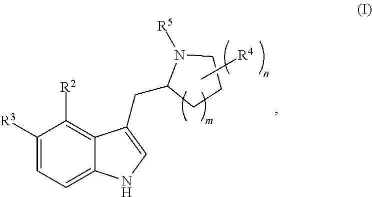

or a pharmaceutically acceptable salt, deuterated form or stereoisomer thereof
wherein:
R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or —C₁₋₆ alkyl;
R² is -halo, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, —O—C₁₋₆ alkyl, —OH, —S—C₁₋₆ alkyl, —S(O)₂—C₁₋₆ alkyl, —SH, —NH—C₁₋₆ alkyl, —N(C₁₋₆ alkyl)₂, —NH₂, —CN, —C(C═O)—C₁₋₆ alkyl, —O(C═O)—C₁₋₆ alkyl, or —NH(C═O)—C₁₋₆ alkyl;
R³ is -halo, —O—C₁₋₆ alkyl, —OH, —S—C₁₋₆ alkyl, —S(O)₂—C₁₋₆ alkyl, —SH, —NH—C₁₋₆ alkyl, —N(C₁₋₆ alkyl)₂, —NH₂, —CN, —C(C═O)—C₁₋₆ alkyl, —O(C═O)—C₁₋₆ alkyl, or —NH(C═O)—C₁₋₆ alkyl;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —OH, —C₁₋₆ alkylene-O—C₁₋₆ alkyl, or —O—C₁₋₆ alkyl;
n is 0, 1, 2, or 3; and
m is 0 or 1,
and wherein, R² and R³ are not both -halo.

In embodiments, the present disclosure provides a compound of formula (II):

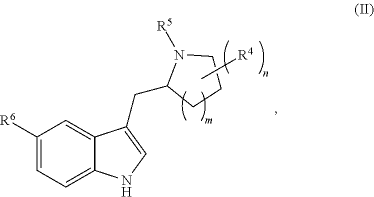

or a pharmaceutically acceptable salt, deuterated form or stereoisomer thereof
wherein:
R⁵ is —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or —C₁₋₆ alkyl;
R⁶ is —O—C₁₋₆ alkyl, —S—C₁₋₆ alkyl, —S(O)₂—C₁₋₆ alkyl, —SH, —N(C₁₋₆ alkyl)₂, —CN, —C(C═O)—C₁₋₆ alkyl, —O(C═O)—C₁₋₆ alkyl, —C₁₋₆ alkylene-OH, or —S(O)—C₁₋₆ alkyl;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —OH, —C₁₋₆ alkylene-O—C₁₋₆ alkyl, or —O—C₁₋₆ alkyl;
n is 0, 1, 2, or 3; and
m is 0 or 1,
and wherein, the compound is not:

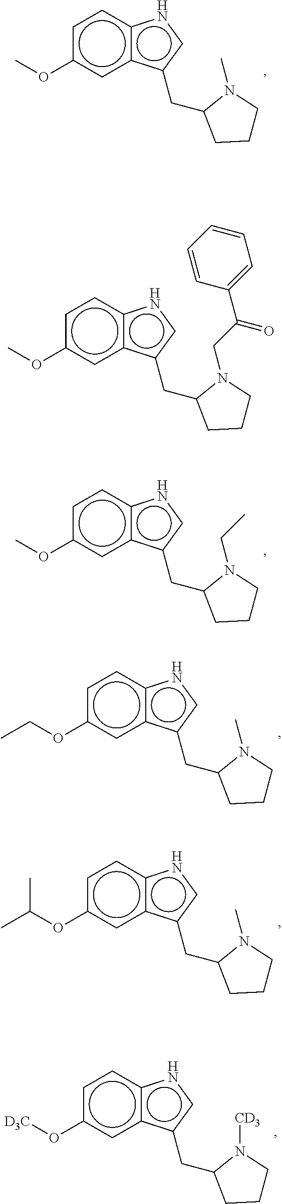

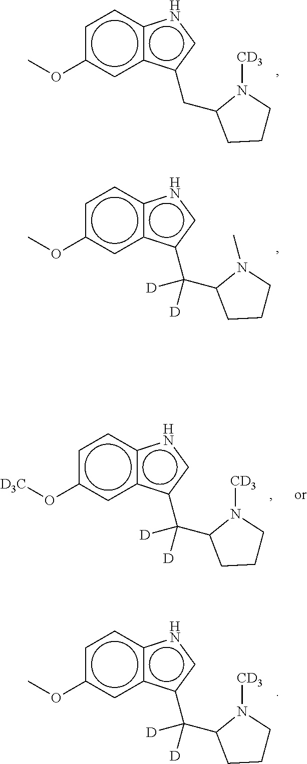

In embodiments, the present disclosure provides a compound of formula (III):

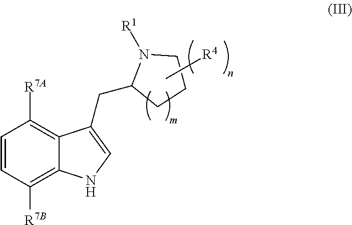

or a pharmaceutically acceptable salt, deuterated form or stereoisomer thereof
wherein:

R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or —C₁₋₆ alkyl;
R^{7A} and R^{7B} are each independently -halo;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —OH, —C₁₋₆ alkylene-O—C₁₋₆ alkyl, or —O—C₁₋₆ alkyl;
n is 0, 1, 2, or 3; and
m is 0 or 1.

In embodiments, the present disclosure provides a compound of formula (IV):

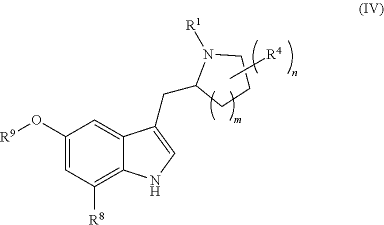

or a pharmaceutically acceptable salt, deuterated form or stereoisomer thereof
wherein:
R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or -C₁₋₆ alkyl;
R⁸ is -halo or -methyl;
R⁹ is —C₁₋₆ alkyl;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —OH, —C₁₋₆ alkylene-O—C₁₋₆ alkyl, or —O—C₁₋₆ alkyl;
n is 0, 1, 2, or 3; and
m is 0 or 1.

In embodiments, the present disclosure provides a compound of formula (V):

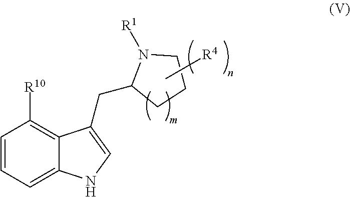

or a pharmaceutically acceptable salt, deuterated form or stereoisomer thereof
wherein:
R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or —C₁₋₆ alkyl;
R¹⁰ is -halo, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, —C₁₋₆ alkylene-O—C₁₋₆ alkyl, —S—C₁₋₆ alkyl, —S(O)₂—C₁₋₆ alkyl, —SH, —NH—C₁₋₆ alkyl, —N(C₁₋₆ alkyl)₂, —NH₂, —CN, —C(C═O)—C₁₋₆ alkyl, —O(C═O)—C₁₋₆ alkyl, or —NH(C═O)—C₁₋₆ alkyl;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —OH, —C₁₋₆ alkylene-O—C₁₋₆ alkyl, or —O—C₁₋₆ alkyl;
n is 0, 1, 2, or 3, and
m is 0 or 1.

In embodiments of formula (I), (III), (IV), and (V), R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more substituents.

In embodiments of formula (I), (III), (IV), and (V), R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or —C₁₋₆ alkyl.

In embodiments of formula (I), (III), (IV), and (V), R¹ is —H, —C₁₋₆ alkyl, —C₂₋₆ alkenyl, or —C₂₋₆ alkynyl; wherein the —C₁₋₆ alkyl, —C₂₋₆ alkenyl, or —C₂₋₆ alkynyl, are optionally substituted with one or more -halo, oxo, —OH, —NH₂, —CN, or —C₁₋₆ alkyl.

In embodiments of formula (I), (III), (IV), and (V), R¹ is —H, -aryl, -heteroaryl, —C₁₋₆ alkylene-aryl, —C₁₋₆ alkylene-heteroaryl, -heterocyclyl, or -cycloalkyl; wherein the -aryl, -heteroaryl, —C₁₋₆ alkylene-aryl, —C₁₋₆ alkylene-heteroaryl, -heterocyclyl, or -cycloalkyl are optionally substituted with one or more -halo, oxo, —OH, —NH₂, —CN, or —C₁₋₆ alkyl.

In embodiments of formula (I), (III), (IV), and (V), R¹ is —H, -aryl, -heteroaryl; wherein the -aryl or -heteroaryl are optionally substituted with one or more -halo, oxo, —OH, —NH₂, —CN, or —C₁₋₆ alkyl.

In embodiments of formula (I), (III), (IV), and (V), R¹ is —H, —C₁₋₆ alkylene-aryl, —C₁₋₆ alkylene-heteroaryl, or -heterocyclyl; wherein the —C₁₋₆ alkylene-aryl, —C₁₋₆ alkylene-heteroaryl, or -heterocyclyl are optionally substituted with one or more -halo, oxo, —OH, —NH₂, —CN, or —C₁₋₆ alkyl.

In embodiments of formula (I), (III), (IV), and (V), R¹ is —H, —C₁₋₆ alkylene-O—C₁₋₆ alkyl, or —O—C₁₋₆ alkyl; wherein the —C₁₋₆ alkylene-O—C₁₋₆ alkyl or —O—C₁₋₆ alkyl are optionally substituted with one or more -halo, oxo, —OH, —NH₂, —CN, or —C₁₋₆ alkyl.

In embodiments of formula (I), (III), (IV), and (V), R¹ is —H.

In embodiments of formula (I), (III), (IV), and (V), R¹ is —C₁₋₆ alkyl, wherein the —C₁₋₆ alkyl is optionally substituted with one or more -halo, oxo, —OH, —NH₂, —CN, or —C₁₋₆ alkyl.

In embodiments of formula (I), (III), (IV), and (V), R¹ is —C₁₋₆ alkyl.

In embodiments of formula (I), (III), (IV), and (V), R¹ is -methyl, -ethyl, -propyl, isopropyl or -butyl. In embodiments, R¹ is -methyl. In embodiments, R¹ is -ethyl. In embodiments, R¹ is -propyl. In embodiments, R¹ is -isopropyl. In embodiments, R¹ is -butyl.

In embodiments of formula (III), R¹ is methyl or butyl.

In embodiments of formula (I), (III), (IV), and (V), R¹ is —C₁₋₆ alkyl or —C₁₋₆ alkylene-heteroaryl, wherein the heteroaryl is optionally substituted with —C₁₋₆ alkyl.

In embodiments of formula (I), (III), (IV), and (V), R¹ is —C₁₋₃ alkylene-5 to 6 membered heteroaryl, wherein the heteroaryl is optionally substituted with a methyl. In embodiments, the —C₁₋₆ alkylene-heteroaryl is

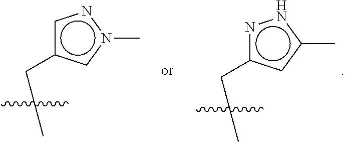

In embodiments, the —C₁₋₆ alkylene-heteroaryl is

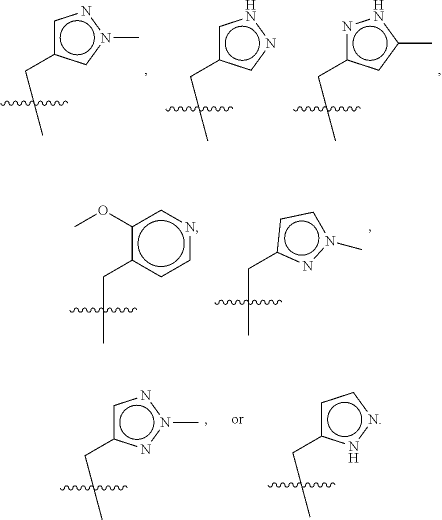

In embodiments of formula (I), (III), (IV), and (V), R¹ is —H, —C₁₋₆ alkyl, or —C₁₋₆ alkylene-heteroaryl.

In embodiments of formula (I), (III), (IV), and (V), R¹ is -heterocyclyl, —C₁₋₆ alkyl, or —C₁₋₆ alkylene-heteroaryl.

In embodiments of formula (I), (III), (IV), and (V), R¹ is —C₁₋₆ alkyl substituted with —OH.

In embodiments of formula (I), (III), (IV), and (V), R¹ is —CH₂CH₂OH.

In embodiments of formula (I), (III), (IV), and (V), R¹ is —CH₂CH₂CH₃.

In embodiments of formula (I), (III), (IV), and (V), R¹ is —C₁₋₆ alkylene-heteroaryl.

In embodiments of formula (I), (III), (IV), and (V), R¹ is —C₁₋₆ alkylene-heteroaryl, wherein the —C₁₋₆ alkylene-heteroaryl is

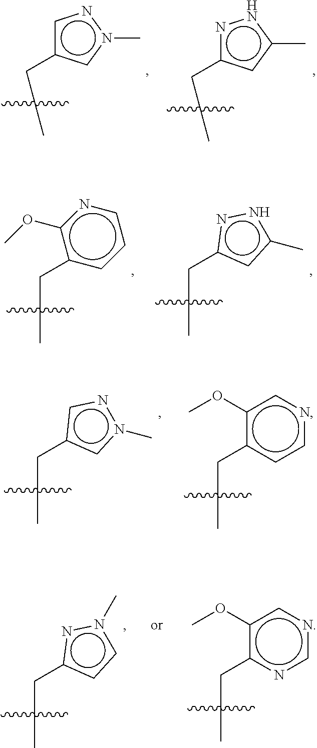

In embodiments of formula (I), (III), (IV), and (V), R¹ is -heterocyclyl. In embodiments, the -heterocyclyl is or

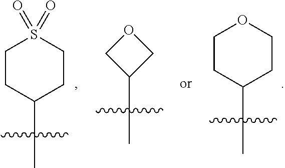

In embodiments of formula (I), (III), (IV), and (V), R¹ is -cycloalkyl. In embodiments, the -cycloalkyl is

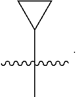 [SMILES: *C1CC1]

In embodiments of formula (I), (III), (IV), and (V), R¹ is —H, -heterocyclyl, -cycloalkyl, —C₁₋₆ alkyl, or —C₁₋₆ alkylene-heteroaryl, wherein the —C₁₋₆ alkyl is optionally substituted with —OH or -halo.

In embodiments of formula (I), (III), (IV), and (V), R¹ is —C₁₋₆ alkyl substituted with -halo.

In embodiments of formula (I), (III), (IV), and (V), R¹ is —C₁₋₆ alkyl substituted with -halo.

In embodiments of formula (I), (III), (IV), and (V), R¹ is —CH₂CH₂F.

In embodiments of formula (I), R¹ is —C₁₋₆ alkyl, —C₁₋₆ alkylene-heteroaryl or -heterocyclyl, wherein the heteroaryl is optionally substituted with —C₁₋₆ alkyl.

In embodiments of formula (I), R¹ is methyl.

In embodiments of formula (I), R¹ is —C₁₋₃ alkylene-5 to 6 membered heteroaryl, wherein the heteroaryl is optionally substituted with a methyl. In embodiments, the —C₁₋₆ alkylene-heteroaryl is

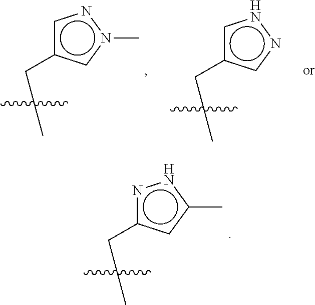

In embodiments of formula (I), R¹ is -heterocyclyl. In embodiments, the -heterocyclyl is

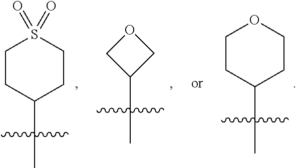

In embodiments of formula (III), R¹ is —C₁₋₆ alkyl.

In embodiments of formula (III), R¹ is methyl or butyl.

In embodiments of formula (IV), R¹ is —H, —C₁₋₆ alkyl, or —C₁₋₆ alkylene-heteroaryl. In embodiments, the —C₁₋₆ alkylene-heteroaryl is

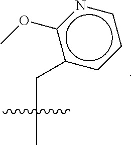

In embodiments of formula (V), R¹ is —H, -heterocyclyl, -cycloalkyl, —C₁₋₆ alkyl, or —C₁₋₆ alkylene-heteroaryl, wherein the —C₁₋₆ alkyl is optionally substituted with —OH or -halo.

In embodiments of formula (V), R¹ is —H, -heterocyclyl, —C₁₋₆ alkyl, or —C₁₋₆ alkylene-heteroaryl, wherein the —C₁₋₆ alkyl is optionally substituted with —OH.

In embodiments, the —C₁₋₆ alkylene-heteroaryl is:

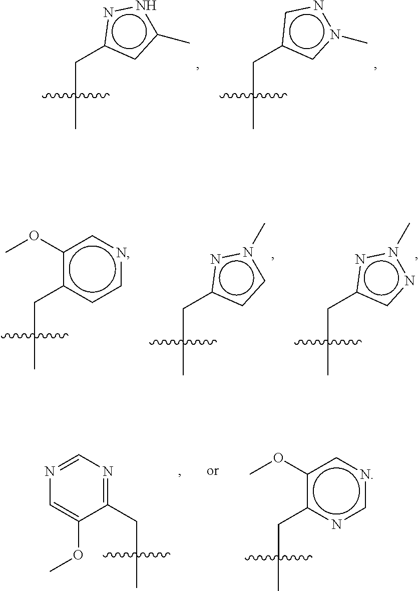

In embodiments of formula (V), R¹ is —C₁₋₆ alkyl substituted with —OH.

In embodiments of formula (V), R¹ is —CH₂CH₂OH.

In embodiments of formula (V), R¹ is —CH₂CH₂CH₃.

In embodiments of formula (V), R¹ is —C₁₋₆ alkyl substituted with -halo.

In embodiments of formula (V), R¹ is —C₁₋₆ alkyl substituted with -halo.

In embodiments or formula (V), R¹ is —CH₂CH₂F.

### R²

In embodiments of formula (I), R² is -halo, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, —O—C₁₋₆ alkyl, —OH, —S—C₁₋₆ alkyl, —S(O)₂—C₁₋₆ alkyl, —SH, —NH—C₁₋₆ alkyl, —N(C₁₋₆ alkyl)₂, —NH₂, —CN, —C(C═O)—C₁₋₆ alkyl, —O(C═O)—C₁₋₆ alkyl, or —NH(C═O)—C₁₋₆ alkyl.

In embodiments of formula (I), R² is -halo, —C₁₋₆ alkyl, —C₂₋₆ alkenyl, or —C₂₋₆ alkynyl.

In embodiments of formula (I), R² is -aryl or -heteroaryl.

In embodiments of formula (I), R² is —C₁₋₆ alkylene-aryl or —C₁₋₆ alkylene-heteroaryl.

In embodiments of formula (I), R² is -heterocyclyl or -cycloalkyl.

In embodiments of formula (I), R² is —C₁₋₆ alkylene-O—C₁₋₆ alkyl or —O—C₁₋₆ alkyl.

In embodiments of formula (I), R² is —OH, —SH, —NH₂, or —CN.

In embodiments of formula (I), R² is —S—C₁₋₆ alkyl, —S(O)₂—C₁₋₆ alkyl, or —SH.

In embodiments of formula (I), R² is —NH—C₁₋₆ alkyl, —N(C₁₋₆ alkyl)₂, or —NH₂.

In embodiments of formula (I), R² is —C(C═O)—C₁₋₆ alkyl, —O(C═O)—C₁₋₆ alkyl, or —NH(C═O)—C₁₋₆ alkyl.

In embodiments of formula (I), R² is -halo.

In embodiments of formula (I), R² is —F, —Cl, —Br, or —I. In embodiments, R² is —Cl, —Br, or —I. In embodiments, R² is —F, —Br, or —I. In embodiments, R² is —Br or —I. In embodiments, R² is —F or —Cl.

In embodiments, of formula (I), R² is —F.

In embodiments, of formula (I), R² is —Cl.

In embodiments, of formula (I), R² is —Br.

In embodiments, of formula (I), R² is —I.

### R³

In embodiments of formula (I), R³ is -halo, —O—C₁₋₆ alkyl, —OH, —S—C₁₋₆ alkyl, —S(O)₂—C₁₋₆ alkyl, —SH, —NH—C₁₋₆ alkyl, —N(C₁₋₆ alkyl)₂, —NH₂, —CN, —C(C═O)—C₁₋₆ alkyl, —O(C═O)—C₁₋₆ alkyl, or —NH(C═O)—C₁₋₆ alkyl.

In embodiments of formula (I), R³ is —O—C₁₋₆ alkyl, —OH, —S—C₁₋₆ alkyl, —S(O)₂—C₁₋₆ alkyl, —SH, —NH—C₁₋₆ alkyl, —N(C₁₋₆ alkyl)₂, —NH₂, —CN, —C(C═O)—C₁₋₆ alkyl, —O(C═O)—C₁₋₆ alkyl, or —NH(C═O)—C₁₋₆ alky.

In embodiments of formula (I), R³ is —NH—C₁₋₆ alkyl, —N(C₁₋₆ alkyl)₂, or —NH₂.

In embodiments of formula (I), R³ is —SH, —NH₂, or —CN.

In embodiments of formula (I), R³ is —C(C═O)—C₁₋₆ alkyl, —O(C═O)—C₁₋₆ alkyl, or —NH(C═O)—C₁₋₆ alkyl.

In embodiments of formula (I), R³ is —S—C₁₋₆ alkyl, —S(O)₂—C₁₋₆ alkyl, or —SH.

In embodiments of formula (I), R³ is -halo or —OH.

In embodiments of formula (I), R³ is —O—C₁₋₆ alkyl.

In embodiments of formula (I), R³ is —O-Me, —O-ethyl, O-propyl, —O-isopropyl, or O-butyl.

In embodiments of formula (I), R³ is —O-Me.

In embodiments of formula (I), R³ is —O-ethyl.

In embodiments of formula (I), R³ is - O-propyl.

In embodiments of formula (I), R³ is —O-isopropyl.

In embodiments of formula (I), R³ is O-butyl.

R² and R³

In embodiments of formula (I), R³ is —O—C₁₋₆ alkyl, —OH, —S—C₁₋₆ alkyl, —S(O)₂—C₁₋₆ alkyl, —SH, —NH—C₁₋₆ alkyl, —N(C₁₋₆ alkyl)₂, —NH₂, —CN, —C(C═O)—C₁₋₆ alkyl, —O(C═O)—C₁₋₆ alkyl, or —NH(C═O)—C₁₋₆ alkyl, and R² is -halo, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, —O—C₁₋₆ alkyl, —OH, —S—C₁₋₆ alkyl, —S(O)₂—C₁₋₆ alkyl, —SH, —NH—C₁₋₆ alkyl, —N(C₁₋₆ alkyl)₂, —NH₂, —CN, —C(C═O)—C₁₋₆ alkyl, —O(C═O)—C₁₋₆ alkyl, or —NH(C═O)—C₁₋₆ alkyl.

In embodiments of formula (I), R² and R³ are not both -halo.

In embodiments of formula (I), R² and R³ are not both —F, —Cl, —Br, or —I.

In embodiments of formula (I), R² and R³ are not both —F.

In embodiments of formula (I), R² and R³ are not both —Cl.

In embodiments of formula (I), R² and R³ are not both —Br.

In embodiments of formula (I), R² and R³ are not both —I.

### R⁴

In embodiments of formula (I), (II), (III), (IV), or (V), R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —OH, —C₁₋₆ alkylene-O—C₁₋₆ alkyl, or —O—C₁₋₆ alkyl.

In embodiments of formula (I), (II), (III), (IV), or (V), R⁴ is —C₁₋₆ alkyl.

In embodiments of formula (I), (II), (III), (IV), or (V), R⁴ is —OH.

In embodiments of formula (I), (II), (III), (IV), or (V), R⁴ is -aryl or -heteroaryl.

In embodiments of formula (I), (II), (III), (IV), or (V), R⁴ is —C₁₋₆ alkylene-aryl, —C₁₋₆ alkylene-heteroaryl, or —C₁₋₆ alkylene-OH.

In embodiments of formula (I), (II), (III), (IV), or (V), R⁴ is —C₁₋₆ alkylene-O—C₁₋₆ alkyl, or —O—C₁₋₆ alkyl.

In embodiments, embodiments of formula (I), (II), (III), (IV), or (V), R⁴ is —O—C₁₋₆ alkyl or —OH.

In embodiments, embodiments of formula (II), R⁴ is —O—C₁₋₆ alkyl or —OH.

### R⁵

In embodiments of formula (II), R⁵ is —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more substituents.

In embodiments of formula (II), R⁵ is —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl; wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or —C₁₋₆ alkyl.

In embodiments of formula (II), R⁵ is —C₁₋₆ alkyl, —C₂₋₆ alkenyl, or —C₂₋₆ alkynyl; wherein the —C₁₋₆ alkyl, —C₂₋₆ alkenyl, or —C₂₋₆ alkynyl are optionally substituted with one or more -halo, oxo, —OH, —NH₂, —CN, or —C₁₋₆ alkyl.

In embodiments of formula (II), R⁵ is -aryl, -heteroaryl, —C₁₋₆ alkylene-aryl, —C₁₋₆ alkylene-heteroaryl, -heterocyclyl, or -cycloalkyl; wherein the -aryl, -heteroaryl, —C₁₋₆ alkylene-aryl, —C₁₋₆ alkylene-heteroaryl, -heterocyclyl, or -cycloalkyl are optionally substituted with one or more -halo, oxo, —OH, —NH₂, —CN, or —C₁₋₆ alkyl.

In embodiments of formula (II), R⁵ is -aryl or -heteroaryl; wherein the -aryl or -heteroaryl are optionally substituted with one or more -halo, oxo, —OH, —NH₂, —CN, or —C₁₋₆ alkyl.

In embodiments of formula (II), R⁵ is —C₁₋₆ alkylene-aryl, —C₁₋₆ alkylene-heteroaryl, or heterocyclyl; wherein the —C₁₋₆ alkylene-aryl, —C₁₋₆ alkylene-heteroaryl, or -heterocyclyl are optionally substituted with one or more -halo, oxo, —OH, —NH₂, —CN, or —C₁₋₆ alkyl.

In embodiments of formula (II), R⁵ is C₁₋₆ alkylene-O—C₁₋₆ alkyl or —O—C₁₋₆ alkyl; wherein C₁₋₆ alkylene-O—C₁₋₆ alkyl or —O—C₁₋₆ alkyl are optionally substituted with one or more -halo, oxo, —OH, —NH₂, —CN, or —C₁₋₆ alkyl.

In embodiments of formula (II), R⁵ is —C₁₋₆ alkyl, —C₁₋₆ alkylene-aryl, —C₁₋₆ alkylene-heteroaryl, or -heterocyclyl, each of which is optionally substituted with -halo or —OH.

In embodiments of formula (II), R⁵ is —C₁₋₆ alkyl, which is optionally substituted with -halo or —OH.

In embodiments of formula (II), R⁵ is —CH₂CH₂F or —CH₂CH₂OH.

In embodiments of formula (II), R⁵ is —CH₂CH₂F.

In embodiments of formula (II), R⁵ is —CH₂CH₂OH.

In embodiments of formula (II), R⁵ is —C₁₋₆ alkylene-aryl,

In embodiments of formula (II), R⁵ is

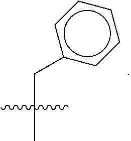

In embodiments of formula (II), R⁵ is —C₁₋₆ alkylene-heteroaryl.

In embodiments of formula (II), R⁵ is —C₁₋₆ alkylene-heteroaryl, wherein the —C₁₋₆ alkylene-heteroaryl is

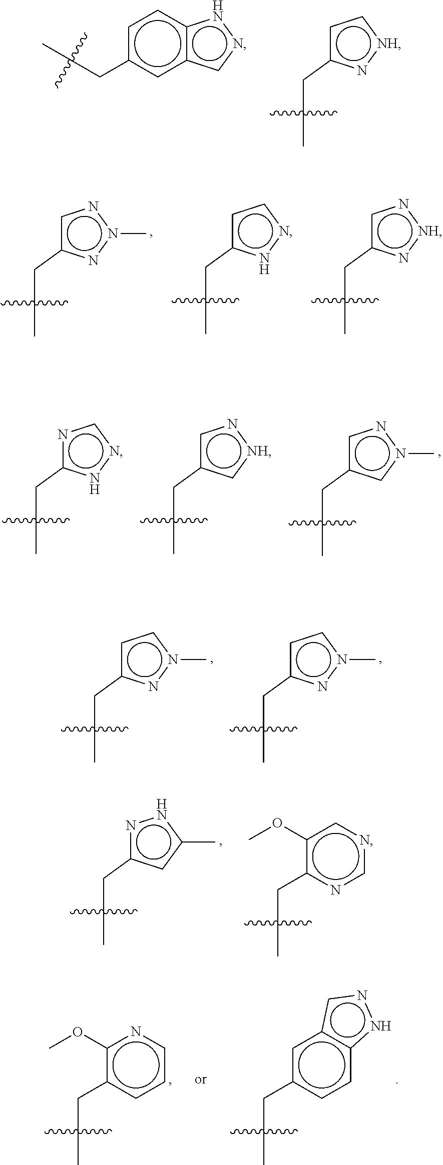

In embodiments of formula (II), R⁵ is -heterocyclyl.

In embodiments of formula (II), R⁵ is -heterocyclyl, wherein the is -heterocyclyl is

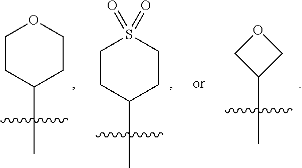

### R⁶

In embodiments of formula (II), R⁶ is —O—C₁₋₆ alkyl, —S—C₁₋₆ alkyl, —S(O)₂—C₁₋₆ alkyl, —SH, —N(C₁₋₆ alkyl)₂, —CN, —C(C═O)—C₁₋₆ alkyl, —O(C═O)—C₁₋₆ alkyl, —C₁₋₆ alkylene-OH, or —S(O)—C₁₋₆ alkyl.

In embodiments of formula (II), R⁶ is —S—C₁₋₆ alkyl, —S(O)₂—C₁₋₆ alkyl, or —SH.

In embodiments of formula (II), R⁶ is —C(C═O)—C₁₋₆ alkyl or —O(C═O)—C₁₋₆ alkyl.

In embodiments of formula (II), R⁶ is —N(C₁₋₆ alkyl)₂ or —CN.

In embodiments of formula (II), R⁶ is —O—C₁₋₆ alkyl or —S—C₁₋₆ alkyl.

In embodiments of formula (II), R⁶ is —O—C₁₋₆ alkyl, —S—C₁₋₆ alkyl, —S(O)—C₁₋₆ alkyl, —S(O)₂—C₁₋₆ alkyl, or —C₁₋₆ alkylene-OH.

In embodiments of formula (II), R⁶ is —O—C₁₋₆ alkyl.

In embodiments of formula (II), R⁶ is —S—C₁₋₆ alkyl.

In embodiments of formula (II), R⁶ is —S(O)—C₁₋₆ alkyl.

In embodiments of formula (II), R⁶ is —S(O)₂—C₁₋₆ alkyl.

In embodiments of formula (II), R⁶ is —C₁₋₆ alkylene-OH.

In embodiments of formula (II), R⁶ is —O-Me, —S-Me, —CH₂—OH, —S(O)-Me, or —S(O)₂-Me.

In embodiments of formula (II), R⁶ is —S-Me.

In embodiments of formula (II), R⁶ is —O-Me.

R^{7A} and R^{7B}

In embodiments of formula (III), R^{7A} and R^{7B} are each independently -halo.

In embodiments of formula (III), R^{7A} and R^{7B} are each independently —F, —Cl, —Br, or —I.

In embodiments of formula (III), R^{7A} and R^{7B} are each independently —F, —Cl, or —Br.

In embodiments of formula (III), R^{7A} and R^{7B} are each independently —F or —Cl.

In embodiments of formula (III), R^{7A} and R^{7B} are each independently —F or —Br.

In embodiments of formula (III), R^{7A} and R^{7B} are both —F.

In embodiments of formula (III), R^{7A} and R^{7B} are both —Cl.

In embodiments of formula (III), R^{7A} and R^{7B} are both —Br.

In embodiments of formula (III), R^{7A} and R^{7B} are both —I.

### R⁸

In embodiments of formula (IV), R⁸ is -halo or -methyl.

In embodiments of formula (IV), R⁸ is -halo.

In embodiments of formula (IV), R⁸ is -methyl.

In embodiments of formula (IV), R⁸ is —F, —Cl, —Br, or —I.

In embodiments of formula (IV), R⁸ is —F, —Cl, or —Br.

In embodiments of formula (IV), R⁸ is —F or —Cl.

In embodiments of formula (IV), R⁸ is —F or —Br.

In embodiments of formula (IV), R⁸ is —F.

In embodiments of formula (IV), R⁸ is —Cl.

In embodiments of formula (IV), R⁸ is —Br.

In embodiments of formula (IV), R⁸ is —I.

In embodiments of formula (IV), R⁸ is —F or -methyl.

### R⁹

In embodiments of formula (IV), R⁹ is —C₁₋₆ alkyl.

In embodiments of formula (IV), R⁹ is -methyl, -ethyl, -propyl, isopropyl or -butyl.

In embodiments of formula (IV), R⁹ is -methyl.

In embodiments of formula (IV), R⁹ is -ethyl.

In embodiments of formula (IV), R⁹ is -propyl.

In embodiments of formula (IV), R⁹ is -isopropyl.

In embodiments of formula (IV), R⁹ is -butyl.

### R¹⁰

In embodiments of formula (V), R¹⁰ is -halo, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, —C₁₋₆ alkylene-O—C₁₋₆ alkyl, —S—C₁₋₆ alkyl, —S(O)₂—C₁₋₆ alkyl, —SH, —NH—C₁₋₆ alkyl, —N(C₁₋₆ alkyl)₂, —NH₂, —CN, —C(C═O)—C₁₋₆ alkyl, —O(C═O)—C₁₋₆ alkyl, or —NH(C═O)—C₁₋₆ alkyl.

In embodiments of formula (V), R¹⁰ is —C₂₋₆ alkenyl or —C₂₋₆ alkynyl.

In embodiments of formula (V), R¹⁰ is -aryl or -heteroaryl.

In embodiments of formula (V), R¹⁰ is —C₁₋₆ alkylene-aryl or —C₁₋₆ alkylene-heteroaryl.

In embodiments of formula (V), R¹⁰ is -aryl, -heteroaryl, or -heterocyclyl.

In embodiments of formula (V), R¹⁰ is —S—C₁₋₆ alkyl, —S(O)₂—C₁₋₆ alkyl, or —SH.

In embodiments of formula (V), R¹⁰ is —NH—C₁₋₆ alkyl, —N(C₁₋₆ alkyl)₂, —NH₂, or —CN.

In embodiments of formula (V), R¹⁰ is —C(C═O)—C₁₋₆ alkyl, —O(C═O)—C₁₋₆ alkyl, or —NH(C═O)—C₁₋₆ alkyl.

In embodiments of formula (V), R¹⁰ is -halo.

In embodiments of formula (V), R¹⁰ is —F, —Cl, —Br, or —I.

In embodiments of formula (V), R¹⁰ is —F, —Cl, or —Br.

In embodiments of formula (V), R¹⁰ is —F or —Cl.

In embodiments of formula (V), R¹⁰ is —F or —Br.

In embodiments of formula (V), R¹⁰ is —F.

In embodiments of formula (V), R¹⁰ is —Cl.

In embodiments of formula (V), R¹⁰ is —Br.

In embodiments of formula (V), R¹⁰ is —I.

n

In embodiments of formula (I), (II), (III), (IV), or (V), n is 0.

In embodiments of formula (I), (II), (III), (IV), or (V), n is 1.

In embodiments of formula (I), (II), (III), (IV), or (V), n is 2.

In embodiments of formula (I), (II), (III), (IV), or (V), n is 3.

m

In embodiments of formula (I), (II), (III), (IV), or (V), m is 0.

In embodiments of formula (I), (II), (III), (IV), or (V), m is 1.

In embodiments of formula (I):

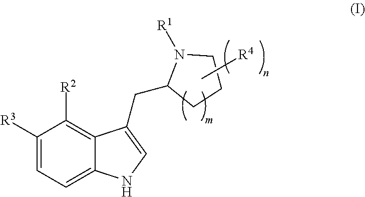

R¹ is —C₁₋₆ alkyl, -cycloalkyl, —C₁₋₆ alkylene-heteroaryl or -heterocyclyl, wherein the heteroaryl is optionally substituted with —C₁₋₆ alkyl and the —C₁₋₆ alkyl is optionally substituted with -halo;
R² is -halo;
R³ is —O—C₁₋₆ alkyl;
n is 0; and
m is 1.

In embodiments, formula (II) is not deuterated.

In embodiments of formula (II), when R⁶ is O-methyl, R⁵ is not -methyl or

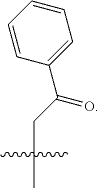 [SMILES: *CC(=O)c1ccccc1]

In embodiments of formula (II), when R⁶ is O-methyl, R⁵ is not -methyl

In embodiments of formula (II), when R⁶ is O-methyl, R⁵ is not

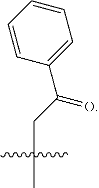 [SMILES: *CC(=O)c1ccccc1]

In embodiments of formula (II):

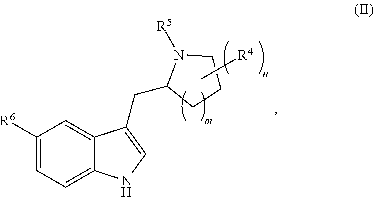

R⁴ is —O—C₁₋₆ alkyl or —OH;
R⁵ is —C₁₋₆ alkyl, —C₁₋₆ alkylene-aryl, —C₁₋₆ alkylene-heteroaryl, or -heterocyclyl, each of which is optionally substituted with -halo or —OH;
R⁶ is —O—C₁₋₆ alkyl, —S—C₁₋₆ alkyl, —S(O)—C₁₋₆ alkyl, —S(O)₂—C₁₋₆ alkyl, —C₁₋₆ alkylene-OH;
n is 0 or 1; and
m is 1.

In embodiments of formula (III):

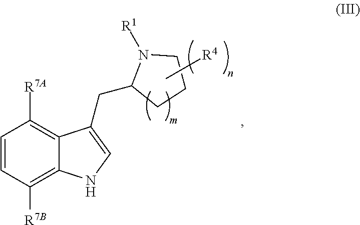

R¹ is —C₁₋₆ alkyl;
R^{7A} and R^{7B} are each independently -halo;
n is 0;
m is 1.

In embodiments of formula (IV):

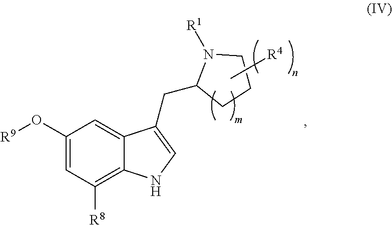

R¹ is —H, —C₁₋₆ alkyl, or —C₁₋₆ alkylene-heteroaryl;
R⁸ is -halo or -methyl;
R⁹ is —C₁₋₆ alkyl;
n is 0; and
m is 1.

In embodiments of formula (V):

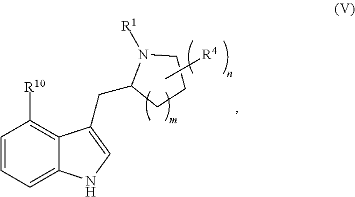

R¹ is —H, —C₁₋₆ alkyl, or —C₁₋₆ alkylene-heteroaryl, wherein the —C₁₋₆ alkyl is optionally substituted with —OH;
R¹⁰ is -halo;
n is 0; and
m is 1.

TABLE A
Various compounds of formula (I)

| Compound | |
|---|---|
| No. | Structure |
| 1 | 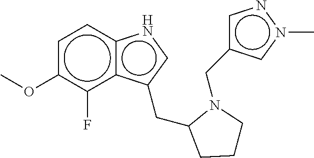 |
| 2 | 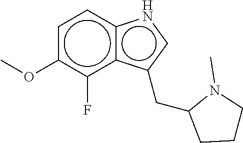 |
| 3 | 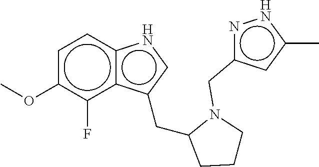 |
| 4 | 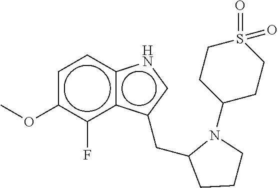 |
| 5 | 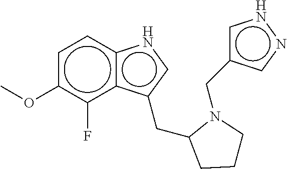 |
| 6 | 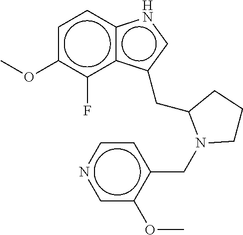 |
| 7 | 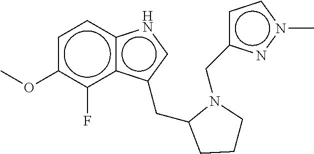 |
| 8 | 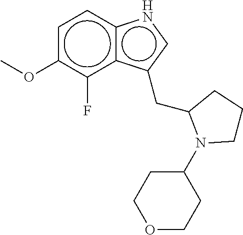 |
| 9 | 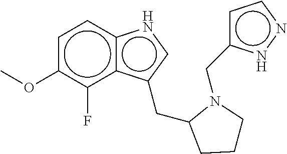 |
| 10 | 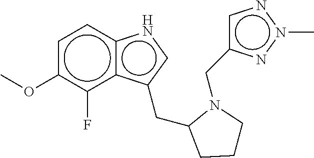 |
| 11 | 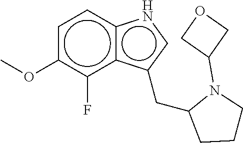 |
| 12 | 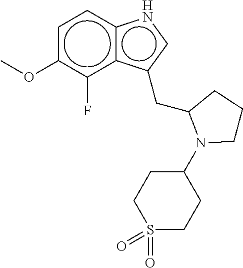 |
| 13 | 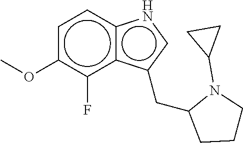 |
| 14 | 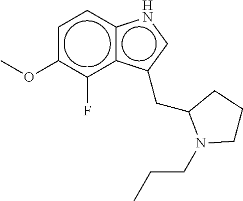 |
| 15 | 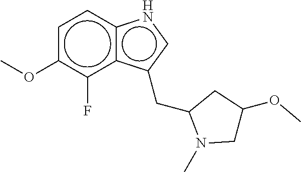 |
| 16 | 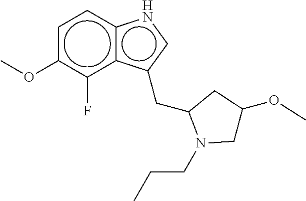 |
| 17 | 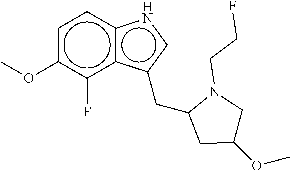 |

TABLE B
Various compounds of formula (I)

| Compound | |
|---|---|
| No. | Structure |
| 1A | 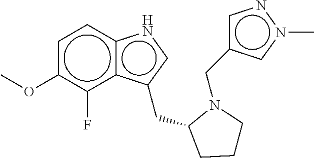 |
| 1B | 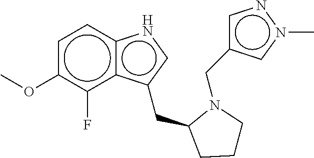 |
| 2A | 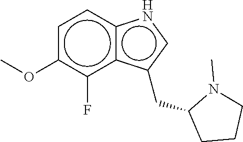 |
| 2B | 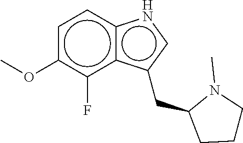 |
| 3A | 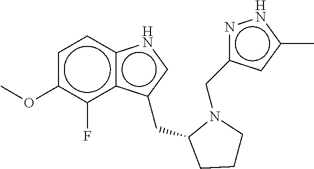 |
| 3B | 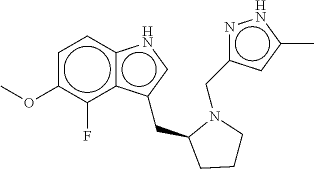 |
| 4A | 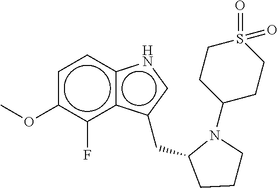 |
| 4B | 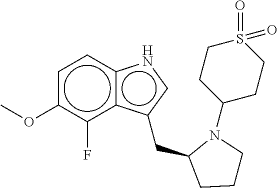 |
| 5A | 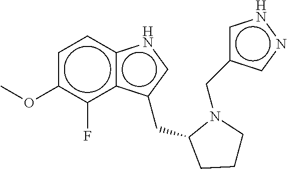 |
| 5B | 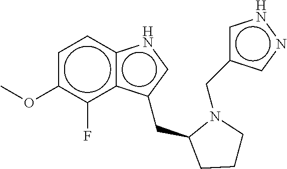 |
| 6A | 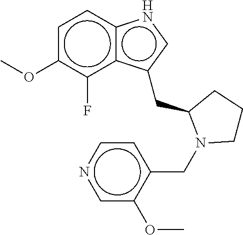 |
| 6B | 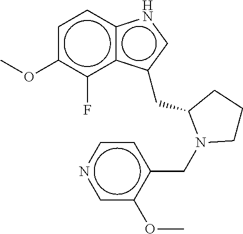 |
| 7A | 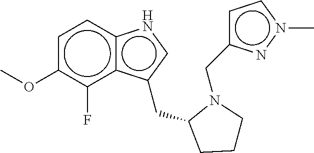 |
| 7B | 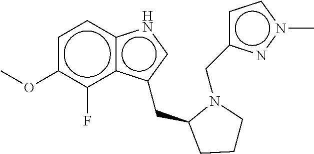 |
| 8A | 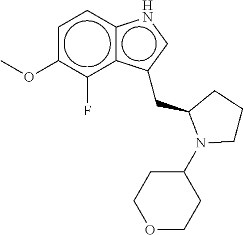 |
| 8B | 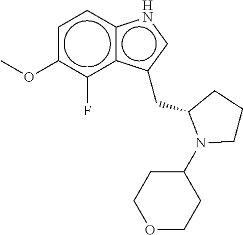 |
| 9A | 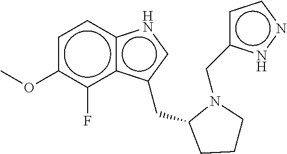 |
| 9B | 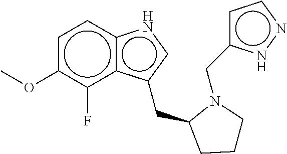 |
| 10A | 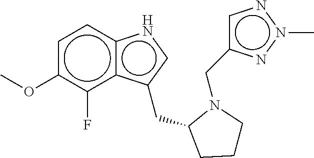 |
| 10B | 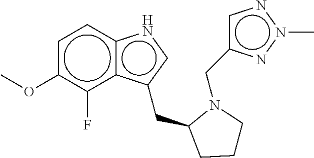 |
| 11A | 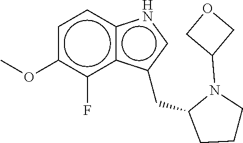 |
| 11B | 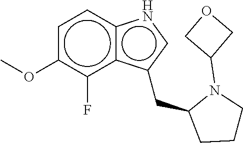 |
| 12A | 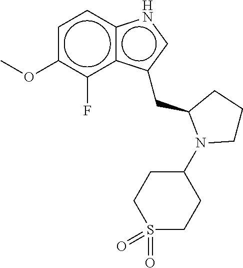 |
| 12B | 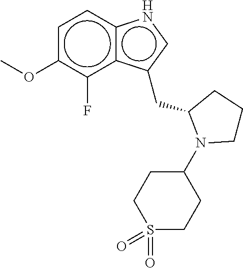 |
| 13A | 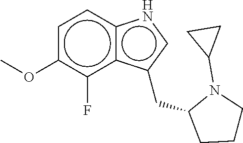 |
| 13B | 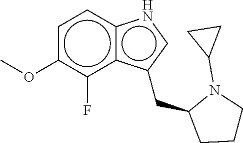 |
| 14A | 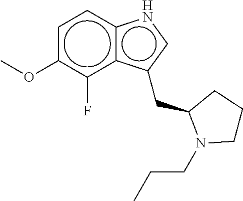 |
| 14B | 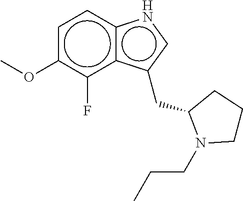 |
| 15A | 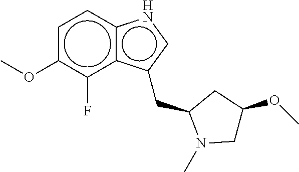 |
| 15B | 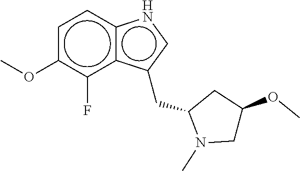 |
| 15C | 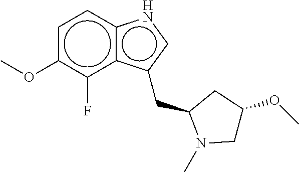 |
| 15D | 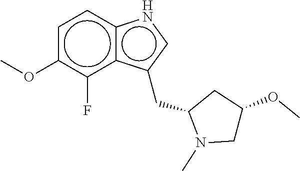 |
| 16A | 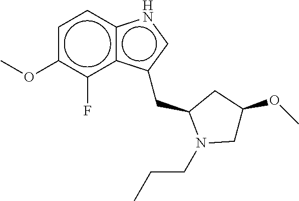 |
| 16B | 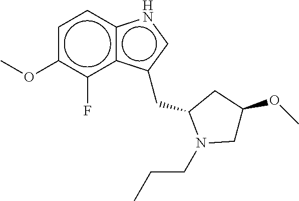 |
| 16C | 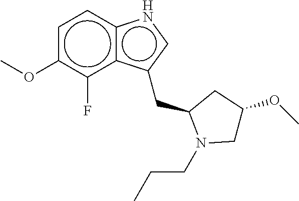 |
| 16D | 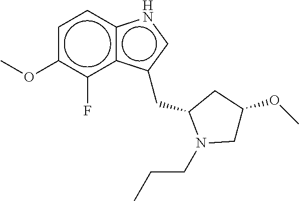 |
| 17A | 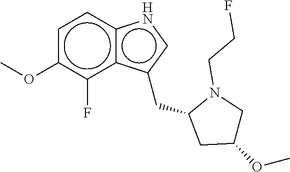 |
| 17B | 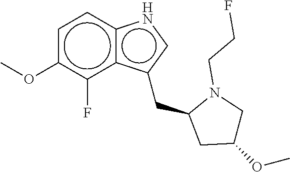 |
| 17C | 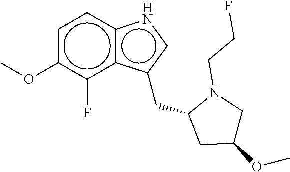 |
| 17D | 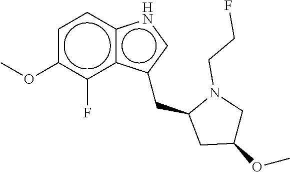 |

TABLE C
Various compounds of formula (II)

| Compound | |
|---|---|
| No. | Structure |
| 18 | 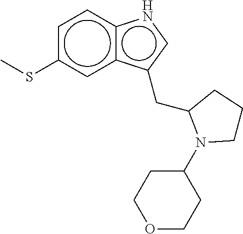 |
| 19 | 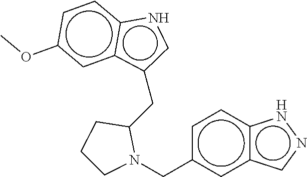 |
| 20 | 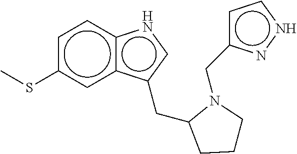 |
| 21 | 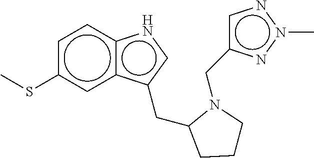 |
| 22 | 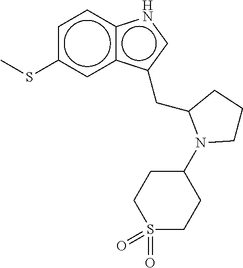 |
| 23 | 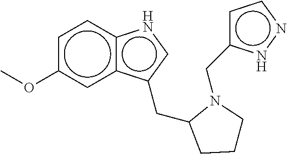 |
| 24 | 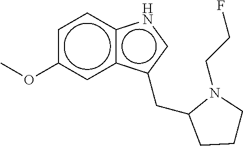 |
| 25 | 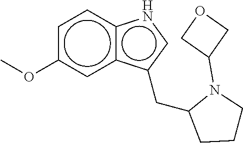 |
| 26 | 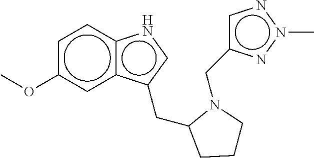 |
| 27 | 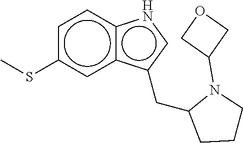 |
| 28 | 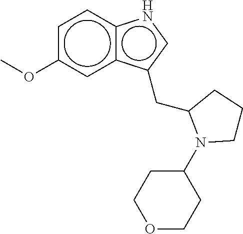 |
| 29 | 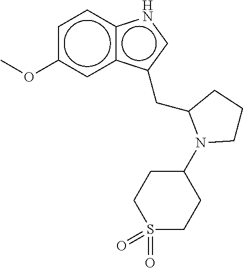 |
| 30 | 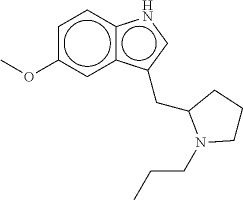 |
| 31 | 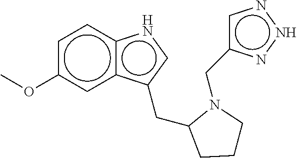 |
| 32 | 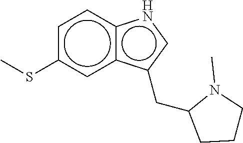 |
| 33 | 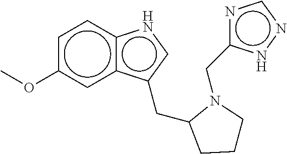 |
| 34 | 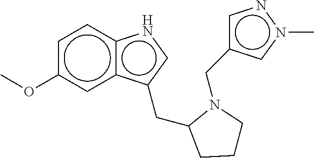 |
| 35 | 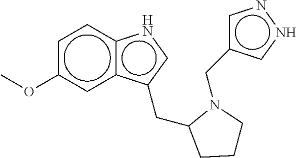 |
| 36 | 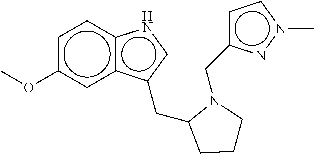 |
| 37 | 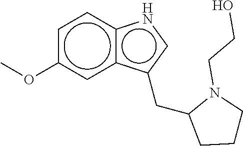 |
| 38 | 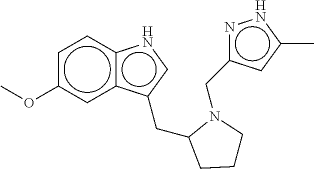 |
| 39 | 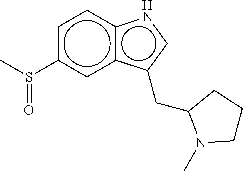 |
| 40 | 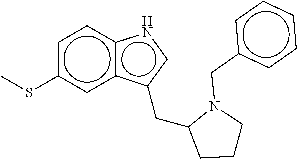 |
| 41 | 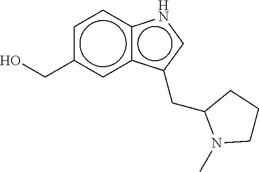 |
| 42 | 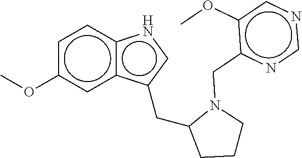 |
| 43 | 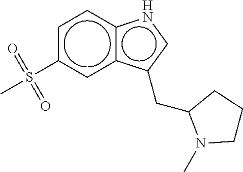 |
| 44 | 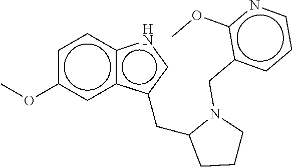 |
| 45 | 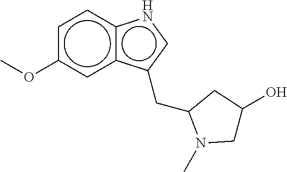 |
| 46 | 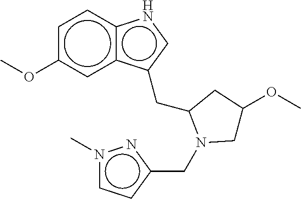 |
| 47 | 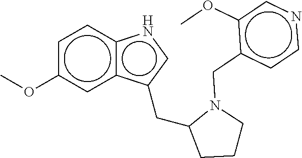 |
| 48 | 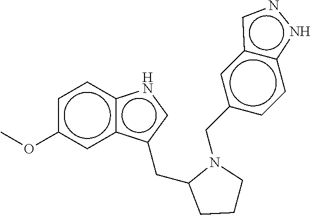 |
| 49 | 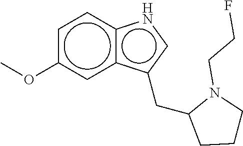 |
| 50 | 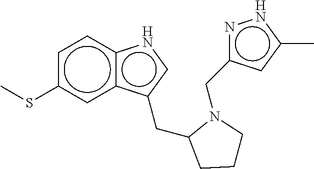 |
| 51 | 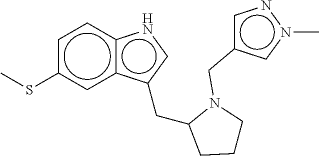 |
| 52 | 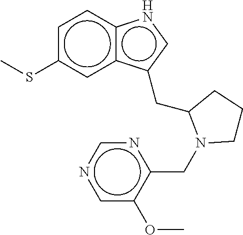 |
| 53 | 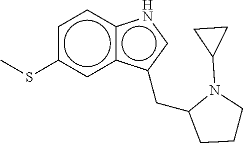 |
| 54 | 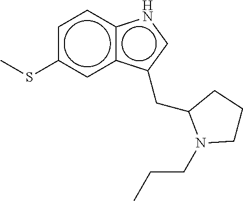 |
| 55 | 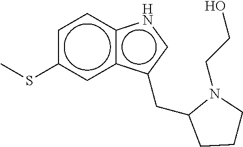 |
| 56 | 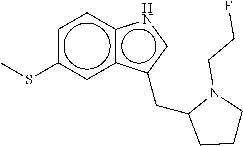 |
| 57 | 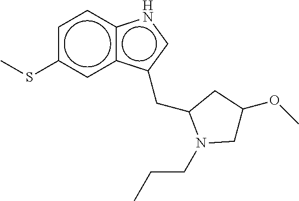 |
| 58 | 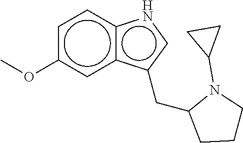 |
| 59 | 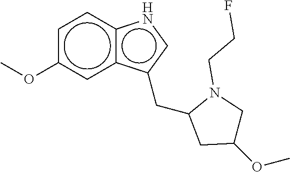 |
| 60 | 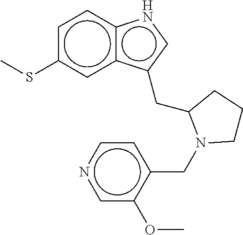 |
| 61 | 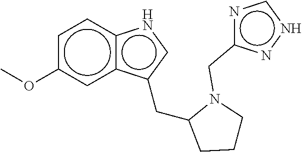 |
| 62 | 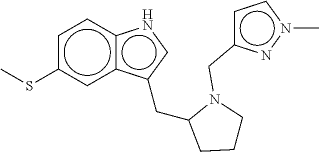 |
| 63 | 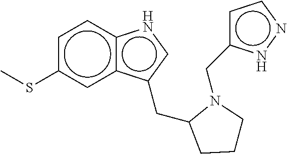 |
| 64 | 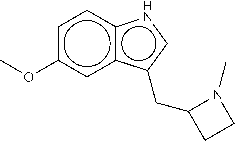 |

TABLE D
Various compounds of formula (II)

| Compound | |
|---|---|
| No. | Structure |
| 18A | 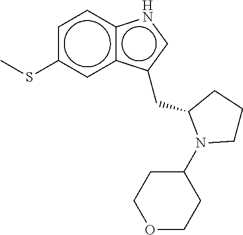 |
| 18B | 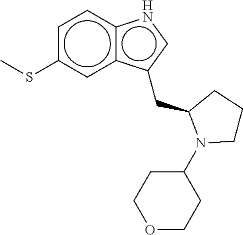 |
| 19A | 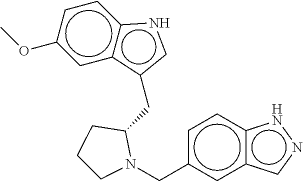 |
| 19B | 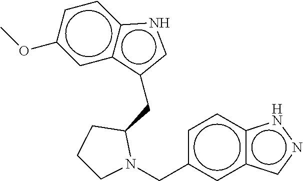 |
| 20A | 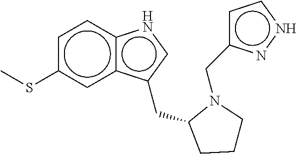 |
| 20B | 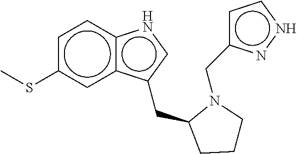 |
| 21A | 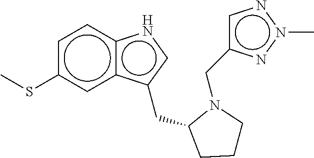 |
| 21B | 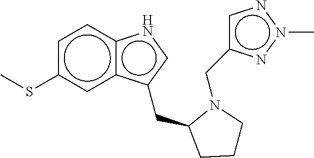 |
| 22A | 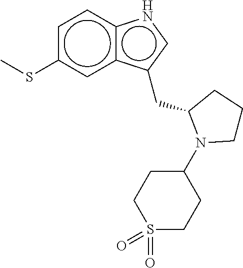 |
| 22B | 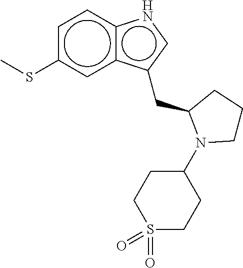 |
| 23A | 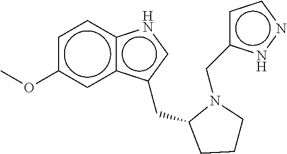 |
| 23B | 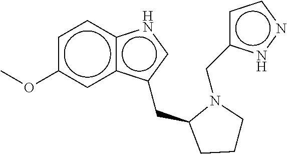 |
| 24A | 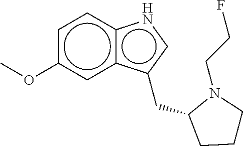 |
| 24B | 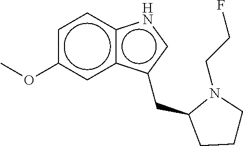 |
| 25A | 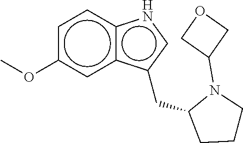 |
| 25B | 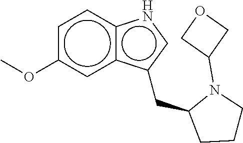 |
| 26A | 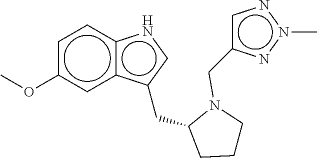 |
| 26B | 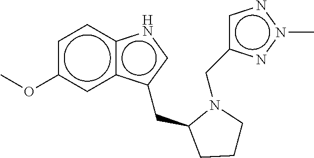 |
| 27A | 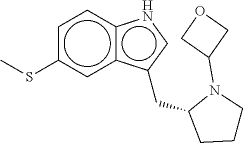 |
| 27B | 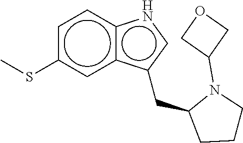 |
| 28A | 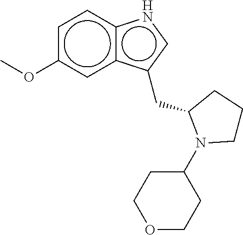 |
| 28B | 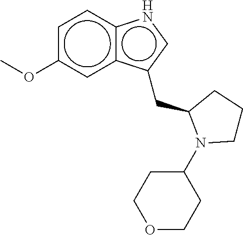 |
| 29A | 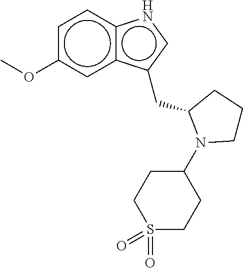 |
| 29B | 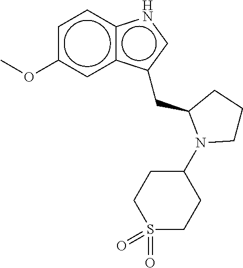 |
| 30A | 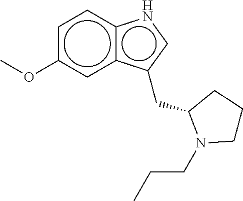 |
| 30B | 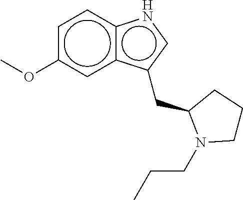 |
| 31A | 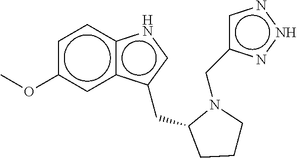 |
| 31B | 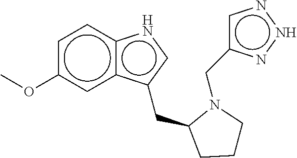 |
| 32A | 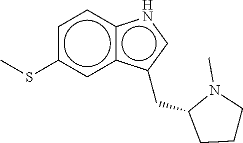 |
| 32B | 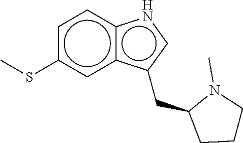 |
| 33A | 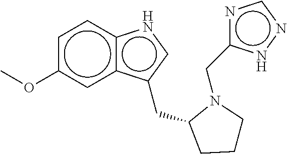 |
| 33B | 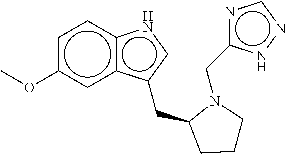 |
| 34A | 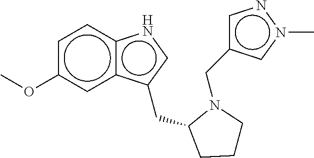 |
| 34B | 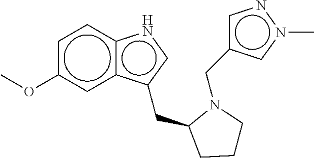 |
| 35A | 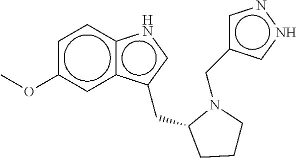 |
| 35B | 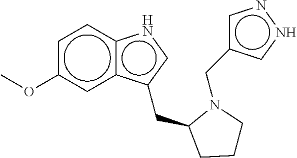 |
| 36A | 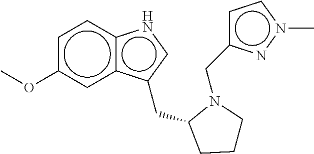 |
| 36B | 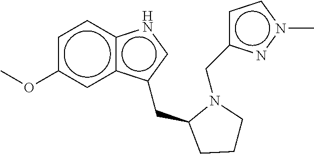 |
| 37A | 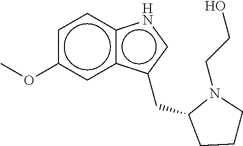 |
| 37B | 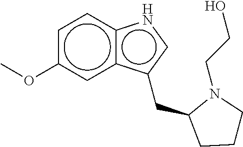 |
| 38A | 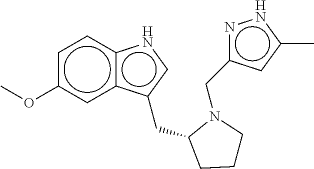 |
| 38B | 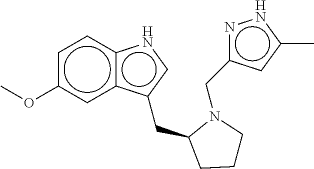 |
| 39A | 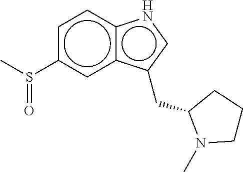 |
| 39B | 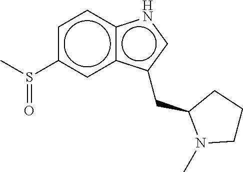 |
| 40A | 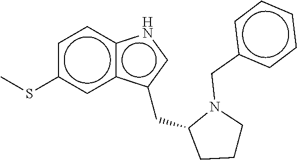 |
| 40B | 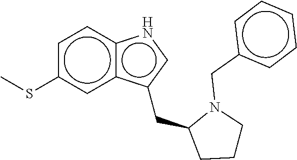 |
| 41A | 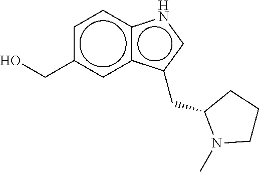 |
| 41B | 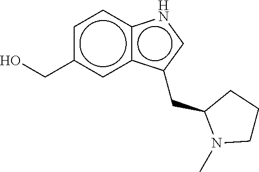 |
| 42A | 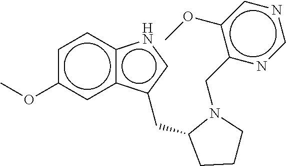 |
| 42B | 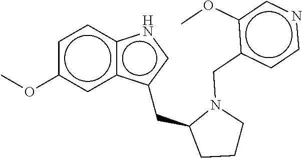 |
| 43A | 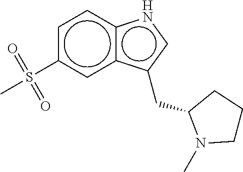 |
| 43B | 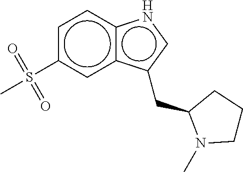 |
| 44A | 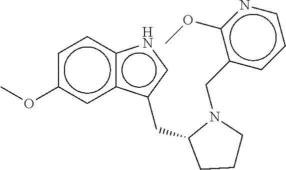 |
| 44B | 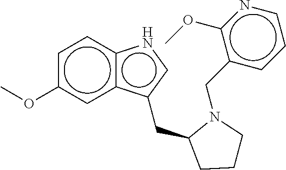 |
| 45A | 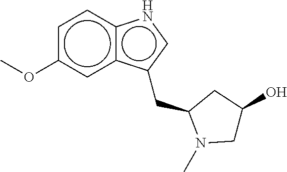 |
| 45B | 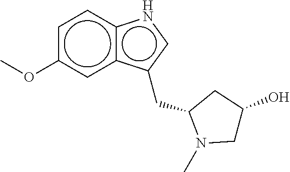 |
| 45C | 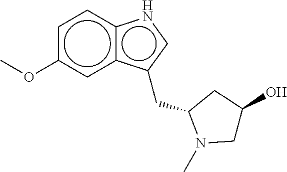 |
| 45D | 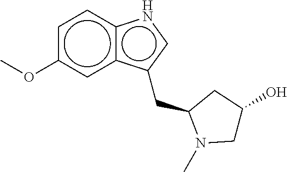 |
| 46A | 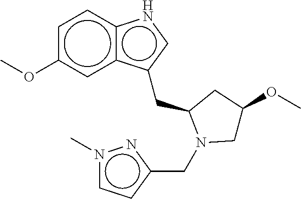 |
| 46B | 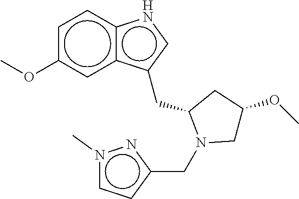 |
| 46C | 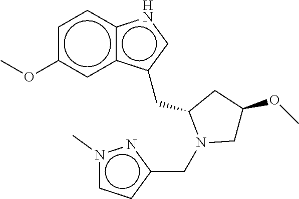 |
| 46D | 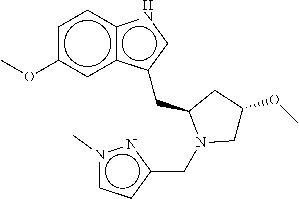 |
| 47A | 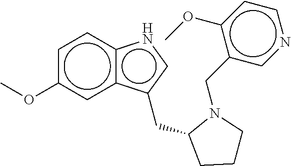 |
| 47B | 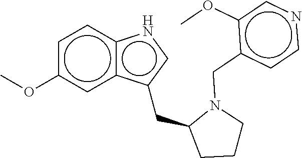 |
| 48A | 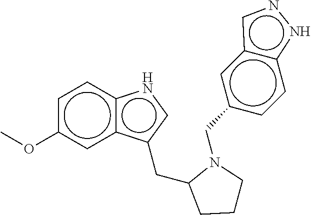 |
| 48B | 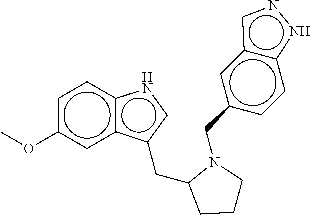 |
| 49A | 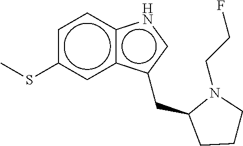 |
| 49B | 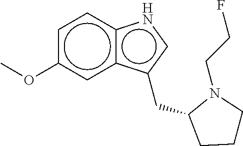 |
| 50A | 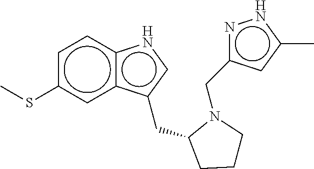 |
| 50B | 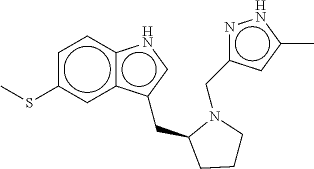 |
| 51A | 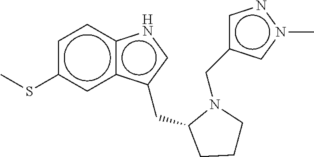 |
| 51B | 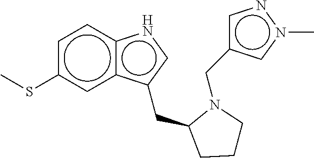 |
| 52A | 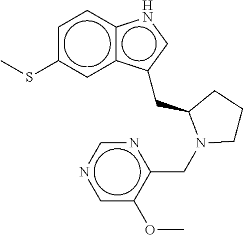 |
| 52B | 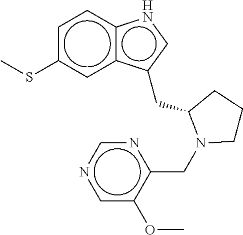 |
| 53A | 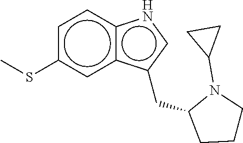 |
| 53B | 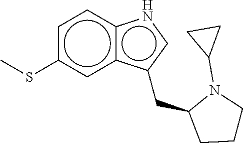 |
| 54A | 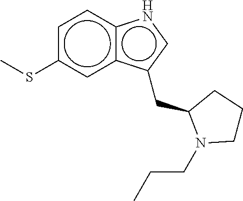 |
| 54B | 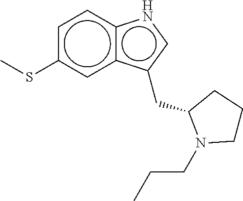 |
| 54C | 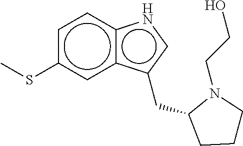 |
| 54D | 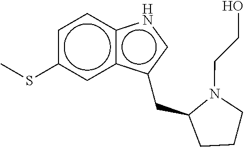 |
| 55A | 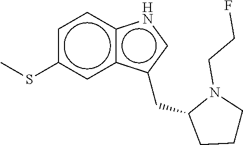 |
| 55B | 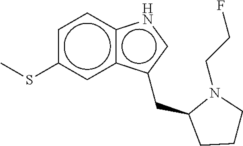 |
| 56A | 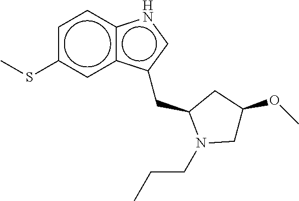 |
| 56B | 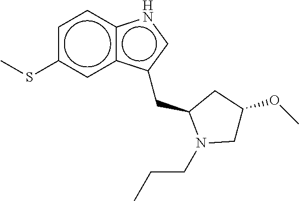 |
| 57A | 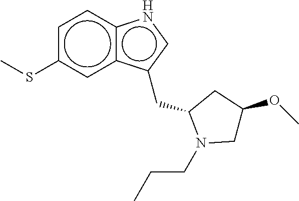 |
| 57B | 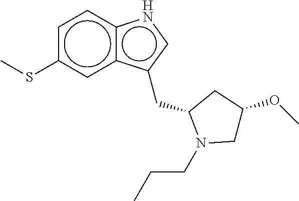 |
| 58A | 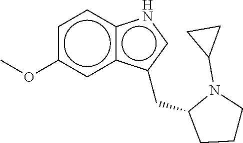 |
| 58B | 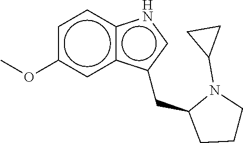 |
| 59A | 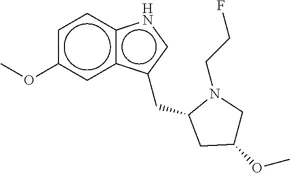 |
| 59B | 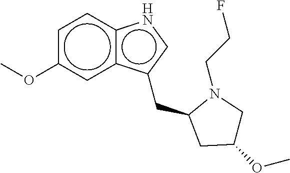 |
| 59C | 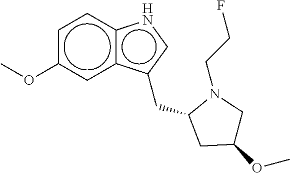 |
| 59D | 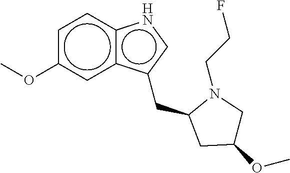 |
| 60A | 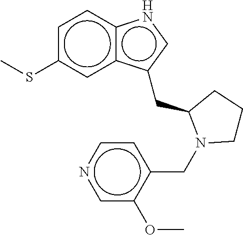 |
| 60B | 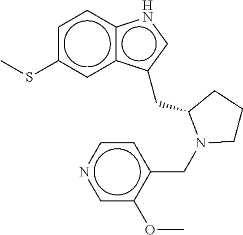 |
| 61A | 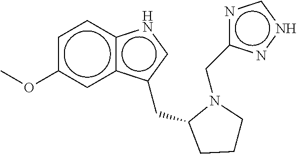 |
| 61B | 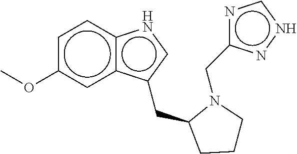 |
| 62A | 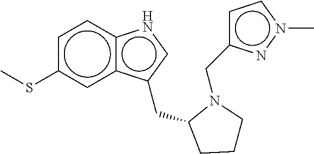 |
| 62B | 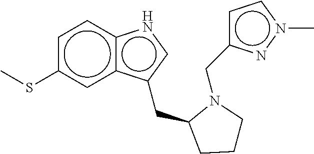 |
| 63A | 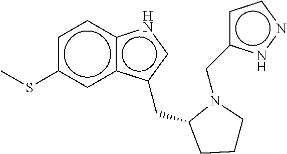 |
| 63B | 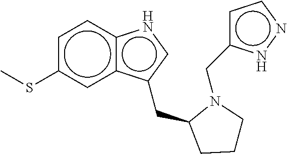 |
| 64A | 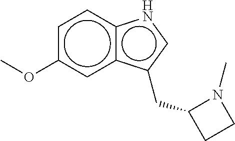 |
| 64B | 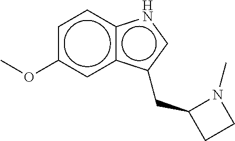 |

TABLE E
Various compounds of formula (III)

| Compound | |
|---|---|
| No. | Structure |
| 65 | 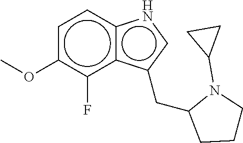 |
| 66 | 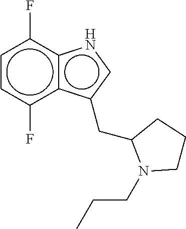 |

TABLE F
Various compounds of formula (III)

| Compound | |
|---|---|
| No. | Structure |
| 65A | 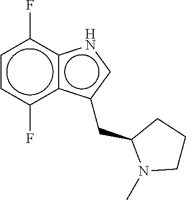 |
| 65B | 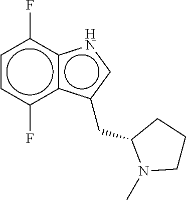 |
| 66A | 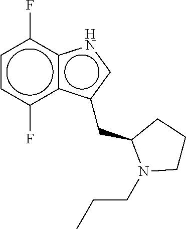 |
| 66B | 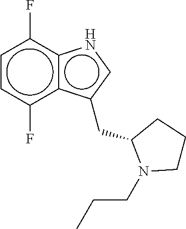 |

TABLE G
Various compounds of formula (IV)

| Compound | |
|---|---|
| No. | Structure |
| 67 | 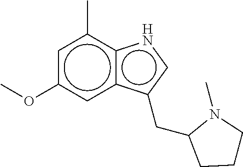 |
| 68 | 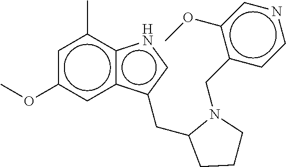 |
| 69 | 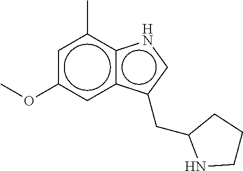 |
| 70 | 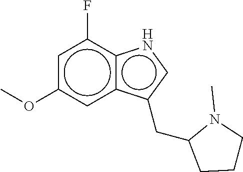 |

TABLE H
Various compounds of formula (IV)

| Compound | |
|---|---|
| No. | Structure |
| 67A | 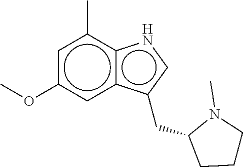 |
| 67B | 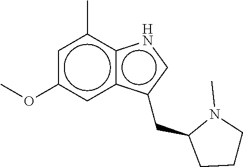 |
| 68A | 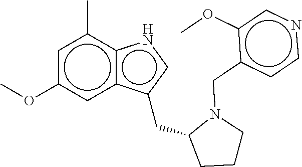 |
| 68B | 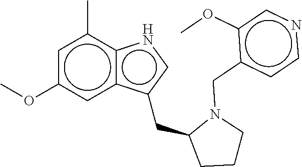 |
| 69A | 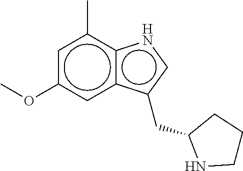 |
| 69B | 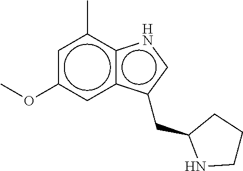 |
| 70A | 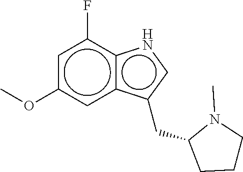 |
| 70B | 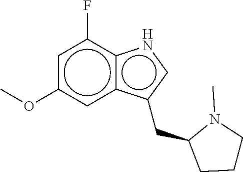 |

TABLE I
Various compounds of formula (V)

| Compound | |
|---|---|
| No. | Structure |
| 71 | 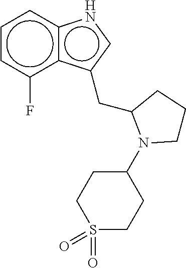 |
| 72 | 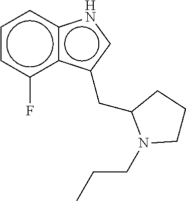 |
| 73 | 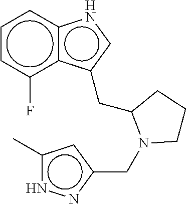 |
| 74 | 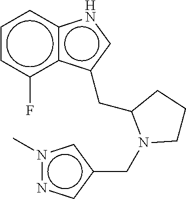 |
| 75 | 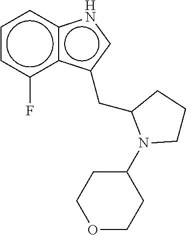 |
| 76 | 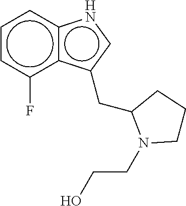 |
| 77 | 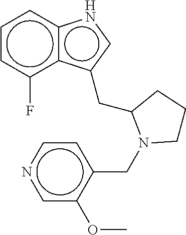 |
| 78 | 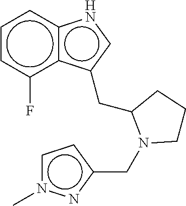 |
| 79 | 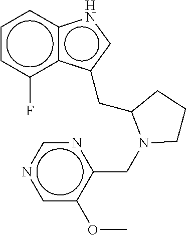 |
| 80 | 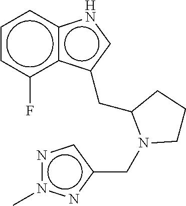 |
| 81 | 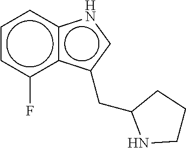 |
| 82 | 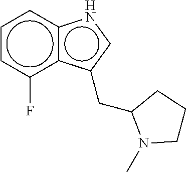 |
| 83 | 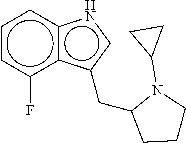 |
| 84 | 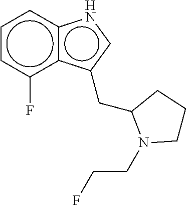 |

TABLE J
Various compounds of formula (V)

| Compound | |
|---|---|
| No. | Structure |
| 71A | 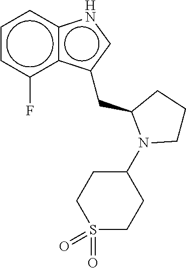 |
| 71B | 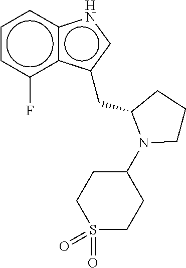 |
| 72A | 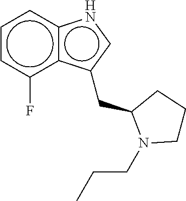 |
| 72B | 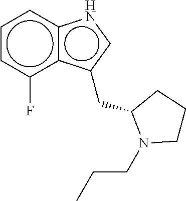 |
| 73A | 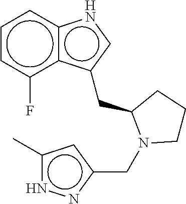 |
| 73B | 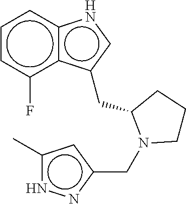 |
| 74A | 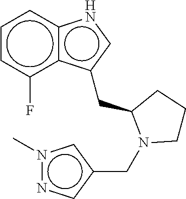 |
| 74B | 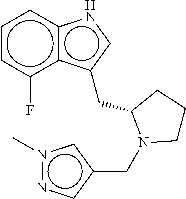 |
| 75A | 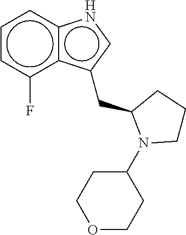 |
| 75B | 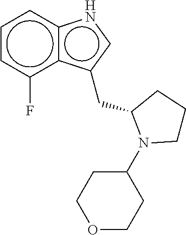 |
| 76A | 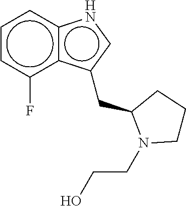 |
| 76B | 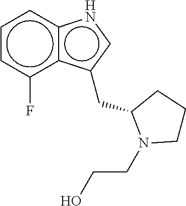 |
| 77A | 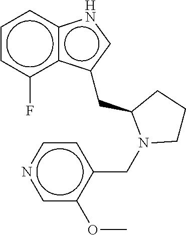 |
| 77B | 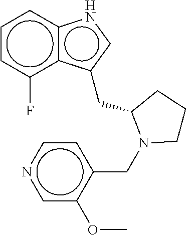 |
| 78A | 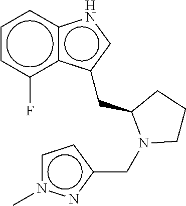 |
| 78B | 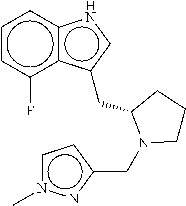 |
| 79A | 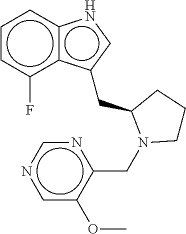 |
| 79B | 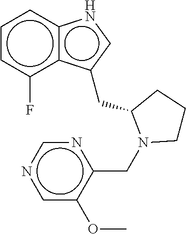 |
| 80A | 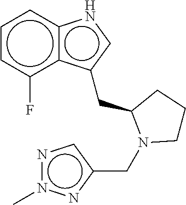 |
| 80B | 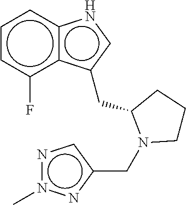 |
| 81A | 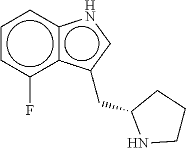 |
| 81B | 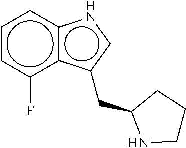 |
| 82A | 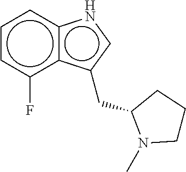 |
| 82B | 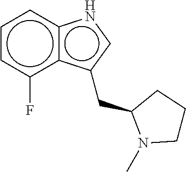 |
| 83A | 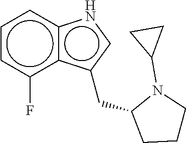 |
| 83B | 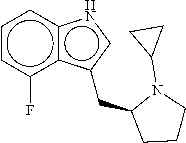 |
| 84A | 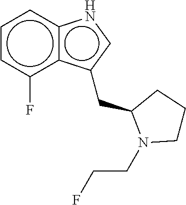 |
| 84A | 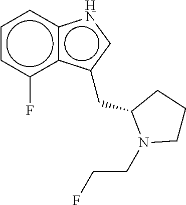 |

TABLE K
Various compounds of the present disclosure

| Compound | |
|---|---|
| No. | Structure |
| 85 | 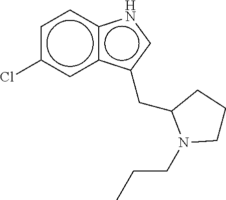 |
| 86 | 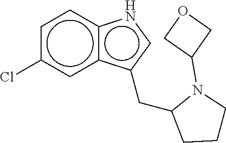 |
| 87 | 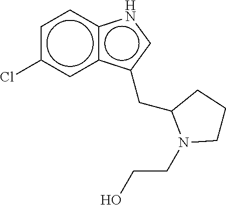 |
| 88 | 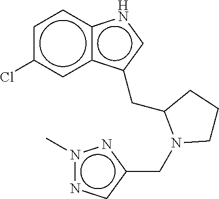 |
| 89 | 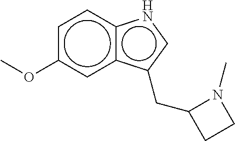 |
| 90 | 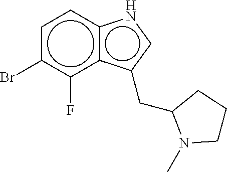 |
| 91 | 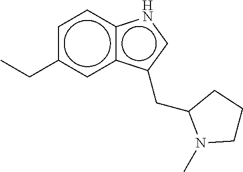 |

TABLE L
Various compounds of the present disclosure

| Compound | |
|---|---|
| No. | Structure |
| 85A | 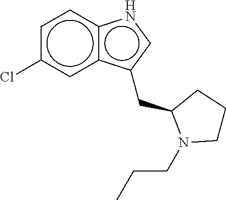 |
| 85B | 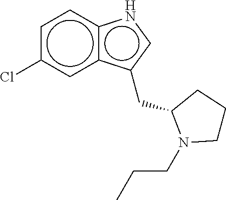 |
| 86A | 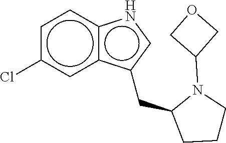 |
| 86B | 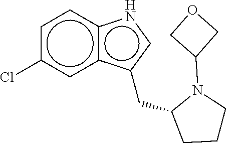 |
| 87A | 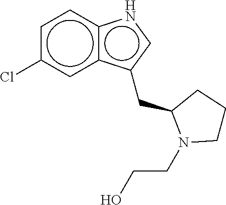 |
| 87B | 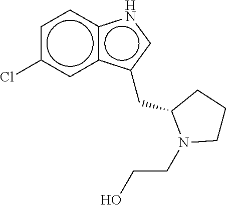 |
| 88A | 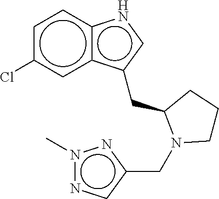 |
| 88B | 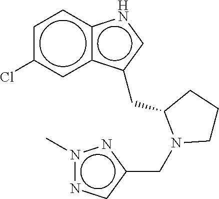 |
| 89A | 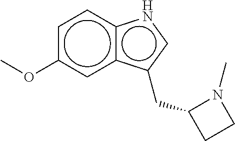 |
| 89B | 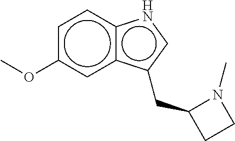 |
| 90A | 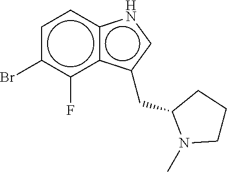 |
| 90B | 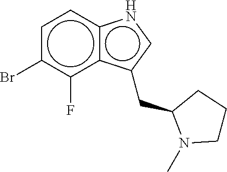 |
| 91A | 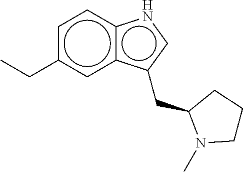 |
| 91B | 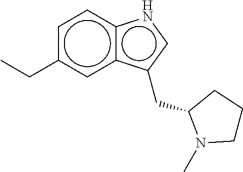 |

### Compositions

The present disclosure provides pharmaceutical compositions comprising at least one compound disclosed herein (e.g., of compound of formula (I), (II), (III), (IV), (V), or Tables A-L or pharmaceutically acceptable salt, deuterated versions, or stereoisomer thereof) and one or more pharmaceutically acceptable excipients.

The compounds provided herein (e.g., of compound of formula (I), (II), (III), (IV), (V), or Tables A-L or pharmaceutically acceptable salt, deuterated versions, or stereoisomer thereof) may be administered as compounds per se or may be formulated as pharmaceutical compositions. The pharmaceutical compositions may comprise one or more pharmaceutically acceptable excipients, such as carriers, diluents, fillers, disintegrants, lubricating agents, binders, colorants, pigments, stabilizers, preservatives, and/or antioxidants.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

### Therapeutic Use

The disclosure further relates to compounds disclosed herein (e.g., of compound of formula (I), (II), (III), (IV), (V), or Tables A-L or pharmaceutically acceptable salt, deuterated versions, or stereoisomer thereof), or a pharmaceutical composition comprising at least one compound disclosed herein (e.g., of compound of formula (I), (II), (III), (IV), (V), or Tables A-L or pharmaceutically acceptable salt, deuterated versions, or stereoisomer thereof), for use in the treatment of a serotonin 5-HT2A receptor associated disease/disorder.

In embodiments, the compounds (e.g., of compound of formula (I), (II), (III), (IV), (V), or Tables A-L or pharmaceutically acceptable salt, deuterated versions, or stereoisomer thereof), may be used in the treatment of an anxiety disorder, attention deficit hyperactivity disorder (ADHD), depression (including treatment resistant depression), cluster headache, diminished drive, burn-out, bore-out, migraine, Parkinson's disease, schizophrenia, an eating disorder (including anorexia nervosa), psychotic disorder, schizophrenia, schizophreniform disorder, schizoaffective disorder, bipolar I disorder, bipolar II disorder, major depressive disorder, psychotic depression, delusional disorders, shared psychotic disorder, Shared paranoia disorder, brief psychotic disorder, paranoid personality disorder, schizoid personality disorder, schizotypal personality disorder, social anxiety disorder, substance-induced anxiety disorder, selective mutism, panic disorder, panic attacks, agoraphobia, posttraumatic stress disorder (PTSD), premenstrual dysphoric disorder (PMDD), and premenstrual syndrome (PMS).

In embodiments, the 5-HT₂A receptor associated disease or disorder is depression. In embodiments, the depression is treatment resistant depression.

In embodiments, the 5-HT₂A receptor associated disease or disorder is an eating disorder. In embodiments, the eating disorder is anorexia nervosa.

In embodiments, the 5-HT₂A receptor associated disease or disorder is an anxiety disorder.

In embodiments, the 5-HT₂A receptor associated disease or disorder is bipolar I disorder.

In embodiments, the 5-HT₂A receptor associated disease or disorder is bipolar II disorder.

In embodiments, the 5-HT₂A receptor associated disease or disorder is major depressive disorder.

In embodiments, the 5-HT₂A receptor associated disease or disorder is posttraumatic stress disorder (PTSD).

### EXAMPLES

The present disclosure is further illustrated by reference to the following Examples. However, it should be noted that these Examples, like the embodiments described above, are illustrative and are not to be construed as restricting the scope of the disclosure in any way.

In embodiments, compounds of the present disclosure can be synthesized using the following methods. General reaction conditions are given, and reaction products can be purified by generally known methods including silica gel chromatography using various organic solvents such as hexane, dichloromethane, ethyl acetate, methanol and the like or preparative reverse phase high pressure liquid chromatography.

### Example 1: Synthesis of (R)-5-methoxy-3-((1-(oxetan-3-yl)pyrrolidin-2-yl)methyl)-1H-indole (25A)

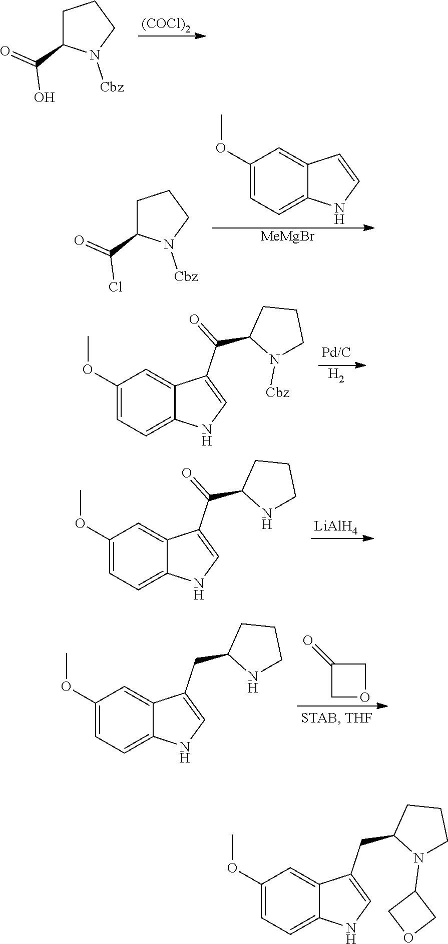

### Step 1: Benzyl (R)-2-(chlorocarbonyl)pyrrolidine-1-carboxylate

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h. The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil.

m/z 264.2 (M−Cl+MeOH)⁺ (ES⁺)

### Step 2: Benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity).

m/z 401.4 (M+Na+H)⁺ (ES+); 377.3 (M−H)⁻ (ES−)

1H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%).

m/z 245.2 (M+H)+ (ES+); 243.2 (M−H)− (ES−)

1H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%).

m/z 231.2 (M+H)+ (ES+); 229.2 (M−H)− (ES−)

¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78%

### Step 5: (R)-5-methoxy-3-((1-(oxetan-3-yl)pyrrolidin-2-yl)methyl)-1H-indole

To a stirred solution of the product from step 4 above (70.0 mg, 1 Eq, 289 μmol) in dry THF (3.00 mL) at rt was added oxetan-3-one (31.2 mg, 1.5 Eq, 433 μmol). The reaction mixture was stirred for 1 h. Sodium triacetoxyborohydride (184 mg, 3 Eq, 866 μmol) was added and the reaction mixture was left to stir for 72 h at rt. The reaction mixture was quenched with sat. aq NaHCO₃ (5 mL) and left to stir. The aqueous suspension was diluted with water (5 mL) and extracted with EtOAc (3×10 mL). The combined organic layers were dried over MgSO₄, filtered and concentrated in vacuo. The crude product was purified by chromatography on silica gel (12 g cartridge, 0-80% (3:1 EtOAc/EtOH+2% NH₄OH) in iso-hexane) to afford (R)-5-methoxy-3-((1-(oxetan-3-yl)pyrrolidin-2-yl)methyl)-1H-indole (41.3 mg, 139 μmol, 48.0%).

m/z 287.2 (M+H)+ (ES+)

¹H NMR (500 MHz, DMSO) δ 10.61 (s, 1H), 7.20 (d, J=8.7 Hz, 1H), 7.08 (d, J=2.4 Hz, 1H), 6.93 (d, J=2.4 Hz, 1H), 6.69 (dd, J=8.7, 2.4 Hz, 1H), 4.65-4.57 (m, 3H), 4.54 (t, J=6.6 Hz, 1H), 3.99 (p, J=6.9 Hz, 1H), 3.75 (s, 3H), 2.99-2.93 (m, 1H), 2.77-2.69 (m, 2H), 2.49-2.42 (m, 1H), 2.37 (q, J=7.9 Hz, 1H), 1.73-1.55 (m, 3H), 1.54-1.42 (m, 1H).

### Example 2: Synthesis of (R)-5-methoxy-3-((1-(tetrahydro-2H-pyran-4-yl)pyrrolidin-2-yl)methyl)-1H-indole (28B)

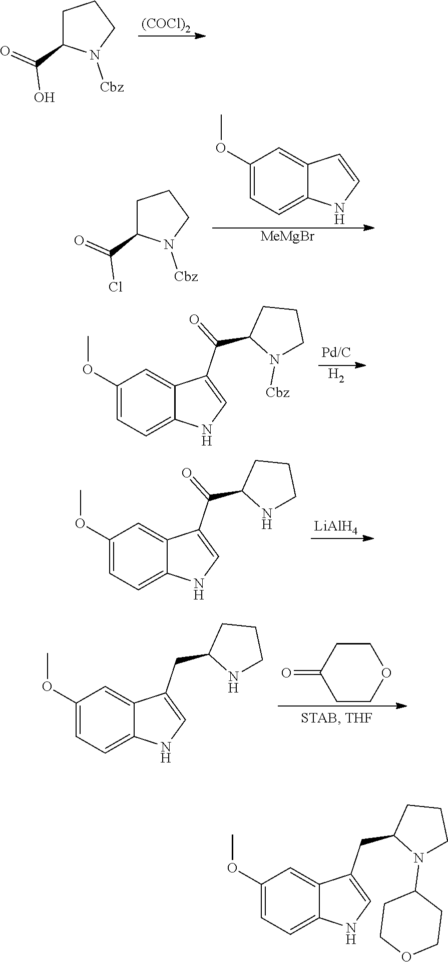

### Step 1: Benzyl (R)-2-(chlorocarbonyl)pyrrolidine-1-carboxylate

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h. The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil.

m/z 264.2 (M−Cl+MeOH)⁺ (ES⁺)

### Step 2: Benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity).

m/z 401.4 (M+Na+H)⁺ (ES+); 377.3 (M−H)⁻ (ES−)

¹H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%).

m/z 245.2 (M+H)+ (ES+); 243.2 (M−H)− (ES−)

1H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%).

m/z 231.2 (M+H)+ (ES+); 229.2 (M−H)− (ES−)

¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78%

### Step 5: (R)-5-methoxy-3-((1-(tetrahydro-2H-pyran-4-yl)pyrrolidin-2-yl)methyl)-1H-indole

To a stirred solution of the product from step 4 above (70.0 mg, 1 Eq, 289 μmol) in dry THF (3.00 mL) at rt was added tetrahydro-4H-thiopyran-4-one 1,1-dioxide (64.2 mg, 1.5 Eq, 433 μmol). The reaction mixture was stirred for 1 h. Sodium triacetoxyborohydride (184 mg, 3 Eq, 866 μmol) was added and the reaction mixture left to stir for 72 h at rt. The reaction mixture was quenched with sat. aq. NaHCO₃ (5 mL) and left to stir. The aqueous suspension was diluted with water (5 mL) and extracted with EtOAc (3×10 mL). The combined organic layers were dried over MgSO₄, filtered and concentrated in vacuo.

The crude product was purified by chromatography on silica gel (12 g cartridge, 0-80% (1:3 EtOH/EtOAc+2% NH₄OH) in iso-hexane) to afford (R)-5-methoxy-3-((1-(tetrahydro-2H-pyran-4-yl)pyrrolidin-2-yl)methyl)-1H-indole (61.9 mg, 188 μmol, 65.2%).

m/z 315.4 (M+H)+ (ES+); 313.5 (M−H)− (ES−)

¹H NMR (500 MHz, DMSO) δ 10.38 (s, 1H), 7.22 (d, J=8.7 Hz, 1H), 7.06 (d, J=2.4 Hz, 1H), 6.98 (d, J=2.4 Hz, 1H), 6.72 (dd, J=8.7, 2.4 Hz, 1H), 3.95-3.86 (m, 2H), 3.76 (s, 3H), 3.32 (dtd, J=21.2, 11.6, 2.2 Hz, 2H), 3.19-3.11 (m, 1H), 2.91-2.78 (m, 3H), 2.62-2.55 (m, 1H), 2.53 (d, J=9.3 Hz, 1H), 1.80-1.71 (m, 2H), 1.71-1.57 (m, 4H), 1.57-1.45 (m, 2H).

### Example 3: Synthesis of (R)-4-(2-((5-methoxy-1H-indol-3-yl)methyl)pyrrolidin-1-yl)tetrahydro-2H-thiopyran 1,1-dioxide (29B)

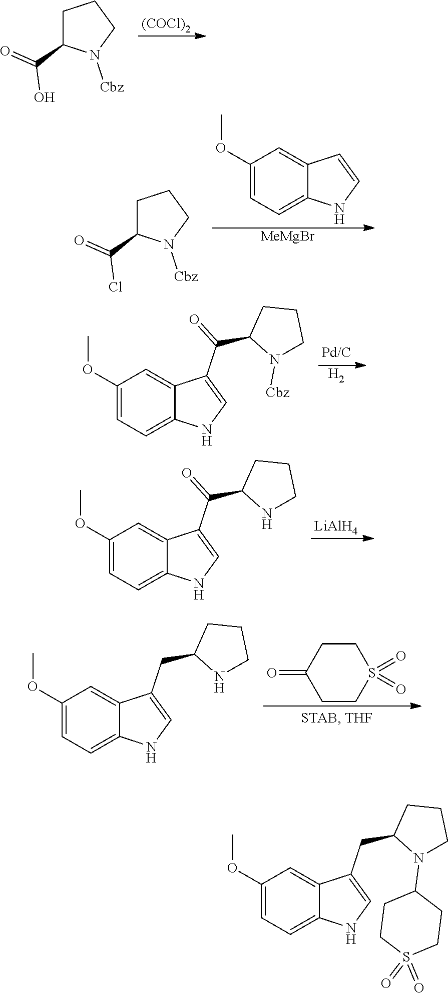

### Step 1: Benzyl (R)-2-(chlorocarbonyl)pyrrolidine-1-carboxylate

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h. The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil.

m/z 264.2 (M−Cl+MeOH)⁺ (ES+)

### Step 2: benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity).

m/z 401.4 (M+Na+H)⁺ (ES+); 377.3 (M−H)⁻ (ES−)

¹H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%).

m/z 245.2 (M+H)+ (ES+); 243.2 (M−H)− (ES−)

1H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%).

m/z 231.2 (M+H)+ (ES+); 229.2 (M−H)− (ES−)

¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78%

### Step 5: (R)-4-(2-((5-methoxy-1H-indol-3-yl)methyl)pyrrolidin-1-yl)tetrahydro-2H-thiopyran-1,1-dioxide

To a stirred solution of the product from step 4 above (70.0 mg, 1 Eq, 289 μmol) in dry THF (3.00 mL) at rt was added tetrahydro-4H-thiopyran-4-one 1,1-dioxide (64.2 mg, 1.5 Eq, 433 μmol). The reaction mixture was stirred for 1 h. Sodium triacetoxyborohydride (184 mg, 3 Eq, 866 μmol) was added and the reaction mixture left to stir for 72 h at rt. The reaction mixture was quenched with sat. aq. NaHCO₃ (5 mL) and left to stir. The aqueous suspension was diluted with water (5 mL) and extracted with EtOAc (3×10 mL). The combined organic layers were dried over MgSO₄, filtered and concentrated in vacuo.

The crude product was purified by chromatography on silica gel (12 g cartridge, 0-80% (1:3 EtOH/EtOAc+2% NH₄OH) in iso-hexane) to afford (R)-4-(2-((5-methoxy-1H-indol-3-yl)methyl)pyrrolidin-1-yl)tetrahydro-2H-thiopyran 1,1-dioxide (46.1 mg, 0.13 mmol, 44

m/z 363.3 (M+H)+ (ES+); 361.1 (M−H)− (ES−)

¹H NMR (500 MHz, DMSO) δ 10.62 (s, 1H), 7.21 (d, J=8.7 Hz, 1H), 7.10 (d, J=2.3 Hz, 1H), 6.96 (d, J=2.4 Hz, 1H), 6.70 (dd, J=8.7, 2.4 Hz, 1H), 3.75 (s, 3H), 3.25-3.13 (m, 2H), 3.13-3.00 (m, 4H), 2.86 (dd, J=14.2, 3.3 Hz, 1H), 2.81 (t, J=8.0 Hz, 1H), 2.60-2.52 (m, 1H), 2.45 (dd, J=14.2, 9.8 Hz, 1H), 2.16-2.03 (m, 3H), 2.03-1.92 (m, 1H), 1.71-1.56 (m, 3H), 1.54-1.43 (m, 1H).

### Example 4: Synthesis of (R)-3-((1-((1H-pyrazol-5-yl)methyl)pyrrolidin-2-yl)methyl)-5-methoxy-1H-indole (23A)

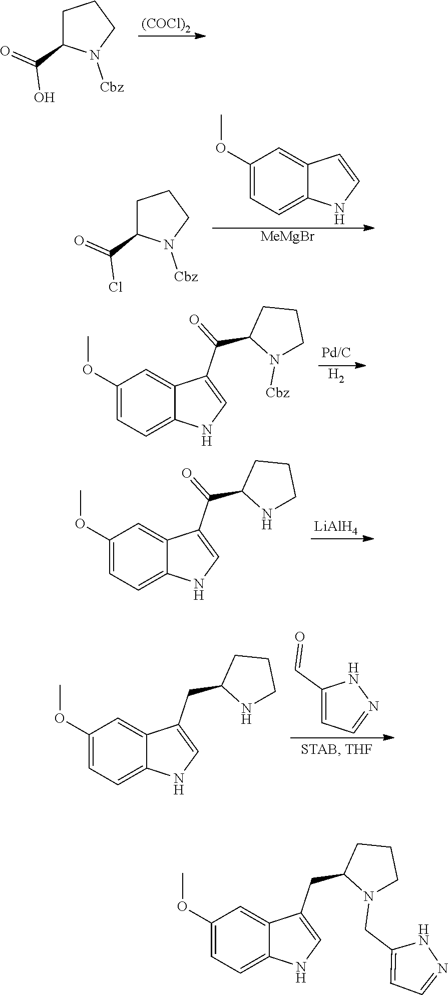

### Step 1: Benzyl (R)-2-(chlorocarbonyl)pyrrolidine-1-carboxylate

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h. The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil.

m/z 264.2 (M−Cl+MeOH)⁺ (ES⁺)

### Step 2: Benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity).

m/z 401.4 (M+Na+H)⁺ (ES+); 377.3 (M−H)⁻ (ES−)

¹H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%).

m/z 245.2 (M+H)+ (ES+); 243.2 (M−H)− (ES−)

1H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%).

m/z 231.2 (M+H)+ (ES+); 229.2 (M−H)− (ES−)

¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78%

### Step 5: (R)-3-((1-((1H-pyrazol-5-yl)methyl)pyrrolidin-2-yl)methyl)-5-methoxy-1H-indole

To a stirred solution of the product from step 4 above (70.0 mg, 1 Eq, 289 μmol) in dry THF (3.00 mL) at rt was added 1H-pyrazole-5-carbaldehyde (41.6 mg, 1.5 Eq, 433 μmol). The reaction mixture was stirred for 1 h. Sodium triacetoxyborohydride (184 mg, 3 Eq, 866 μmol) was added and the reaction mixture left to stir for 72 h at rt. The reaction mixture was quenched with sat. aq. NaHCO₃ (5 mL) and left to stir. The aqueous suspension was diluted with water (5 mL) and extracted with EtOAc (3×10 mL). The combined organic layers were dried over MgSO₄, filtered and concentrated in vacuo. The crude product was purified by chromatography on silica gel (12 g cartridge, 0-80% (1:3 EtOH/EtOAc+2% NH₄OH) in iso-hexane) to afford (R)-3-((1-((1H-pyrazol-5-yl)methyl)pyrrolidin-2-yl)methyl)-5-methoxy-1H-indole (52.0 mg, 0.16 mmol, 57%).

m/z 311.3 (M+H)+ (ES+); 309.3 (M−H)− (ES−)

¹H NMR (500 MHz, DMSO) δ 12.52 (s, 1H) 10.59 (s, 1H), 7.53 (s, 1H), 7.19 (d, J=8.7 Hz, 1H), 7.09 (d, J=2.3 Hz, 1H), 6.95 (d, J=2.4 Hz, 1H), 6.69 (dd, J=8.7, 2.5 Hz, 1H), 6.17 (d, J=2.0 Hz, 1H), 4.01 (d, J=13.7 Hz, 1H), 3.75 (s, 3H), 3.55 (d, J=13.6 Hz, 1H), 3.02 (dd, J=14.0, 3.7 Hz, 1H), 2.89 (t, J=8.0 Hz, 1H), 2.70 (s, 1H), 2.47 (d, J=9.5 Hz, 1H), 2.27 (d, J=8.4 Hz, 1H), 1.70-1.60 (m, 1H), 1.60-1.50 (m, 2H), 1.50-1.41 (m, 1H).

### Example 5: Synthesis of (R)-5-methoxy-3-((1-((2-methyl-2H-1,2,3-triazol-4-yl)methyl)pyrrolidin-2-yl)methyl)-1H-indole (26A)

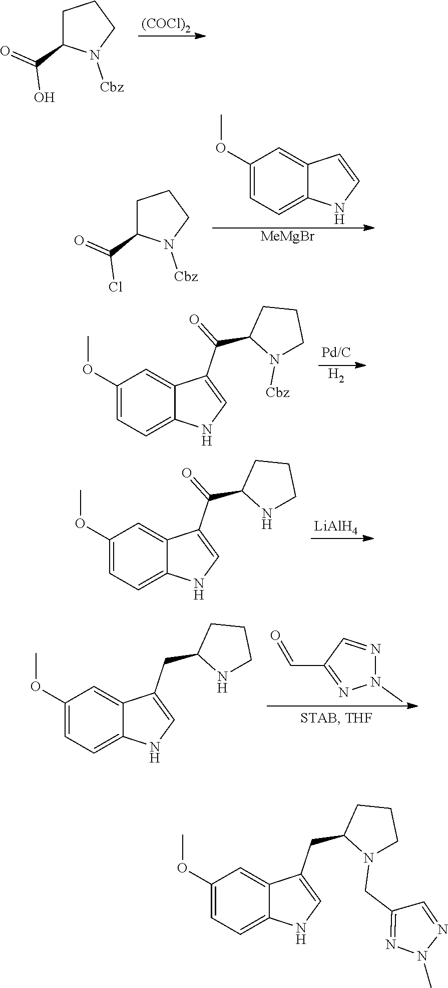

### Step 1: Benzyl (R)-2-(chlorocarbonyl)pyrrolidine-1-carboxylate

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h. The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil.

m/z 264.2 (M−Cl+MeOH)⁺ (ES⁺)

### Step 2: benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity).

m/z 401.4 (M+Na+H)⁺ (ES+); 377.3 (M−H)⁻ (ES−)

¹H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%).

m/z 245.2 (M+H)+ (ES+); 243.2 (M−H)− (ES−)

1H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%).

m/z 231.2 (M+H)+ (ES+); 229.2 (M−H)− (ES−)

¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78%

### Step 5: (R)-5-methoxy-3-((1-((2-methyl-2H-1,2,3-triazol-4-yl)methyl)pyrrolidin-2-yl)methyl)-1H-indole

To a stirred solution of the product from step 4 above (84.0 mg, 1 Eq, 237 μmol) in dry THF (3.00 mL) at rt was added 2-methyl-2H-1,2,3-triazole-4-carbaldehyde (33.3 mg, 1.2 Eq, 284 μmol). The reaction mixture was stirred for 1 h. Sodium triacetoxyborohydride (151 mg, 3 Eq, 711 μmol) was added and the reaction mixture left to stir for 20 h at rt. The reaction mixture was quenched with sat. aq. NaHCO₃ (5 mL) and left to stir. The aqueous suspension was diluted with water (5 mL) and extracted with EtOAc (3×10 mL). The combined organic layers were dried over MgSO₄, filtered and concentrated in vacuo. The crude product was purified by chromatography on silica gel (12 g cartridge, 0-80% (1:3 EtOH/EtOAc+2% NH₄OH) in iso-hexane) to afford (R)-5-methoxy-3-((1-((2-methyl-2H-1,2,3-triazol-4-yl)methyl)pyrrolidin-2-yl)methyl)-1H-indole (21.0 mg, 61 μmol, 26%).

m/z 326.6 (M+H)+ (ES+)

¹H NMR (500 MHz, DMSO) δ 10.59 (s, 1H), 7.62 (s, 1H), 7.20 (d, J=8.7 Hz, 1H), 7.10 (d, J=2.4 Hz, 1H), 6.99 (d, J=2.4 Hz, 1H), 6.70 (dd, J=8.7, 2.4 Hz, 1H), 4.11 (s, 3H), 4.06 (d, J=13.8 Hz, 1H), 3.75 (s, 3H), 3.59 (d, J=13.9 Hz, 1H), 3.04 (dd, J=14.1, 3.8 Hz, 1H), 2.89 (t, J=8.0 Hz, 1H), 2.73-2.65 (m, 1H), 2.53 (s, 1H), 2.27 (d, J=8.8 Hz, 1H), 1.72-1.61 (m, 1H), 1.61-1.51 (m, 2H), 1.51-1.42 (m, 1H). One proton obscured by DMSO peak, observed in HSQC.

### Example 6: Synthesis of (R)-4-fluoro-3-((1-((5-methyl-1H-pyrazol-3-yl)methyl)pyrrolidin-2-yl)methyl)-1H-indole (73A)

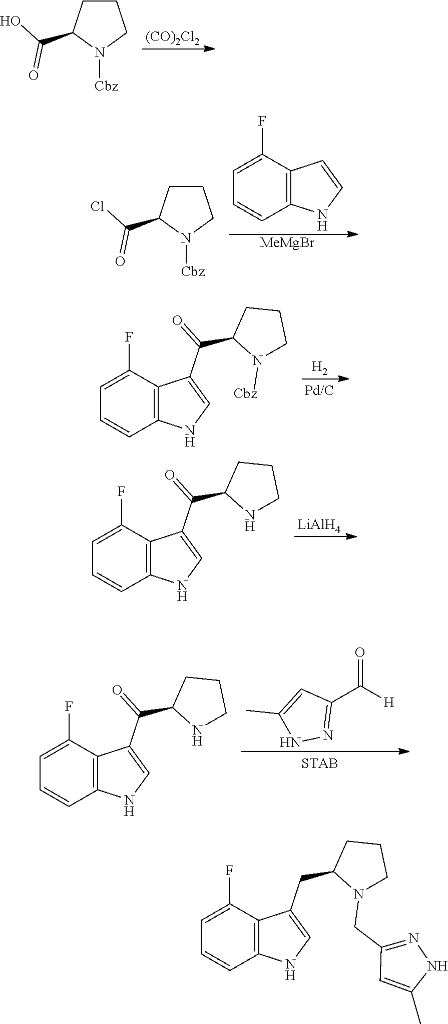

### Step 1: Benzyl (R)-2-(chlorocarbonyl)pyrrolidine-1-carboxylate

To a stirred solution of ((benzyloxy)carbonyl)-D-proline (1.90 g, 1 Eq, 7.62 mmol) in dry DCM (53.0 mL) under a nitrogen atmosphere at 0 °C was added oxalyl chloride (2.90 g, 2.00 mL, 3 Eq, 22.9 mmol) and catalytic DMF. The reaction mixture was stirred at 0 °C for 3 hours. oxalyl chloride (2.90 g, 2.00 mL, 3 Eq, 22.9 mmol) was added and the mixture was stirred at 0 °C for 1 hour and then the solvent was removed under reduced pressure to afford the product (2.04 g, 7.62 mmol, 100%, 100% Purity) as a yellow oil.

m/z 264.2 (M+MeOH−Cl+H)+ (ES+)

### Step 2: Benzyl (R)-2-(4-fluoro-1H-indole-3-carbonyl)pyrrolidine-1-carboxylate

To a stirred solution of 4-fluoro-1H-indole (2.00 g, 2 Eq, 14.8 mmol) in dry Toluene (100 mL) under a nitrogen atmosphere at 0 °C was added methylmagnesium bromide (1.76 g, 4.93 mL, 3.00 molar, 2 Eq, 14.8 mmol) dropwise over 10 min and the mixture was stirred at the same temperature for 3 hours. Freshly synthesised benzyl (R)-2-(chlorocarbonyl)pyrrolidine-1-carboxylate (1.98 g, 1 Eq, 7.40 mmol) in Toluene (10.0 mL) was added dropwise to the stirring solution of the deprotonated indole and the reaction mixture was stirred at room temperature for 16 hours.

The reaction mixture was quenched with sat. sodium bicarbonate (150 mL) at 0 °C and left to stir for 1 h, warming to room temperature. The two phases were separated and the aqueous phase was extracted with EtOAc (3×100 mL). The combined organic phases were washed with brine (50 mL), dried over MgSO₄, filtered and the solvent removed under reduced pressure. The crude product was purified by flash chromatography on silica gel (40 g column, 0-70% EtOAc in hexanes, load (silica)) to afford the product (1.66 g, 4.0 mmol, 54%, 88% Purity).

m/z 367.0 (M+H)+ (ES+)

### Step 3: (R)-4-fluoro-3-prolyl-1H-indole

To a mixture of benzyl (R)-2-(4-fluoro-1H-indole-3-carbonyl)pyrrolidine-1-carboxylate (1.66 g, 88% Wt, 1 Eq, 3.99 mmol) in EtOH (25.0 mL) was added palladium on carbon (166 mg, 0.391 Eq, 1.56 mmol) and the mixture was placed under a 1 bar hydrogen atmosphere. The mixture was stirred at room temperature for 16 hours. The reaction was vented and purged with nitrogen. The mixture was filtered to remove the Pd and the solvent removed under reduced pressure to afford (R)-4-fluoro-3-prolyl-1H-indole (0.920 g, 3.9 mmol, 98%, 99% Purity).

m/z 233.0 (M+H)+ (ES+)

### Step 4: (R)-4-fluoro-3-(pyrrolidin-2-ylmethyl)-1H-indole

To a mixture of (R)-4-fluoro-3-prolyl-1H-indole (0.940 g, 1 Eq, 4.05 mmol) in 2-Methyltetrahydrofuran (13.5 mL) at 0 °C was added lithium aluminum hydride (2.4 M in THF) (614 mg, 6.75 mL, 2.40 molar, 4 Eq, 16.2 mmol) dropwise. After for 10 min, the reaction was stirred at 66 °C for 5 hours and then at room temperature for 12 hours. The mixture was cooled down to 0 °C, diluted with THF (20 mL) and water (0.5 mL), 2.0 M NaOH (0.5 mL) and water (1.5 mL) were added followed by Na₂SO₄ (ca 2 g). The mixture was stirred for 45 min and filtered. The filtrated was concentrated under reduced pressure and the crude product purified by flash chromatography on silica gel (12 g column, 0-100% (20% (0.7 M in MeOH) in dichloromethane) in dichloromethane, liquid load (dichloromethane)) to afford the product (430 mg, 1.9 mmol, 46%, 94% Purity) after being triturated with MTBE.

m/z 219.2 (M+H)+ (ES+)

¹H NMR (500 MHz, DMSO) δ 1.26-1.37 (m, 1H), 1.52-1.73 (m, 3H), 2.65-2.80 (m, 2H), 2.81-2.91 (m, 2H), 3.19-3.26 (m, 1H), 6.64-6.70 (m, 1H), 6.95-7.01 (m, 1H), 7.14 (d, J=8.3 Hz, 2H), 11.04-11.08 (m, 1H)—NH missing due to a fast solvent exchange

¹⁹F NMR (471 MHz, DMSO) δ −124.47.

### Step 5: (R)-4-fluoro-3-((1-((5-methyl-1H-pyrazol-3-yl)methyl)pyrrolidin-2-yl)methyl)-1H-indole

A mixture of (R)-4-fluoro-3-(pyrrolidin-2-ylmethyl)-1H-indole (60.0 mg, 1 Eq, 275 μmol) and 5-methyl-1H-pyrazole-3-carbaldehyde (45.4 mg, 1.5 Eq, 412 μmol) in THF (2.50 mL) was stirred for 1 hour at room temperature. sodium triacetoxyborohydride (175 mg, 3 Eq, 825 μmol) was added and the mixture was stirred at room temperature for 16 hours.

The reaction was quenched with sat. aq. NaHCO₃ (10 mL) and the mixture was extracted with EtOAc (3×5 mL). The combined organic phases were washed with brine (5 mL), dried over MgSO₄, filtered and the solvent removed under reduced pressure. The crude product was purified by flash chromatography on silica gel (4 g column, 0-60% (10% (0.7 M NH₃ in MeOH) in dichloromethane) in dichloromethane, liquid load (dichloromethane)) to afford the product (43.0 mg, 0.13 mmol, 48%, 96% Purity).

m/z 313.2 (M+H)+ (ES+); m/z 311.2 (M−H)− (ES−).

¹H NMR (500 MHz, DMSO) δ 1.44-1.71 (m, 4H), 2.16 (s, 4H), 2.56-2.73 (m, 2H), 2.87 (s, 1H), 3.18 (d, J=13.9 Hz, 1H), 3.94 (d, J=13.6 Hz, 1H), 5.89 (s, 1H), 6.69 (dd, J=11.6, 7.7 Hz, 1H), 6.94-7.03 (m, 1H), 7.06-7.25 (m, 2H), 11.08 (s, 1H), 12.09 (s, 1H)—1H under the water residual peak.

¹⁹F NMR (471 MHz, DMSO) δ −124.33.

### Example 7: Synthesis of (R)-4-fluoro-3-((1-((1-methyl-1H-pyrazol-3-yl)methyl)pyrrolidin-2-yl)methyl)-1H-indole (78A)

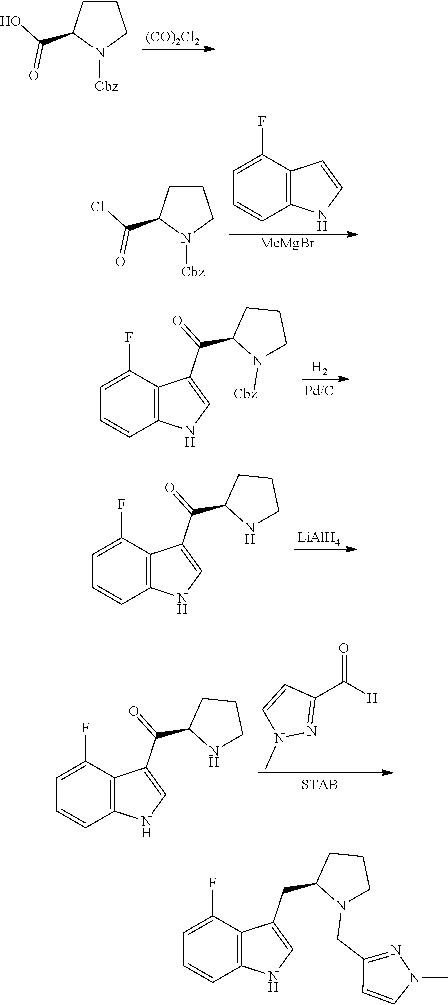

### Step 1: benzyl (R)-2-(chlorocarbonyl)pyrrolidine-1-carboxylate

To a stirred solution of ((benzyloxy)carbonyl)-D-proline (1.90 g, 1 Eq, 7.62 mmol) in dry DCM (53.0 mL) under a nitrogen atmosphere at 0 °C was added oxalyl chloride (2.90 g, 2.00 mL, 3 Eq, 22.9 mmol) and catalytic DMF. The reaction mixture was stirred at 0 °C for 3 hours. oxalyl chloride (2.90 g, 2.00 mL, 3 Eq, 22.9 mmol) was added and the mixture was stirred at 0 °C for 1 hour and then the solvent was removed under reduced pressure to afford the product (2.04 g, 7.62 mmol, 100%, 100% Purity) as a yellow oil.

m/z 264.2 (M+MeOH−Cl+H)+ (ES+)

### Step 2: benzyl (R)-2-(4-fluoro-1H-indole-3-carbonyl)pyrrolidine-1-carboxylate

To a stirred solution of 4-fluoro-1H-indole (2.00 g, 2 Eq, 14.8 mmol) in dry Toluene (100 mL) under a nitrogen atmosphere at 0 °C was added methylmagnesium bromide (1.76 g, 4.93 mL, 3.00 molar, 2 Eq, 14.8 mmol) dropwise over 10 min and the mixture was stirred at the same temperature for 3 hours. Freshly synthesised benzyl (R)-2-(chlorocarbonyl)pyrrolidine-1-carboxylate (1.98 g, 1 Eq, 7.40 mmol) in Toluene (10.0 mL) was added dropwise to the stirring solution of the deprotonated indole and the reaction mixture was stirred at room temperature for 16 hours.

The reaction mixture was quenched with sat. sodium bicarbonate (150 mL) at 0 °C and left to stir for 1 h, warming to room temperature. The two phases were separated and the aqueous phase was extracted with EtOAc (3×100 mL). The combined organic phases were washed with brine (50 mL), dried over MgSO₄, filtered and the solvent removed under reduced pressure. The crude product was purified by flash chromatography on silica gel (40 g column, 0-70% EtOAc in hexanes, load (silica)) to afford the product (1.66 g, 4.0 mmol, 54%, 88% Purity).

m/z 367.0 (M+H)+ (ES+)

### Step 3: (R)-4-fluoro-3-prolyl-1H-indole

To a mixture of benzyl (R)-2-(4-fluoro-1H-indole-3-carbonyl)pyrrolidine-1-carboxylate (1.66 g, 88% Wt, 1 Eq, 3.99 mmol) in EtOH (25.0 mL) was added palladium on carbon (166 mg, 0.391 Eq, 1.56 mmol) and the mixture was placed under a 1 bar hydrogen atmosphere. The mixture was stirred at room temperature for 16 hours. The reaction was vented and purged with nitrogen. The mixture was filtered to remove the Pd and the solvent removed under reduced pressure to afford (R)-4-fluoro-3-prolyl-1H-indole (0.920 g, 3.9 mmol, 98%, 99% Purity).

m/z 233.0 (M+H)+ (ES+)

### Step 4: (R)-4-fluoro-3-(pyrrolidin-2-ylmethyl)-1H-indole

To a mixture of (R)-4-fluoro-3-prolyl-1H-indole (0.940 g, 1 Eq, 4.05 mmol) in 2-Methyltetrahydrofuran (13.5 mL) at 0 °C was added lithium aluminum hydride (2.4 M in THF) (614 mg, 6.75 mL, 2.40 molar, 4 Eq, 16.2 mmol) dropwise. After for 10 min, the reaction was stirred at 66 °C for 5 hours and then at room temperature for 12 hours. The mixture was cooled down to 0 °C, diluted with THF (20 mL) and water (0.5 mL), 2.0 M NaOH (0.5 mL) and water (1.5 mL) were added followed by Na₂SO₄ (ca 2 g). The mixture was stirred for 45 min and filtered. The filtrated was concentrated under reduced pressure and the crude product purified by flash chromatography on silica gel (12 g column, 0-100% (20% (0.7 M in MeOH) in dichloromethane) in dichloromethane, liquid load (dichloromethane)) to afford the product (430 mg, 1.9 mmol, 46%, 94% Purity) after being triturated with MTBE.

m/z 219.2 (M+H)+ (ES+)

¹H NMR (500 MHz, DMSO) δ 1.26-1.37 (m, 1H), 1.52-1.73 (m, 3H), 2.65-2.80 (m, 2H), 2.81-2.91 (m, 2H), 3.19-3.26 (m, 1H), 6.64-6.70 (m, 1H), 6.95-7.01 (m, 1H), 7.14 (d, J=8.3 Hz, 2H), 11.04-11.08 (m, 1H)—NH missing due to a fast solvent exchange

¹⁹F NMR (471 MHz, DMSO) δ −124.47.

### Step 5: (R)-4-fluoro-3-((1-((1-methyl-1H-pyrazol-3-yl)methyl)pyrrolidin-2-yl)methyl)-1H-indole

A mixture of (R)-4-fluoro-3-(pyrrolidin-2-ylmethyl)-1H-indole (60.0 mg, 1 Eq, 275 μmol) and 1-methyl-1H-pyrazole-3-carbaldehyde (45.4 mg, 1.5 Eq, 412 μmol) in THF (2.50 mL) was stirred for 1 hour at room temperature. sodium triacetoxyborohydride (175 mg, 3 Eq, 825 μmol) was added and the mixture was stirred at room temperature for 16 hours.

The reaction was quenched with sat. aq. NaHCO₃ (10 mL) and the mixture was extracted with EtOAc (3×5 mL). The combined organic phases were washed with brine (5 mL), dried over MgSO₄, filtered and the solvent removed under reduced pressure. The crude product was purified by flash chromatography on silica gel (4 g column, 0-60% (10% (0.7 M NH3 in MeOH) in dichloromethane) in dichloromethane, liquid load (dichloromethane)) to afford (59.0 mg, 0.19 mmol, 67%, 98% Purity).

m/z 313.2 (M+H)+ (ES+); m/z 311.0 (M−H)− (ES−).

¹H NMR (500 MHz, DMSO) δ 1.45-1.71 (m, 4H), 2.16-2.20 (m, 1H), 2.56-2.65 (m, 1H), 2.65-2.75 (m, 1H), 2.84-2.92 (m, 1H), 3.19 (d, J=13.6 Hz, 1H), 3.78 (s, 3H), 3.95 (d, J=13.3 Hz, 1H), 6.10 (s, 1H), 6.69 (dd, J=11.6, 7.7 Hz, 1H), 6.95-7.03 (m, 1H), 7.12-7.18 (m, 2H), 7.56 (s, 1H), 11.08 (s, 1H).—1H under water residual peak.

¹⁹F NMR (471 MHz, DMSO) δ −124.35.

### Example 8: Synthesis of (R)-4-(2-((4-fluoro-1H-indol-3-yl)methyl)pyrrolidin-1-yl)tetrahydro-2H-thiopyran 1,1-dioxide (71A)

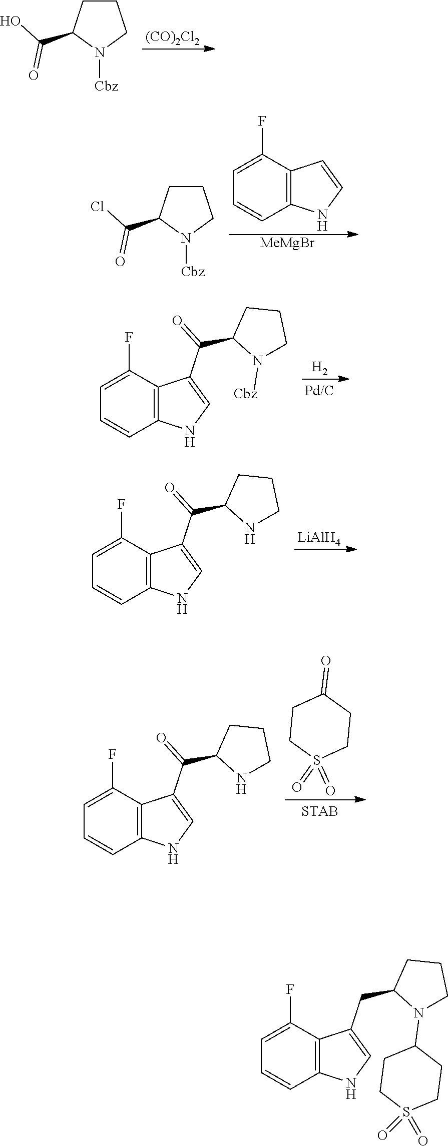

### Step 1: benzyl (R)-2-(chlorocarbonyl)pyrrolidine-1-carboxylate

To a stirred solution of ((benzyloxy)carbonyl)-D-proline (1.90 g, 1 Eq, 7.62 mmol) in dry DCM (53.0 mL) under a nitrogen atmosphere at 0 °C was added oxalyl chloride (2.90 g, 2.00 mL, 3 Eq, 22.9 mmol) and catalytic DMF. The reaction mixture was stirred at 0 °C for 3 hours. oxalyl chloride (2.90 g, 2.00 mL, 3 Eq, 22.9 mmol) was added and the mixture was stirred at 0 °C for 1 hour and then the solvent was removed under reduced pressure to afford the product (2.04 g, 7.62 mmol, 100%, 100% Purity) as a yellow oil.

m/z 264.2 (M+MeOH−Cl+H)+ (ES+)

### Step 2: benzyl (R)-2-(4-fluoro-1H-indole-3-carbonyl)pyrrolidine-1-carboxylate

To a stirred solution of 4-fluoro-1H-indole (2.00 g, 2 Eq, 14.8 mmol) in dry Toluene (100 mL) under a nitrogen atmosphere at 0 °C was added methylmagnesium bromide (1.76 g, 4.93 mL, 3.00 molar, 2 Eq, 14.8 mmol) dropwise over 10 min and the mixture was stirred at the same temperature for 3 hours. Freshly synthesised benzyl (R)-2-(chlorocarbonyl)pyrrolidine-1-carboxylate (1.98 g, 1 Eq, 7.40 mmol) in Toluene (10.0 mL) was added dropwise to the stirring solution of the deprotonated indole and the reaction mixture was stirred at room temperature for 16 hours.

The reaction mixture was quenched with sat. sodium bicarbonate (150 mL) at 0 °C and left to stir for 1 h, warming to room temperature. The two phases were separated and the aqueous phase was extracted with EtOAc (3×100 mL). The combined organic phases were washed with brine (50 mL), dried over MgSO₄, filtered and the solvent removed under reduced pressure. The crude product was purified by flash chromatography on silica gel (40 g column, 0-70% EtOAc in hexanes, load (silica)) to afford the product (1.66 g, 4.0 mmol, 54%, 88% Purity).

m/z 367.0 (M+H)+ (ES+)

### Step 3: (R)-4-fluoro-3-prolyl-1H-indole

To a mixture of benzyl (R)-2-(4-fluoro-1H-indole-3-carbonyl)pyrrolidine-1-carboxylate (1.66 g, 88% Wt, 1 Eq, 3.99 mmol) in EtOH (25.0 mL) was added palladium on carbon (166 mg, 0.391 Eq, 1.56 mmol) and the mixture was placed under a 1 bar hydrogen atmosphere. The mixture was stirred at room temperature for 16 hours. The reaction was vented and purged with nitrogen. The mixture was filtered to remove the Pd and the solvent removed under reduced pressure to afford (R)-4-fluoro-3-prolyl-1H-indole (0.920 g, 3.9 mmol, 98%, 99% Purity).

m/z 233.0 (M+H)+ (ES+)

### Step 4: (R)-4-fluoro-3-(pyrrolidin-2-ylmethyl)-1H-indole

To a mixture of (R)-4-fluoro-3-prolyl-1H-indole (0.940 g, 1 Eq, 4.05 mmol) in 2-Methyltetrahydrofuran (13.5 mL) at 0 °C was added lithium aluminum hydride (2.4 M in THF) (614 mg, 6.75 mL, 2.40 molar, 4 Eq, 16.2 mmol) dropwise. After for 10 min, the reaction was stirred at 66 °C for 5 hours and then at room temperature for 12 hours. The mixture was cooled down to 0 °C, diluted with THF (20 mL) and water (0.5 mL), 2.0 M NaOH (0.5 mL) and water (1.5 mL) were added followed by Na₂SO₄ (ca 2 g). The mixture was stirred for 45 min and filtered. The filtrated was concentrated under reduced pressure and the crude product purified by flash chromatography on silica gel (12 g column, 0-100% (20% (0.7 M in MeOH) in dichloromethane) in dichloromethane, liquid load (dichloromethane)) to afford the product (430 mg, 1.9 mmol, 46%, 94% Purity) after being triturated with MTBE.

m/z 219.2 (M+H)+ (ES+)

¹H NMR (500 MHz, DMSO) δ 1.26-1.37 (m, 1H), 1.52-1.73 (m, 3H), 2.65-2.80 (m, 2H), 2.81-2.91 (m, 2H), 3.19-3.26 (m, 1H), 6.64-6.70 (m, 1H), 6.95-7.01 (m, 1H), 7.14 (d, J=8.3 Hz, 2H), 11.04-11.08 (m, 1H)—NH missing due to a fast solvent exchange

¹⁹F NMR (471 MHz, DMSO) δ −124.47.

### Step 5: (R)-4-(2-((4-fluoro-1H-indol-3-yl)methyl)pyrrolidin-1-yl)tetrahydro-2H-thiopyran 1,1-dioxide

A mixture of (R)-4-fluoro-3-(pyrrolidin-2-ylmethyl)-1H-indole (60.0 mg, 1 Eq, 275 μmol) and tetrahydro-4H-thiopyran-4-one 1,1-dioxide (48.9 mg, 1.2 Eq, 330 μmol) in THF (2.50 mL) was stirred for 1 hour at room temperature. sodium triacetoxyborohydride (175 mg, 3 Eq, 825 μmol) was added and the mixture was stirred at room temperature for 16 hours.

The reaction was quenched with sat. aq. NaHCO₃ (10 mL) and the mixture was extracted with EtOAc (3×5 mL). The combined organic phases were washed with brine (5 mL), dried over MgSO₄, filtered and the solvent removed under reduced pressure. The crude product was purified by flash chromatography on silica gel (4 g column, 0-60% (10% (0.7 M NH3 in MeOH) in dichloromethane) in dichloromethane, liquid load (dichloromethane)) to afford the product (54.0 mg, 0.15 mmol, 55%, 98% Purity).

m/z 351.2 (M+H)+ (ES+); m/z 349.2 (M−H)− (ES−)

¹H NMR (500 MHz, DMSO) δ 1.47-1.72 (m, 4H), 1.91-2.13 (m, 4H), 2.53-2.60 (m, 1H), 2.78-2.84 (m, 1H), 2.98-3.05 (m, 2H), 3.05-3.13 (m, 4H), 3.15-3.24 (m, 1H), 6.69 (dd, J=11.6, 7.7 Hz, 1H), 6.95-7.03 (m, 1H), 7.13-7.18 (m, 2H), 11.10 (s, 1H)—1H under the DMSO residual peak.

¹⁹F NMR (471 MHz, DMSO) δ −124.47.

### Example 9: Synthesis of (R)-4-fluoro-3-((1-(tetrahydro-2H-pyran-4-yl)pyrrolidin-2-yl)methyl)-1H-indole (75A)

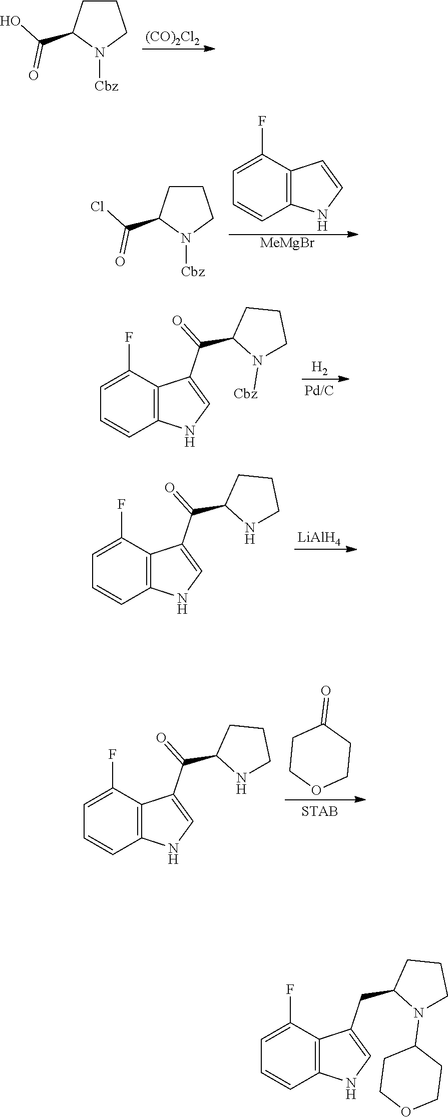

### Step 1: Benzyl (R)-2-(chlorocarbonyl)pyrrolidine-1-carboxylate

To a stirred solution of ((benzyloxy)carbonyl)-D-proline (1.90 g, 1 Eq, 7.62 mmol) in dry DCM (53.0 mL) under a nitrogen atmosphere at 0 °C was added oxalyl chloride (2.90 g, 2.00 mL, 3 Eq, 22.9 mmol) and catalytic DMF. The reaction mixture was stirred at 0 °C for 3 hours. oxalyl chloride (2.90 g, 2.00 mL, 3 Eq, 22.9 mmol) was added and the mixture was stirred at 0 °C for 1 hour and then the solvent was removed under reduced pressure to afford the product (2.04 g, 7.62 mmol, 100%, 100% Purity) as a yellow oil.

m/z 264.2 (M+MeOH−Cl+H)+ (ES+)

### Step 2: Benzyl (R)-2-(4-fluoro-1H-indole-3-carbonyl)pyrrolidine-1-carboxylate

To a stirred solution of 4-fluoro-1H-indole (2.00 g, 2 Eq, 14.8 mmol) in dry Toluene (100 mL) under a nitrogen atmosphere at 0 °C was added methylmagnesium bromide (1.76 g, 4.93 mL, 3.00 molar, 2 Eq, 14.8 mmol) dropwise over 10 min and the mixture was stirred at the same temperature for 3 hours. Freshly synthesised benzyl (R)-2-(chlorocarbonyl)pyrrolidine-1-carboxylate (1.98 g, 1 Eq, 7.40 mmol) in Toluene (10.0 mL) was added dropwise to the stirring solution of the deprotonated indole and the reaction mixture was stirred at room temperature for 16 hours.

The reaction mixture was quenched with sat. sodium bicarbonate (150 mL) at 0 °C and left to stir for 1 h, warming to room temperature. The two phases were separated and the aqueous phase was extracted with EtOAc (3×100 mL). The combined organic phases were washed with brine (50 mL), dried over MgSO₄, filtered and the solvent removed under reduced pressure. The crude product was purified by flash chromatography on silica gel (40 g column, 0-70% EtOAc in hexanes, load (silica)) to afford the product (1.66 g, 4.0 mmol, 54%, 88% Purity).

m/z 367.0 (M+H)+ (ES+)

### Step 3: (R)-4-fluoro-3-prolyl-1H-indole

To a mixture of benzyl (R)-2-(4-fluoro-1H-indole-3-carbonyl)pyrrolidine-1-carboxylate (1.66 g, 88% Wt, 1 Eq, 3.99 mmol) in EtOH (25.0 mL) was added palladium on carbon (166 mg, 0.391 Eq, 1.56 mmol) and the mixture was placed under a 1 bar hydrogen atmosphere. The mixture was stirred at room temperature for 16 hours. The reaction was vented and purged with nitrogen. The mixture was filtered to remove the Pd and the solvent removed under reduced pressure to afford (R)-4-fluoro-3-prolyl-1H-indole (0.920 g, 3.9 mmol, 98%, 99% Purity).

m/z 233.0 (M+H)+ (ES+)

### Step 4: (R)-4-fluoro-3-(pyrrolidin-2-ylmethyl)-1H-indole

To a mixture of (R)-4-fluoro-3-prolyl-1H-indole (0.940 g, 1 Eq, 4.05 mmol) in 2-Methyltetrahydrofuran (13.5 mL) at 0 °C was added lithium aluminum hydride (2.4 M in THF) (614 mg, 6.75 mL, 2.40 molar, 4 Eq, 16.2 mmol) dropwise. After for 10 min, the reaction was stirred at 66 °C for 5 hours and then at room temperature for 12 hours. The mixture was cooled down to 0 °C, diluted with THF (20 mL) and water (0.5 mL), 2.0 M NaOH (0.5 mL) and water (1.5 mL) were added followed by Na₂SO₄ (ca 2 g). The mixture was stirred for 45 min and filtered. The filtrated was concentrated under reduced pressure and the crude product purified by flash chromatography on silica gel (12 g column, 0-100% (20% (0.7 M in MeOH) in dichloromethane) in dichloromethane, liquid load (dichloromethane)) to afford the product (430 mg, 1.9 mmol, 46%, 94% Purity) after being triturated with MTBE.

m/z 219.2 (M+H)+ (ES+)

¹H NMR (500 MHz, DMSO) δ 1.26-1.37 (m, 1H), 1.52-1.73 (m, 3H), 2.65-2.80 (m, 2H), 2.81-2.91 (m, 2H), 3.19-3.26 (m, 1H), 6.64-6.70 (m, 1H), 6.95-7.01 (m, 1H), 7.14 (d, J=8.3 Hz, 2H), 11.04-11.08 (m, 1H)—NH missing due to a fast solvent exchange

¹⁹F NMR (471 MHz, DMSO) δ −124.47.

### Step 5: (R)-4-fluoro-3-((1-(tetrahydro-2H-pyran-4-yl)pyrrolidin-2-yl)methyl)-1H-indole

A mixture of (R)-4-fluoro-3-(pyrrolidin-2-ylmethyl)-1H-indole (60.0 mg, 1 Eq, 275 μmol) and tetrahydro-4H-pyran-4-one (33.0 mg, 30.5 μL, 1.2 Eq, 330 μmol) in THF (2.50 mL) was stirred for 1 hour at room temperature. sodium triacetoxyborohydride (175 mg, 3 Eq, 825 μmol) was added and the mixture was stirred at room temperature for 16 hours.

The reaction was quenched with sat. aq. NaHCO₃ (10 mL) and the mixture was extracted with EtOAc (3×5 mL). The combined organic phases were washed with brine (5 mL), dried over MgSO₄, filtered and the solvent removed under reduced pressure. The crude product was purified by flash chromatography on silica gel (4 g column, 0-60% (10% (0.7 M NH3 in MeOH) in dichloromethane) in dichloromethane, liquid load (dichloromethane)) to afford the product (53.0 mg, 0.17 mmol, 62%, 97% Purity).

m/z 303.2 (M+H)+ (ES+); m/z 301.2 (M−H)− (ES−).

¹H NMR (500 MHz, DMSO) δ 1.40-1.78 (m, 8H), 2.50-2.57 (m, 2H), 2.76-2.88 (m, 1H), 2.95-3.02 (m, 1H), 3.05-3.17 (m, 1H), 3.23-3.31 (m, 2H), 3.84-3.93 (m, 2H), 6.68 (dd, J=11.7, 7.7 Hz, 1H), 6.95-7.02 (m, 1H), 7.12-7.16 (m, 2H), 11.08 (s, 1H).—1H under the DMSO residual peak.

¹⁹F NMR (471 MHz, DMSO) δ −124.43.

### Example 10: Synthesis of (R)-4-fluoro-3-((1-((2-methyl-2H-1,2,3-triazol-4-yl)methyl)pyrrolidin-2-yl)methyl)-1H-indole (80A)

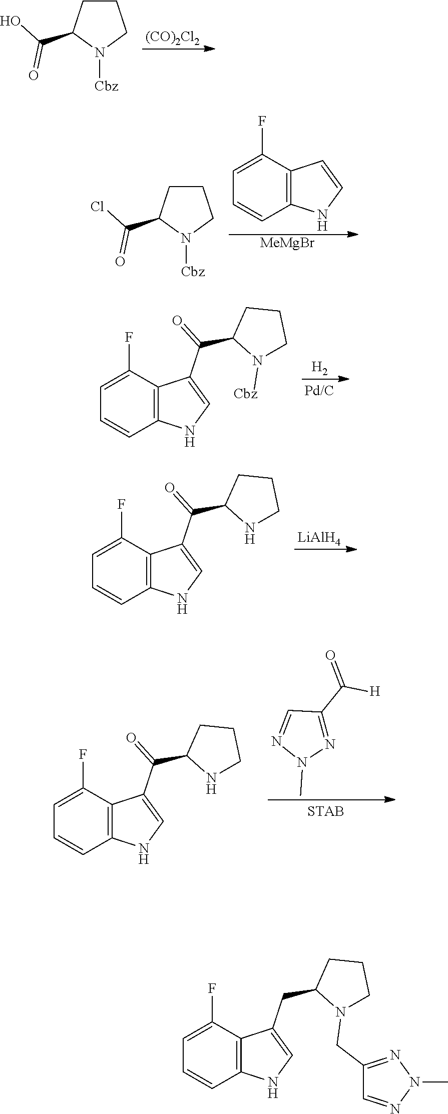

### Step 1: benzyl (R)-2-(chlorocarbonyl)pyrrolidine-1-carboxylate

To a stirred solution of ((benzyloxy)carbonyl)-D-proline (1.90 g, 1 Eq, 7.62 mmol) in dry DCM (53.0 mL) under a nitrogen atmosphere at 0 °C was added oxalyl chloride (2.90 g, 2.00 mL, 3 Eq, 22.9 mmol) and catalytic DMF. The reaction mixture was stirred at 0 °C for 3 hours. oxalyl chloride (2.90 g, 2.00 mL, 3 Eq, 22.9 mmol) was added and the mixture was stirred at 0 °C for 1 hour and then the solvent was removed under reduced pressure to afford the product (2.04 g, 7.62 mmol, 100%, 100% Purity) as a yellow oil.

m/z 264.2 (M+MeOH−Cl+H)+ (ES+)

### Step 2: Benzyl (R)-2-(4-fluoro-1H-indole-3-carbonyl)pyrrolidine-1-carboxylate

To a stirred solution of 4-fluoro-1H-indole (2.00 g, 2 Eq, 14.8 mmol) in dry Toluene (100 mL) under a nitrogen atmosphere at 0 °C was added methylmagnesium bromide (1.76 g, 4.93 mL, 3.00 molar, 2 Eq, 14.8 mmol) dropwise over 10 min and the mixture was stirred at the same temperature for 3 hours. Freshly synthesised benzyl (R)-2-(chlorocarbonyl)pyrrolidine-1-carboxylate (1.98 g, 1 Eq, 7.40 mmol) in Toluene (10.0 mL) was added dropwise to the stirring solution of the deprotonated indole and the reaction mixture was stirred at room temperature for 16 hours.

The reaction mixture was quenched with sat. sodium bicarbonate (150 mL) at 0 °C and left to stir for 1 h, warming to room temperature. The two phases were separated and the aqueous phase was extracted with EtOAc (3×100 mL). The combined organic phases were washed with brine (50 mL), dried over MgSO₄, filtered and the solvent removed under reduced pressure. The crude product was purified by flash chromatography on silica gel (40 g column, 0-70% EtOAc in hexanes, load (silica)) to afford the product (1.66 g, 4.0 mmol, 54%, 88% Purity).

m/z 367.0 (M+H)+ (ES+)

### Step 3: (R)-4-fluoro-3-prolyl-1H-indole

To a mixture of benzyl (R)-2-(4-fluoro-1H-indole-3-carbonyl)pyrrolidine-1-carboxylate (1.66 g, 88% Wt, 1 Eq, 3.99 mmol) in EtOH (25.0 mL) was added palladium on carbon (166 mg, 0.391 Eq, 1.56 mmol) and the mixture was placed under a 1 bar hydrogen atmosphere. The mixture was stirred at room temperature for 16 hours. The reaction was vented and purged with nitrogen. The mixture was filtered to remove the Pd and the solvent removed under reduced pressure to afford (R)-4-fluoro-3-prolyl-1H-indole (0.920 g, 3.9 mmol, 98%, 99% Purity).

m/z 233.0 (M+H)+ (ES+)

### Step 4: (R)-4-fluoro-3-(pyrrolidin-2-ylmethyl)-1H-indole

To a mixture of (R)-4-fluoro-3-prolyl-1H-indole (0.940 g, 1 Eq, 4.05 mmol) in 2-Methyltetrahydrofuran (13.5 mL) at 0 °C was added lithium aluminum hydride (2.4 M in THF) (614 mg, 6.75 mL, 2.40 molar, 4 Eq, 16.2 mmol) dropwise. After for 10 min, the reaction was stirred at 66 °C for 5 hours and then at room temperature for 12 hours. The mixture was cooled down to 0 °C, diluted with THF (20 mL) and water (0.5 mL), 2.0 M NaOH (0.5 mL) and water (1.5 mL) were added followed by Na₂SO₄ (ca 2 g). The mixture was stirred for 45 min and filtered. The filtrated was concentrated under reduced pressure and the crude product purified by flash chromatography on silica gel (12 g column, 0-100% (20% (0.7 M in MeOH) in dichloromethane) in dichloromethane, liquid load (dichloromethane)) to afford the product (430 mg, 1.9 mmol, 46%, 94% Purity) after being triturated with MTBE.

m/z 219.2 (M+H)+ (ES+)

¹H NMR (500 MHz, DMSO) δ 1.26-1.37 (m, 1H), 1.52-1.73 (m, 3H), 2.65-2.80 (m, 2H), 2.81-2.91 (m, 2H), 3.19-3.26 (m, 1H), 6.64-6.70 (m, 1H), 6.95-7.01 (m, 1H), 7.14 (d, J=8.3 Hz, 2H), 11.04-11.08 (m, 1H)—NH missing due to a fast solvent exchange

¹⁹F NMR (471 MHz, DMSO) δ −124.47.

### Step 5: (R)-4-fluoro-3-((1-((2-methyl-2H-1,2,3-triazol-4-yl)methyl)pyrrolidin-2-yl)methyl)-1H-indole

A mixture of (R)-4-fluoro-3-(pyrrolidin-2-ylmethyl)-1H-indole (60.0 mg, 1 Eq, 275 μmol) and 2-methyl-2H-1,2,3-triazole-4-carbaldehyde (36.6 mg, 1.2 Eq, 330 μmol) in THF (2.50 mL) was stirred for 1 hour at room temperature. sodium triacetoxyborohydride (175 mg, 3 Eq, 825 μmol) was added and the mixture was stirred at room temperature for 16 hours.

The reaction was quenched with sat. aq. NaHCO₃ (10 mL) and the mixture was extracted with EtOAc (3×5 mL). The combined organic phases were washed with brine (5 mL), dried over MgSO₄, filtered and the solvent removed under reduced pressure. The crude product was purified by flash chromatography on silica gel (4 g column, 0-60% (10% (0.7 M NH3 in MeOH) in dichloromethane) in dichloromethane, liquid load (dichloromethane)) to afford the product (41.0 mg, 0.13 mmol, 47%, 99% Purity) as a colourless oil.

m/z 314.4 (M+H)+ (ES+); m/z 312.2 (M−H)− (ES−).

¹H NMR (500 MHz, DMSO) δ 1.45-1.70 (m, 4H), 2.17-2.26 (m, 1H), 2.61 (dd, J=13.7, 9.1 Hz, 1H), 2.67-2.76 (m, 1H), 2.87 (ddd, J=9.5, 6.9, 2.6 Hz, 1H), 3.18 (dd, J=13.7, 3.6 Hz, 1H), 3.46 (d, J=13.8 Hz, 1H), 4.04 (d, J=13.8 Hz, 1H), 4.09 (s, 3H), 6.69 (ddd, J=11.6, 7.8, 0.8 Hz, 1H), 6.99 (td, J=7.9, 5.1 Hz, 1H), 7.13-7.19 (m, 2H), 7.57 (s, 1H), 11.08 (s, 1H).

¹⁹F NMR (471 MHz, DMSO) δ −124.37.

### Example 11: Synthesis of (R)-4-fluoro-3-((1-((5-methoxypyrimidin-4-yl)methyl)pyrrolidin-2-yl)methyl)-1H-indole (79A)

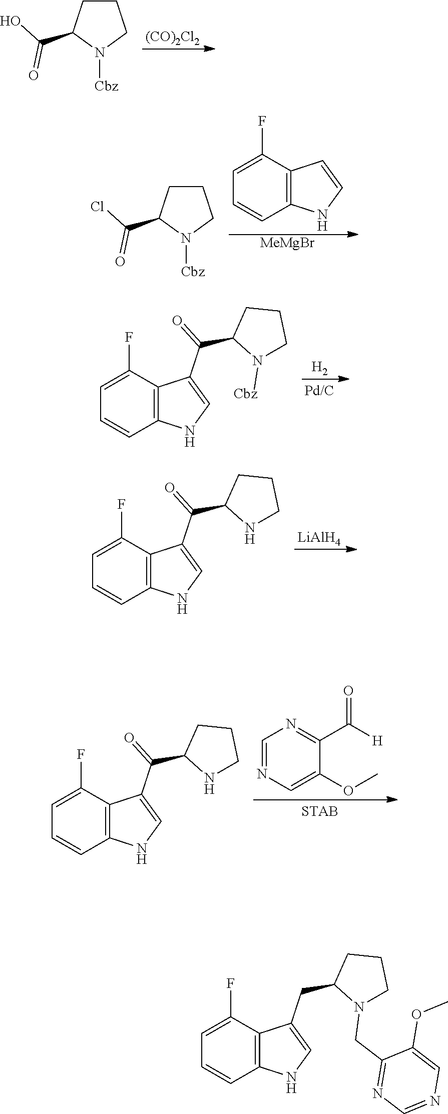

### Step 1: benzyl (R)-2-(chlorocarbonyl)pyrrolidine-1-carboxylate

To a stirred solution of ((benzyloxy)carbonyl)-D-proline (1.90 g, 1 Eq, 7.62 mmol) in dry DCM (53.0 mL) under a nitrogen atmosphere at 0 °C was added oxalyl chloride (2.90 g, 2.00 mL, 3 Eq, 22.9 mmol) and catalytic DMF. The reaction mixture was stirred at 0 °C for 3 hours. oxalyl chloride (2.90 g, 2.00 mL, 3 Eq, 22.9 mmol) was added and the mixture was stirred at 0 °C for 1 hour and then the solvent was removed under reduced pressure to afford the product (2.04 g, 7.62 mmol, 100%, 100% Purity) as a yellow oil.

m/z 264.2 (M+MeOH−Cl+H)+ (ES+)

### Step 2: benzyl (R)-2-(4-fluoro-1H-indole-3-carbonyl)pyrrolidine-1-carboxylate

To a stirred solution of 4-fluoro-1H-indole (2.00 g, 2 Eq, 14.8 mmol) in dry Toluene (100 mL) under a nitrogen atmosphere at 0 °C was added methylmagnesium bromide (1.76 g, 4.93 mL, 3.00 molar, 2 Eq, 14.8 mmol) dropwise over 10 min and the mixture was stirred at the same temperature for 3 hours. Freshly synthesised benzyl (R)-2-(chlorocarbonyl)pyrrolidine-1-carboxylate (1.98 g, 1 Eq, 7.40 mmol) in Toluene (10.0 mL) was added dropwise to the stirring solution of the deprotonated indole and the reaction mixture was stirred at room temperature for 16 hours.

The reaction mixture was quenched with sat. sodium bicarbonate (150 mL) at 0 °C and left to stir for 1 h, warming to room temperature. The two phases were separated and the aqueous phase was extracted with EtOAc (3×100 mL). The combined organic phases were washed with brine (50 mL), dried over MgSO₄, filtered and the solvent removed under reduced pressure. The crude product was purified by flash chromatography on silica gel (40 g column, 0-70% EtOAc in hexanes, load (silica)) to afford the product (1.66 g, 4.0 mmol, 54%, 88% Purity).

m/z 367.0 (M+H)+ (ES+)

### Step 3: (R)-4-fluoro-3-prolyl-1H-indole

To a mixture of benzyl (R)-2-(4-fluoro-1H-indole-3-carbonyl)pyrrolidine-1-carboxylate (1.66 g, 88% Wt, 1 Eq, 3.99 mmol) in EtOH (25.0 mL) was added palladium on carbon (166 mg, 0.391 Eq, 1.56 mmol) and the mixture was placed under a 1 bar hydrogen atmosphere. The mixture was stirred at room temperature for 16 hours. The reaction was vented and purged with nitrogen. The mixture was filtered to remove the Pd and the solvent removed under reduced pressure to afford (R)-4-fluoro-3-prolyl-1H-indole (0.920 g, 3.9 mmol, 98%, 99% Purity).

m/z 233.0 (M+H)+ (ES+)

### Step 4: (R)-4-fluoro-3-(pyrrolidin-2-ylmethyl)-1H-indole

To a mixture of (R)-4-fluoro-3-prolyl-1H-indole (0.940 g, 1 Eq, 4.05 mmol) in 2-Methyltetrahydrofuran (13.5 mL) at 0 °C was added lithium aluminum hydride (2.4 M in THF) (614 mg, 6.75 mL, 2.40 molar, 4 Eq, 16.2 mmol) dropwise. After for 10 min, the reaction was stirred at 66 °C for 5 hours and then at room temperature for 12 hours. The mixture was cooled down to 0 °C, diluted with THF (20 mL) and water (0.5 mL), 2.0 M NaOH (0.5 mL) and water (1.5 mL) were added followed by Na₂SO₄ (ca 2 g). The mixture was stirred for 45 min and filtered. The filtrated was concentrated under reduced pressure and the crude product purified by flash chromatography on silica gel (12 g column, 0-100% (20% (0.7 M in MeOH) in dichloromethane) in dichloromethane, liquid load (dichloromethane)) to afford the product (430 mg, 1.9 mmol, 46%, 94% Purity) after being triturated with MTBE.

m/z 219.2 (M+H)+ (ES+)

¹H NMR (500 MHz, DMSO) δ 1.26-1.37 (m, 1H), 1.52-1.73 (m, 3H), 2.65-2.80 (m, 2H), 2.81-2.91 (m, 2H), 3.19-3.26 (m, 1H), 6.64-6.70 (m, 1H), 6.95-7.01 (m, 1H), 7.14 (d, J=8.3 Hz, 2H), 11.04-11.08 (m, 1H)—NH missing due to a fast solvent exchange

¹⁹F NMR (471 MHz, DMSO) δ −124.47.

### Step 5: (R)-4-fluoro-3-((1-((5-methoxypyrimidin-4-yl)methyl)pyrrolidin-2-yl)methyl)-1H-indole

A mixture of (R)-4-fluoro-3-(pyrrolidin-2-ylmethyl)-1H-indole (35.0 mg, 1 Eq, 160 μmol) and 5-methoxypyrimidine-4-carbaldehyde (24.0 mg, 1.08 Eq, 174 μmol) in THF (1.50 mL) was stirred for 1 hour at room temperature. sodium triacetoxyborohydride (102 mg, 3 Eq, 481 μmol) was added and the mixture was stirred at room temperature for 16 hours.

The reaction was quenched with sat. aq. NaHCO₃ (10 mL) and the mixture was extracted with EtOAc (3×5 mL). The combined organic phases were washed with brine (5 mL), dried over MgSO₄, filtered and the solvent removed under reduced pressure. The crude product was purified by flash chromatography on silica gel (4 g column, 0-60% (10% (0.7 M NH3 in MeOH) in dichloromethane) in dichloromethane, liquid load (dichloromethane)) to afford the product (33.0 mg, 96 μmol, 60%, 99% Purity) as a light yellow oil.

m/z 341.2 (M+H)+ (ES+); m/z 339.2 (M−H)− (ES−).

¹H NMR (500 MHz, DMSO) δ 1.44-1.53 (m, 1H), 1.54-1.60 (m, 2H), 1.62-1.73 (m, 1H), 2.29-2.36 (m, 1H), 2.59 (dd, J=14.0, 9.3 Hz, 1H), 2.78-2.88 (m, 1H), 2.94-3.00 (m, 1H), 3.20-3.28 (m, 1H), 3.46 (d, J=13.4 Hz, 1H), 3.93 (s, 3H), 4.23 (d, J=13.4 Hz, 1H), 6.68 (dd, J=11.6, 7.7 Hz, 1H), 6.96-7.02 (m, 1H), 7.12-7.17 (m, 2H), 8.52 (s, 1H), 8.74 (s, 1H), 11.07 (s, 1H).

¹⁹F NMR (471 MHz, DMSO) δ −124.47.

### Example 12: Synthesis of (R)-4-fluoro-3-((1-((1-methyl-1H-pyrazol-4-yl)methyl)pyrrolidin-2-yl)methyl)-1H-indole (74A)

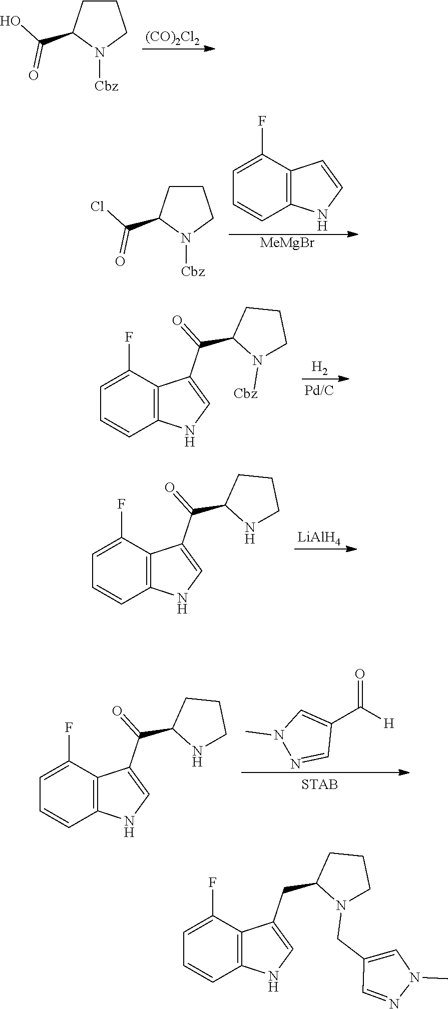

### Step 1: benzyl (R)-2-(chlorocarbonyl)pyrrolidine-1-carboxylate

To a stirred solution of ((benzyloxy)carbonyl)-D-proline (1.90 g, 1 Eq, 7.62 mmol) in dry DCM (53.0 mL) under a nitrogen atmosphere at 0 °C was added oxalyl chloride (2.90 g, 2.00 mL, 3 Eq, 22.9 mmol) and catalytic DMF. The reaction mixture was stirred at 0 °C for 3 hours. oxalyl chloride (2.90 g, 2.00 mL, 3 Eq, 22.9 mmol) was added and the mixture was stirred at 0 °C for 1 hour and then the solvent was removed under reduced pressure to afford the product (2.04 g, 7.62 mmol, 100%, 100% Purity) as a yellow oil.

m/z 264.2 (M+MeOH−Cl+H)+ (ES+)

### Step 2: benzyl (R)-2-(4-fluoro-1H-indole-3-carbonyl)pyrrolidine-1-carboxylate

To a stirred solution of 4-fluoro-1H-indole (2.00 g, 2 Eq, 14.8 mmol) in dry Toluene (100 mL) under a nitrogen atmosphere at 0 °C was added methylmagnesium bromide (1.76 g, 4.93 mL, 3.00 molar, 2 Eq, 14.8 mmol) dropwise over 10 min and the mixture was stirred at the same temperature for 3 hours. Freshly synthesised benzyl (R)-2-(chlorocarbonyl)pyrrolidine-1-carboxylate (1.98 g, 1 Eq, 7.40 mmol) in Toluene (10.0 mL) was added dropwise to the stirring solution of the deprotonated indole and the reaction mixture was stirred at room temperature for 16 hours.

The reaction mixture was quenched with sat. sodium bicarbonate (150 mL) at 0 °C and left to stir for 1 h, warming to room temperature. The two phases were separated and the aqueous phase was extracted with EtOAc (3×100 mL). The combined organic phases were washed with brine (50 mL), dried over MgSO₄, filtered and the solvent removed under reduced pressure. The crude product was purified by flash chromatography on silica gel (40 g column, 0-70% EtOAc in hexanes, load (silica)) to afford the product (1.66 g, 4.0 mmol, 54%, 88% Purity).

m/z 367.0 (M+H)+ (ES+)

### Step 3: (R)-4-fluoro-3-prolyl-1H-indole

To a mixture of benzyl (R)-2-(4-fluoro-1H-indole-3-carbonyl)pyrrolidine-1-carboxylate (1.66 g, 88% Wt, 1 Eq, 3.99 mmol) in EtOH (25.0 mL) was added palladium on carbon (166 mg, 0.391 Eq, 1.56 mmol) and the mixture was placed under a 1 bar hydrogen atmosphere. The mixture was stirred at room temperature for 16 hours. The reaction was vented and purged with nitrogen. The mixture was filtered to remove the Pd and the solvent removed under reduced pressure to afford (R)-4-fluoro-3-prolyl-1H-indole (0.920 g, 3.9 mmol, 98%, 99% Purity).

m/z 233.0 (M+H)+ (ES+)

### Step 4: (R)-4-fluoro-3-(pyrrolidin-2-ylmethyl)-1H-indole

To a mixture of (R)-4-fluoro-3-prolyl-1H-indole (0.940 g, 1 Eq, 4.05 mmol) in 2-Methyltetrahydrofuran (13.5 mL) at 0 °C was added lithium aluminum hydride (2.4 M in THF) (614 mg, 6.75 mL, 2.40 molar, 4 Eq, 16.2 mmol) dropwise. After for 10 min, the reaction was stirred at 66 °C for 5 hours and then at room temperature for 12 hours. The mixture was cooled down to 0 °C, diluted with THF (20 mL) and water (0.5 mL), 2.0 M NaOH (0.5 mL) and water (1.5 mL) were added followed by Na₂SO₄ (ca 2 g). The mixture was stirred for 45 min and filtered. The filtrated was concentrated under reduced pressure and the crude product purified by flash chromatography on silica gel (12 g column, 0-100% (20% (0.7 M in MeOH) in dichloromethane) in dichloromethane, liquid load (dichloromethane)) to afford the product (430 mg, 1.9 mmol, 46%, 94% Purity) after being triturated with MTBE.

m/z 219.2 (M+H)+ (ES+)

¹H NMR (500 MHz, DMSO) δ 1.26-1.37 (m, 1H), 1.52-1.73 (m, 3H), 2.65-2.80 (m, 2H), 2.81-2.91 (m, 2H), 3.19-3.26 (m, 1H), 6.64-6.70 (m, 1H), 6.95-7.01 (m, 1H), 7.14 (d, J=8.3 Hz, 2H), 11.04-11.08 (m, 1H)—NH missing due to a fast solvent exchange

¹⁹F NMR (471 MHz, DMSO) δ −124.47.

### Step 5: (R)-4-fluoro-3-((1-((1-methyl-1H-pyrazol-4-yl)methyl)pyrrolidin-2-yl)methyl)-1H-indole

A mixture of (R)-4-fluoro-3-(pyrrolidin-2-ylmethyl)-1H-indole (40.0 mg, 1 Eq, 183 μmol) and 1-methyl-1H-pyrazole-4-carbaldehyde (30.3 mg, 1.5 Eq, 275 μmol) in THF (2.50 mL) was stirred for 1 hour at room temperature. sodium triacetoxyborohydride (117 mg, 3 Eq, 550 μmol) was added and the mixture was stirred at room temperature for 16 hours.

The reaction was quenched with sat. aq. NaHCO₃ (10 mL) and the mixture was extracted with EtOAc (3×5 mL). The combined organic phases were washed with brine (5 mL), dried over MgSO₄, filtered and the solvent removed under reduced pressure. The crude product was purified by flash chromatography on silica gel (4 g column, 0-60% (10% (0.7 M NH3 in MeOH) in dichloromethane) in dichloromethane, liquid load (dichloromethane)) to afford the product (35.0 mg, 0.11 mmol, 59%, 97% Purity).

m/z 313.2 (M+H)+ (ES+); m/z 311.2 (M−H)− (ES−).

¹H NMR (500 MHz, DMSO) δ 1.44-1.67 (m, 4H), 2.08-2.17 (m, 1H), 2.56-2.67 (m, 2H), 2.82-2.89 (m, 1H), 3.17 (d, J=11.1 Hz, 1H), 3.23 (d, J=13.5 Hz, 1H), 3.78 (s, 3H), 3.85 (d, J=13.5 Hz, 1H), 6.69 (dd, J=11.7, 7.8 Hz, 1H), 6.95-7.03 (m, 1H), 7.13-7.18 (m, 2H), 7.27 (s, 1H), 7.52 (s, 1H), 11.07 (s, 1H).

¹⁹F NMR (471 MHz, DMSO) δ −124.30.

### Example 13: Synthesis of (R)-2-(2-((4-fluoro-1H-indol-3-yl)methyl)pyrrolidin-1-yl)ethan-1-ol (76A)

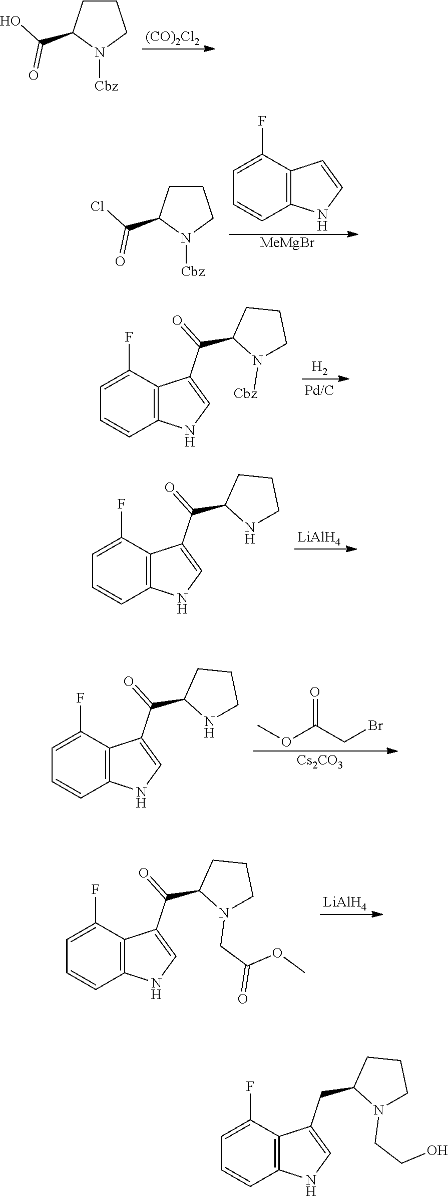

### Step 1: benzyl (R)-2-(chlorocarbonyl)pyrrolidine-1-carboxylate

To a stirred solution of ((benzyloxy)carbonyl)-D-proline (1.90 g, 1 Eq, 7.62 mmol) in dry DCM (53.0 mL) under a nitrogen atmosphere at 0 °C was added oxalyl chloride (2.90 g, 2.00 mL, 3 Eq, 22.9 mmol) and catalytic DMF. The reaction mixture was stirred at 0 °C for 3 hours. oxalyl chloride (2.90 g, 2.00 mL, 3 Eq, 22.9 mmol) was added and the mixture was stirred at 0 °C for 1 hour and then the solvent was removed under reduced pressure to afford the product (2.04 g, 7.62 mmol, 100%, 100% Purity) as a yellow oil.

m/z 264.2 (M+MeOH−Cl+H)+ (ES+)

### Step 2: benzyl (R)-2-(4-fluoro-1H-indole-3-carbonyl)pyrrolidine-1-carboxylate

To a stirred solution of 4-fluoro-1H-indole (2.00 g, 2 Eq, 14.8 mmol) in dry Toluene (100 mL) under a nitrogen atmosphere at 0 °C was added methylmagnesium bromide (1.76 g, 4.93 mL, 3.00 molar, 2 Eq, 14.8 mmol) dropwise over 10 min and the mixture was stirred at the same temperature for 3 hours. Freshly synthesised benzyl (R)-2-(chlorocarbonyl)pyrrolidine-1-carboxylate (1.98 g, 1 Eq, 7.40 mmol) in Toluene (10.0 mL) was added dropwise to the stirring solution of the deprotonated indole and the reaction mixture was stirred at room temperature for 16 hours.

The reaction mixture was quenched with sat. sodium bicarbonate (150 mL) at 0 °C and left to stir for 1 h, warming to room temperature. The two phases were separated and the aqueous phase was extracted with EtOAc (3×100 mL). The combined organic phases were washed with brine (50 mL), dried over MgSO₄, filtered and the solvent removed under reduced pressure. The crude product was purified by flash chromatography on silica gel (40 g column, 0-70% EtOAc in hexanes, load (silica)) to afford the product (1.66 g, 4.0 mmol, 54%, 88% Purity).

m/z 367.0 (M+H)+ (ES+)

### Step 3: (R)-4-fluoro-3-prolyl-1H-indole

To a mixture of benzyl (R)-2-(4-fluoro-1H-indole-3-carbonyl)pyrrolidine-1-carboxylate (1.66 g, 88% Wt, 1 Eq, 3.99 mmol) in EtOH (25.0 mL) was added palladium on carbon (166 mg, 0.391 Eq, 1.56 mmol) and the mixture was placed under a 1 bar hydrogen atmosphere. The mixture was stirred at room temperature for 16 hours. The reaction was vented and purged with nitrogen. The mixture was filtered to remove the Pd and the solvent removed under reduced pressure to afford (R)-4-fluoro-3-prolyl-1H-indole (0.920 g, 3.9 mmol, 98%, 99% Purity).

m/z 233.0 (M+H)+ (ES+)

### Step 4: (R)-4-fluoro-3-(pyrrolidin-2-ylmethyl)-1H-indole

To a mixture of (R)-4-fluoro-3-prolyl-1H-indole (0.940 g, 1 Eq, 4.05 mmol) in 2-Methyltetrahydrofuran (13.5 mL) at 0 °C was added lithium aluminum hydride (2.4 M in THF) (614 mg, 6.75 mL, 2.40 molar, 4 Eq, 16.2 mmol) dropwise. After for 10 min, the reaction was stirred at 66 °C for 5 hours and then at room temperature for 12 hours. The mixture was cooled down to 0 °C, diluted with THF (20 mL) and water (0.5 mL), 2.0 M NaOH (0.5 mL) and water (1.5 mL) were added followed by Na₂SO₄ (ca 2 g). The mixture was stirred for 45 min and filtered. The filtrated was concentrated under reduced pressure and the crude product purified by flash chromatography on silica gel (12 g column, 0-100% (20% (0.7 M in MeOH) in dichloromethane) in dichloromethane, liquid load (dichloromethane)) to afford the product (430 mg, 1.9 mmol, 46%, 94% Purity) after being triturated with MTBE.

m/z 219.2 (M+H)+ (ES+)

¹H NMR (500 MHz, DMSO) δ 1.26-1.37 (m, 1H), 1.52-1.73 (m, 3H), 2.65-2.80 (m, 2H), 2.81-2.91 (m, 2H), 3.19-3.26 (m, 1H), 6.64-6.70 (m, 1H), 6.95-7.01 (m, 1H), 7.14 (d, J=8.3 Hz, 2H), 11.04-11.08 (m, 1H)—NH missing due to a fast solvent exchange

¹⁹F NMR (471 MHz, DMSO) δ −124.47.

### Step 5: methyl (R)-2-(2-((4-fluoro-1H-indol-3-yl)methyl)pyrrolidin-1-yl)acetate

To a mixture of (R)-4-fluoro-3-(pyrrolidin-2-ylmethyl)-1H-indole (83.0 mg, 1 Eq, 380 μmol) and cesium carbonate (186 mg, 1.5 Eq, 570 μmol) in THF (2.00 mL) was added methyl bromoacetate (87.3 mg, 52.6 μL, 1.5 Eq, 570 μmol) and the mixture was stirred at room temperature for 16 hours. The mixture was diluted with water (20 mL) and extracted with EtOAc (10 mL). The aqueous phase was further extracted with EtOAc (2×10 mL) and the combined organic phases were washed with brine (10 mL), dried over MgSO₄, filtered and the solvent removed under reduced pressure.

The crude product was loaded onto a SCX resin cartridge and the cartridge was washed with MeOH (4 CV). The product was eluted with 0.7 M NH₃ in MeOH (4 CV), the solvent was removed under pressure to afford the product (84.0 mg, 0.26 mmol, 68%, 90% Purity) as a colourless oil.

m/z 291.0 (M+H)+ (ES+); m/z 289.0 (M−H)− (ES−).

¹⁹F NMR (471 MHz, DMSO) δ −124.63.

### Step 6: (R)-2-(2-((4-fluoro-1H-indol-3-yl)methyl)pyrrolidin-1-yl)ethan-1-ol

To a mixture of methyl (R)-2-(2-((4-fluoro-1H-indol-3-yl)methyl)pyrrolidin-1-yl)acetate (83.0 mg, 1 Eq, 286 μmol) in 2-Methyltetrahydrofuran (2.00 mL) at 0 °C was added lithium aluminum hydride (32.5 mg, 357 μL, 2.40 molar, 3 Eq, 858 μmol) dropwise and the mixture was stirred at the same temperature for 30 min and then the mixture was stirred at room temperature for 45 min. The reaction mixture was cooled to 0 °C, diluted with THF (10 mL) and quenched with H2O (1.5 mL), 2.0 M NaOH (0.5 mL) and Na₂SO₄ (ca 2 g). The mixture was stirred for 10 min before it was filtered and the organic solvent removed under reduced pressure. The crude product was purified by flash chromatography on silica gel (4 g column, 0-90% (10% (0.7 M NH3 in MeOH) in dichloromethane) in dichloromethane, liquid load (dichloromethane)) to afford the product (43.0 mg, 0.16 mmol, 56%, 98% Purity) as a colourless oil.

m/z 263.4 (M+H)+ (ES+); m/z 261.2 (M−H)− (ES−).

¹H NMR (500 MHz, DMSO) δ 1.39-1.53 (m, 1H), 1.53-1.67 (m, 3H), 2.12-2.20 (m, 1H), 2.25-2.33 (m, 1H), 2.59-2.72 (m, 1H), 2.95-3.04 (m, 1H), 3.06-3.13 (m, 2H), 3.43-3.57 (m, 2H), 4.40 (s, 1H), 6.68 (dd, J=11.6, 7.7 Hz, 1H), 6.95-7.03 (m, 1H), 7.12-7.17 (m, 2H), 11.06 (s, 1H)—1H under DMSO residual peak.

¹⁹F NMR (471 MHz, DMSO) δ −124.43.

### Example 14: Synthesis of (R)-4-fluoro-3-(pyrrolidin-2-ylmethyl)-1H-indole (81B)

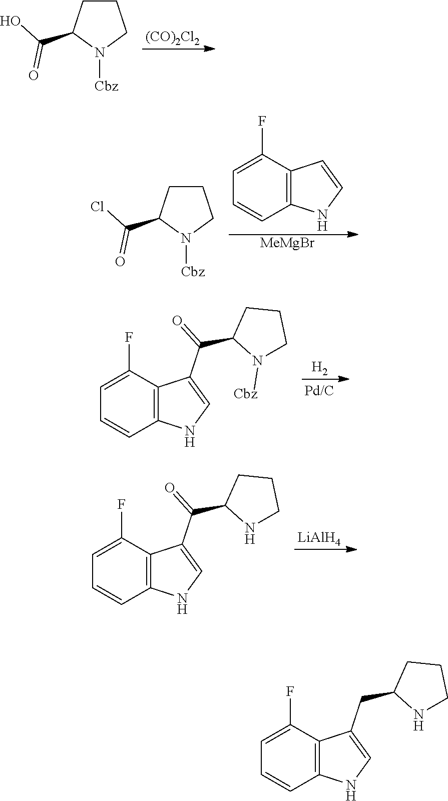

### Step 1: Benzyl (R)-2-(chlorocarbonyl)pyrrolidine-1-carboxylate

To a stirred solution of ((benzyloxy)carbonyl)-D-proline (1.90 g, 1 Eq, 7.62 mmol) in dry DCM (53.0 mL) under a nitrogen atmosphere at 0 °C was added oxalyl chloride (2.90 g, 2.00 mL, 3 Eq, 22.9 mmol) and catalytic DMF. The reaction mixture was stirred at 0 °C for 3 hours. oxalyl chloride (2.90 g, 2.00 mL, 3 Eq, 22.9 mmol) was added and the mixture was stirred at 0 °C for 1 hour and then the solvent was removed under reduced pressure to afford the product (2.04 g, 7.62 mmol, 100%, 100% Purity) as a yellow oil.

m/z 264.2 (M+MeOH−Cl+H)+ (ES+)

### Step 2: Benzyl (R)-2-(4-fluoro-1H-indole-3-carbonyl)pyrrolidine-1-carboxylate

To a stirred solution of 4-fluoro-1H-indole (2.00 g, 2 Eq, 14.8 mmol) in dry Toluene (100 mL) under a nitrogen atmosphere at 0 °C was added methylmagnesium bromide (1.76 g, 4.93 mL, 3.00 molar, 2 Eq, 14.8 mmol) dropwise over 10 min and the mixture was stirred at the same temperature for 3 hours. Freshly synthesised benzyl (R)-2-(chlorocarbonyl)pyrrolidine-1-carboxylate (1.98 g, 1 Eq, 7.40 mmol) in Toluene (10.0 mL) was added dropwise to the stirring solution of the deprotonated indole and the reaction mixture was stirred at room temperature for 16 hours.

The reaction mixture was quenched with sat. sodium bicarbonate (150 mL) at 0 °C and left to stir for 1 h, warming to room temperature. The two phases were separated and the aqueous phase was extracted with EtOAc (3×100 mL). The combined organic phases were washed with brine (50 mL), dried over MgSO₄, filtered and the solvent removed under reduced pressure. The crude product was purified by flash chromatography on silica gel (40 g column, 0-70% EtOAc in hexanes, load (silica)) to afford the product (1.66 g, 4.0 mmol, 54%, 88% Purity).

m/z 367.0 (M+H)+ (ES+)

### Step 3: (R)-4-fluoro-3-prolyl-1H-indole

To a mixture of benzyl (R)-2-(4-fluoro-1H-indole-3-carbonyl)pyrrolidine-1-carboxylate (1.66 g, 88% Wt, 1 Eq, 3.99 mmol) in EtOH (25.0 mL) was added palladium on carbon (166 mg, 0.391 Eq, 1.56 mmol) and the mixture was placed under a 1 bar hydrogen atmosphere. The mixture was stirred at room temperature for 16 hours. The reaction was vented and purged with nitrogen. The mixture was filtered to remove the Pd and the solvent removed under reduced pressure to afford (R)-4-fluoro-3-prolyl-1H-indole (0.920 g, 3.9 mmol, 98%, 99% Purity).

m/z 233.0 (M+H)+ (ES+)

### Step 4: (R)-4-fluoro-3-(pyrrolidin-2-ylmethyl)-1H-indole

To a mixture of (R)-4-fluoro-3-prolyl-1H-indole (0.940 g, 1 Eq, 4.05 mmol) in 2-Methyltetrahydrofuran (13.5 mL) at 0 °C was added lithium aluminum hydride (2.4 M in THF) (614 mg, 6.75 mL, 2.40 molar, 4 Eq, 16.2 mmol) dropwise. After for 10 min, the reaction was stirred at 66 °C for 5 hours and then at room temperature for 12 hours. The mixture was cooled down to 0 °C, diluted with THF (20 mL) and water (0.5 mL), 2.0 M NaOH (0.5 mL) and water (1.5 mL) were added followed by Na₂SO₄ (ca 2 g). The mixture was stirred for 45 min and filtered. The filtrated was concentrated under reduced pressure and the crude product purified by flash chromatography on silica gel (12 g column, 0-100% (20% (0.7 M in MeOH) in dichloromethane) in dichloromethane, liquid load (dichloromethane)) to afford the product (430 mg, 1.9 mmol, 46%, 94% Purity) after being triturated with MTBE.

m/z 219.2 (M+H)+ (ES+)

¹H NMR (500 MHz, DMSO) δ 1.26-1.37 (m, 1H), 1.52-1.73 (m, 3H), 2.65-2.80 (m, 2H), 2.81-2.91 (m, 2H), 3.19-3.26 (m, 1H), 6.64-6.70 (m, 1H), 6.95-7.01 (m, 1H), 7.14 (d, J=8.3 Hz, 2H), 11.04-11.08 (m, 1H)—NH missing due to a fast solvent exchange

¹⁹F NMR (471 MHz, DMSO) δ −124.47.

### Example 15: Synthesis of (R)-5-methoxy-3-((1-((1-methyl-1H-pyrazol-4-yl)methyl)pyrrolidin-2-yl)methyl)-1H-indole (34A)

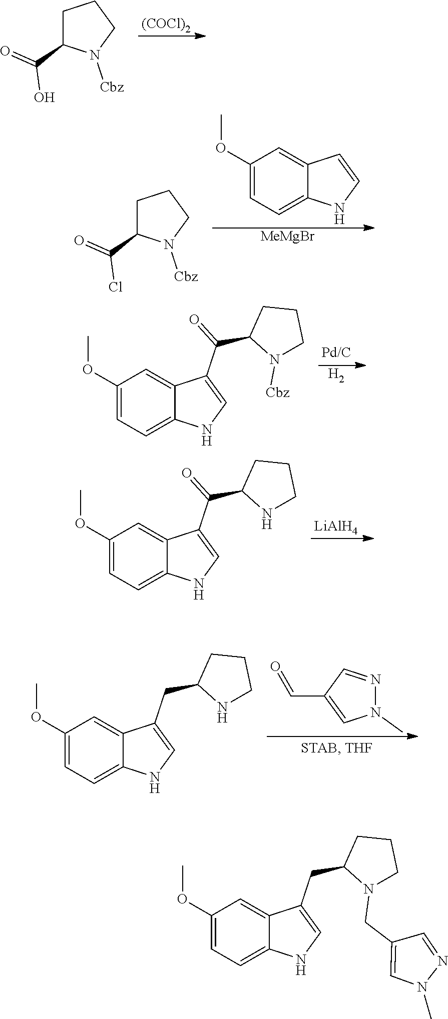

### Step 1: Benzyl (R)-2-(chlorocarbonyl)pyrrolidine-1-carboxylate

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h.

The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil.

m/z 264.2 (M−Cl+MeOH)+ (ES+)

### Step 2: Benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity).

m/z 401.4 (M+Na+H)+ (ES+); 377.3 (M−H)− (ES−)

¹H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%).

m/z 245.2 (M+H)+ (ES+); 243.2 (M−H)− (ES−)

1H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%).

m/z 231.2 (M+H)+ (ES+); 229.2 (M−H)− (ES−)

¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78.

### Step 5: (R)-5-methoxy-3-((1-((1-methyl-1H-pyrazol-4-yl)methyl)pyrrolidin-2-yl)methyl)-1H-indole

To a stirred solution of the product from step 4 above (50.0 mg, 1 Eq, 217 μmol) in dry THF (2.00 mL) at rt was added 1-methyl-1H-pyrazole-4-carbaldehyde (28.7 mg, 1.2 Eq, 261 μmol). The reaction mixture was stirred for 1 h. Sodium triacetoxyborohydride (138 mg, 3 Eq, 651 μmol) was added and the reaction mixture left to stir for 20 h at rt. The reaction mixture was quenched with sat. aq. NaHCO₃ (3 mL) and left to stir for 20 min. The aqueous suspension was diluted with water (5 mL) and extracted with EtOAc (3×10 mL). The combined organic layers were dried over Na₂SO₄, filtered and concentrated in vacuo.

The crude product was purified by chromatography on silica gel (12 g cartridge, 0-60% (1:3 EtOH/EtOAc+2% NH₄OH) in iso-hexane) to afford title compound (37.1 mg, 0.11 mmol, 50%) as a colourless gum.

m/z 325.0 (M+H)+ (ES+)

¹H NMR (500 MHz, DMSO) δ 10.59 (s, 1H), 7.55 (s, 1H), 7.32 (s, 1H), 7.20 (d, J=8.7 Hz, 1H), 7.09 (d, J=2.1 Hz, 1H), 6.94 (d, J=2.5 Hz, 1H), 6.69 (dd, J=8.7, 2.4 Hz, 1H), 3.87 (d, J=13.5 Hz, 1H), 3.79 (s, 3H), 3.75 (s, 3H), 3.32 (d, J=13.6 Hz, 1H), 3.02 (dd, J=14.0, 3.7 Hz, 1H), 2.90-2.84 (m, 1H), 2.66-2.58 (m, 1H), 2.54-2.46 (m, 1H), 2.21-2.14 (m, 1H), 1.70-1.61 (m, 1H), 1.61-1.40 (m, 3H). One proton obscured by DMSO peak, one proton obscured by H₂O peak, both observed on HSQC.

### Example 16: Synthesis of (R)-5-methoxy-3-((1-((1-methyl-1H-pyrazol-3-yl)methyl)pyrrolidin-2-yl)methyl)-1H-indole (36A)

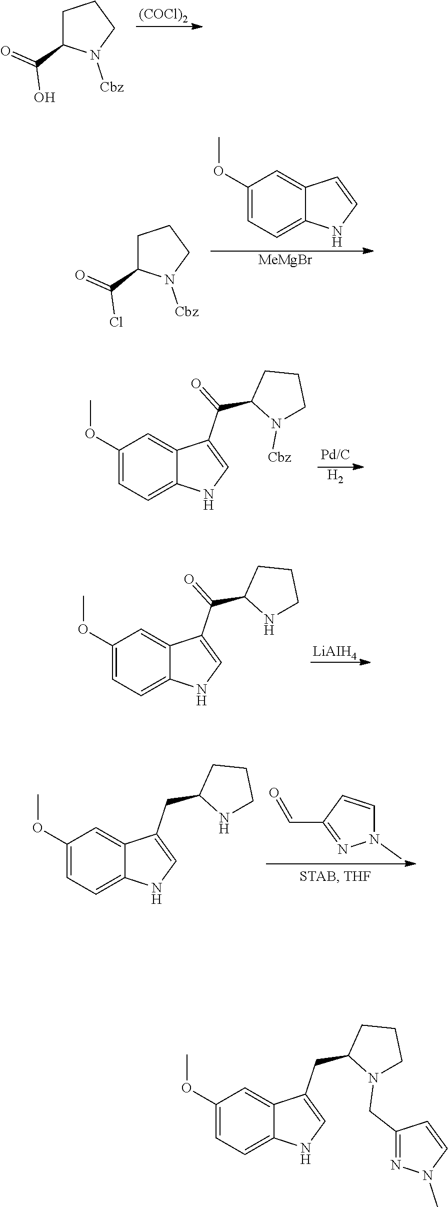

### Step 1: Benzyl (R)-2-(chlorocarbonyl)pyrrolidine-1-carboxylate

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h.

The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil.

m/z 264.2 (M−Cl+MeOH)+ (ES+)

### Step 2: benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity).

m/z 401.4 (M+Na+H)+ (ES+); 377.3 (M−H)− (ES−)

¹H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%).

m/z 245.2 (M+H)+ (ES+); 243.2 (M−H)− (ES−)

1H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%).

m/z 231.2 (M+H)+ (ES+); 229.2 (M−H)− (ES−)

¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78%

### Step 5: (R)-5-methoxy-3-((1-((1-methyl-1H-pyrazol-3-yl)methyl)pyrrolidin-2-yl)methyl)-1H-indole

To a stirred solution of the product from step 4 above (84.0 mg, 1 Eq, 237 μmol) in dry THF (3.00 mL) at rt was added 1-methyl-1H-pyrazole-3-carbaldehyde (33.0 mg, 1.2 Eq, 284 μmol). The reaction mixture was stirred for 1 h. Sodium triacetoxyborohydride (151 mg, 3 Eq, 711 μmol) was added and the reaction mixture left to stir for 20 h at rt. The reaction mixture was quenched with sat. aq. NaHCO₃ (5 mL) and left to stir for 20 min. The aqueous suspension was diluted with water (5 mL) and extracted with EtOAc (3×10 mL). The combined organic layers were dried over Na₂SO₄, filtered and concentrated in vacuo.

The crude product was dissolved in 1.7 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130A, 5 μm, 30 mm×100 mm, flow rate 40 mL min−1 eluting with a 0.3% Ammonia in water-MeCN gradient over 12.5 mins using UV across all wavelengths with PDA as well as a QDA and ELS detector. At-column dilution pump gives 2 mL min−1 Methanol over the entire method, which is included in the following MeCN percentages. Gradient information: 0.0-0.5 min, 20% MeCN; 0.5-10.5 min, ramped from 20% MeCN to 50% MeCN; 10.5-10.6 min, ramped from 50% MeCN to 100% MeCN; 10.6-12.5 min, held at 100% MeCN. The clean fractions were evaporated in a freeze-dryer.

The clean fractions were dissolved in MeCN and transferred into a vial before concentrating in vacuo to afford (R)-5-methoxy-3-((1-((1-methyl-1H-pyrazol-3-yl)methyl)pyrrolidin-2-yl)methyl)-1H-indole (34.2 mg, 103 μmol, 43.3%).

m/z 325.4 (M+H)+ (ES+)

¹H NMR (500 MHz, DMSO) δ 10.57 (s, 1H), 7.58 (d, J=2.1 Hz, 1H), 7.20 (d, J=8.7 Hz, 1H), 7.08 (d, J=2.1 Hz, 1H), 6.99 (d, J=2.4 Hz, 1H), 6.70 (dd, J=8.7, 2.4 Hz, 1H), 6.14 (d, J=2.2 Hz, 1H), 3.94 (d, J=13.4 Hz, 1H), 3.79 (s, 3H), 3.75 (s, 3H), 3.46 (d, J=13.4 Hz, 1H), 3.03 (dd, J=13.9, 3.6 Hz, 1H), 2.92-2.86 (m, 1H), 2.73-2.65 (m, 1H), 2.49-2.43 (m, 1H), 2.30-2.22 (m, 1H), 1.68-1.58 (m, 1H), 1.58-1.49 (m, 2H), 1.49-1.40 (m, 1H).

### Example 17: Synthesis of (R)-5-methoxy-3-((1-((5-methyl-1H-pyrazol-3-yl)methyl)pyrrolidin-2-yl)methyl)-1H-indole (38A)

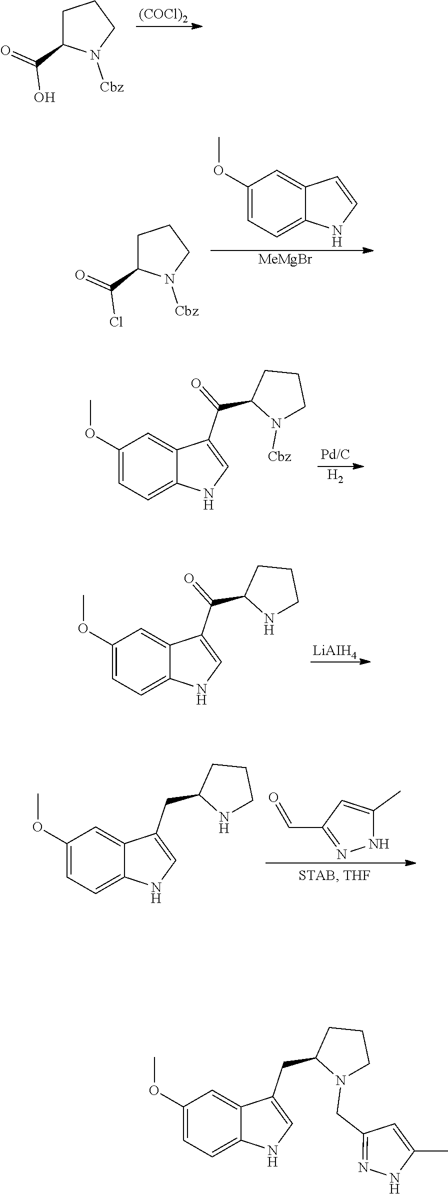

### Step 1: Benzyl (R)-2-(chlorocarbonyl)pyrrolidine-1-carboxylate (4235-103)

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h. The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil.

m/z 264.2 (M−Cl+MeOH)+ (ES+)

### Step 2: Benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity).

m/z 401.4 (M+Na+H)+ (ES+); 377.3 (M−H)− (ES−)

¹H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%).

m/z 245.2 (M+H)+ (ES+); 243.2 (M−H)− (ES−)

1H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%).

m/z 231.2 (M+H)+ (ES+); 229.2 (M−H)− (ES−)

¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78%

### Step 5: (R)-5-methoxy-3-((1-((5-methyl-1H-pyrazol-3-yl)methyl)pyrrolidin-2-yl)methyl)-1H-indole

To a stirred solution of (R)-5-methoxy-3-(pyrrolidin-2-ylmethyl)-1H-indole (84.0 mg, 1 Eq, 237 μmol) in dry THF (3.00 mL) at rt was added 5-methyl-1H-pyrazole-3-carbaldehyde (33.0 mg, 1.2 Eq, 284 μmol). The reaction mixture was stirred for 1 h. Sodium triacetoxyborohydride (151 mg, 3 Eq, 711 μmol) was added and the reaction mixture left to stir for 20 h at rt. The reaction mixture was quenched with sat. aq. NaHCO₃ (5 mL) and left to stir for 20 min. The aqueous suspension was diluted with water (5 mL) and extracted with EtOAc (3×10 mL). The combined organic layers were dried over Na₂SO₄, filtered and concentrated in vacuo.

The crude product was dissolved in 1.7 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130A, 5 μm, 30 mm×100 mm, flow rate 40 mL min−1 eluting with a 0.3% Ammonia in water-MeCN gradient over 12.5 mins using UV across all wavelengths with PDA as well as a QDA and ELS detector. At-column dilution pump gives 2 mL min−1 Methanol over the entire method, which is included in the following MeCN percentages. Gradient information: 0.0-0.5 min, 20% MeCN; 0.5-10.5 min, ramped from 20% MeCN to 50% MeCN; 10.5-10.6 min, ramped from 50% MeCN to 100% MeCN; 10.6-12.5 min, held at 100% MeCN. The clean fractions were evaporated in a Freeze-dryer. The clean fractions were dissolved in MeCN and transferred into a vial before concentrating in vacuo to afford (R)-5-methoxy-3-((1-((5-methyl-1H-pyrazol-3-yl)methyl)pyrrolidin-2-yl)methyl)-1H-indole (31.1 mg, 92.3 μmol, 38.9%) as a colourless foam.

m/z 325.3 (M+H)+ (ES+)

¹H NMR (500 MHz, DMSO) δ 12.15 (s, 1H), 10.58 (s, 1H), 7.19 (d, J=8.7 Hz, 1H), 7.09 (d, J=2.2 Hz, 1H), 6.98-6.94 (m, 1H), 6.69 (dd, J=8.7, 2.5 Hz, 1H), 5.92 (s, 1H), 3.93 (d, J=13.6 Hz, 1H), 3.75 (s, 3H), 3.47 (d, J=13.6 Hz, 1H), 3.02 (dd, J=14.1, 3.7 Hz, 1H), 2.92-2.85 (m, 1H), 2.69 (s, 1H), 2.48-2.43 (m, 1H), 2.33-2.22 (m, 1H), 2.17 (s, 3H), 1.69-1.49 (m, 3H), 1.49-1.38 (m, 1H).

### Example 18: Synthesis of (R)-5-methoxy-3-((1-((5-methoxypyrimidin-4-yl)methyl)pyrrolidin-2-yl)methyl)-1H-indole, Formic Acid (42A)

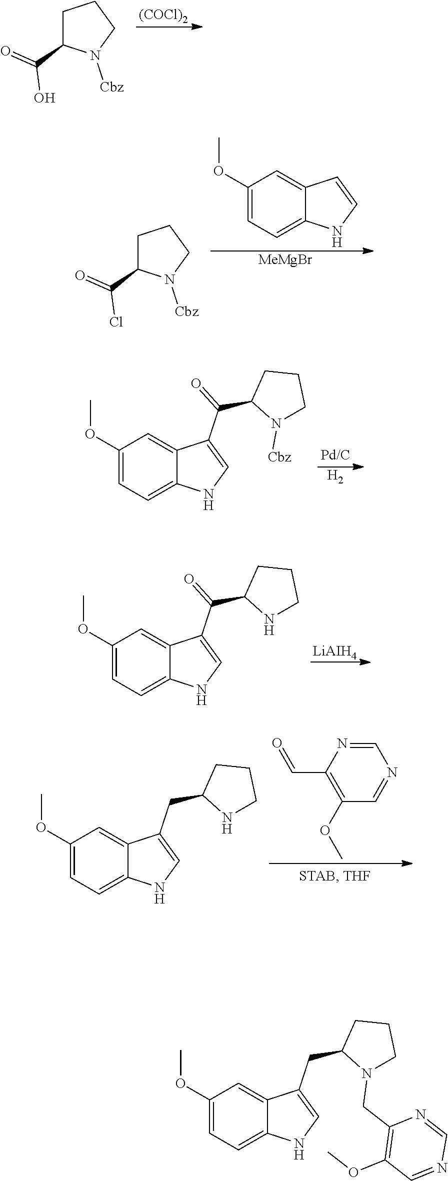

### Step 1: Benzyl (R)-2-(chlorocarbonyl)pyrrolidine-1-carboxylate

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h. The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil.

m/z 264.2 (M−Cl+MeOH)+ (ES+)

### Step 2: Benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity).

m/z 401.4 (M+Na+H)+ (ES+); 377.3 (M−H)− (ES−)

¹H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%).

m/z 245.2 (M+H)+ (ES+); 243.2 (M−H)− (ES−)

1H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%).

m/z 231.2 (M+H)+ (ES+); 229.2 (M−H)− (ES−)

¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78%

### Step 5: (R)-5-methoxy-3-((1-((5-methoxypyrimidin-4-yl)methyl)pyrrolidin-2-yl)methyl)-1H-indole, Formic Acid

To a stirred solution of the product from step 4 above (50.0 mg, 0.990 Eq, 215 μmol) in dry THF (2.00 mL) at rt was added 5-methoxypyrimidine-4-carbaldehyde (30.0 mg, 1 Eq, 217 μmol). The reaction mixture was stirred for 1 h. Sodium triacetoxyhydroborate (138 mg, 3 Eq, 652 μmol) was added and the reaction mixture left to stir for 20 h at rt. The reaction mixture was quenched with sat. aq. NaHCO₃ (3 mL) and left to stir for 20 min. The aqueous suspension was diluted with water (5 mL) and extracted with EtOAc (3×10 mL). The combined organic layers were dried over Na₂SO₄, filtered and concentrated in vacuo.

The crude product was purified by chromatography on silica gel (12 g cartridge, 0-70% (1:3 EtOH/EtOAc+2% NH₄OH) in iso-hexane). The crude product was re-purified by chromatography on RP Flash C18 (12 g cartridge, 0-10% (MeCN+0.1% formic acid) in (water+0.1% formic acid)) to afford (R)-5-methoxy-3-((1-((5-methoxypyrimidin-4-yl)methyl)pyrrolidin-2-yl)methyl)-1H-indole, Formic Acid (16.4 mg, 39.6 μmol, 18.2%).

m/z 353.2 (M+H)+ (ES+)

¹H NMR (500 MHz, DMSO) δ 10.59 (s, 1H), 8.76 (s, 1H), 8.54 (s, 1H), 8.16 (s, 1H), 7.20 (d, J=8.7 Hz, 1H), 7.10 (d, J=2.2 Hz, 1H), 7.00 (d, J=2.4 Hz, 1H), 6.70 (dd, J=8.7, 2.4 Hz, 1H), 4.22 (d, J=13.5 Hz, 1H), 3.93 (s, 3H), 3.74 (s, 3H), 3.61 (d, J=13.4 Hz, 1H), 3.06 (dd, J=14.3, 3.7 Hz, 1H), 3.03-2.95 (m, 1H), 2.91-2.84 (m, 1H), 2.56-2.52 (m, 1H), 2.37 (q, J=8.6 Hz, 1H), 1.74-1.62 (m, 1H), 1.62-1.52 (m, 2H), 1.50-1.40 (m, 1H). One exchangeable proton not observed.

### Example 19: Synthesis of (R)-2-(2-((5-methoxy-1H-indol-3-yl)methyl)pyrrolidin-1-yl)ethan-1-ol (37A)

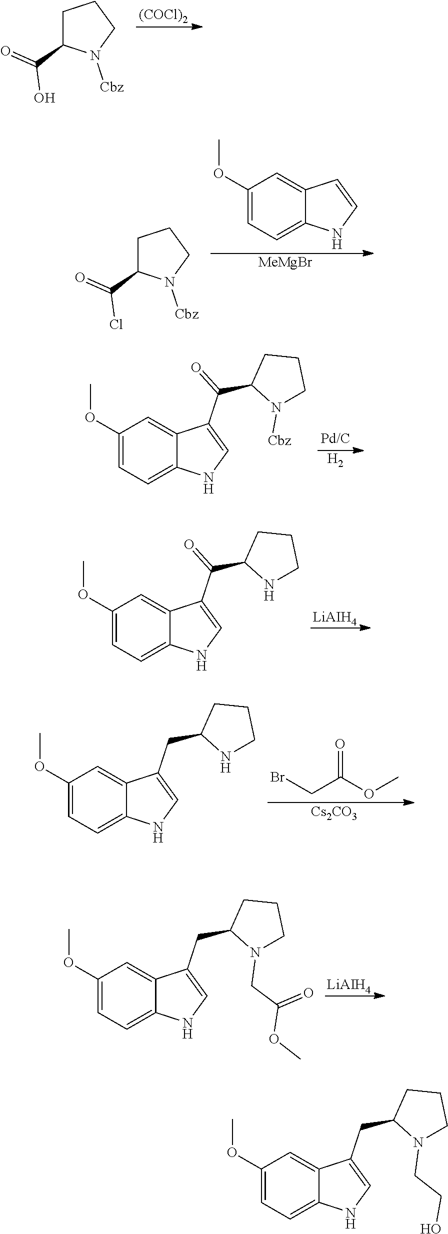

### Step 1: Benzyl (R)-2-(chlorocarbonyl)pyrrolidine-1-carboxylate (4235-103)

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h. The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil.

m/z 264.2 (M−Cl+MeOH)+ (ES+)

### Step 2: benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity).

m/z 401.4 (M+Na+H)+ (ES+); 377.3 (M−H)− (ES−)

¹H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%).

m/z 245.2 (M+H)+ (ES+); 243.2 (M−H)− (ES−)

1H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%).

m/z 231.2 (M+H)+ (ES+); 229.2 (M−H)− (ES−)

¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78%

### Step 5: methyl (R)-2-(2-((5-methoxy-1H-indol-3-yl)methyl)pyrrolidin-1-yl)acetate

To a stirred solution of the product from step 4 above (70.0 mg, 1 Eq, 304 μmol) and cesium carbonate (198 mg, 2 Eq, 608 μmol) in dry THF (3.00 mL) at rt was added methyl 2-bromoacetate (43.2 μL, 1.5 Eq, 456 μmol). The reaction mixture was stirred for 18 h.

The reaction mixture was quenched with sat. aq. NaHCO₃ (5 mL). The resulting aqueous suspension was extracted with EtOAc (2×10 mL). The collected organic layers were dried over Na₂SO₄, filtered and concentrated in vacuo.

The crude product was purified by chromatography on silica gel (12 g cartridge, 0-10% (0.7 M NH3/MeOH) in DCM) to afford the sub-titled product (71.8 mg, 0.22 mmol, 73%) as a colourless oil.

m/z 303.2 (M+H)+ (ES+)

¹H NMR (500 MHz, DMSO) δ 10.60 (s, 1H), 7.20 (d, J=8.7 Hz, 1H), 7.08 (d, J=2.3 Hz, 1H), 6.99 (d, J=2.4 Hz, 1H), 6.70 (dd, J=8.7, 2.4 Hz, 1H), 3.76 (s, 3H), 3.69 (d, J=16.6 Hz, 1H), 3.63 (s, 3H), 3.30 (d, J=16.6 Hz, 1H), 3.11-3.04 (m, 1H), 2.94 (dd, J=13.9, 3.9 Hz, 1H), 2.91-2.84 (m, 1H), 2.48-2.38 (m, 2H), 1.77-1.67 (m, 1H), 1.67-1.56 (m, 2H), 1.52-1.42 (m, 1H).

### Step 6: (R)-2-(2-((5-methoxy-1H-indol-3-yl)methyl)pyrrolidin-1-yl)ethan-1-ol

To a stirred suspension of the product from step 5 above (71.8 mg, 1 Eq, 221 μmol) in dry 2-Methyltetrahydrofuran (3.00 mL) under a nitrogen atmosphere at 0 °C was added lithium aluminum hydride (2.4 M in THF) (460 μL, 2.40 molar, 5 Eq, 1.10 mmol). The reaction mixture was heated to 60 °C and stirred for 30 min.

The mixture was cooled to 0 °C and quenched with 3:1 THF/water mixture (4 mL). Na₂SO₄ (5 g) was added to the suspension and stirred at rt for 1 h. The suspension was filtered through celite, washing with EtOAc (100 mL). The filtrate was concentrated in vacuo.

The crude product was purified by chromatography on silica gel (4 g cartridge, 0-10% (0.7 M NH₃/MeOH) in DCM) to afford (R)-2-(2-((5-methoxy-1H-indol-3-yl)methyl)pyrrolidin-1-yl)ethan-1-ol (30.9 mg, 0.11 mmol, 50%) as a pale orange gum.

m/z 275.0 (M+H)+ (ES+)

¹H NMR (500 MHz, DMSO) δ 10.59 (s, 1H), 7.20 (d, J=8.7 Hz, 1H), 7.08 (d, J=2.3 Hz, 1H), 6.97 (d, J=2.5 Hz, 1H), 6.69 (dd, J=8.7, 2.4 Hz, 1H), 4.41 (t, J=5.4 Hz, 1H), 3.75 (s, 3H), 3.56-3.49 (m, 2H), 3.11-3.05 (m, 1H), 3.02 (dt, J=12.0, 7.1 Hz, 1H), 2.95 (dd, J=14.1, 3.5 Hz, 1H), 2.62 (s, 1H), 2.48-2.42 (m, 1H), 2.32 (dt, J=12.1, 6.2 Hz, 1H), 2.16 (q, J=8.5 Hz, 1H), 1.70-1.54 (m, 3H), 1.46-1.39 (m, 1H).

### Example 20: Synthesis of (R)-4-fluoro-3-((1-((3-methoxypyridin-4-yl)methyl)pyrrolidin-2-yl)methyl)-1H-indole (77A)

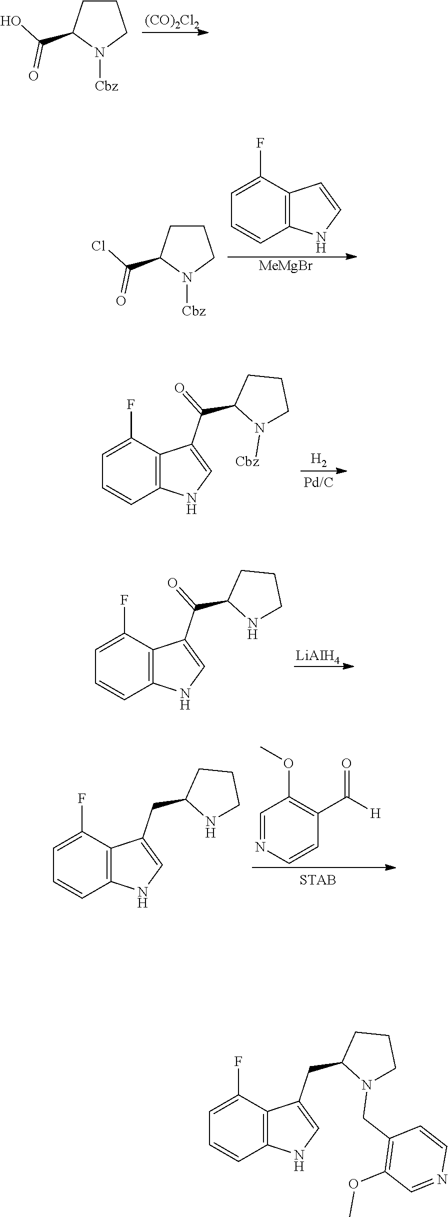

### Step 1: benzyl (R)-2-(chlorocarbonyl)pyrrolidine-1-carboxylate

To a stirred solution of ((benzyloxy)carbonyl)-D-proline (1.90 g, 1 Eq, 7.62 mmol) in dry DCM (53.0 mL) under a nitrogen atmosphere at 0 °C was added oxalyl chloride (2.90 g, 2.00 mL, 3 Eq, 22.9 mmol) and catalytic DMF. The reaction mixture was stirred at 0 °C for 3 hours. oxalyl chloride (2.90 g, 2.00 mL, 3 Eq, 22.9 mmol) was added and the mixture was stirred at 0 °C for 1 hour and then the solvent was removed under reduced pressure to afford the product (2.04 g, 7.62 mmol, 100%, 100% Purity) as a yellow oil.

m/z 264.2 (M+MeOH−Cl+H)+ (ES+)

### Step 2: benzyl (R)-2-(4-fluoro-1H-indole-3-carbonyl)pyrrolidine-1-carboxylate

To a stirred solution of 4-fluoro-1H-indole (2.00 g, 2 Eq, 14.8 mmol) in dry Toluene (100 mL) under a nitrogen atmosphere at 0 °C was added methylmagnesium bromide (1.76 g, 4.93 mL, 3.00 molar, 2 Eq, 14.8 mmol) dropwise over 10 min and the mixture was stirred at the same temperature for 3 hours. Freshly synthesised benzyl (R)-2-(chlorocarbonyl)pyrrolidine-1-carboxylate (1.98 g, 1 Eq, 7.40 mmol) in Toluene (10.0 mL) was added dropwise to the stirring solution of the deprotonated indole and the reaction mixture was stirred at room temperature for 16 hours.

The reaction mixture was quenched with sat. sodium bicarbonate (150 mL) at 0 °C and left to stir for 1 h, warming to room temperature. The two phases were separated and the aqueous phase was extracted with EtOAc (3×100 mL). The combined organic phases were washed with brine (50 mL), dried over MgSO₄, filtered and the solvent removed under reduced pressure. The crude product was purified by flash chromatography on silica gel (40 g column, 0-70% EtOAc in hexanes, load (silica)) to afford the product (1.66 g, 4.0 mmol, 54%, 88% Purity).

m/z 367.0 (M+H)+ (ES+)

### Step 3: (R)-4-fluoro-3-prolyl-1H-indole

To a mixture of benzyl (R)-2-(4-fluoro-1H-indole-3-carbonyl)pyrrolidine-1-carboxylate (1.66 g, 88% Wt, 1 Eq, 3.99 mmol) in EtOH (25.0 mL) was added palladium on carbon (166 mg, 0.391 Eq, 1.56 mmol) and the mixture was placed under a 1 bar hydrogen atmosphere. The mixture was stirred at room temperature for 16 hours. The reaction was vented and purged with nitrogen. The mixture was filtered to remove the Pd and the solvent removed under reduced pressure to afford (R)-4-fluoro-3-prolyl-1H-indole (0.920 g, 3.9 mmol, 98%, 99% Purity).

m/z 233.0 (M+H)+ (ES+)

### Step 4: (R)-4-fluoro-3-(pyrrolidin-2-ylmethyl)-1H-indole

To a mixture of (R)-4-fluoro-3-prolyl-1H-indole (0.940 g, 1 Eq, 4.05 mmol) in 2-Methyltetrahydrofuran (13.5 mL) at 0 °C was added lithium aluminum hydride (2.4 M in THF) (614 mg, 6.75 mL, 2.40 molar, 4 Eq, 16.2 mmol) dropwise. After for 10 min, the reaction was stirred at 66 °C for 5 hours and then at room temperature for 12 hours. The mixture was cooled down to 0 °C, diluted with THF (20 mL) and water (0.5 mL), 2.0 M NaOH (0.5 mL) and water (1.5 mL) were added followed by Na₂SO₄ (ca 2 g). The mixture was stirred for 45 min and filtered. The filtrated was concentrated under reduced pressure and the crude product purified by flash chromatography on silica gel (12 g column, 0-100% (20% (0.7 M in MeOH) in dichloromethane) in dichloromethane, liquid load (dichloromethane)) to afford the product (430 mg, 1.9 mmol, 46%, 94% Purity) after being triturated with MTBE.

m/z 219.2 (M+H)+ (ES+)

¹H NMR (500 MHz, DMSO) δ 1.26-1.37 (m, 1H), 1.52-1.73 (m, 3H), 2.65-2.80 (m, 2H), 2.81-2.91 (m, 2H), 3.19-3.26 (m, 1H), 6.64-6.70 (m, 1H), 6.95-7.01 (m, 1H), 7.14 (d, J=8.3 Hz, 2H), 11.04-11.08 (m, 1H)—NH missing due to a fast solvent exchange

¹⁹F NMR (471 MHz, DMSO) δ −124.47.

### Step 5: (R)-4-fluoro-3-((1-((3-methoxypyridin-4-yl)methyl)pyrrolidin-2-yl)methyl)-1H-indole

A mixture of (R)-4-fluoro-3-(pyrrolidin-2-ylmethyl)-1H-indole (45.0 mg, 85% Wt, 1 Eq, 175 μmol) and 3-methoxyisonicotinaldehyde (31.0 mg, 1.29 Eq, 226 μmol) in THF (1.50 mL) was stirred for 1 hour at room temperature. sodium triacetoxyborohydride (131 mg, 3.53 Eq, 618 μmol) was added and the mixture was stirred at room temperature for 16 hours.

The reaction was quenched with sat. aq. NaHCO₃ (10 mL) and the mixture was extracted with EtOAc (3×5 mL). The combined organic phases were washed with brine (5 mL), dried over MgSO₄, filtered and the solvent removed under reduced pressure. The crude product was purified by flash chromatography on silica gel (4 g column, 0-60% (10% (0.7 M NH3 in MeOH) in dichloromethane) in dichloromethane, liquid load (dichloromethane)) to afford the product that did not meet the purity criteria, thus it was dissolved in 1.4 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130A, 5 μm, 30 mm×100 mm, flow rate 40 mL min⁻¹ eluting with a 0.3% Ammonia in water-MeCN gradient over 17.5 mins using UV across all wavelengths with PDA as well as a QDA and ELS detector. At-column dilution pump gives 2 mL min⁻¹ Methanol over the entire method, which is included in the following MeCN percentages. Gradient information: 0.0-0.5 min, 30% MeCN; 0.5-15.5 min, ramped from 30% MeCN to 60% MeCN; 15.5-15.6 min, ramped from 60% MeCN to 100% MeCN; 15.6-17.5 min, held at 100% MeCN. The clean fractions were evaporated in a Freeze-dryer to afford (R)-4-fluoro-3-((1-((3-methoxypyridin-4-yl)methyl)pyrrolidin-2-yl)methyl)-1H-indole (9.00 mg, 26 μmol, 15%, 99% Purity).

m/z 340.2 (M+H)+ (ES+).

¹H NMR (500 MHz, DMSO) δ 1.50-1.75 (m, 2H), 2.09-2.18 (m, 1H), 2.62 (dd, J=13.9, 9.1 Hz, 1H), 2.77-2.85 (m, 1H), 2.90-2.97 (m, 1H), 3.15 (dd, J=14.0, 3.7 Hz, 1H), 3.44 (d, J=15.5 Hz, 1H), 3.89 (s, 3H), 3.98 (d, J=15.4 Hz, 1H), 6.68 (dd, J=11.6, 7.8 Hz, 1H), 6.95-7.03 (m, 1H), 7.12-7.17 (m, 2H), 7.39 (d, J=4.6 Hz, 1H), 8.18 (d, J=4.7 Hz, 1H), 8.29 (s, 1H), 11.07 (s, 1H)—2H under DMSO residual peak

¹⁹F NMR (471 MHz, DMSO) δ −124.40.

### Example 21: Synthesis of (R)-4-fluoro-3-((1-propylpyrrolidin-2-yl)methyl)-1H-indole (72A)

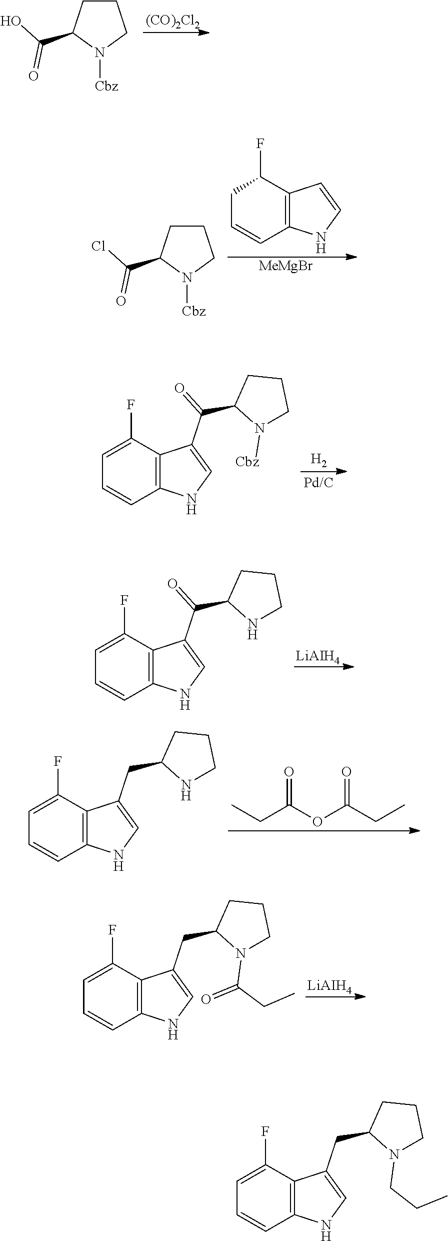

### Step 1: benzyl (R)-2-(chlorocarbonyl)pyrrolidine-1-carboxylate

To a stirred solution of ((benzyloxy)carbonyl)-D-proline (1.90 g, 1 Eq, 7.62 mmol) in dry DCM (53.0 mL) under a nitrogen atmosphere at 0 °C was added oxalyl chloride (2.90 g, 2.00 mL, 3 Eq, 22.9 mmol) and catalytic DMF. The reaction mixture was stirred at 0 °C for 3 hours. oxalyl chloride (2.90 g, 2.00 mL, 3 Eq, 22.9 mmol) was added and the mixture was stirred at 0 °C for 1 hour and then the solvent was removed under reduced pressure to afford the product (2.04 g, 7.62 mmol, 100%, 100% Purity) as a yellow oil.

m/z 264.2 (M+MeOH−Cl+H)+ (ES+)

### Step 2: benzyl (R)-2-(4-fluoro-1H-indole-3-carbonyl)pyrrolidine-1-carboxylate

To a stirred solution of 4-fluoro-1H-indole (2.00 g, 2 Eq, 14.8 mmol) in dry Toluene (100 mL) under a nitrogen atmosphere at 0 °C was added methylmagnesium bromide (1.76 g, 4.93 mL, 3.00 molar, 2 Eq, 14.8 mmol) dropwise over 10 min and the mixture was stirred at the same temperature for 3 hours. Freshly synthesised benzyl (R)-2-(chlorocarbonyl)pyrrolidine-1-carboxylate (1.98 g, 1 Eq, 7.40 mmol) in Toluene (10.0 mL) was added dropwise to the stirring solution of the deprotonated indole and the reaction mixture was stirred at room temperature for 16 hours.

The reaction mixture was quenched with sat. sodium bicarbonate (150 mL) at 0 °C and left to stir for 1 h, warming to room temperature. The two phases were separated and the aqueous phase was extracted with EtOAc (3×100 mL). The combined organic phases were washed with brine (50 mL), dried over MgSO₄, filtered and the solvent removed under reduced pressure. The crude product was purified by flash chromatography on silica gel (40 g column, 0-70% EtOAc in hexanes, load (silica)) to afford the product (1.66 g, 4.0 mmol, 54%, 88% Purity).

m/z 367.0 (M+H)+ (ES+)

### Step 3: (R)-4-fluoro-3-prolyl-1H-indole

To a mixture of benzyl (R)-2-(4-fluoro-1H-indole-3-carbonyl)pyrrolidine-1-carboxylate (1.66 g, 88% Wt, 1 Eq, 3.99 mmol) in EtOH (25.0 mL) was added palladium on carbon (166 mg, 0.391 Eq, 1.56 mmol) and the mixture was placed under a 1 bar hydrogen atmosphere. The mixture was stirred at room temperature for 16 hours. The reaction was vented and purged with nitrogen. The mixture was filtered to remove the Pd and the solvent removed under reduced pressure to afford (R)-4-fluoro-3-prolyl-1H-indole (0.920 g, 3.9 mmol, 98%, 99% Purity).

m/z 233.0 (M+H)+ (ES+)

### Step 4: (R)-4-fluoro-3-(pyrrolidin-2-ylmethyl)-1H-indole

To a mixture of (R)-4-fluoro-3-prolyl-1H-indole (0.940 g, 1 Eq, 4.05 mmol) in 2-Methyltetrahydrofuran (13.5 mL) at 0 °C was added lithium aluminum hydride (2.4 M in THF) (614 mg, 6.75 mL, 2.40 molar, 4 Eq, 16.2 mmol) dropwise. After for 10 min, the reaction was stirred at 66 °C for 5 hours and then at room temperature for 12 hours. The mixture was cooled down to 0 °C, diluted with THF (20 mL) and water (0.5 mL), 2.0 M NaOH (0.5 mL) and water (1.5 mL) were added followed by Na₂SO₄ (ca 2 g). The mixture was stirred for 45 min and filtered. The filtrated was concentrated under reduced pressure and the crude product purified by flash chromatography on silica gel (12 g column, 0-100% (20% (0.7 M in MeOH) in dichloromethane) in dichloromethane, liquid load (dichloromethane)) to afford the product (430 mg, 1.9 mmol, 46%, 94% Purity) after being triturated with MTBE.

m/z 219.2 (M+H)+ (ES+)

¹H NMR (500 MHz, DMSO) δ 1.26-1.37 (m, 1H), 1.52-1.73 (m, 3H), 2.65-2.80 (m, 2H), 2.81-2.91 (m, 2H), 3.19-3.26 (m, 1H), 6.64-6.70 (m, 1H), 6.95-7.01 (m, 1H), 7.14 (d, J=8.3 Hz, 2H), 11.04-11.08 (m, 1H)—NH missing due to a fast solvent exchange

¹⁹F NMR (471 MHz, DMSO) δ −124.47.

### Step 5: (R)-1-(2-((4-fluoro-1H-indol-3-yl)methyl)pyrrolidin-1-yl)propan-1-one

To a mixture of (R)-4-fluoro-3-(pyrrolidin-2-ylmethyl)-1H-indole (85.0 mg, 1 Eq, 389 μmol) and triethylamine (158 mg, 217 μL, 4 Eq, 1.56 mmol) in THF (4.00 mL) at 0 °C was added propionic anhydride (101 mg, 100 μL, 2 Eq, 779 μmol) and the mixture was stirred at room temperature for 16 hours. The reaction was quenched with water (10 mL) and the mixture extracted with EtOAc (3×10 mL). The combined organic phases were washed with brine (10 mL), dried over MgSO₄, filtered and the solvent removed under reduced pressure to afford (R)-1-(2-((4-fluoro-1H-indol-3-yl)methyl)pyrrolidin-1-yl)propan-1-one (111 mg, 0.36 mmol, 94%, 90% Purity) as a brown oil. Product was taken to the next step without further purification.

m/z 340.2 (M+H)+ (ES+).

### Step 6: (R)-4-fluoro-3-((1-propylpyrrolidin-2-yl)methyl)-1H-indole

To a mixture of (R)-1-(2-((4-fluoro-1H-indol-3-yl)methyl)pyrrolidin-1-yl)propan-1-one (0.111 g, 1 Eq, 405 μmol) in 2-Methyltetrahydrofuran (4.00 mL) at 0 °C was added lithium aluminum hydride (61.4 mg, 674 μL, 2.40 molar, 4 Eq, 1.62 mmol) dropwise and the mixture was stirred at 66 °C for 2 hours. The mixture was cooled to 0 °C, diluted with THF (10 mL) and quenched with water (1.5 mL) and 2 M NaOH (0.5 mL) and Na₂SO₄. The mixture was stirred at that temperature for 10 min and filtered and washed with THF. The filtrate was concentrated under reduced pressure and the crude product was dissolved in 2 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130A, 5 μm, 30 mm×100 mm, flow rate 40 mL min⁻¹ eluting with a 0.3% Ammonia in water-MeCN gradient over 17.5 mins using UV across all wavelengths with PDA as well as a QDA and ELS detector. At-column dilution pump gives 2 mL min⁻¹ Methanol over the entire method, which is included in the following MeCN percentages. Gradient information: 0.0-0.5 min, 40% MeCN; 0.5-15.5 min, ramped from 40% MeCN to 70% MeCN; 15.5-15.6 min, ramped from 70% MeCN to 100% MeCN; 15.6-17.5 min, held at 100% MeCN. The clean fractions were evaporated in a Freeze-dryer. The sample was freeze dried and the residue was dissolved in dichloromethane (2 mL) and washed with water (2 mL). The organic phase was dried over MgSO₄, filtered and the solvent removed under reduced pressure to afford (R)-4-fluoro-3-((1-propylpyrrolidin-2-yl)methyl)-1H-indole (28.0 mg, 0.10 mmol, 26%, 97% Purity).

m/z 261.2 (M+H)+ (ES+).

¹H NMR (500 MHz, DMSO) δ 0.89 (t, J=7.4 Hz, 3H), 1.38-1.68 (m, 4H), 2.03-2.19 (m, 2H), 2.52-2.59 (m, 1H), 2.76-2.85 (m, 1H), 3.02-3.14 (m, 2H), 6.68 (dd, J=11.6, 7.8 Hz, 1H), 6.95-7.02 (m, 1H), 7.06-7.24 (m, 2H), 11.06 (s, 1H). 2H under the DMSO residual peak.

¹⁹F NMR (471 MHz, DMSO) δ −124.37.

### Example 22: Synthesis of (R)-5-chloro-3-((1-(oxetan-3-yl)pyrrolidin-2-yl)methyl)-1H-indole (27A)

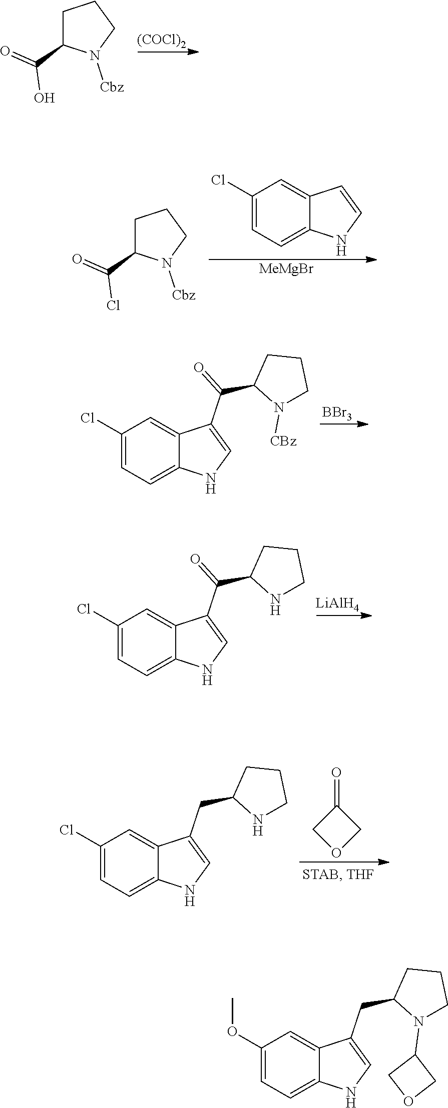

### Step 1: Benzyl (R)-2-(chlorocarbonyl)pyrrolidine-1-carboxylate

To a stirred solution of ((benzyloxy)carbonyl)-D-proline (1.70 g, 1 Eq, 6.83 mmol) in dry DCM (50 mL) under a nitrogen atmosphere at 0 °C was added oxalyl dichloride 2.60 g, 1.79 mL, 3 Eq, 20.5 mmol) and catalytic DMF. The reaction mixture was stirred at rt for 2.5 h, concentrated in vacuo and the resulting crude suspended in iso-hexane (3×20 mL), and concentrated in vacuo to afford sub-title compound (1.83 g, 6.5 mmol, 95%, 95% purity) as a pale-yellow oil. m/z 264.2 (M−Cl+MeOH)+ (ES+).

### Step 2: benzyl (R)-2-(5-chloro-1H-indole-3-carbonyl)pyrrolidine-1-carboxylate

To a stirred solution of 5-chloro-1H-indole (2.07 g, 2 Eq, 13.7 mmol) in dry Toluene (90 mL) under a nitrogen atmosphere at 0 °C was added MeMgBr in diethyl ether (1.63 g, 4.55 mL, 3.00 molar, 2 Eq, 13.7 mmol) dropwise over 5 min. The reaction mixture was stirred at rt for 2.5 h, solution of product from step 1 above (1.83 g, 0.95 Eq, 6.49 mmol) in dry Toluene (10.0 mL) was added dropwise and the reaction mixture was stirred for 18 h. The reaction mixture was quenched with sat. aq. NaHCO₃ (100 mL) at 0 °C and left to stir at rt for 30 min.

The resulting suspension was diluted with water (100 mL) and extracted with EtOAc (3×100 mL) and the collected organic layers were dried over MgSO₄, filtered and concentrated in vacuo. The crude product was purified by chromatography on silica gel (120 g cartridge, 0-75% EtOAc/iso-hexane). The resulting product was triturated with MTBE and filtered off to afford sub-title compound (1.09 g, 2.85 mmol, 24%, 100% Purity). m/z 383.00 (Cl³⁷), 381.00 (Cl³⁵) (M−H)− (ES−). ¹H NMR (500 MHz, DMSO) δ 12.22 (s, 1H), 12.19 (s, 1H), 8.54 (d, J=3.0 Hz, 1H), 8.50 (d, J=3.0 Hz, 1H), 8.18 (s, 1H), 8.15 (s, OH), 7.54-7.49 (m, 2H), 7.40-7.36 (m, 4H), 7.26 (td, J=6.7, 3.5 Hz, 2H), 7.12 (t, J=7.2 Hz, 1H), 7.07-6.98 (m, 4H), 5.24-5.13 (m, 2H), 5.11-5.02 (m, 2H), 5.01-4.86 (m, 2H), 3.58-3.47 (m, 4H), 2.35 (m, 2H), 1.93-1.79 (m, 6H). Product is present as a ~1:1 mixture of tautomers with the spectrum possessing multiple regions of overlapping. Therefore, spectrum has been quoted as a mixture of both species.

### Step 3: (R)-5-chloro-3-prolyl-1H-indole

To a solution of the product from step 2 above (1.09 g, 1 Eq, 2.85 mmol) in dry DCM (10.00 mL) under a nitrogen atmosphere at 0 °C was added BBr₃ in heptane (2.14 g, 8.54 mL, 1.00 molar, 3 Eq, 8.54 mmol) drop-wise and the resulting suspension was stirred for 30 min then warmed to rt and stirred for a further 4 h. The solution was cooled down to 0 °C, a further 1 eq. of BBr₃ in heptane (0.71 g, 2.85 mL, 1.00 molar) added, and the reaction mixture then warmed to rt and left to stir overnight. MeOH (5 mL) was added drop-wise and the mixture was stirred for 30 min. The mixture was loaded onto a cartridge of Si-TsOH (ca 5.0 g) and washed with MeOH (3×10 mL). The product was eluted with 0.7 M NH₃ in MeOH (4×10 mL) and concentrated under reduced pressure to sub-title compound (0.720 g, 2.8 mmol, 97%, 95% Purity). m/z 251.2 (Cl³⁷), 249.2 (Cl³⁵) (M+H)+ (ES+). ¹H NMR (500 MHz, DMSO) δ 8.56 (d, J=1.8 Hz, 1H), 8.15 (m, 1H), 7.54 (dd, J=8.7, 1.7 Hz, 1H), 7.28 (m, 1H), 4.78-4.71 (m, 1H), 3.21-3.17 (m, 1H), 3.08-2.98 (m, 1H), 2.38 (q, J=8.2 Hz, 1H), 1.90-1.82 (m, 1H), 1.82-1.72 (m, 2H). Note: 2 Exchangeable protons not observed.

### Step 4: (R)-5-chloro-3-(pyrrolidin-2-ylmethyl)-1H-indole

To a stirred suspension of the product from step 3 (0.720 g, 95% Wt, 1 Eq, 2.75 mmol) in dry tetrahydrofuran (18.0 mL) under a nitrogen atmosphere at 0 °C was added LiAlH₄ in THF (546 mg, 6.00 mL, 2.40 molar, 5.24 Eq, 14.4 mmol). The reaction mixture was heated to 60 °C and stirred for 3 h. Following this, the reaction was cooled to 0 °C, diluted with TBME (15 mL) then water (0.32 mL) added followed by 2 M aq NaOH (0.32 mL), water (1.00 mL) and MgSO₄. The mixture was warmed to rt and stirred for 30 min then filtered washing with EtOAc (2×10 mL) and the filtrate was concentrated in vacuo. The resulting product was dissolved in MeOH (ca 5 mL) and loaded onto a cartridge of Si-TsOH (ca 5 g) washing with MeOH (3×10 mL). The cartridge was eluted with 0.7 M NH₃/MeOH (3×10 mL) and concentrated under reduced pressure to afford sub-title compound (0.324 g, 0.95 mmol, 35%, 69% Purity). m/z 237.20 (Cl³⁷), 235.20 (Cl³⁵) (M+H)+ (ES+). ¹H NMR (500 MHz, DMSO) δ 10.98 (s, 1H), 7.55 (d, J=2.1 Hz, 1H), 7.33 (d, J=8.5 Hz, 1H), 7.24-7.21 (m, 1H), 7.04 (m, 1H), 3.22-3.15 (m, 1H), 2.91-2.83 (m, 1H), 2.68 (m, 2H), 1.76-1.61 (m, 2H), 1.61-1.51 (m, 3H), 1.32-1.21 (m, 1H). Exchangeable pyrrolidine NH not observed.

### Step 5: (R)-5-chloro-3-((1-(oxetan-3-yl)pyrrolidin-2-yl)methyl)-1H-indole

To a solution of the product from step 4 above (100 mg, 70% Wt, 1 Eq, 298 μmol) in dry THF (3.00 mL) at rt was added oxetan-3-one (32.9 mg, 29.3 μL, 98% Wt, 1.5 Eq, 447 μmol). The reaction mixture was stirred for 1.5 h, sodium triacetoxyborohydride (190 mg, 3 Eq, 895 μmol) added, and left to stir overnight. The reaction mixture was quenched with sat. sodium bicarbonate solution (5 mL) and left to stir for 1 h. The aqueous suspension was diluted with water (5 mL) and extracted with EtOAc (3×10 mL). The combined organic layers were dried over MgSO₄, filtered and concentrated in vacuo. The crude product was purified by chromatography on silica gel (12 g cartridge, 0-50% (1:3 EtOH/EtOAc+2% NH₄OH) in iso-hexane) to afford sub-title compound (56.01 mg, 0.18 mmol, 59%, 92% Purity).

Further purification was achieved by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130A, 5 μm, 30 mm×100 mm, flow rate 40 mL min−1 eluting with a 0.1% Formic acid in water-MeCN gradient over 12.5 mins using UV across all wavelengths with PDA as well as a QDA and ELS detector. At-column dilution pump gives 2 mL min−1 Methanol over the entire method, which is included in the following MeCN percentages. Gradient information: 0.0-0.5 min, 10% MeCN; 0.5-10.5 min, ramped from 10% MeCN to 32.5% MeCN; 10.5-10.6 min, ramped from 32.5% MeCN to 100% MeCN; 10.6-12.5 min, held at 100% MeCN. The clean fractions were evaporated in a Genevac, dissolved in DCM, treated with Aq. saturated sodium hydrogen carbonate solution, and the DCM layer passed through a phase separator to afford sub-title compound (26.51 mg, 90 μmol, 30%, 99% Purity). m/z 293.20 (Cl³⁷), 291.20 (Cl³⁵) (M+H)+ (ES+). ¹H NMR (500 MHz, DMSO) δ 11.00 (s, 1H), 7.50 (s, 1H), 7.33 (d, J=8.6 Hz, 1H), 7.23 (d, J=2.4 Hz, 1H), 7.04 (dd, J=8.7, 2.0 Hz, 1H), 4.61 (t, J=6.4 Hz, 2H), 4.57 (t, J=6.3 Hz, 1H), 4.52 (t, J=6.6 Hz, 1H), 3.98 (p, J=6.9 Hz, 1H), 2.96 (t, J=8.3 Hz, 1H), 2.78-2.68 (m, 2H), 2.48-2.44 (m, 1H), 2.37 (q, J=8.0 Hz, 1H), 1.70-1.57 (m, 3H), 1.46 (m, 1H).

The sample was dissolved in MeOH to 1 mg/mL and screened by SFC (Waters UPC², with a PDA and a QDA detector). The optimal conditions are using a Chiralpak IH column, 4.6×250 mm, 5 μm, with mobile phase 15% Methanol (28 mM NH₃), 85% CO₂. The flow rate was 4 mL/min. The run time was 9.0 mins. Sample is racemic.

### Example 23: Synthesis of (R)-2-(2-((5-chloro-1H-indol-3-yl)methyl)pyrrolidin-1-yl)ethan-1-ol (26A)

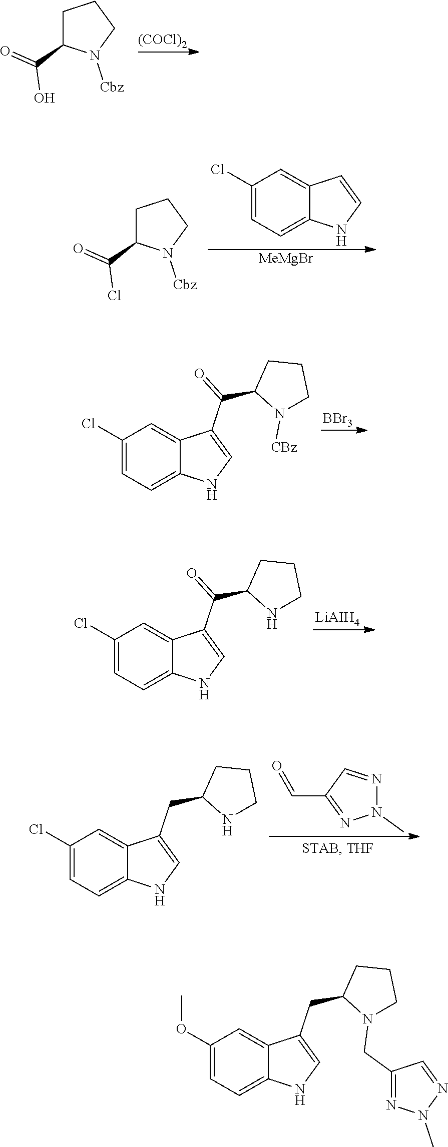

### Step 1: Benzyl (R)-2-(chlorocarbonyl)pyrrolidine-1-carboxylate

To a stirred solution of ((benzyloxy)carbonyl)-D-proline (1.70 g, 1 Eq, 6.83 mmol) in dry DCM (50 mL) under a nitrogen atmosphere at 0 °C was added oxalyl dichloride 2.60 g, 1.79 mL, 3 Eq, 20.5 mmol) and catalytic DMF. The reaction mixture was stirred at rt for 2.5 h, concentrated in vacuo and the resulting crude suspended in iso-hexane (3×20 mL), and concentrated in vacuo to afford sub-title compound (1.83 g, 6.5 mmol, 95%, 95% purity) as a pale-yellow oil. m/z 264.2 (M−Cl+MeOH)+ (ES+).

### Step 2: benzyl (R)-2-(5-chloro-1H-indole-3-carbonyl)pyrrolidine-1-carboxylate

To a stirred solution of 5-chloro-1H-indole (2.07 g, 2 Eq, 13.7 mmol) in dry Toluene (90 mL) under a nitrogen atmosphere at 0 °C was added MeMgBr in diethyl ether (1.63 g, 4.55 mL, 3.00 molar, 2 Eq, 13.7 mmol) dropwise over 5 min. The reaction mixture was stirred at rt for 2.5 h, solution of product from step 1 above (1.83 g, 0.95 Eq, 6.49 mmol) in dry Toluene (10.0 mL) was added dropwise and the reaction mixture was stirred for 18 h. The reaction mixture was quenched with sat. aq. NaHCO₃ (100 mL) at 0 °C and left to stir at rt for 30 min. The resulting suspension was diluted with water (100 mL) and extracted with EtOAc (3×100 mL) and the collected organic layers were dried over MgSO₄, filtered and concentrated in vacuo. The crude product was purified by chromatography on silica gel (120 g cartridge, 0-75% EtOAc/iso-hexane). The resulting product was triturated with MTBE and filtered off to afford sub-title compound (1.09 g, 2.85 mmol, 24%, 100% Purity). m/z 383.00 (Cl³⁷), 381.00 (Cl³⁵) (M−H)− (ES−). ¹H NMR (500 MHz, DMSO) δ 12.22 (s, 1H), 12.19 (s, 1H), 8.54 (d, J=3.0 Hz, 1H), 8.50 (d, J=3.0 Hz, 1H), 8.18 (s, 1H), 8.15 (s, OH), 7.54-7.49 (m, 2H), 7.40-7.36 (m, 4H), 7.26 (td, J=6.7, 3.5 Hz, 2H), 7.12 (t, J=7.2 Hz, 1H), 7.07-6.98 (m, 4H), 5.24-5.13 (m, 2H), 5.11-5.02 (m, 2H), 5.01-4.86 (m, 2H), 3.58-3.47 (m, 4H), 2.35 (m, 2H), 1.93-1.79 (m, 6H). Product is present as a ~1:1 mixture of tautomers with the spectrum possessing multiple regions of overlapping. Therefore, spectrum has been quoted as a mixture of both species.

### Step 3: (R)-5-chloro-3-prolyl-1H-indole

To a solution of the product from step 2 above (1.09 g, 1 Eq, 2.85 mmol) in dry DCM (10.00 mL) under a nitrogen atmosphere at 0 °C was added BBr₃ in heptane (2.14 g, 8.54 mL, 1.00 molar, 3 Eq, 8.54 mmol) drop-wise and the resulting suspension was stirred for 30 min then warmed to rt and stirred for a further 4 h. The solution was cooled down to 0 °C, a further 1 eq. of BBr₃ in heptane (0.71 g, 2.85 mL, 1.00 molar) added, and the reaction mixture then warmed to rt and left to stir overnight. MeOH (5 mL) was added drop-wise and the mixture was stirred for 30 min. The mixture was loaded onto a cartridge of Si-TsOH (ca 5.0 g) and washed with MeOH (3×10 mL). The product was eluted with 0.7 M NH₃ in MeOH (4×10 mL) and concentrated under reduced pressure to sub-title compound (0.720 g, 2.8 mmol, 97%, 95% Purity). m/z 251.2 (Cl³⁷), 249.2 (Cl³⁵) (M+H)+ (ES+). ¹H NMR (500 MHz, DMSO) δ 8.56 (d, J=1.8 Hz, 1H), 8.15 (m, 1H), 7.54 (dd, J=8.7, 1.7 Hz, 1H), 7.28 (m, 1H), 4.78-4.71 (m, 1H), 3.21-3.17 (m, 1H), 3.08-2.98 (m, 1H), 2.38 (q, J=8.2 Hz, 1H), 1.90-1.82 (m, 1H), 1.82-1.72 (m, 2H). 2 Exchangeable protons not observed.

The sample was dissolved in MeOH to 1 mg/mL and screened by SFC (Waters UPC², with a PDA and a QDA detector). The optimal conditions are using a Chiralpak IH column, 4.6×250 mm, 5 μm, with mobile phase 15% Methanol (28 mM NH₃), 85% CO₂. The flow rate was 4 mL/min. The run time was 9.0 mins. Sample is racemic.

### Step 4: (R)-5-chloro-3-(pyrrolidin-2-ylmethyl)-1H-indole

To a stirred suspension of the product from step 3 (0.720 g, 95% Wt, 1 Eq, 2.75 mmol) in dry tetrahydrofuran (18.0 mL) under a nitrogen atmosphere at 0 °C was added LiAlH₄ in THF (546 mg, 6.00 mL, 2.40 molar, 5.24 Eq, 14.4 mmol). The reaction mixture was heated to 60 °C and stirred for 3 h. Following this, the reaction was cooled to 0 °C, diluted with TBME (15 mL) then water (0.32 mL) added followed by 2 M aq NaOH (0.32 mL), water (1.00 mL) and MgSO₄. The mixture was warmed to rt and stirred for 30 min then filtered washing with EtOAc (2×10 mL) and the filtrate was concentrated in vacuo. The resulting product was dissolved in MeOH (ca 5 mL) and loaded onto a cartridge of Si-TsOH (ca 5 g) washing with MeOH (3×10 mL). The cartridge was eluted with 0.7 M NH₃/MeOH (3×10 mL) and concentrated under reduced pressure to afford sub-title compound (0.324 g, 0.95 mmol, 35%, 69% Purity). m/z 237.20 (Cl³⁷), 235.20 (Cl³⁵) (M+H)+ (ES+). ¹H NMR (500 MHz, DMSO) δ 10.98 (s, 1H), 7.55 (d, J=2.1 Hz, 1H), 7.33 (d, J=8.5 Hz, 1H), 7.24-7.21 (m, 1H), 7.04 (m, 1H), 3.22-3.15 (m, 1H), 2.91-2.83 (m, 1H), 2.68 (m, 2H), 1.76-1.61 (m, 2H), 1.61-1.51 (m, 3H), 1.32-1.21 (m, 1H). Exchangeable pyrrolidine NH not observed.

### Step 5: (R)-2-(2-((5-chloro-1H-indol-3-yl)methyl)pyrrolidin-1-yl)ethan-1-ol

To a solution of the product from step 4 above (100 mg, 70% Wt, 1 Eq, 298 μmol) in dry THF (3.00 mL) at rt was added 2-methyl-2H-1,2,3-triazole-4-carbaldehyde (33.1 mg, 1 Eq, 298 μmol). The reaction mixture was stirred for 1.5 h, sodium triacetoxyborohydride (190 mg, 3 Eq, 895 μmol) added, and then left to stir overnight. The reaction mixture was quenched with sat. sodium bicarbonate solution (5 mL) and left to stir. The aqueous suspension was diluted with water (5 mL) and extracted with EtOAc (3×10 mL). The combined organic layers were dried over MgSO₄, filtered and concentrated in vacuo. The crude product was purified by chromatography on silica gel (12 g cartridge, 0-50% (1:3 EtOH/EtOAc+2% NH₄OH) in iso-hexane) to afford sub-title compound (51.38 mg, 0.14 mmol, 47%, 90% Purity) as an oil.

Further purification was achieved by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130A, 5 μm, 30 mm×100 mm, flow rate 40 mL min−1 eluting with a 0.3% Ammonia in water-MeCN gradient over 12.5 mins using UV across all wavelengths with PDA as well as a QDA and ELS detector. At-column dilution pump gives 2 mL min−1 Methanol over the entire method, which is included in the following MeCN percentages. Gradient information: 0.0-0.5 min, 32.5% MeCN; 0.5-10.5 min, ramped from 32.5% MeCN to 62.5% MeCN; 10.5-10.6 min, ramped from 62.5% MeCN to 100% MeCN; 10.6-12.5 min, held at 100% MeCN. The clean fractions were evaporated in a Genevac to afford sub-title compound (29.3 mg, 88 μmol, 29%, 99% Purity). m/z 333.20 (Cl³⁷), 330.20 (Cl³⁵) (M+H)+ (ES+). ¹H NMR (500 MHz, DMSO) δ 10.98 (s, 1H), 7.61 (s, 1H), 7.57 (d, J=2.1 Hz, 1H), 7.33 (d, J=8.6 Hz, 1H), 7.24 (s, 1H), 7.04 (dd, J=8.6, 2.0 Hz, 1H), 4.14 (s, 3H), 4.04 (d, J=14.0 Hz, 1H), 3.63 (d, J=14.0 Hz, 1H), 3.05 (dd, J=13.9, 3.7 Hz, 1H), 2.90-2.87 (m, 1H), 2.70-2.63 (m, 1H), 2.55-2.52 (m, 1H), 2.26 (q, J=8.5 Hz, 1H), 1.66-1.39 (m, 4H).

### Example 24: Synthesis of (R)-3-((1-((2H-1,2,3-triazol-4-yl)methyl)pyrrolidin-2-yl)methyl)-5-methoxy-1H-indole (31A)

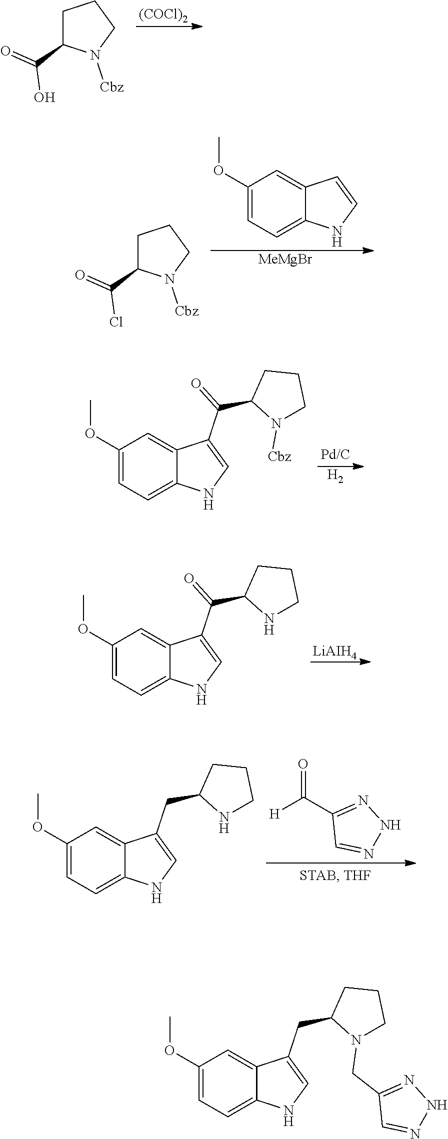

### Step 1: Benzyl (R)-2-(chlorocarbonyl)pyrrolidine-1-carboxylate

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h.

The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil. m/z 264.2 (M−Cl+MeOH)⁺ (ES⁺).

### Step 2: Benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity). m/z 401.4 (M+Na+H)+ (ES+); 377.3 (M−H)− (ES−). ¹H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%). m/z 245.2 (M+H)+ (ES+); 243.2 (M−H)− (ES−). 1H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%). m/z 231.2 (M+H)+ (ES+); 229.2 (M−H)− (ES−). ¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78%.

### Step 5: (R)-3-((1-((2H-1,2,3-triazol-4-yl)methyl)pyrrolidin-2-yl)methyl)-5-methoxy-1H-indole

A mixture of (R)-5-methoxy-3-(pyrrolidin-2-ylmethyl)-1H-indole (84.0 mg, 1 Eq, 365 μmol) and in THF (2.00 mL) was stirred at room temperature for 30 min and sodium triacetoxyborohydride (232 mg, 3 Eq, 1.09 mmol) was added and the mixture was stirred at room temperature for 16 hours. The reaction was quenched with 0.7 M NH₃ in MeOH (5 mL) and the solvent was removed under reduced pressure. The crude product was purified by flash chromatography on silica gel (4 g column, 0-70% (10% (0.7 M NH3 in MeOH) in dichloromethane, in dichloromethane, liquid load (dichloromethane)). m/z 312.2 (M+H)+ (ES+). ¹H NMR (500 MHz, DMSO) δ 1.39-1.69 (m, 3H), 2.21-2.33 (m, 1H), 2.67-2.78 (m, 1H), 2.84-2.94 (m, 1H), 2.99-3.07 (m, 1H), 3.14-3.19 (m, 2H), 3.63-3.70 (m, 1H), 3.76 (s, 3H), 4.09 (d, J=14.1 Hz, 1H), 6.69 (dd, J=8.8, 2.4 Hz, 1H), 6.98 (s, 1H), 7.09 (s, 1H), 7.19 (d, J=8.7 Hz, 1H), 7.68 (s, 1H), 10.59 (s, 1H)—NH triazole missing.

### Example 25: Synthesis of (R)-3-((1-((1H-1,2,4-triazol-5-yl)methyl)pyrrolidin-2-yl)methyl)-5-methoxy-1H-indole (33A)

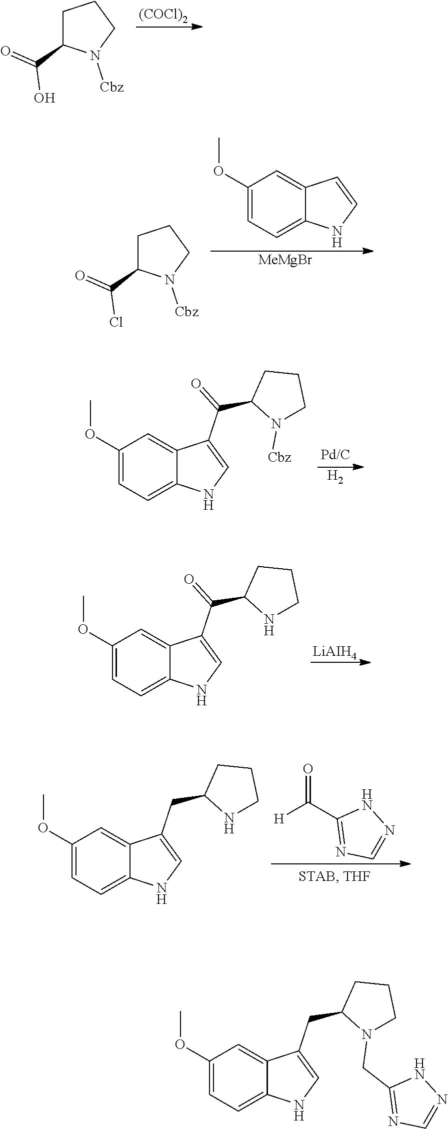

### Step 1: Benzyl (R)-2-(chlorocarbonyl)pyrrolidine-1-carboxylate

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h. The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil. m/z 264.2 (M−Cl+MeOH)+ (ES+).

### Step 2: Benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity). m/z 401.4 (M+Na+H)+ (ES+); 377.3 (M−H)− (ES−). ¹H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%). m/z 245.2 (M+H)+ (ES+); 243.2 (M−H)− (ES−). ¹H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%). m/z 231.2 (M+H)+ (ES+); 229.2 (M−H)− (ES−). ¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78%.

### Step 5: (R)-3-((1-((1H-1,2,4-triazol-5-yl)methyl)pyrrolidin-2-yl)methyl)-5-methoxy-1H-indole

A mixture of (R)-5-methoxy-3-(pyrrolidin-2-ylmethyl)-1H-indole (84.0 mg, 1 Eq, 365 μmol) and 1H-1,2,4-triazole-5-carbaldehyde (42.5 mg, 1.2 Eq, 438 μmol) in THF was stirred at room temperature for 30 min and REACTANT was added and the mixture was stirred at room temperature for 16 hours. The reaction was quenched with 0.7 M NH₃ in MeOH (5 mL) and the solvent was removed under reduced pressure. The crude product was purified by flash chromatography on silica gel (4 g column, 0-70% (10% (0.7 M NH3 in MeOH) in dichloromethane, in dichloromethane, liquid load (dichloromethane)) to afford (R)-3-((1-((1H-1,2,4-triazol-5-yl)methyl)pyrrolidin-2-yl)methyl)-5-methoxy-1H-indole (55.0 mg, 0.16 mmol, 45%, 92% Purity). m/z 312.1 (M+H)+ (ES+). ¹H NMR (500 MHz, DMSO) δ 1.38-1.69 (m, 4H), 2.33-2.46 (m, 2H), 2.74-2.86 (m, 1H), 2.88-2.98 (m, 1H), 2.98-3.08 (m, 1H), 3.71-3.84 (m, 4H), 4.00-4.14 (m, 1H), 6.68 (dd, J=8.7, 2.4 Hz, 1H), 7.03 (s, 1H), 7.09 (d, J=2.1 Hz, 1H), 7.19 (d, J=8.7 Hz, 1H), 7.88 (s, 1H), 10.58 (s, 1H), 13.

### Example 26: Synthesis of (R)-3-((1-((1H-pyrazol-4-yl)methyl)pyrrolidin-2-yl)methyl)-5-methoxy-1H-indole (35A)

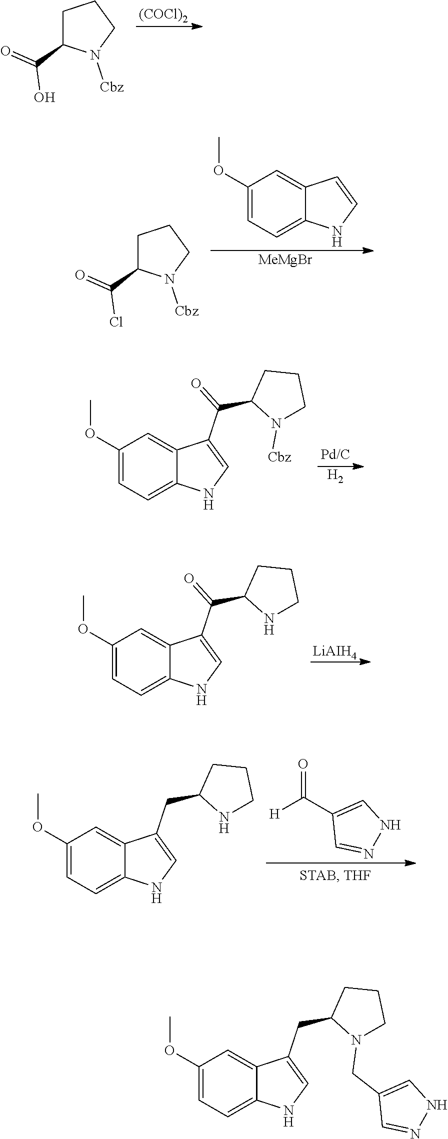

### Step 1: Benzyl (R)-2-(chlorocarbonyl)pyrrolidine-1-carboxylate

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h. The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil. m/z 264.2 (M−Cl+MeOH)⁺ (ES⁺).

### Step 2: Benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity). m/z 401.4 (M+Na+H)⁺ (ES+); 377.3 (M−H)− (ES−). ¹H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%). m/z 245.2 (M+H)+ (ES+); 243.2 (M−H)− (ES−). ¹H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%). m/z 231.2 (M+H)+ (ES+); 229.2 (M−H)− (ES−). ¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78%

### Step 5: (R)-3-((1-((1H-pyrazol-4-yl)methyl)pyrrolidin-2-yl)methyl)-5-methoxy-1H-indole

A mixture of (R)-5-methoxy-3-(pyrrolidin-2-ylmethyl)-1H-indole (84.0 mg, 1 Eq, 365 μmol) and 1H-pyrazole-4-carbaldehyde (42.1 mg, 1.2 Eq, 438 μmol) in THF (2.00 mL) was stirred at room temperature for 30 min and sodium triacetoxyborohydride (232 mg, 3 Eq, 1.09 mmol) was added and the mixture was stirred at room temperature for 16 hours. The reaction was quenched with 0.7 M NH₃ in MeOH (5 mL) and the solvent was removed under reduced pressure. The crude product was purified by flash chromatography on silica gel (4 g column, 0-70% (10% (0.7 M NH3 in MeOH) in dichloromethane, in dichloromethane, liquid load (dichloromethane)) to afford (R)-3-((1-((1H-pyrazol-4-yl)methyl)pyrrolidin-2-yl)methyl)-5-methoxy-1H-indole (36.0 mg, 0.11 mmol, 30%, 94% Purity). m/z 311.1 (M+H)+ (ES+). ¹H NMR (500 MHz, DMSO) δ 1.40-1.70 (m, 4H), 2.13-2.26 (m, 1H), 2.58-2.73 (m, 1H), 2.82-2.96 (m, 1H), 3.04 (d, J=13.8 Hz, 1H), 3.17 (d, J=5.4 Hz, 1H), 3.38 (s, 1H), 3.75 (s, 3H), 3.92 (d, J=13.5 Hz, 1H), 6.69 (dd, J=8.7, 2.5 Hz, 1H), 6.93 (d, J=2.4 Hz, 1H), 7.10 (s, 1H), 7.20 (d, J=8.7 Hz, 1H), 7.39-7.64 (m, 2H), 10.60 (s, 1H)—NH pyrazole missing.

### Example 27: Synthesis of (R)-5-methoxy-3-((1-((2-methoxypyridin-3-yl)methyl)pyrrolidin-2-yl)methyl)-1H-indole (44A)

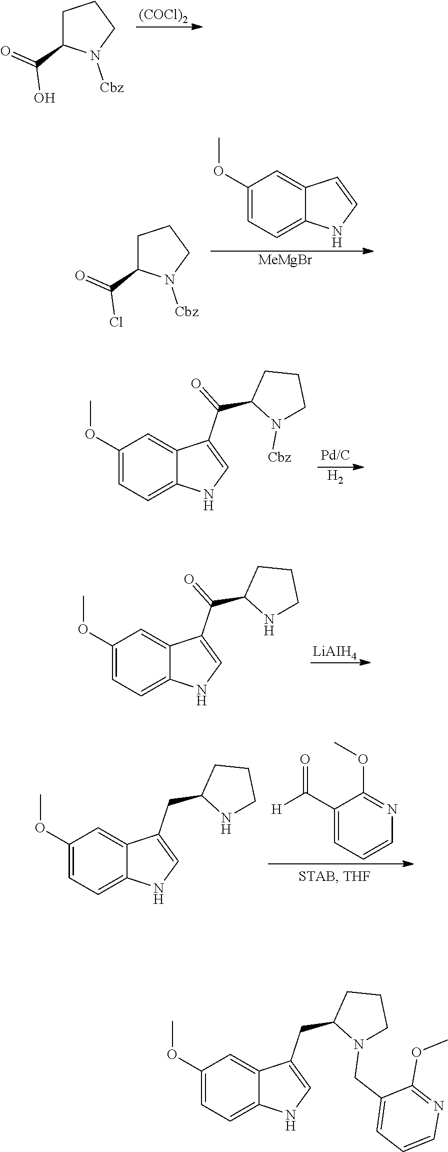

### Step 1: Benzyl (R)-2-(chlorocarbonyl)pyrrolidine-1-carboxylate

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h. The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil. m/z 264.2 (M−Cl+MeOH)⁺ (ES⁺).

### Step 2: Benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity). m/z 401.4 (M+Na+H)+ (ES+); 377.3 (M−H)− (ES−). ¹H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%). m/z 245.2 (M+H)+ (ES+); 243.2 (M−H)− (ES−). ¹H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%). m/z 231.2 (M+H)+ (ES+); 229.2 (M−H)− (ES−). ¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78%.

### Step 5: (R)-5-methoxy-3-((1-((2-methoxypyridin-3-yl)methyl)pyrrolidin-2-yl)methyl)-1H-indole

A mixture of (R)-5-methoxy-3-(pyrrolidin-2-ylmethyl)-1H-indole (84.0 mg, 1 Eq, 365 μmol) and 2-methoxynicotinaldehyde (60.0 mg, 1.2 Eq, 438 μmol) in THF (2.00 mL) was stirred at room temperature for 30 min and sodium triacetoxyborohydride (232 mg, 3 Eq, 1.09 mmol) was added and the mixture was stirred at room temperature for 16 hours. The reaction was quenched with 0.7 M NH3 in MeOH (5 mL) and the solvent was removed under reduced pressure. The crude product was purified by flash chromatography on silica gel (4 g column, 0-70% (10% (0.7 M NH3 in MeOH) in dichloromethane, in dichloromethane, liquid load (dichloromethane)) to afford (R)-5-methoxy-3-((1-((2-methoxypyridin-3-yl)methyl)pyrrolidin-2-yl)methyl)-1H-indole (43.0 mg, 0.12 mmol, 32%, 96% Purity) as a colourless oil. m/z 352.1 (M+H)+ (ES+). ¹H NMR (500 MHz, DMSO) δ 1.45-1.67 (m, 3H), 1.68-1.79 (m, 1H), 2.12-2.21 (m, 1H), 2.56 (dd, J=14.2, 9.0 Hz, 1H), 2.74-2.83 (m, 1H), 2.85-2.96 (m, 1H), 3.02 (dd, J=14.1, 3.8 Hz, 1H), 3.41 (d, J=14.6 Hz, 1H), 3.73 (s, 3H), 3.87 (s, 3H), 4.01 (d, J=14.5 Hz, 1H), 6.69 (dd, J=8.7, 2.4 Hz, 1H), 6.95 (d, J=2.4 Hz, 1H), 6.98 (dd, J=7.2, 4.9 Hz, 1H), 7.10 (d, J=2.3 Hz, 1H), 7.20 (d, J=8.6 Hz, 1H), 7.73 (dd, J=7.3, 1.9 Hz, 1H), 8.05 (dd, J=4.8, 1.9 Hz, 1H), 10.60 (s, 1H).

### Example 28: Synthesis of (R)-3-((1-(2-Fluoroethyl)pyrrolidin-2-yl)methyl)-5-methoxy-1H-indole (49A)

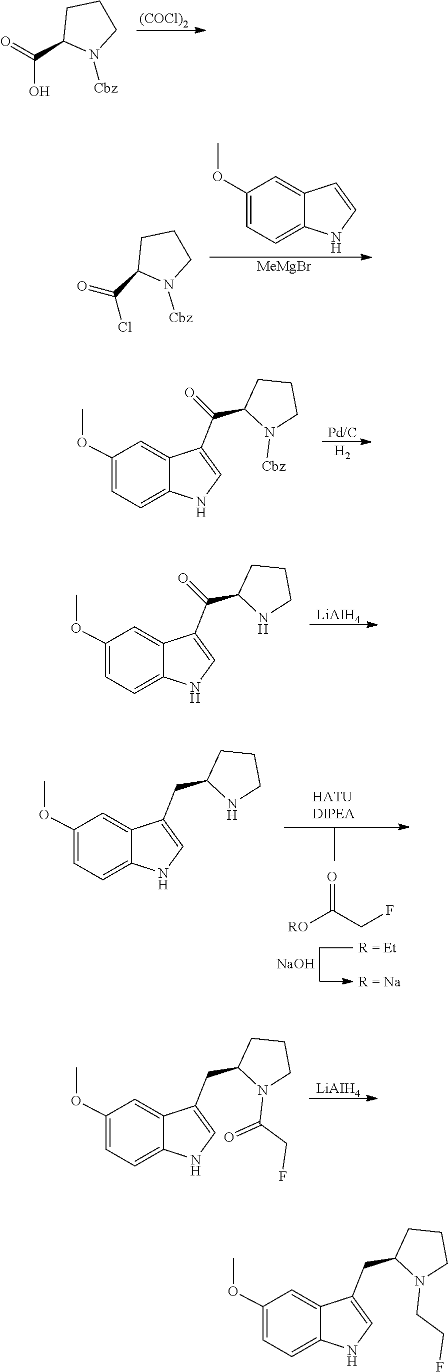

### Step 1: Benzyl (R)-2-(chlorocarbonyl)pyrrolidine-1-carboxylate

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h. The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil. m/z 264.2 (M−Cl+MeOH)⁺ (ES⁺).

### Step 2: Benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity). m/z 401.4 (M+Na+H)⁺ (ES+); 377.3 (M−H)⁻ (ES−). ¹H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-Methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%). m/z 245.2 (M+H)+ (ES+); 243.2 (M−H)− (ES−). ¹H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-Methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%). m/z 231.2 (M+H)+ (ES+); 229.2 (M−H)− (ES−). ¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78%.

### Step 5: Sodium 2-fluoroacetate

To a stirred solution of ethyl 2-fluoroacetate (500 mg, 455 μL, 1 Eq, 4.71 mmol) in EtOH (5.00 mL) at 0 °C was added NaOH (207 mg, 1.1 Eq, 5.18 mmol). The reaction mixture was stirred for 18 h, after which isohexane (30 mL) was added. The reaction was cooled to 0 °C and filtered to give the sub-title compound (391 mg, 3.9 mmol, 82%). ¹H NMR (500 MHz, DMSO) δ 4.34 (d, J=1.9 Hz, 1H), 4.24 (s, 1H). ¹⁹F NMR (471 MHz, DMSO) δ −210.70-−210.82 (m)

### Step 6: (R)-2-Fluoro-1-(2-((5-methoxy-1H-indol-3-yl)methyl)pyrrolidin-1-yl)ethan-1-one

To a stirred solution of the product from step 4 above (200 mg, 93% Wt, 1 Eq, 808 μmol) in dry DCM (5.00 mL) under a nitrogen atmosphere was added HATU (461 mg, 1.5 Eq, 1.21 mmol), DIPEA (313 mg, 422 μL, 3 Eq, 2.42 mmol) and the product from step 5 above (121 mg, 1.5 Eq, 1.21 mmol). The reaction mixture was stirred over the weekend at rt. The reaction mixture was diluted DCM (20 mL), and the organic phase washed with 1M HCl (20 mL), sat. NaHCO₃ (20 mL) and brine (20 mL). The organic phase was dried (Na₂SO₄), filtered and concentrated in vacuo. The crude product was purified by chromatography on silica gel (12 g cartridge, 0-100% (2% NH₄OH in 3:1 EtOAc/EtOH) in hexane) to afford the sub-title compound (168 mg, 0.50 mmol, 62%). m/z 290.0 (M+H)+ (ES+).

### Step 7: (R)-3-((1-(2-Fluoroethyl)pyrrolidin-2-yl)methyl)-5-methoxy-1H-indole

To a stirred suspension of the product from step 6 above (168 mg, 87% Wt, 1 Eq, 503 μmol) in dry 2-Me-THF (5.00 mL) under a nitrogen atmosphere at 0 °C was added LiAlH₄ (2.4 M in THF) (76.4 mg, 839 μL, 2.40 molar, 4 Eq, 2.01 mmol). The reaction mixture was heated to 60 °C and stirred for 1 h. The mixture was cooled to 0 °C and quenched with 3:1 THF/water mixture (4 mL). Na₂SO₄ (5 g) was added to the suspension and stirred at rt for 1 h. The suspension was filtered through celite, washing with EtOAc (~100 mL). The filtrate was filtered and concentrated in vacuo. The crude material was dissolved in DMSO (1.9 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130A, 5 μm, 30 mm×100 mm, flow rate 40 mL min−1 eluting with a 0.3% ammonia in water-MeCN gradient over 17.5 mins using UV across all wavelengths with PDA as well as a QDA and ELS detector. At-column dilution pump gives 2 mL min−1 MeOH over the entire method, which is included in the following MeCN percentages. Gradient information: 0.0-0.5 min, 22.5% MeCN; 0.5-15.5 min, ramped from 22. 5% MeCN to 52.5% MeCN; 15.5-15.6 min, ramped from 52.5% MeCN to 100% MeCN; 15.6-17.5 min, held at 100% MeCN. The clean fractions were evaporated in a freeze-dryer. The fractions were re-concentrated in vacuo from MeCN to afford the title compound (11.5 mg, 38.9 μmol, 7.72%) as an orange gum. m/z 277.0 (M+H)⁺ (ES+); 275.0 (M−H)⁻ (ES−). ¹H NMR (500 MHz, DMSO) δ 10.63-10.55 (m, 1H), 7.20 (d, J=8.7 Hz, 1H), 7.08 (d, J=2.2 Hz, 1H), 6.98 (d, J=2.4 Hz, 1H), 6.69 (dd, J=8.7, 2.4 Hz, 1H), 4.64-4.55 (m, 1H), 4.55-4.46 (m, 1H), 3.75 (s, 3H), 3.27-3.14 (m, 1H), 3.14-3.08 (m, 1H), 2.95 (dd, J=14.2, 3.7 Hz, 1H), 2.68 (qd, J=7.1, 3.5 Hz, 1H), 2.61-2.51 (m, 1H), 2.49-2.44 (m, 1H), 2.22 (q, J=8.6 Hz, 1H), 1.72-1.55 (m, 3H), 1.49-1.42 (m, 1H). ¹⁹F NMR (471 MHz, DMSO) δ −217.52.

### Example 29: Synthesis of (R)-5-(methylthio)-3-((1-(oxetan-3-yl)pyrrolidin-2-yl)methyl)-1H-indole (27A)

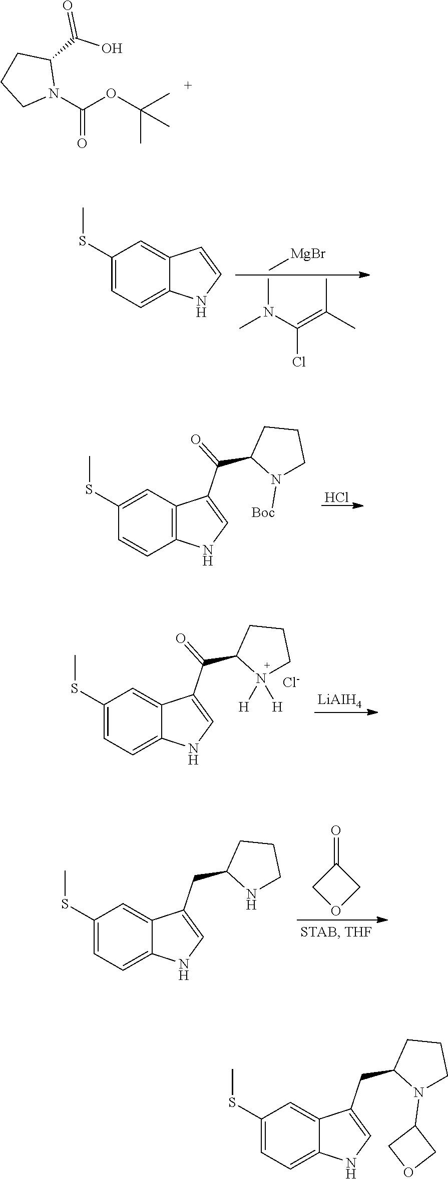

### Step 1: tert-butyl (R)-2-(5-(methylthio)-1H-indole-3-carbonyl)pyrrolidine-1-carboxylate

A solution of 5-methylthio-indole (4.17 g, 2 Eq, 24.8 mmol) in dry toluene (40.00 mL) at 0 °C was treated with MeMgBr in Et₂O (2.96 g, 8.26 mL, 3.00 molar, 2 Eq, 24.8 mmol) and the mixture was stirred for 30 min before being warmed to rt and stirred for a further 30 min. Simultaneously, ((benzyloxy)carbonyl)-D-proline (2.75 g, 1 Eq, 12.4 mmol) in DCM (40.00 mL) at rt was treated with 1-chloro-N,N,2-trimethylprop-1-en-1-amine (1.82 g, 1.80 mL, 1.1 Eq, 13.6 mmol) and the resulting solution was stirred at rt for 45 min before being added dropwise to the solution of deprotonated indole. The reaction mixture was stirred at rt for 18 h before the mixture was treated with sat. aq. NaHCO₃ solution (80 mL). The aq. layer was extracted with EtOAc (3×75 mL) before the pooled organics were washed with brine (40 mL), dried (MgSO₄) and concentrated in vacuo. The crude product was purified by chromatography on silica gel (120 g cartridge, 0-100% EtOAc/iso-hexane) and the product then triturated from MTBE to afford the sub-title compound (1.06 g, 2.94 mmol, 24%). m/z 260.82 (M+H−CO₂tBu)⁺ (ES+)

¹H NMR 500 MHz, DMSO) δ 12.01 (s, 2H), 8.45-8.41 (m, 1H), 8.41 (m, 1H), 8.14-8.11 (m, 1H), 8.11-8.08 (m, 1H), 7.45-7.43 (m, 1H), 7.44-7.42 (m, 1H), 7.21-7.18 (m, 1H), 7.19-7.17 (m, 1H), 5.10 (dd, J=8.9, 3.7 Hz, 1H), 5.06-5.01 (m, 1H), 3.49-3.36 (m, 4H), 2.34-2.21 (m, 2H), 1.88-1.74 (m, 6H), 1.39 (s, 9H), 1.13 (s, 9H). Product is present as a ~1:1 mixture of tautomers with the spectra possessing multiple regions of overlapping.

Therefore, spectrum has been quoted as a mixture of both species. Due to overlap with residual DMSO peak, both tautomer's SMe peaks are obscured.

### Step 2: (R)-2-(5-(methylthio)-1H-indole-3-carbonyl)pyrrolidin-1-ium

To a solution of the product from step 1 above (1.06 g, 1 Eq, 2.94 mmol) in DCM (30.0 mL) and MeOH at rt was added HCl in IPA (1.07 g, 4.90 mL, 6.00 molar, 10 Eq, 29.4 mmol) dropwise. The reaction mixture was left to stir for 18 h and then concentrated in vacuo to afford sub-title compound (893 mg, 3.01 mmol, 102%). m/z 261.12 (M+H)⁺ (ES⁺). ¹H NMR (500 MHz, DMSO) δ 12.51-12.47 (m, 1H), 8.58 (d, J=3.2 Hz, 1H), 8.09 (d, J=1.9 Hz, 1H), 7.50 (d, J=8.4 Hz, 1H), 7.24 (dd, J=8.4, 1.9 Hz, 1H), 5.06 (s, 1H), 3.29 (d, J=11.1 Hz, 2H), 2.71-2.50 (m, 2H), 2.04-1.85 (m, 3H). SMe peak is not observed due to overlap with residual DMSO solvent peak.

### Step 3: (R)-5-(methylthio)-3-(pyrrolidin-2-ylmethyl)-1H-indole

To a stirred suspension of the product from step 2 above (873 mg, 1 Eq, 2.94 mmol) in dry 2-methyltetrahydrofuran (30.00 mL) under a nitrogen atmosphere at 0 °C was added lithium aluminium hydride in THF (558 mg, 6.13 mL, 2.40 molar, 5 Eq, 14.7 mmol). The reaction mixture was heated to 60 °C and stirred for 5 hours. LCMS analysis indicated that all the starting material had been consumed but the reaction had stalled at the partially reduced indole.

The mixture was cooled to 0 °C, diluted with TBME (15 mL) then water (0.5 mL) was added followed by 2 M aq. NaOH (0.5 mL) and water (1.5 mL). The mixture was warmed to rt and stirred for 30 min then filtered washing with EtOAc (2×10 mL) and the filtrate was concentrated in vacuo. The resulting product was dissolved in dry 2-Methyltetrahydrofuran (30.00 mL), cooled to 0 °C, and lithium aluminum hydride in THF (335 mg, 3.68 mL, 2.40 molar, 3 Eq, 8.82 mmol) added dropwise. The reaction mixture was heated to 60 °C and stirred for 2 hours. The mixture was cooled to 0 °C, diluted with TBME (15 mL) then water (0.5 mL) was added followed by 2 M aq. NaOH (0.5 mL), water (1.5 mL), MgSO₄ (ca 2.0 g). The mixture was warmed to rt and stirred for 30 min then filtered washing with EtOAc (2×10 mL) and the filtrate was concentrated in vacuo. The resulting product was dissolved in MeOH (ca 10 mL) and loaded onto a cartridge of Si-TsOH (ca 5 g) washing with MeOH (3×10 mL). The cartridge was eluted with 0.7 M NH3/MeOH (3×10 mL) and concentrated under reduced pressure to afford sub-title compound (426.1 mg, 1.2 mmol, 45%, 72% Purity). m/z 247.00 (M+H)⁺. ¹H NMR (500 MHz, DMSO) δ 10.83 (s, 1H), 7.49 (s, 1H), 7.29 (d, J=8.4 Hz, 1H), 7.15 (s, 1H), 7.06 (dd, J=8.4, 1.8 Hz, 1H), 3.23-3.17 (m, 1H), 2.91-2.84 (m, 1H), 2.76 (dd, J=14.2, 6.3 Hz, 1H), 2.72-2.64 (m, 2H), 2.45 (s, 3H), 1.77-1.52 (m, 4H), 1.34-1.22 (m, 1H).

### Step 4: (R)-5-(methylthio)-3-((1-(oxetan-3-yl)pyrrolidin-2-yl)methyl)-1H-indole

To a stirred solution of the product from step 3 above (84.0 mg, 72% Wt, 1 Eq, 245 μmol) in dry THF (3 mL) at rt was added oxetan-3-one (23.5 mg, 20.9 μL, 98% Wt, 1.3 Eq, 319 μmol). The reaction mixture was stirred for 2 hours, sodium triacetoxyborohydride (156 mg, 3 Eq, 736 μmol) added, and then left to stir overnight. The reaction mixture was quenched with sat. sodium bicarbonate solution (5 mL) and left to stir. The aqueous suspension was diluted with water (5 mL) and extracted with EtOAc (3×10 mL). The combined organic layers were dried over MgSO₄, filtered and concentrated in vacuo. The crude product was purified by chromatography on silica gel (12 g cartridge, 0-10% (0.7 M NH₃/MeOH) in DCM) to afford sub-title compound (47.1 mg, 156 μmol, 63.4%, 100% Purity). m/z 302.91 (M+H)⁺. ¹H NMR (500 MHz, DMSO) 10.84 (s, 1H), 7.42 (s, 1H), 7.28 (d, J=8.4 Hz, 1H), 7.15 (d, J=2.2 Hz, 1H), 7.05 (dd, J=8.3, 1.8 Hz, 1H), 4.61 (dt, J=16.0, 6.2 Hz, 3H), 4.53 (t, J=6.6 Hz, 1H), 3.99 (p, J=6.8 Hz, 1H), 3.01-2.92 (m, 1H), 2.78-2.69 (m, 2H), 2.49-2.44 (m, 4H), 2.41-2.33 (m, 1H), 1.72-1.57 (m, 3H), 1.52-1.42 (m, 1H).

### Example 30: Synthesis of (R)-4-(2-((5-(methylthio)-1H-indol-3-yl)methyl)pyrrolidin-1-yl)tetrahydro-2H-thiopyran 1,1-dioxide (22B)

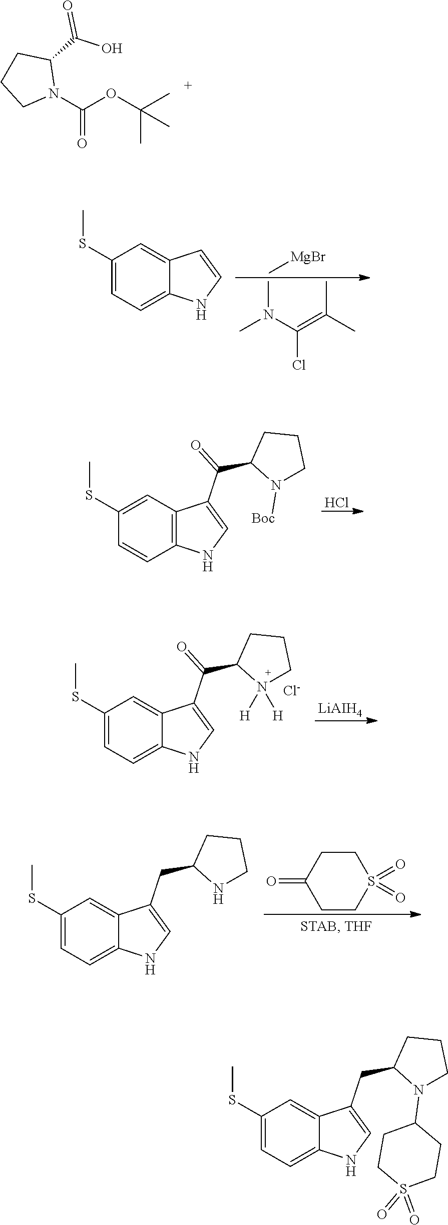

### Step 1: tert-butyl (R)-2-(5-(methylthio)-1H-indole-3-carbonyl)pyrrolidine-1-carboxylate

A solution of 5-methylthio-indole (4.17 g, 2 Eq, 24.8 mmol) in dry toluene (40.00 mL) at 0 °C was treated with MeMgBr in Et₂O (2.96 g, 8.26 mL, 3.00 molar, 2 Eq, 24.8 mmol) and the mixture was stirred for 30 min before being warmed to rt and stirred for a further 30 min. Simultaneously, ((benzyloxy)carbonyl)-D-proline (2.75 g, 1 Eq, 12.4 mmol) in DCM (40.00 mL) at rt was treated with 1-chloro-N,N,2-trimethylprop-1-en-1-amine (1.82 g, 1.80 mL, 1.1 Eq, 13.6 mmol) and the resulting solution was stirred at rt for 45 min before being added dropwise to the solution of deprotonated indole. The reaction mixture was stirred at rt for 18 h before the mixture was treated with sat. aq. NaHCO₃ solution (80 mL). The aq. layer was extracted with EtOAc (3×75 mL) before the pooled organics were washed with brine (40 mL), dried (MgSO₄) and concentrated in vacuo. The crude product was purified by chromatography on silica gel (120 g cartridge, 0-100% EtOAc/iso-hexane) and the product then triturated from MTBE to afford the sub-title compound (1.06 g, 2.94 mmol, 24%). m/z 260.82 (M+H-CO₂tBu)⁺ (ES+). ¹H NMR 500 MHz, DMSO) δ 12.01 (s, 2H), 8.45-8.41 (m, 1H), 8.41 (m, 1H), 8.14-8.11 (m, 1H), 8.11-8.08 (m, 1H), 7.45-7.43 (m, 1H), 7.44-7.42 (m, 1H), 7.21-7.18 (m, 1H), 7.19-7.17 (m, 1H), 5.10 (dd, J=8.9, 3.7 Hz, 1H), 5.06-5.01 (m, 1H), 3.49-3.36 (m, 4H), 2.34-2.21 (m, 2H), 1.88-1.74 (m, 6H), 1.39 (s, 9H), 1.13 (s, 9H). Product is present as a ~1:1 mixture of tautomers with the spectra possessing multiple regions of overlapping. Therefore, spectrum has been quoted as a mixture of both species. Due to overlap with residual DMSO peak, both tautomer's SMe peaks are obscured.

### Step 2: (R)-2-(5-(methylthio)-1H-indole-3-carbonyl)pyrrolidin-1-ium

To a solution of the product from step 1 above (1.06 g, 1 Eq, 2.94 mmol) in DCM (30.0 mL) and MeOH at rt was added HCl in IPA (1.07 g, 4.90 mL, 6.00 molar, 10 Eq, 29.4 mmol) dropwise. The reaction mixture was left to stir for 18 h and then concentrated in vacuo to afford sub-title compound (893 mg, 3.01 mmol, 102%). m/z 261.12 (M+H)⁺ (ES⁺). ¹H NMR (500 MHz, DMSO) δ 12.51-12.47 (m, 1H), 8.58 (d, J=3.2 Hz, 1H), 8.09 (d, J=1.9 Hz, 1H), 7.50 (d, J=8.4 Hz, 1H), 7.24 (dd, J=8.4, 1.9 Hz, 1H), 5.06 (s, 1H), 3.29 (d, J=11.1 Hz, 2H), 2.71-2.50 (m, 2H), 2.04-1.85 (m, 3H). SMe peak is not observed due to overlap with residual DMSO solvent peak.

### Step 3: (R)-5-(methylthio)-3-(pyrrolidin-2-ylmethyl)-1H-indole

To a stirred suspension of the product from step 2 above (873 mg, 1 Eq, 2.94 mmol) in dry 2-methyltetrahydrofuran (30.00 mL) under a nitrogen atmosphere at 0 °C was added lithium aluminium hydride in THF (558 mg, 6.13 mL, 2.40 molar, 5 Eq, 14.7 mmol). The reaction mixture was heated to 60 °C and stirred for 5 hours. LCMS analysis indicated that all the starting material had been consumed but the reaction had stalled at the partially reduced indole.

The mixture was cooled to 0 °C, diluted with TBME (15 mL) then water (0.5 mL) was added followed by 2 M aq. NaOH (0.5 mL) and water (1.5 mL). The mixture was warmed to rt and stirred for 30 min then filtered washing with EtOAc (2×10 mL) and the filtrate was concentrated in vacuo. The resulting product was dissolved in dry 2-Methyltetrahydrofuran (30.00 mL), cooled to 0 °C, and lithium aluminum hydride in THF (335 mg, 3.68 mL, 2.40 molar, 3 Eq, 8.82 mmol) added dropwise. The reaction mixture was heated to 60 °C and stirred for 2 hours. The mixture was cooled to 0 °C, diluted with TBME (15 mL) then water (0.5 mL) was added followed by 2 M aq. NaOH (0.5 mL), water (1.5 mL), MgSO₄ (ca 2.0 g). The mixture was warmed to rt and stirred for 30 min then filtered washing with EtOAc (2×10 mL) and the filtrate was concentrated in vacuo. The resulting product was dissolved in MeOH (ca 10 mL) and loaded onto a cartridge of Si-TsOH (ca 5 g) washing with MeOH (3×10 mL). The cartridge was eluted with 0.7 M NH₃/MeOH (3×10 mL) and concentrated under reduced pressure to afford sub-title compound (426.1 mg, 1.2 mmol, 45%, 72% Purity). m/z 247.00 (M+H)⁺. ¹H NMR (500 MHz, DMSO) δ 10.83 (s, 1H), 7.49 (s, 1H), 7.29 (d, J=8.4 Hz, 1H), 7.15 (s, 1H), 7.06 (dd, J=8.4, 1.8 Hz, 1H), 3.23-3.17 (m, 1H), 2.91-2.84 (m, 1H), 2.76 (dd, J=14.2, 6.3 Hz, 1H), 2.72-2.64 (m, 2H), 2.45 (s, 3H), 1.77-1.52 (m, 4H), 1.34-1.22 (m, 1H).

### Step 4: (R)-4-(2-((5-(methylthio)-1H-indol-3-yl)methyl)pyrrolidin-1-yl)tetrahydro-2H-thiopyran 1,1-dioxide

To a stirred solution of the product from step 3 above (84.0 mg, 72% Wt, 1 Eq, 245 μmol) in dry THF (3 mL) at rt was added tetrahydro-4H-thiopyran-4-one 1,1-dioxide (47.3 mg, 1.3 Eq, 319 μmol). The reaction mixture was stirred for 2 hours, sodium triacetoxyhydroborate (156 mg, 3 Eq, 736 μmol) added, and left to stir overnight at rt. The reaction mixture was quenched with sat. sodium bicarbonate solution (5 mL) and left to stir. The aqueous suspension was diluted with water (5 mL) and extracted with EtOAc (3×10 mL). The combined organic layers were dried over MgSO₄, filtered, and concentrated in vacuo. The crude product was purified by chromatography on silica gel (12 g cartridge, 0-10% (0.7 M NH₃/MeOH) in DCM) to afford sub-title compound (65.4 mg, 173 μmol, 70.4%). m/z 379.28 (M+H)⁺. ¹H NMR (500 MHz, DMSO) δ 10.85 (s, 1H), 7.45 (d, J=1.8 Hz, 1H), 7.29 (d, J=8.4 Hz, 1H), 7.16 (s, 1H), 7.05 (dd, J=8.5, 1.7 Hz, 1H), 3.24-3.13 (m, 2H), 3.12-3.00 (m, 4H), 2.88 (d, J=14.2 Hz, 1H), 2.82 (d, J=8.0 Hz, 1H), 2.58-2.53 (m, 1H), 2.46 (s, 3H), 2.16-2.02 (m, 3H), 2.02-1.88 (m, 2H), 1.67-1.56 (m, 3H), 1.52-1.45 (m, 1H).

### Example 31: Synthesis of (R)-5-methoxy-3-((1-((4-methoxypyridin-3-yl)methyl)pyrrolidin-2-yl)methyl)-1H-indole, Formic Acid (47A)

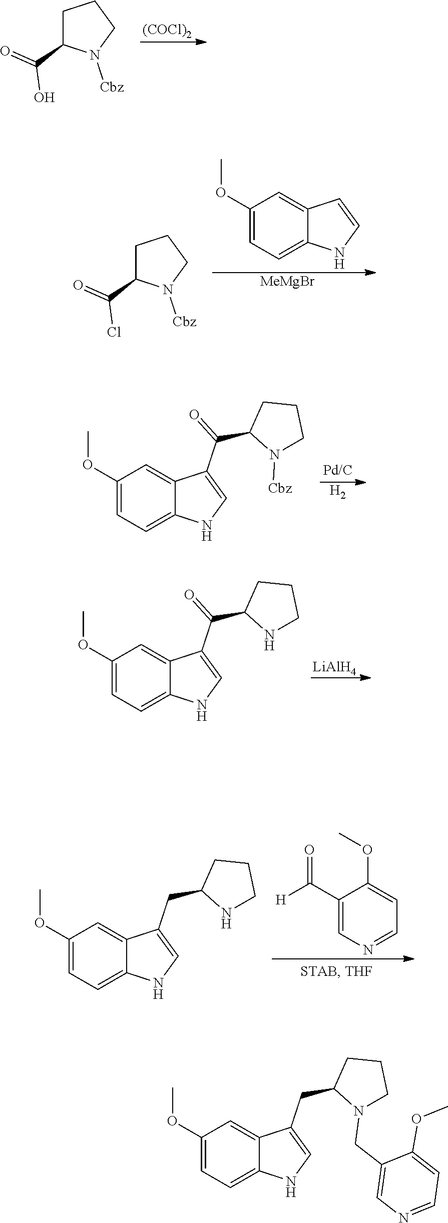

### Step 1: Benzyl (R)-2-(chlorocarbonyl)pyrrolidine-1-carboxylate

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h. The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil. m/z 264.2 (M−Cl+MeOH)⁺ (ES⁺)

### Step 2: benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity). m/z 401.4 (M+Na+H)⁺ (ES+); 377.3 (M−H)⁻ (ES−). ¹H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%). m/z 245.2 (M+H)+ (ES+); 243.2 (M−H)− (ES−). 1H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%). m/z 231.2 (M+H)+ (ES+); 229.2 (M−H)− (ES−). ¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78%

### Step 5: (R)-5-methoxy-3-((1-((4-methoxypyridin-3-yl)methyl)pyrrolidin-2-yl)methyl)-1H-indole, Formic Acid

A mixture of (R)-5-methoxy-3-(pyrrolidin-2-ylmethyl)-1H-indole (84.0 mg, 1 Eq, 365 μmol) and 4-methoxynicotinaldehyde (60.0 mg, 1.2 Eq, 438 μmol) in THF (2.00 mL) was stirred at room temperature for 30 min before sodium triacetoxyborohydride (232 mg, 3 Eq, 1.09 mmol) was added and the mixture was stirred at room temperature for 16 hours. The reaction mixture was quenched with 0.7 M NH₃ in MeOH (5 mL), filtered and the solvent was removed under reduced pressure. The crude product was purified by reverse phase chromatography.

The crude product was purified by chromatography on RP Flash C18 (12 g cartridge, 5-50% (MeCN+0.1% formic acid) in (water+0.1% formic acid)) to afford (R)-5-methoxy-3-((1-((4-methoxypyridin-3-yl)methyl)pyrrolidin-2-yl)methyl)-1H-indole, Formic Acid (8.00 mg, 20 μmol, 5.5%, 99% Purity) as a colourless oil. m/z 352.2 (M+H)+ (ES+). ¹H NMR (500 MHz, DMSO) δ 1.41-1.64 (m, 2H), 1.66-1.77 (m, 1H), 2.14-2.23 (m, 1H), 2.56 (dd, J=14.2, 8.8 Hz, 1H), 2.73-2.80 (m, 1H), 2.85-2.91 (m, 1H), 3.04 (dd, J=14.2, 3.7 Hz, 1H), 3.24-3.40 (m, 1H), 3.45 (d, J=13.8 Hz, 1H), 3.74 (s, 3H), 3.86 (s, 3H), 4.05 (d, J=13.8 Hz, 1H), 6.69 (dd, J=8.7, 2.4 Hz, 1H), 6.97 (d, J=2.4 Hz, 1H), 7.03 (d, J=5.5 Hz, 1H), 7.10 (d, J=2.2 Hz, 1H), 7.20 (d, J=8.7 Hz, 1H), 8.29-8.47 (m, 2H), 10.60 (s, 1H).

### Example 32: Synthesis of (R)-5-((2-((5-methoxy-1H-indol-3-yl)methyl)pyrrolidin-1-yl)methyl)-1H-indazole, Formic Acid (48A)

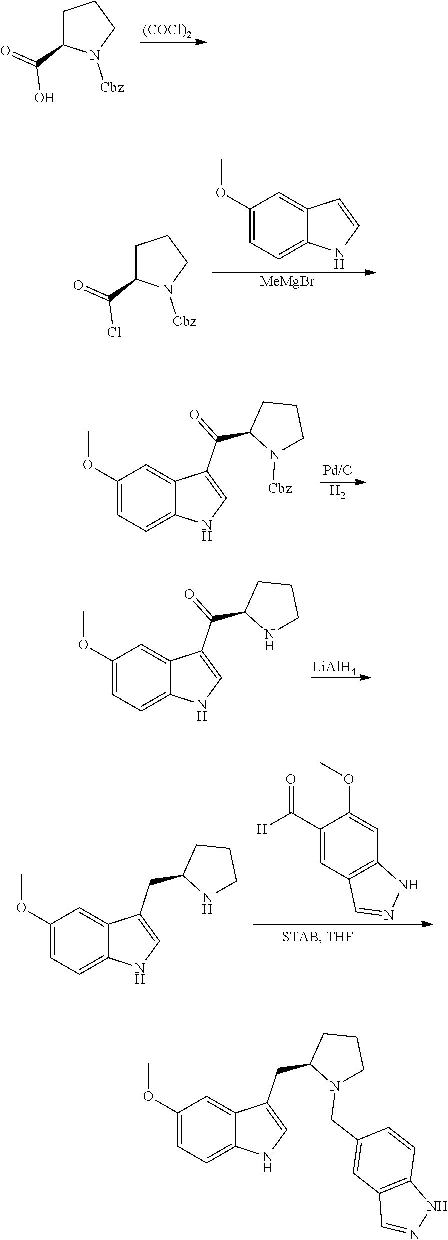

### Step 1: Benzyl (R)-2-(chlorocarbonyl)pyrrolidine-1-carboxylate

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h. The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil. m/z 264.2 (M−Cl+MeOH)⁺ (ES⁺).

### Step 2: Benzyl (R)-2-(5-methoxy-1H-indole-3-carbonyl)pyrrolidine-1-carboxylate

To a stirred solution of 5-methoxy-1H-indole (5.00 g, 2 Eq, 34.0 mmol) in dry Toluene (225 mL) under a nitrogen atmosphere at 0 °C was added MeMgBr in diethyl ether (4.05 g, 11.3 mL, 3.00 molar, 2 Eq, 34.0 mmol) dropwise over 10 min. The reaction mixture was stirred at rt for 3 h. A solution of product from step 1 above (4.83 g, 1.06 Eq, 18.0 mmol) in dry Toluene (25.0 mL) was added dropwise and the reaction mixture was stirred for 2 h. The reaction mixture was quenched with sat. aq. NaHCO₃ (250 mL) at 0 °C and left to stir at rt for 30 min. The resulting suspension was diluted with water (200 mL) and extracted with EtOAc (2×300 mL) and the collected organic layers were dried over Na₂SO₄, filtered and concentrated in vacuo. The crude product was purified by chromatography on silica gel (120 g cartridge, 0-70% EtOAc/iso-hexane). The resulting product was triturated with MTBE and filtered off to afford sub-title compound (1.57 g, 4.1 mmol, 24%, 99% Purity). m/z 401.4 (M+Na+H)⁺ (ES+); 377.3 (M−H)⁻ (ES−). ¹H NMR (500 MHz, DMSO) δ 11.90 (s, 1H), 8.38 (d, J=9.6 Hz, 1H), 7.76-7.67 (m, 1H), 7.42-7.35 (m, 3H), 7.35-7.12 (m, 1H), 7.11-7.00 (m, 2H), 6.89-6.82 (m, 1H), 5.25-5.14 (m, 1H), 5.07 (q, J=12.8 Hz, 1H), 4.95 (q, J=13.3 Hz, 1H), 3.78 (d, J=5.2 Hz, 3H), 3.58-3.44 (m, 2H), 2.43-2.26 (m, 1H), 1.95-1.78 (m, 3H).

### Step 3: (R)-5-methoxy-3-prolyl-1H-indole

A stirred suspension of product from step 2 above (1.57 g, 1 Eq, 4.11 mmol) and Pd/C (87 L) (353 mg, 5% Wt, 0.04 Eq, 166 μmol) in EtOH (20.0 mL) under a nitrogen atmosphere at rt was charged with Hydrogen (1 bar). The reaction mixture was stirred at rt for 19 h. The reaction mixture was filtered through glass microfibres, rinsing with MeOH (200 mL). The solvent was removed in vacuo to afford sub-title compound (907 mg, 3.6 mmol, 89%). m/z 245.2 (M+H)+ (ES+); 243.2 (M−H)− (ES−). ¹H NMR (500 MHz, DMSO) δ 11.88 (s, 1H), 8.31 (s, 1H), 7.71 (d, J=2.5 Hz, 1H), 7.36 (d, J=8.8 Hz, 1H), 6.84 (dd, J=8.8, 2.5 Hz, 1H), 4.35 (dd, J=8.5, 6.0 Hz, 1H), 3.78 (s, 3H), 3.05 (dt, J=12.0, 6.0 Hz, 1H), 2.73 (dt, J=10.5, 6.5 Hz, 1H), 2.26-2.05 (m, 1H), 1.77-1.67 (m, 1H), 1.67-1.52 (m, 2H). One exchangeable proton not observed.

### Step 4: (R)-5-methoxy-3-(pyrrolidin-2-ylmethyl)-1H-indole

To a stirred suspension of the product from step 3 above (907 mg, 1 Eq, 3.64 mmol) in dry 2-MeTHF (20.0 mL) under a nitrogen atmosphere at 0 °C was added LiAlH₄ (2.4 M in THF) (6.96 mL, 2.40 molar, 4.59 Eq, 16.7 mmol). The reaction mixture was heated to 60 °C and stirred for 2.5 h. The reaction mixture was cooled to 0 °C and quenched with 3:1 THF/water (15 mL). Na₂SO₄ (10 g) was added to the suspension and stirred at rt for 15 min. The suspension was filtered through celite, washing with EtOAc (200 mL). The filtrate was concentrated in vacuo. The crude product was purified by chromatography on silica gel (24 g cartridge, 0-100% (3:1 EtOAc/EtOH+2% NH₄OH) in iso-hexane) to afford the sub-title compound (742 mg, 2.79 mmol, 76%). m/z 231.2 (M+H)+ (ES+); 229.2 (M−H)− (ES−). ¹H NMR (500 MHz, DMSO) δ 10.59 (s, 1H), 7.20 (d, J=8.7 Hz, 1H), 7.09 (d, J=2.4 Hz, 1H), 6.98 (d, J=2.4 Hz, 1H), 6.69 (dd, J=8.7, 2.4 Hz, 1H), 3.75 (s, 3H), 3.21 (p, J=6.8 Hz, 1H), 2.93-2.84 (m, 1H), 2.79-2.60 (m, 3H), 1.78-1.62 (m, 2H), 1.62-1.53 (m, 1H), 1.36-1.25 (m, 1H). One Exchangeable proton not observed.

The material was dissolved in MeOH to 1 mg/mL and screened by SFC (Waters UPC2, with a PDA and a QDA detector). The optimal conditions are using a Chiralpak IH column, 4.6×250 mm, 5 μm, with mobile phase 20% Methanol (28 mM NH₃), 80% CO2. The flow rate was 4 mL/min. The run time was 4.5 mins. EE of 97.78%.

### Step 5: (R)-5-((2-((5-methoxy-1H-indol-3-yl)methyl)pyrrolidin-1-yl)methyl)-1H-indazole, Formic Acid

A mixture of (R)-5-methoxy-3-(pyrrolidin-2-ylmethyl)-1H-indole (84.0 mg, 1 Eq, 365 μmol) and 1H-indazole-5-carbaldehyde (64.0 mg, 1.2 Eq, 438 μmol) in THF (2.00 mL) was stirred at room temperature for 30 min before sodium triacetoxyborohydride (232 mg, 3 Eq, 1.09 mmol) was added and the mixture was stirred at room temperature for 16 hours. The reaction mixture was quenched with 0.7 M NH₃ in MeOH (5 mL), filtered and the solvent was removed under reduced pressure. The crude product was purified by reverse phase chromatography.

The crude product was purified by chromatography on RP Flash C18 (12 g cartridge, 5-50% (MeCN+0.1% formic acid) in (water+0.1% formic acid)) to afford (R)-5-((2-((5-methoxy-1H-indol-3-yl)methyl)pyrrolidin-1-yl)methyl)-1H-indazole, Formic Acid (22.0 mg, 52 μmol, 14%, 96% Purity). m/z 361.3 (M+H)+ (ES+). ¹H NMR (500 MHz, DMSO) δ 1.48-1.68 (m, 3H), 1.71-1.81 (m, 1H), 2.23-2.32 (m, 1H), 2.61 (dd, J=14.1, 9.2 Hz, 1H), 2.85 (s, 2H), 3.07 (dd, J=14.0, 3.8 Hz, 1H), 3.48 (d, J=12.9 Hz, 1H), 3.70 (s, 3H), 4.25 (d, J=12.8 Hz, 1H), 6.69 (dd, J=8.7, 2.4 Hz, 1H), 6.92 (d, J=2.4 Hz, 1H), 7.12 (d, J=2.3 Hz, 1H), 7.20 (d, J=8.7 Hz, 1H), 7.39 (d, J=8.5 Hz, 1H), 7.49 (d, J=8.6 Hz, 1H), 7.70 (s, 1H), 8.02 (s, 1H), 8.20 (s, 1H), 10.62 (s, 1H), 13.00 (s, 1H)—1H extra due to salt formation with formic acid.

### Example 33: Synthesis of methyl (R)-2-(2-((5-chloro-1H-indol-3-yl)methyl)pyrrolidin-1-yl)acetate (87A)

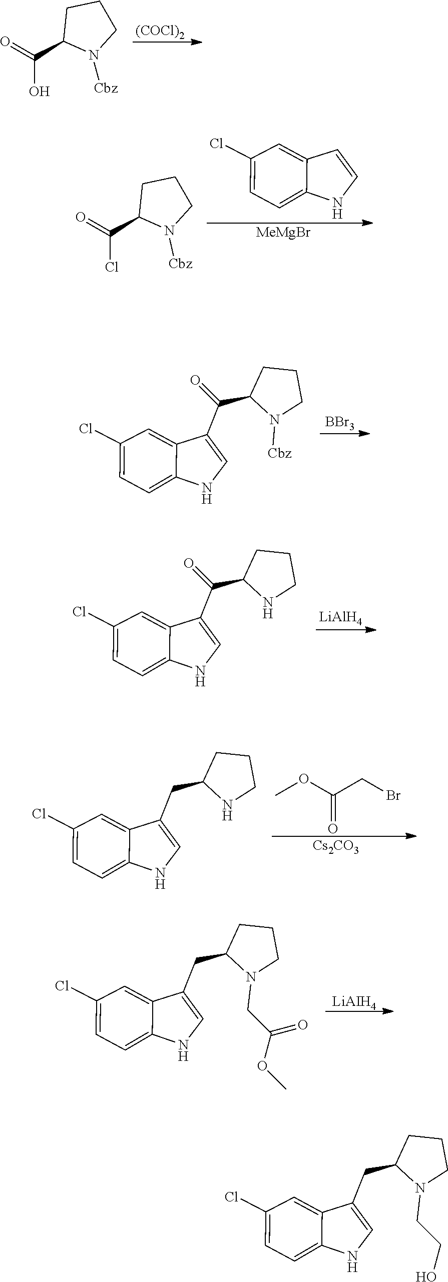

### Step 1: Benzyl (R)-2-(chlorocarbonyl)pyrrolidine-1-carboxylate

To a stirred solution of ((benzyloxy)carbonyl)-D-proline (1.70 g, 1 Eq, 6.83 mmol) in dry DCM (50 mL) under a nitrogen atmosphere at 0 °C was added oxalyl dichloride 2.60 g, 1.79 mL, 3 Eq, 20.5 mmol) and catalytic DMF. The reaction mixture was stirred at rt for 2.5 h, concentrated in vacuo and the resulting crude suspended in iso-hexane (3×20 mL), and concentrated in vacuo to afford sub-title compound (1.83 g, 6.5 mmol, 95%, 95% purity) as a pale-yellow oil. m/z 264.2 (M−Cl+MeOH)⁺ (ES⁺).

### Step 2: Benzyl (R)-2-(5-chloro-1H-indole-3-carbonyl)pyrrolidine-1-carboxylate

To a stirred solution of 5-chloro-1H-indole (2.07 g, 2 Eq, 13.7 mmol) in dry Toluene (90 mL) under a nitrogen atmosphere at 0 °C was added MeMgBr in diethyl ether (1.63 g, 4.55 mL, 3.00 molar, 2 Eq, 13.7 mmol) dropwise over 5 min. The reaction mixture was stirred at rt for 2.5 h, solution of product from step 1 above (1.83 g, 0.95 Eq, 6.49 mmol) in dry Toluene (10.0 mL) was added dropwise and the reaction mixture was stirred for 18 h. The reaction mixture was quenched with sat. aq. NaHCO₃ (100 mL) at 0 °C and left to stir at rt for 30 min. The resulting suspension was diluted with water (100 mL) and extracted with EtOAc (3×100 mL) and the collected organic layers were dried over MgSO₄, filtered and concentrated in vacuo. The crude product was purified by chromatography on silica gel (120 g cartridge, 0-75% EtOAc/iso-hexane). The resulting product was triturated with MTBE and filtered off to afford sub-title compound (1.09 g, 2.85 mmol, 24%, 100% Purity). m/z 383.00 (Cl³⁷), 381.00 (Cl³⁵) (M−H)− (ES−). ¹H NMR (500 MHz, DMSO) δ 12.22 (s, 1H), 12.19 (s, 1H), 8.54 (d, J=3.0 Hz, 1H), 8.50 (d, J=3.0 Hz, 1H), 8.18 (s, 1H), 8.15 (s, OH), 7.54-7.49 (m, 2H), 7.40-7.36 (m, 4H), 7.26 (td, J=6.7, 3.5 Hz, 2H), 7.12 (t, J=7.2 Hz, 1H), 7.07-6.98 (m, 4H), 5.24-5.13 (m, 2H), 5.11-5.02 (m, 2H), 5.01-4.86 (m, 2H), 3.58-3.47 (m, 4H), 2.35 (m, 2H), 1.93-1.79 (m, 6H). Product is present as a ~1:1 mixture of tautomers with the spectrum possessing multiple regions of overlapping. Therefore, spectrum has been quoted as a mixture of both species.

### Step 3: (R)-5-chloro-3-prolyl-1H-indole

To a solution of the product from step 2 above (1.09 g, 1 Eq, 2.85 mmol) in dry DCM (10.00 mL) under a nitrogen atmosphere at 0 °C was added BBr₃ in heptane (2.14 g, 8.54 mL, 1.00 molar, 3 Eq, 8.54 mmol) drop-wise and the resulting suspension was stirred for 30 min then warmed to rt and stirred for a further 4 h. The solution was cooled down to 0 °C, a further 1 eq. of BBr₃ in heptane (0.71 g, 2.85 mL, 1.00 molar) added, and the reaction mixture then warmed to rt and left to stir overnight. MeOH (5 mL) was added drop-wise and the mixture was stirred for 30 min. The mixture was loaded onto a cartridge of Si-TsOH (ca 5.0 g) and washed with MeOH (3×10 mL). The product was eluted with 0.7 M NH₃ in MeOH (4×10 mL) and concentrated under reduced pressure to sub-title compound (0.720 g, 2.8 mmol, 97%, 95% Purity). m/z 251.2 (Cl³⁷), 249.2 (Cl³⁵) (M+H)⁺ (ES⁺). ¹H NMR (500 MHz, DMSO) δ 8.56 (d, J=1.8 Hz, 1H), 8.15 (m, 1H), 7.54 (dd, J=8.7, 1.7 Hz, 1H), 7.28 (m, 1H), 4.78-4.71 (m, 1H), 3.21-3.17 (m, 1H), 3.08-2.98 (m, 1H), 2.38 (q, J=8.2 Hz, 1H), 1.90-1.82 (m, 1H), 1.82-1.72 (m, 2H). Note: 2 Exchangeable protons not observed.

The sample was dissolved in MeOH to 1 mg/mL and screened by SFC (Waters UPC², with a PDA and a QDA detector). The optimal conditions are using a Chiralpak IH column, 4.6×250 mm, 5 μm, with mobile phase 15% Methanol (28 mM NH₃), 85% CO₂. The flow rate was 4 mL/min. The run time was 9.0 mins. Sample is racemic.

### Step 4: (R)-5-chloro-3-(pyrrolidin-2-ylmethyl)-1H-indole

To a stirred suspension of the product from step 3 (0.720 g, 95% Wt, 1 Eq, 2.75 mmol) in dry tetrahydrofuran (18.0 mL) under a nitrogen atmosphere at 0 °C was added LiAlH₄ in THF (546 mg, 6.00 mL, 2.40 molar, 5.24 Eq, 14.4 mmol). The reaction mixture was heated to 60 °C and stirred for 3 h. Following this, the reaction was cooled to 0 °C, diluted with TBME (15 mL) then water (0.32 mL) added followed by 2 M aq NaOH (0.32 mL), water (1.00 mL) and MgSO₄. The mixture was warmed to rt and stirred for 30 min then filtered washing with EtOAc (2×10 mL) and the filtrate was concentrated in vacuo. The resulting product was dissolved in MeOH (ca 5 mL) and loaded onto a cartridge of Si-TsOH (ca 5 g) washing with MeOH (3×10 mL). The cartridge was eluted with 0.7 M NH₃/MeOH (3×10 mL) and concentrated under reduced pressure to afford sub-title compound (0.324 g, 0.95 mmol, 35%, 69% Purity). m/z 237.20 (Cl³⁷), 235.20 (Cl³⁵) (M+H)+ (ES+). ¹H NMR (500 MHz, DMSO) δ 10.98 (s, 1H), 7.55 (d, J=2.1 Hz, 1H), 7.33 (d, J=8.5 Hz, 1H), 7.24-7.21 (m, 1H), 7.04 (m, 1H), 3.22-3.15 (m, 1H), 2.91-2.83 (m, 1H), 2.68 (m, 2H), 1.76-1.61 (m, 2H), 1.61-1.51 (m, 3H), 1.32-1.21 (m, 1H). Exchangeable pyrrolidine NH not observed.

### Step 5: methyl (R)-2-(2-((5-chloro-1H-indol-3-yl)methyl)pyrrolidin-1-yl)acetate

To a solution of the product from step 4 above (94.0 mg, 69% Wt, 1 Eq, 276 μmol) and cesium carbonate (180 mg, 2 Eq, 553 μmol) in dry THF (3.00 mL) at rt was added methyl 2-bromoacetate (63.4 mg, 39.2 μL, 1.5 Eq, 414 μmol). The reaction mixture was stirred for 18 hours and then quenched with sat. sodium bicarbonate (5 mL). The resulting aqueous suspension was extracted with EtOAc (2×10 mL), the collected organic layers dried over MgSO₄ and concentrated in vacuo. The mixture was loaded onto a cartridge of Si-TsOH (ca 1.5 g) and washed with MeOH (3×10 mL). The product was eluted with 0.7 M NH₃ in MeOH (3×10 mL) and concentrated under reduced pressure to afford sub-title compound (53.0 mg, 0.12 mmol, 43%, 68% Purity) as a brown oil. m/z 309.01 (Cl³⁷), 307. 12 (Cl³⁵) (M+H)⁺ (ES⁺). ¹H NMR (500 MHz, DMSO) δ 10.99 (s, 1H), 7.54 (d, J=2.1 Hz, 1H), 7.33 (d, J=8.5 Hz, 1H), 7.22 (d, J=2.2 Hz, 1H), 7.03 (dd, J=8.6, 2.1 Hz, 1H), 3.72-3.66 (m, 1H), 3.63 (s, 3H), 3.31-3.25 (m, 2H), 2.95 (dd, J=14.0, 3.9 Hz, 1H), 2.88-2.81 (m, 1H), 2.49-2.45 (m, 1H), 2.40 (q, J=8.4 Hz, 1H), 1.74-1.56 (m, 3H), 1.49-1.40 (m, 1H).

### Step 6: (R)-2-(2-((5-chloro-1H-indol-3-yl)methyl)pyrrolidin-1-yl)ethan-1-ol

To a solution of the product from step 5 above (53.0 mg, 68% Wt, 1 Eq, 117 μmol) in 2-methyltetrahydrofuran (2.00 mL) at 0 °C was added lithium aluminium hydride (14 mg, 0.15 mL, 2.40 molar, 3.1 Eq, 0.36 mmol) dropwise. The mixture was stirred for 30 min, warmed to rt, and stirred for a further 45 minutes. The reaction mixture was cooled to 0 °C, diluted with THF (10 mL) and quenched with H₂O (1.5 mL), 2.0 M NaOH (0.5 mL) and MgSO₄ (ca 2 g) added. The mixture was stirred for 10 min, filtered under gravity, then concentrated in vacuo. The crude product was purified by flash chromatography on silica gel (12 g column, 0-90% (10% (0.7 M NH₃ in MeOH) in dichloromethane) in dichloromethane) to afford sub-title compound (37.5 mg, 0.11 mmol, 95%, 83% Purity) as a colourless oil.

Further purification was achieved by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130A, 5 μm, 30 mm×100 mm, flow rate 40 mL min⁻¹ eluting with a 0.3% Ammonia in water-MeCN gradient over 12.5 mins using UV across all wavelengths with PDA as well as a QDA and ELS detector. At-column dilution pump gives 2 mL min⁻¹ Methanol over the entire method, which is included in the following MeCN percentages. Gradient information: 0.0-0.5 min, 27.5% MeCN; 0.5-10.5 min, ramped from 27.5% MeCN to 57.5% MeCN; 10.5-10.6 min, ramped from 57.5% MeCN to 100% MeCN; 10.6-12.5 min, held at 100% MeCN. The clean fractions were evaporated in a Freeze-dryer, to afford sub-title compound (4.95 mg, 17.8 μmol, 15.1%, 100% Purity). m/z 281.09 (Cl³⁷), 279.13 (Cl³⁵) (M+H)⁺ (ES⁺). ¹H NMR (500 MHz, DMSO) δ 10.99 (s, 1H), 7.53-7.49 (m, 1H), 7.33 (d, J=8.5 Hz, 1H), 7.23 (s, 1H), 7.04 (d, J=8.5 Hz, 1H), 4.42 (t, J=5.4 Hz, 1H), 3.59-3.47 (m, 2H), 3.10-3.04 (m, 1H), 3.04-2.92 (m, 2H), 2.49-2.44 (m, 1H), 2.31 (m, 1H), 2.20-2.11 (m, 1H), 1.68-1.53 (m, 3H), 1.44-1.34 (m, 1H). Exchangeable OH peak not observed.

### Example 34: Synthesis of (R)-5-(methylthio)-3-((1-(tetrahydro-2H-pyran-4-yl)pyrrolidin-2-yl)methyl)-1H-indole (21A)

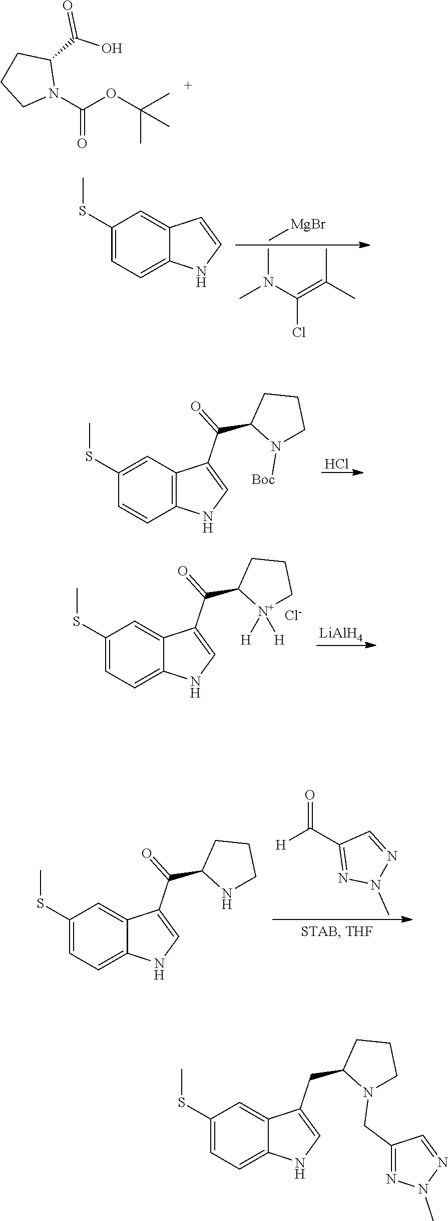

### Step 1: tert-butyl (R)-2-(5-(methylthio)-1H-indole-3-carbonyl)pyrrolidine-1-carboxylate

A solution of 5-methylthio-indole (4.17 g, 2 Eq, 24.8 mmol) in dry toluene (40.00 mL) at 0 °C was treated with MeMgBr in Et₂O (2.96 g, 8.26 mL, 3.00 molar, 2 Eq, 24.8 mmol) and the mixture was stirred for 30 min before being warmed to rt and stirred for a further 30 min. Simultaneously, ((benzyloxy)carbonyl)-D-proline (2.75 g, 1 Eq, 12.4 mmol) in DCM (40.00 mL) at rt was treated with 1-chloro-N,N,2-trimethylprop-1-en-1-amine (1.82 g, 1.80 mL, 1.1 Eq, 13.6 mmol) and the resulting solution was stirred at rt for 45 min before being added dropwise to the solution of deprotonated indole. The reaction mixture was stirred at rt for 18 h before the mixture was treated with sat. aq. NaHCO₃ solution (80 mL). The aq. layer was extracted with EtOAc (3×75 mL) before the pooled organics were washed with brine (40 mL), dried (MgSO₄) and concentrated in vacuo. The crude product was purified by chromatography on silica gel (120 g cartridge, 0-100% EtOAc/iso-hexane) and the product then triturated from MTBE to afford the sub-title compound (1.06 g, 2.94 mmol, 24%). m/z 260.82 (M+H−CO₂tBu)⁺ (ES⁺). ¹H NMR 500 MHz, DMSO) δ 12.01 (s, 2H), 8.45-8.41 (m, 1H), 8.41 (m, 1H), 8.14-8.11 (m, 1H), 8.11-8.08 (m, 1H), 7.45-7.43 (m, 1H), 7.44-7.42 (m, 1H), 7.21-7.18 (m, 1H), 7.19-7.17 (m, 1H), 5.10 (dd, J=8.9, 3.7 Hz, 1H), 5.06-5.01 (m, 1H), 3.49-3.36 (m, 4H), 2.34-2.21 (m, 2H), 1.88-1.74 (m, 6H), 1.39 (s, 9H), 1.13 (s, 9H). Product is present as a ~1:1 mixture of tautomers with the spectra possessing multiple regions of overlapping. Therefore, spectrum has been quoted as a mixture of both species. Due to overlap with residual DMSO peak, both tautomer's SMe peaks are obscured.

### Step 2: (R)-2-(5-(methylthio)-1H-indole-3-carbonyl)pyrrolidin-1-ium

To a solution of the product from step 1 above (1.06 g, 1 Eq, 2.94 mmol) in DCM (30.0 mL) and MeOH at rt was added HCl in IPA (1.07 g, 4.90 mL, 6.00 molar, 10 Eq, 29.4 mmol) dropwise. The reaction mixture was left to stir for 18 h and then concentrated in vacuo to afford sub-title compound (893 mg, 3.01 mmol, 102%). m/z 261.12 (M+H)⁺ (ES⁺). ¹H NMR (500 MHz, DMSO) δ 12.51-12.47 (m, 1H), 8.58 (d, J=3.2 Hz, 1H), 8.09 (d, J=1.9 Hz, 1H), 7.50 (d, J=8.4 Hz, 1H), 7.24 (dd, J=8.4, 1.9 Hz, 1H), 5.06 (s, 1H), 3.29 (d, J=11.1 Hz, 2H), 2.71-2.50 (m, 2H), 2.04-1.85 (m, 3H). SMe peak is not observed due to overlap with residual DMSO solvent peak.

### Step 3: (R)-5-(methylthio)-3-(pyrrolidin-2-ylmethyl)-1H-indole

To a stirred suspension of the product from step 2 above (873 mg, 1 Eq, 2.94 mmol) in dry 2-methyltetrahydrofuran (30.00 mL) under a nitrogen atmosphere at 0 °C was added lithium aluminium hydride in THF (558 mg, 6.13 mL, 2.40 molar, 5 Eq, 14.7 mmol). The reaction mixture was heated to 60 °C and stirred for 5 hours. LCMS analysis indicated that all the starting material had been consumed but the reaction had stalled at the partially reduced indole.

The mixture was cooled to 0 °C, diluted with TBME (15 mL) then water (0.5 mL) was added followed by 2 M aq. NaOH (0.5 mL) and water (1.5 mL). The mixture was warmed to rt and stirred for 30 min then filtered washing with EtOAc (2×10 mL) and the filtrate was concentrated in vacuo. The resulting product was dissolved in dry 2-Methyltetrahydrofuran (30.00 mL), cooled to 0 °C, and lithium aluminum hydride in THF (335 mg, 3.68 mL, 2.40 molar, 3 Eq, 8.82 mmol) added dropwise. The reaction mixture was heated to 60 °C and stirred for 2 hours. The mixture was cooled to 0 °C, diluted with TBME (15 mL) then water (0.5 mL) was added followed by 2 M aq. NaOH (0.5 mL), water (1.5 mL), MgSO₄ (ca 2.0 g). The mixture was warmed to rt and stirred for 30 min then filtered washing with EtOAc (2×10 mL) and the filtrate was concentrated in vacuo. The resulting product was dissolved in MeOH (ca 10 mL) and loaded onto a cartridge of Si-TsOH (ca 5 g) washing with MeOH (3×10 mL). The cartridge was eluted with 0.7 M NH₃/MeOH (3×10 mL) and concentrated under reduced pressure to afford sub-title compound (426.1 mg, 1.2 mmol, 45%, 72% Purity). m/z 247.00 (M+H)⁺. ¹H NMR (500 MHz, DMSO) δ 10.83 (s, 1H), 7.49 (s, 1H), 7.29 (d, J=8.4 Hz, 1H), 7.15 (s, 1H), 7.06 (dd, J=8.4, 1.8 Hz, 1H), 3.23-3.17 (m, 1H), 2.91-2.84 (m, 1H), 2.76 (dd, J=14.2, 6.3 Hz, 1H), 2.72-2.64 (m, 2H), 2.45 (s, 3H), 1.77-1.52 (m, 4H), 1.34-1.22 (m, 1H).

### Step 4: (R)-5-(methylthio)-3-((1-(tetrahydro-2H-pyran-4-yl)pyrrolidin-2-yl)methyl)-1H-indole

To a stirred solution of the product from step 3 above (84.0 mg, 72% Wt, 1 Eq, 245 μmol) in dry THF (3 mL) at rt was added 2-methyl-2H-1,2,3-triazole-4-carbaldehyde (37.3 mg, 95% Wt, 1.3 Eq, 319 μmol). The reaction mixture was stirred for 2 hours, sodium triacetoxyborohydride (156 mg, 3 Eq, 736 μmol) added, and then left to stir overnight. The reaction mixture was quenched with sat. sodium bicarbonate solution (5 mL) and left to stir. The aqueous suspension was diluted with water (5 mL) and extracted with EtOAc (3×10 mL). The combined organic layers were dried over MgSO₄, filtered and concentrated in vacuo. The crude product was purified by chromatography on silica gel (12 g cartridge, 0-10% (0.7 M NH₃/MeOH) in DCM) to afford sub-title compound (52.9 mg, 0.15 mmol, 60%, 95% Purity). m/z 341.97 (M+H)⁺. ¹H NMR (500 MHz, DMSO) δ 10.84 (s, 1H), 7.61 (s, 1H), 7.52 (d, J=1.8 Hz, 1H), 7.29 (d, J=8.4 Hz, 1H), 7.16 (s, 1H), 7.07 (dd, J=8.4, 1.8 Hz, 1H), 4.14 (s, 3H), 4.05 (d, J=13.9 Hz, 1H), 3.61 (d, J=14.0 Hz, 1H), 3.07 (dd, J=14.0, 3.8 Hz, 1H), 2.73-2.64 (m, 1H), 2.58-2.48 (m, 2H), 2.46 (s, 3H), 2.31-2.22 (m, 1H), 1.71-1.42 (m, 4H).

### Example 35: Synthesis of 5-Methoxy-3-(((2S)-4-methoxy-1-((1-methyl-1H-pyrazol-3-yl)methyl)pyrrolidin-2-yl)methyl)-1H-indole (46A)

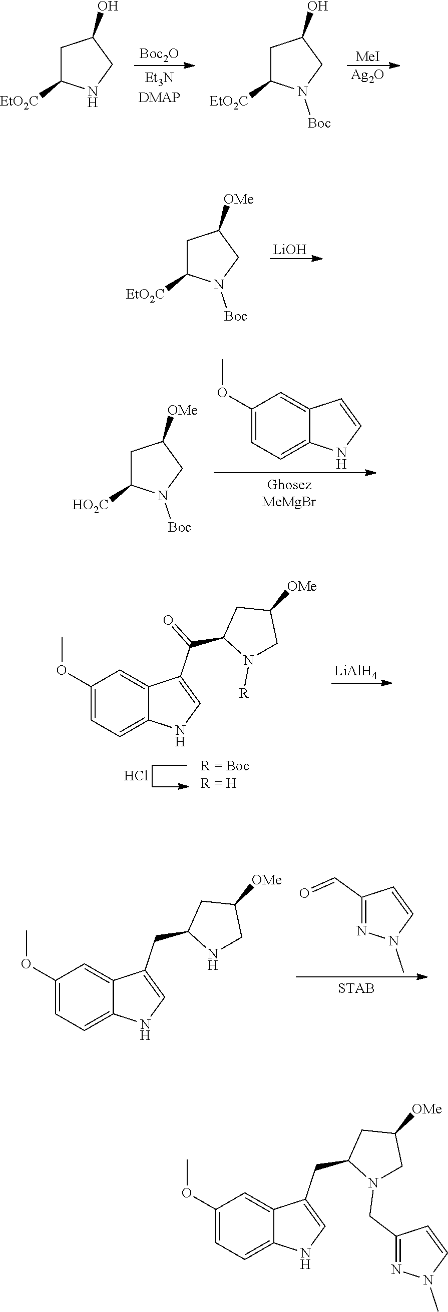

### Step 1: 1-(tert-Butyl) 2-methyl (2R,4R)-4-hydroxypyrrolidine-1,2-dicarboxylate

To a solution of methyl (2R,4R)-4-hydroxy-2-pyrrolidinecarboxylate hydrochloride (5.00 g, 1 Eq, 27.5 mmol) and Boc₂O (7.21 g, 7.59 mL, 1.2 Eq, 33.0 mmol) in DCM (60.0 mL) was added drop-wise Et₃N (6.13 g, 8.44 mL, 2.2 Eq, 60.6 mmol) followed by DMAP (336 mg, 0.1 Eq, 2.75 mmol) and the mixture was stirred at rt for 18 h. 10 wt % aq. Citric acid (50 mL) was added, the mixture was stirred for 15 min then the phases were separated and the aq. phase extracted with DCM (50 mL). The combined organics were washed with brine (50 mL), dried (MgSO₄), filtered and concentrated in vacuo to afford the sub-title compound (6.71 g, 27 mmol, 98%). ¹H NMR (500 MHz, CDCl3) δ 4.47-4.23 (m, 2H), 3.82-3.76 (m, 3H), 3.76-3.45 (m, 2H), 2.40-2.25 (m, 1H), 2.14-2.02 (m, 1H), 1.43 (app d, 9H). One exchangeable proton not observed.

### Step 2: 1-(tert-Butyl) 2-methyl (2R,4R)-4-methoxypyrrolidine-1,2-dicarboxylate

To a solution of the product from step 1 above (6.71 g, 1 Eq, 27.4 mmol) in MeCN (100.0 mL) were added Mel (11.6 g, 5.13 mL, 3 Eq, 82.1 mmol) and silver oxide (9.51 g, 1.5 Eq, 41.0 mmol). The mixture was stirred for 24 h then filtered through celite washing with MeCN (2×20 mL) and concentrated in vacuo. The crude product was purified by chromatography on silica gel (80 g cartridge, 0-100% EtOAc in isohexane) to afford the sub-title compound (1.45 g, 5.3 mmol, 19%) as a colourless oil. m/z 160.6 (M-Boc+H)⁺ (ES+). ¹H NMR (500 MHz, CDCl3) δ 4.45-4.37 (m, 0.4H), 4.30 (dd, J=8.8, 4.0 Hz, 0.6H), 3.97-3.89 (m, 1H), 3.76 (d, J=2.1 Hz, 5H), 3.72 (s, 0.52H), 3.65 (dd, J=11.6, 5.5 Hz, 0.6H), 3.58 (dd, J=11.6, 5.4 Hz, 0.4H), 3.53-3.43 (m, 1H), 3.27 (s, 3H), 2.39-2.18 (m, 2H), 1.47 (s, 4H), 1.42 (s, 5H).

### Step 3: (2R,4R)-1-(tert-Butoxycarbonyl)-4-methoxypyrrolidine-2-carboxylic acid

To a solution of the product from step 2 above (1.45 g, 1 Eq, 5.59 mmol) in THF (20.0 mL) was added a suspension of LiOH (670 mg, 5 Eq, 28.0 mmol) in water (2.00 mL) and the mixture was stirred at rt for 18 h. The mixture was diluted with water (50 mL) and extracted with EtOAc (25 mL). The aq. phase was acidified to ph 1-2 by addition of 1 M aq. HCl then extracted with EtOAc (3×25 mL). The combined organics were washed with water (50 mL), brine (50 mL), dried (MgSO₄), filtered and concentrated in vacuo to afford the sub-title compound (968 mg, 3.7 mmol, 67%). ¹H NMR (500 MHz, CDCl₃) δ 4.56-4.20 (m, 1H), 3.96 (s, 1H), 3.73-3.43 (m, 2H), 3.33 (s, 3H), 2.87-2.63 (m, 0.5H), 2.34 (s, 1H), 2.17-2.00 (m, 0.5H), 1.48 (s, 9H). Note: One exchangeable proton not observed.

### Step 4: tert-Butyl (2R)-4-methoxy-2-(5-methoxy-1H-indole-3-carbonyl)pyrrolidine-1-carboxylate

To a stirred solution of 5-methoxy-1H-indole (1.16 g, 2.00 Eq, 7.88 mmol) in dry toluene (35.0 mL) under a nitrogen atmosphere at 0 °C was added MeMgBr (940 mg, 2.63 mL, 3.00 molar, 2.00 Eq, 7.88 mmol) dropwise over 5 min and left to stir. In a separate vial, the product from step 3 above (968 mg, 1 Eq, 3.95 mmol) dissolved in dry DCM (5.00 mL) under a nitrogen atmosphere was treated with 1-chloro-N,N,2-trimethylprop-1-en-1-amine (539 mg, 534 μL, 1.02 Eq, 4.04 mmol). After 30 minutes, the acid chloride solution was added dropwise to the stirring solution of the deprotonated indole at 0 °C. The reaction mixture was stirred for 18 h. The reaction mixture was quenched with sat. NaHCO₃ (50 mL) at 0 °C and left to stir for 1 h, warming to rt. The resulting suspension was extracted with EtOAc (3×50 mL) and the collected organic layers were dried (Na₂SO₄) and filtered and concentrated in vacuo. The crude product was purified by chromatography on silica gel (80 g cartridge, 0-100% (1:3 EtOH/EtOAc+2% NH40H) in isohexane) to afford the sub-title compound (68.9 mg, 0.18 mmol, 4.5%). m/z 274.9 (M-Boc+H)⁺ (ES+); 373.2 (M−H)⁻ (ES−). ¹H NMR (500 MHz, DMSO) δ 11.85 (s, 1H), 8.29 (d, J=5.4 Hz, 1H), 7.69 (dd, J=12.0, 2.6 Hz, 1H), 7.37 (dd, J=8.8, 1.7 Hz, 1H), 6.85 (dt, J=8.8, 2.4 Hz, 1H), 5.07-4.96 (m, 1H), 3.99 (p, J=6.4 Hz, 1H), 3.80-3.75 (m, 4H), 3.26-3.13 (m, 1H), 3.19 (s, 2H), 3.16 (s, 1H), 2.71-2.61 (m, 1H), 1.80-1.71 (m, 1H), 1.40 (s, 4H), 1.13 (s, 5H).

### Step 5: (5-Methoxy-1H-indol-3-yl)(4-methoxypyrrolidin-2-yl)methanone, HCl

To a solution of the product from step 4 above (68.0 mg, 97% Wt, 1 Eq, 176 μmol) in DCM (2.50 mL) and MeOH (500 μL) at rt was added HCl in IPA (65.6 mg, 300 μL, 6.00 molar, 10.2 Eq, 1.80 mmol) dropwise. The reaction mixture was left to stir for 18 h. The reaction mixture was concentrated in vacuo to afford the sub-title compound (65.6 mg, 0.19 mmol, 110%). m/z 275.1 (M+H)⁺ (ES+); 273.2 (M−H)⁻ (ES−), ¹H NMR (500 MHz, DMSO) δ 12.31 (s, 1H), 9.87 (s, 1H), 8.79 (s, 1H), 8.42 (d, J=3.3 Hz, 1H), 7.66 (d, J=2.5 Hz, 1H), 7.44 (d, J=8.8 Hz, 1H), 6.92 (dd, J=8.8, 2.5 Hz, 1H), 5.07 (dd, J=10.5, 5.8 Hz, 1H), 4.13 (s, 1H), 3.80 (s, 3H), 3.48 (d, J=19.1 Hz, 1H), 3.31 (d, J=11.7 Hz, 1H), 3.16 (s, 3H), 2.84 (ddd, J=15.3, 10.4, 5.4 Hz, 1H), 2.08 (d, J=14.2 Hz, 1H).

### Step 6: 5-Methoxy-3-(((2S)-4-methoxypyrrolidin-2-yl)methyl)-1H-indole

To a stirred suspension of the product from step 5 above (65.6 mg, 89% Wt, 1 Eq, 188 μmol) in dry 2-Me-THF (2.00 mL) under a nitrogen atmosphere at 0 °C was added LiAlH₄ (2.4 M in THF) (28.5 mg, 313 μL, 2.40 molar, 4 Eq, 751 μmol). The reaction mixture was heated to 60 °C and stirred for 2.5 h. The mixture was cooled to 0 °C and quenched with 4:1 THF/water mixture (10 mL). Na₂SO₄ (5 g) was added to the suspension and stirred at rt overnight. The suspension was filtered through celite, washing with EtOAc (~200 mL). The filtrate was filtered and concentrated in vacuo to afford a crude brown foam. The foam was triturated with MTBE to afford the sub-title compound (40.0 mg, 0.10 mmol, 54%). m/z 261.3 (M+H)⁺ (ES+).

### Step 7: 5-methoxy-3-(((2S)-4-methoxy-1-((1-methyl-1H-pyrazol-3-yl)methyl)pyrrolidin-2-yl)methyl)-1H-indole

To a stirred solution of the product from step 6 above (40.0 mg, 65% Wt, 1 Eq, 99.9 μmol) in dry THF (2.00 mL) at rt was added 1-methyl-1H-pyrazole-3-carbaldehyde (17.4 mg, 95% Wt, 1.5 Eq, 150 μmol). The reaction mixture was stirred for 1 h. NaBH(OAc)₃ (63.5 mg, 3 Eq, 300 μmol) was added and the reaction mixture left to stir for 20 h at rt. The reaction mixture was quenched with sat. aq NaHCO₃ (5 mL) and left to stir for 20 min. The aq. suspension was extracted with EtOAc (3×5 mL). The combined organic layers were washed with brine (20 mL), dried (Na₂SO₄), filtered and concentrated in vacuo. The crude product was dissolved in DMSO (1.4 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130 Å, 5 μm, 30 mm×100 mm, flow rate 40 mL min−1 eluting with a 0.3% ammonia in water-MeCN gradient over 17.5 mins using UV across all wavelengths with PDA as well as a QDA and ELS detector. At-column dilution pump gives 2 mL min−1 MeOH over the entire method, which is included in the following MeCN percentages. Gradient information: 0.0-0.5 min, 15% MeCN; 0.5-15.5 min, ramped from 15% MeCN to 45% MeCN; 15.5-15.6 min, ramped from 45% MeCN to 100% MeCN; 15.6-17.5 min, held at 100% MeCN. The clean fractions were evaporated in a freeze-dryer to the title compound (5.70 mg, 15 μmol, 15%) as a gum. m/z 355.1 (M+H)⁺ (ES+). ¹H NMR (500 MHz, DMSO) δ 10.63-10.55 (m, 1H), 7.59 (d, J=2.1 Hz, 1H), 7.20 (d, J=8.7 Hz, 1H), 7.10 (d, J=2.1 Hz, 1H), 6.99 (d, J=2.4 Hz, 1H), 6.70 (dd, J=8.7, 2.4 Hz, 1H), 6.14 (d, J=2.0 Hz, 1H), 3.94 (d, J=13.6 Hz, 1H), 3.80 (s, 3H), 3.75 (s, 3H), 3.70-3.63 (m, 1H), 3.44 (d, J=13.5 Hz, 1H), 3.14-3.09 (m, 1H), 2.93 (d, J=10.6 Hz, 1H), 2.70-2.62 (m, 1H), 2.54 (dd, J=13.9, 9.4 Hz, 1H), 2.39 (dd, J=10.7, 6.2 Hz, 1H), 1.95 (dt, J=13.9, 7.2 Hz, 1H), 1.44 (ddd, J=12.9, 8.4, 4.0 Hz, 1H).

### Example 36: Synthesis of (R)-5-(methylthio)-3-((1-(tetrahydro-2H-pyran-4-yl)pyrrolidin-2-yl)methyl)-1H-indole (18B)

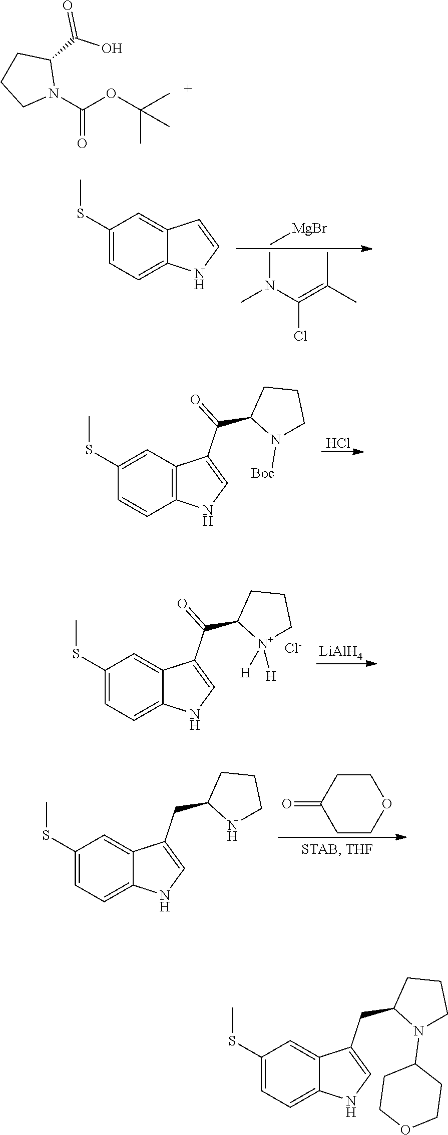

### Step 1: tert-butyl (R)-2-(5-(methylthio)-1H-indole-3-carbonyl)pyrrolidine-1-carboxylate

A solution of 5-methylthio-indole (4.17 g, 2 Eq, 24.8 mmol) in dry toluene (40.00 mL) at 0 °C was treated with MeMgBr in Et₂O (2.96 g, 8.26 mL, 3.00 molar, 2 Eq, 24.8 mmol) and the mixture was stirred for 30 min before being warmed to rt and stirred for a further 30 min. Simultaneously, ((benzyloxy)carbonyl)-D-proline (2.75 g, 1 Eq, 12.4 mmol) in DCM (40.00 mL) at rt was treated with 1-chloro-N,N,2-trimethylprop-1-en-1-amine (1.82 g, 1.80 mL, 1.1 Eq, 13.6 mmol) and the resulting solution was stirred at rt for 45 min before being added dropwise to the solution of deprotonated indole. The reaction mixture was stirred at rt for 18 h before the mixture was treated with sat. aq. NaHCO₃ solution (80 mL). The aq. layer was extracted with EtOAc (3×75 mL) before the pooled organics were washed with brine (40 mL), dried (MgSO₄) and concentrated in vacuo. The crude product was purified by chromatography on silica gel (120 g cartridge, 0-100% EtOAc/iso-hexane) and the product then triturated from MTBE to afford the sub-title compound (1.06 g, 2.94 mmol, 24%). m/z 260.82 (M+H−CO₂tBu)⁺ (ES+). ¹H NMR 500 MHz, DMSO) δ 12.01 (s, 2H), 8.45-8.41 (m, 1H), 8.41 (m, 1H), 8.14-8.11 (m, 1H), 8.11-8.08 (m, 1H), 7.45-7.43 (m, 1H), 7.44-7.42 (m, 1H), 7.21-7.18 (m, 1H), 7.19-7.17 (m, 1H), 5.10 (dd, J=8.9, 3.7 Hz, 1H), 5.06-5.01 (m, 1H), 3.49-3.36 (m, 4H), 2.34-2.21 (m, 2H), 1.88-1.74 (m, 6H), 1.39 (s, 9H), 1.13 (s, 9H). Product is present as a ~1:1 mixture of tautomers with the spectra possessing multiple regions of overlapping. Therefore, spectrum has been quoted as a mixture of both species. Due to overlap with residual DMSO peak, both tautomer's SMe peaks are obscured.

### Step 2: (R)-2-(5-(methylthio)-1H-indole-3-carbonyl)pyrrolidin-1-ium

To a solution of the product from step 1 above (1.06 g, 1 Eq, 2.94 mmol) in DCM (30.0 mL) and MeOH at rt was added HCl in IPA (1.07 g, 4.90 mL, 6.00 molar, 10 Eq, 29.4 mmol) dropwise. The reaction mixture was left to stir for 18 h and then concentrated in vacuo to afford sub-title compound (893 mg, 3.01 mmol, 102%). m/z 261.12 (M+H)⁺ (ES+). ¹H NMR (500 MHz, DMSO) δ 12.51-12.47 (m, 1H), 8.58 (d, J=3.2 Hz, 1H), 8.09 (d, J=1.9 Hz, 1H), 7.50 (d, J=8.4 Hz, 1H), 7.24 (dd, J=8.4, 1.9 Hz, 1H), 5.06 (s, 1H), 3.29 (d, J=11.1 Hz, 2H), 2.71-2.50 (m, 2H), 2.04-1.85 (m, 3H). SMe peak is not observed due to overlap with residual DMSO solvent peak.

### Step 3: (R)-5-(methylthio)-3-(pyrrolidin-2-ylmethyl)-1H-indole

To a stirred suspension of the product from step 2 above (873 mg, 1 Eq, 2.94 mmol) in dry 2-methyltetrahydrofuran (30.00 mL) under a nitrogen atmosphere at 0 °C was added lithium aluminium hydride in THF (558 mg, 6.13 mL, 2.40 molar, 5 Eq, 14.7 mmol). The reaction mixture was heated to 60 °C and stirred for 5 hours. LCMS analysis indicated that all the starting material had been consumed but the reaction had stalled at the partially reduced indole.

The mixture was cooled to 0 °C, diluted with TBME (15 mL) then water (0.5 mL) was added followed by 2 M aq. NaOH (0.5 mL) and water (1.5 mL). The mixture was warmed to rt and stirred for 30 min then filtered washing with EtOAc (2×10 mL) and the filtrate was concentrated in vacuo. The resulting product was dissolved in dry 2-Methyltetrahydrofuran (30.00 mL), cooled to 0 °C, and lithium aluminium hydride in THF (335 mg, 3.68 mL, 2.40 molar, 3 Eq, 8.82 mmol) added dropwise. The reaction mixture was heated to 60 °C and stirred for 2 hours. The mixture was cooled to 0 °C, diluted with TBME (15 mL) then water (0.5 mL) was added followed by 2 M aq. NaOH (0.5 mL), water (1.5 mL), MgSO₄ (ca 2.0 g). The mixture was warmed to rt and stirred for 30 min then filtered washing with EtOAc (2×10 mL) and the filtrate was concentrated in vacuo. The resulting product was dissolved in MeOH (ca 10 mL) and loaded onto a cartridge of Si-TsOH (ca 5 g) washing with MeOH (3×10 mL). The cartridge was eluted with 0.7 M NH3/MeOH (3×10 mL) and concentrated under reduced pressure to afford sub-title compound (426.1 mg, 1.2 mmol, 45%, 72% Purity). m/z 247.00 (M+H). ¹H NMR (500 MHz, DMSO) δ 10.83 (s, 1H), 7.49 (s, 1H), 7.29 (d, J=8.4 Hz, 1H), 7.15 (s, 1H), 7.06 (dd, J=8.4, 1.8 Hz, 1H), 3.23-3.17 (m, 1H), 2.91-2.84 (m, 1H), 2.76 (dd, J=14.2, 6.3 Hz, 1H), 2.72-2.64 (m, 2H), 2.45 (s, 3H), 1.77-1.52 (m, 4H), 1.34-1.22 (m, 1H).

### Step 4: (R)-5-(methylthio)-3-((1-(tetrahydro-2H-pyran-4-yl)pyrrolidin-2-yl)methyl)-1H-indole

To a stirred solution of the product from step 3 above (84.0 mg, 72% Wt, 1 Eq, 245 μmol) in dry THF (3 mL) at rt was added tetrahydro-4H-pyran-4-one (32.6 mg, 30.1 μL, 98% Wt, 1.3 Eq, 319 μmol). The reaction mixture was stirred for 2 hours, sodium triacetoxyborohydride (156 mg, 3 Eq, 736 μmol) added and then left to stir overnight. The reaction mixture was quenched with sat. sodium bicarbonate solution (5 mL) and left to stir. The aqueous suspension was diluted with water (5 mL) and extracted with EtOAc (3×10 mL). The combined organic layers were dried over MgSO₄, filtered and concentrated in vacuo. The crude product was first purified by chromatography on silica gel (12 g cartridge, 0-10% (0.7 M NH₃/MeOH) in DCM), and then further purified by chromatography on C18 (13 g cartridge, 5-20% (MeCN+0.1% formic acid) in (water+0.1% formic acid)) to afford sub-title compound (20.4 mg, 61.7 μmol, 25.1%, 100% Purity). m/z 331.18 (M+H)⁺. ¹H NMR (500 MHz, DMSO) δ 10.83 (s, 1H), 7.43 (s, 1H), 7.28 (d, J=8.4 Hz, 1H), 7.14 (s, 1H), 7.04 (d, J=8.4 Hz, 1H), 3.89 (d, J=11.4 Hz, 2H), 3.38-3.26 (m, 2H), 3.12-3.04 (m, 1H), 2.88-2.76 (m, 3H), 2.57-2.46 (m, 5H), 1.83-1.71 (m, 2H), 1.66-1.55 (m, 4H), 1.52-1.43 (m, 2H).

### Example 37: Synthesis of (R)-3-((1-((1H-pyrazol-3-yl)methyl)pyrrolidin-2-yl)methyl)-5-(methylthio)-1H-indole (20A)

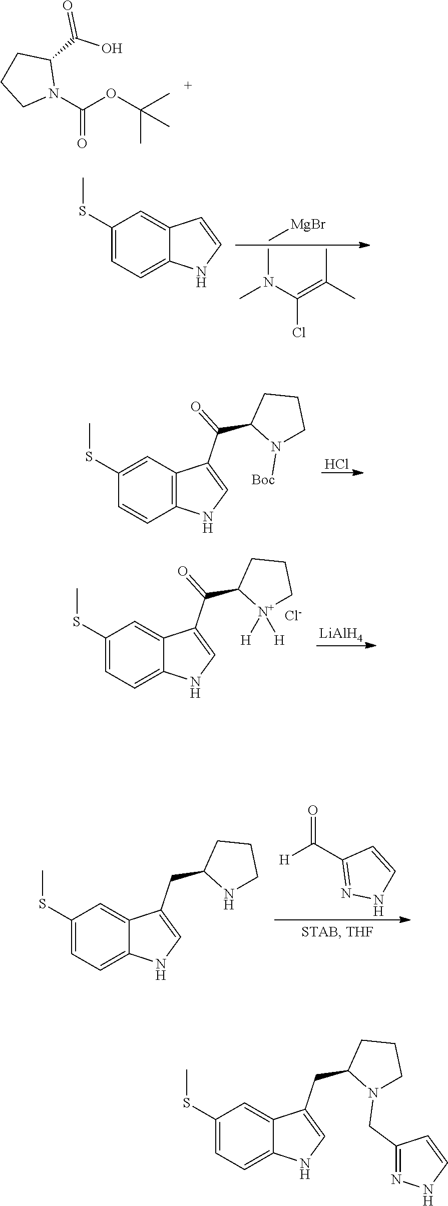

### Step 1: tert-butyl (R)-2-(5-(methylthio)-1H-indole-3-carbonyl)pyrrolidine-1-carboxylate

A solution of 5-methylthio-indole (4.17 g, 2 Eq, 24.8 mmol) in dry toluene (40.00 mL) at 0 °C was treated with MeMgBr in Et₂O (2.96 g, 8.26 mL, 3.00 molar, 2 Eq, 24.8 mmol) and the mixture was stirred for 30 min before being warmed to rt and stirred for a further 30 min. Simultaneously, ((benzyloxy)carbonyl)-D-proline (2.75 g, 1 Eq, 12.4 mmol) in DCM (40.00 mL) at rt was treated with 1-chloro-N,N,2-trimethylprop-1-en-1-amine (1.82 g, 1.80 mL, 1.1 Eq, 13.6 mmol) and the resulting solution was stirred at rt for 45 min before being added dropwise to the solution of deprotonated indole. The reaction mixture was stirred at rt for 18 h before the mixture was treated with sat. aq. NaHCO₃ solution (80 mL). The aq. layer was extracted with EtOAc (3×75 mL) before the pooled organics were washed with brine (40 mL), dried (MgSO₄) and concentrated in vacuo. The crude product was purified by chromatography on silica gel (120 g cartridge, 0-100% EtOAc/iso-hexane) and the product then triturated from MTBE to afford the sub-title compound (1.06 g, 2.94 mmol, 24%). m/z 260.82 (M+H−CO₂tBu)⁺ (ES+) ¹H NMR 500 MHz, DMSO) δ 12.01 (s, 2H), 8.45-8.41 (m, 1H), 8.41 (m, 1H), 8.14-8.11 (m, 1H), 8.11-8.08 (m, 1H), 7.45-7.43 (m, 1H), 7.44-7.42 (m, 1H), 7.21-7.18 (m, 1H), 7.19-7.17 (m, 1H), 5.10 (dd, J=8.9, 3.7 Hz, 1H), 5.06-5.01 (m, 1H), 3.49-3.36 (m, 4H), 2.34-2.21 (m, 2H), 1.88-1.74 (m, 6H), 1.39 (s, 9H), 1.13 (s, 9H). Product is present as a ~1:1 mixture of tautomers with the spectra possessing multiple regions of overlapping. Therefore, spectrum has been quoted as a mixture of both species. Due to overlap with residual DMSO peak, both tautomer's SMe peaks are obscured.

### Step 2: (R)-2-(5-(methylthio)-1H-indole-3-carbonyl)pyrrolidin-1-ium

To a solution of the product from step 1 above (1.06 g, 1 Eq, 2.94 mmol) in DCM (30.0 mL) and MeOH at rt was added HCl in IPA (1.07 g, 4.90 mL, 6.00 molar, 10 Eq, 29.4 mmol) dropwise. The reaction mixture was left to stir for 18 h and then concentrated in vacuo to afford sub-title compound (893 mg, 3.01 mmol, 102%). m/z 261.12 (M+H)⁺ (ES⁺) ¹H NMR (500 MHz, DMSO) δ 12.51-12.47 (m, 1H), 8.58 (d, J=3.2 Hz, 1H), 8.09 (d, J=1.9 Hz, 1H), 7.50 (d, J=8.4 Hz, 1H), 7.24 (dd, J=8.4, 1.9 Hz, 1H), 5.06 (s, 1H), 3.29 (d, J=11.1 Hz, 2H), 2.71-2.50 (m, 2H), 2.04-1.85 (m, 3H). SMe peak is not observed due to overlap with residual DMSO solvent peak.

### Step 3: (R)-5-(methylthio)-3-(pyrrolidin-2-ylmethyl)-1H-indole

To a stirred suspension of the product from step 2 above (873 mg, 1 Eq, 2.94 mmol) in dry 2-methyltetrahydrofuran (30.00 mL) under a nitrogen atmosphere at 0 °C was added lithium aluminium hydride in THF (558 mg, 6.13 mL, 2.40 molar, 5 Eq, 14.7 mmol). The reaction mixture was heated to 60 °C and stirred for 5 hours. LCMS analysis indicated that all the starting material had been consumed but the reaction had stalled at the partially reduced indole.

The mixture was cooled to 0 °C, diluted with TBME (15 mL) then water (0.5 mL) was added followed by 2 M aq. NaOH (0.5 mL) and water (1.5 mL). The mixture was warmed to rt and stirred for 30 min then filtered washing with EtOAc (2×10 mL) and the filtrate was concentrated in vacuo. The resulting product was dissolved in dry 2-Methyltetrahydrofuran (30.00 mL), cooled to 0 °C, and lithium aluminium hydride in THF (335 mg, 3.68 mL, 2.40 molar, 3 Eq, 8.82 mmol) added dropwise. The reaction mixture was heated to 60 °C and stirred for 2 hours. The mixture was cooled to 0 °C, diluted with TBME (15 mL) then water (0.5 mL) was added followed by 2 M aq. NaOH (0.5 mL), water (1.5 mL), MgSO₄ (ca 2.0 g). The mixture was warmed to rt and stirred for 30 min then filtered washing with EtOAc (2×10 mL) and the filtrate was concentrated in vacuo. The resulting product was dissolved in MeOH (ca 10 mL) and loaded onto a cartridge of Si-TsOH (ca 5 g) washing with MeOH (3×10 mL). The cartridge was eluted with 0.7 M NH₃/MeOH (3×10 mL) and concentrated under reduced pressure to afford sub-title compound (426.1 mg, 1.2 mmol, 45%, 72% Purity). m/z 247.00 (M+H)⁺. ¹H NMR (500 MHz, DMSO) δ 10.83 (s, 1H), 7.49 (s, 1H), 7.29 (d, J=8.4 Hz, 1H), 7.15 (s, 1H), 7.06 (dd, J=8.4, 1.8 Hz, 1H), 3.23-3.17 (m, 1H), 2.91-2.84 (m, 1H), 2.76 (dd, J=14.2, 6.3 Hz, 1H), 2.72-2.64 (m, 2H), 2.45 (s, 3H), 1.77-1.52 (m, 4H), 1.34-1.22 (m, 1H).

### Step 4: (R)-3-((1-((1H-pyrazol-3-yl)methyl)pyrrolidin-2-yl)methyl)-5-(methylthio)-1H-indole

To a stirred solution of the product from step 3 above (84.0 mg, 72% Wt, 1 Eq, 245 μmol) in dry THF (3 mL) at rt was added 1H-pyrazole-3-carbaldehyde (31.6 mg, 97% Wt, 1.30 Eq, 0.319 mmol). The reaction mixture was stirred for 2 hours, sodium triacetoxyhydroborate (156 mg, 2.99 Eq, 0.735 mmol) added, and then left to stir overnight. The reaction mixture was quenched with sat. sodium bicarbonate solution (5 mL) and left to stir. The aqueous suspension was diluted with water (5 mL) and extracted with EtOAc (3×10 mL). The combined organic layers were dried over MgSO₄, filtered and concentrated in vacuo. The crude product was first purified by chromatography on silica gel (12 g cartridge, 0-10% (0.7 M NH₃/MeOH) in DCM) and then further purified by chromatography on C18 (13 g cartridge, 5-20% (MeCN+0.1% formic acid) in (water+0.1% formic acid)) to afford sub-title compound (25.6 mg, 78 μmol, 32%, 99% Purity). m/z 327.21 (M+H)⁺. ¹H NMR (500 MHz, DMSO) δ 12.59 (s, 1H), 10.83 (s, 1H), 7.53 (s, 1H), 7.49 (s, 1H), 7.28 (d, J=8.4 Hz, 1H), 7.18-7.13 (m, 1H), 7.05 (dd, J=8.4, 1.8 Hz, 1H), 6.17 (d, J=1.9 Hz, 1H), 4.02 (d, J=13.6 Hz, 1H), 3.52 (d, J=13.6 Hz, 1H), 3.05 (dd, J=14.1, 3.7 Hz, 1H), 2.92-2.84 (m, 1H), 2.69-2.65 (m, 1H), 2.56-2.50 (m, 1H), 2.48 (s, 3H), 2.31-2.21 (m, 1H), 1.69-1.62 (m, 1H), 1.61-1.50 (m, 2H), 1.45 (m, 1H).

### Example 38: Synthesis of (R)-4-Fluoro-5-methoxy-3-((1-((5-methyl-1H-pyrazol-3-yl)methyl)pyrrolidin-2-yl)methyl)-1H-indole (3A)

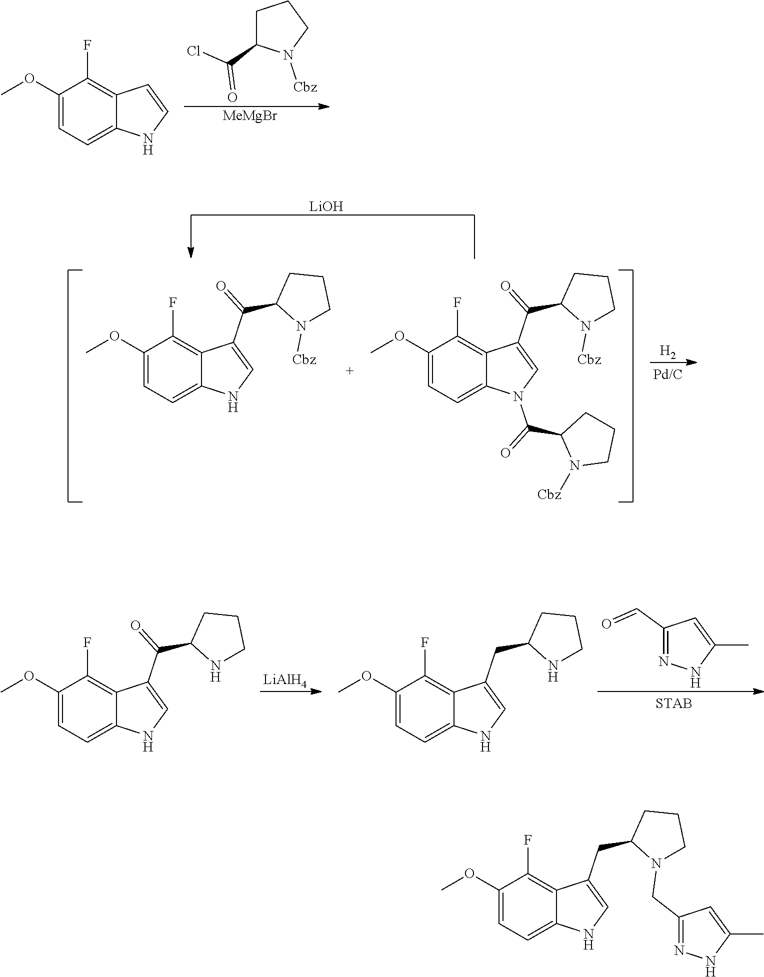

### Step 1: Benzyl (R)-2-(4-fluoro-5-methoxy-1H-indole-3-carbonyl)pyrrolidine-1-carboxylate

To a solution of 4-fluoro-5-methoxy-1H-indole (1.00 g, 1.25 Eq, 6.05 mmol) in toluene (20.00 mL) cooled to 0 °C was added drop-wise MeMgBr in Et₂O (753 mg, 2.10 mL, 3.00 molar, 1.3 Eq, 6.31 mmol). After 30 min the mixture was warmed to rt and stirred for a further 90 min. A solution of benzyl (R)-2-(chlorocarbonyl)pyrrolidine-1-carboxylate (1.30 g, 1 Eq, 4.86 mmol) in toluene (5.00 mL) was added drop-wise and stirring was continued for a further 1 h. The mixture was poured into sat. aq. NH₄Cl (100 mL) and extracted with EtOAc (3×50 mL). The combined organics were washed with water (50 mL), brine (50 mL), dried (MgSO₄), filtered and concentrated in vacuo to afford a brown gum. The crude product was purified by chromatography on silica gel (24 g cartridge, 0-100% EtOAc in isohexane, eluted at 80%) to afford the sub-title compound, contaminated with benzyl 2-(3-(((benzyloxy)carbonyl)-D-prolyl)-4-fluoro-5-methoxy-1H-indole-1-carbonyl)pyrrolidine-1-carboxylate (1.29 g, 3.25 mmol, 67.0%). This material was dissolved in THF (20.0 mL) and water (4.0 mL) to which was added LiOH (390 mg, 5 Eq, 16.3 mmol) and the mixture was stirred at rt for 5 h. The mixture was poured into sat. aq. NH₄Cl (200 mL) and extracted with EtOAc (3×25 mL). The combined organics were washed with water (50 mL), brine (50 mL), dried (MgSO₄), filtered and concentrated in vacuo. The crude product was purified by chromatography on silica gel (24 g cartridge, 0-100% EtOAc in isohexane, eluted at 80%) to afford the sub-title compound (576 mg, 1.2 mmol, 25%). Note: yield calculated over 2 steps. m/z 397.3 (M+H)⁺ (ES+).

### Step 2: (R)-4-Fluoro-5-methoxy-3-prolyl-1H-indole

A stirred suspension of the product from step 1 above (576 mg, 83% Wt, 1 Eq, 1.21 mmol) and Pd/C (87 L) (128 mg, 5% Wt, 0.05 Eq, 60.3 μmol) in EtOH (7.50 mL) under a nitrogen atmosphere at rt and the vessel was charged with H2 (2.44 mg, 1 Eq, 1.21 mmol). The reaction mixture was stirred at rt for 18 h. The reaction mixture was filtered through glass microfibres, rinsing with MeOH (250 mL). The resulting product was redissolved in MeOH (7.00 mL) and Pd/C (87 L) (128 mg, 5% Wt, 0.05 Eq, 60.3 μmol) was added. Under a nitrogen atmosphere at rt, the vessel was charged with H₂ (2.44 mg, 1 Eq, 1.21 mmol). The reaction mixture was stirred at rt for 6 h. The reaction mixture was filtered through glass microfibres, rinsing with MeOH (150 mL). The solvent was removed in vacuo to afford the sub-title compound (360 mg, 1.2 mmol, 100%). m/z 263.0 (M+H)⁺ (ES+); 261.2 (M−H)⁻ (ES−). ¹H NMR (500 MHz, DMSO) δ 8.36 (s, 1H), 7.22 (d, J=8.8 Hz, 1H), 7.12 (dd, J=8.7, 7.3 Hz, 1H), 4.41 (dd, J=8.6, 6.0 Hz, 1H), 3.83 (s, 3H), 3.04 (dt, J=10.3, 6.1 Hz, 1H), 2.73 (dt, J=10.4, 6.6 Hz, 1H), 2.19-2.09 (m, 1H), 1.77-1.66 (m, 1H), 1.66-1.56 (m, 2H). 2 exchangeable protons not observed. ¹⁹F NMR (471 MHz, DMSO) δ −131.67.

### Step 3: (R)-4-Fluoro-5-methoxy-3-(pyrrolidin-2-ylmethyl)-1H-indol

To a stirred suspension of the product from step 2 above (360 mg, 90% Wt, 1 Eq, 1.24 mmol) in dry 2-Me-THF (10.0 mL) under a nitrogen atmosphere at 0 °C was added LiAlH₄ (2.4 M in THF) (188 mg, 2.06 mL, 2.40 molar, 4 Eq, 4.94 mmol). The reaction mixture was heated to 70 °C and stirred. The mixture was cooled to 0 °C and quenched with 4:1 THF/water mixture (10 mL). Na₂SO₄ (10 g) was added to the suspension and stirred at rt for 1 h. The suspension was filtered through celite, washing with EtOAc (~200 mL). The filtrate was filtered and concentrated in vacuo to afford the sub-title compound (383 mg, 0.77 mmol, 62%) as a crude brown oil. m/z 249.3 (M+H)+ (ES+).

### Step 4: (R)-4-Fluoro-5-methoxy-3-((1-((5-methyl-1H-pyrazol-3-yl)methyl)pyrrolidin-2-yl)methyl)-1H-indo

To a stirred solution of the product from step 3 above (80.0 mg, 50% Wt, 1 Eq, 161 μmol) in dry THF (2.00 mL) at rt was added 5-methyl-1H-pyrazole-3-carbaldehyde (22.4 mg, 95% Wt, 1.2 Eq, 193 μmol). The reaction mixture was stirred for 30 min. NaBH(OAc)₃ (102 mg, 3 Eq, 483 μmol) was added and the reaction mixture left to stir for 18 h at rt. The reaction mixture was quenched with sat. aq. NaHCO₃ (5 mL) and left to stir for 20 min. The aq. suspension was diluted with water (5 mL) and extracted with EtOAc (3×10 mL). The combined organic layers were dried (Na₂SO₄), filtered and concentrated in vacuo. The crude product was purified by chromatography on silica gel (12 g cartridge, 0-10% (0.7 M NH₃/MeOH) in DCM) to afford product. The crude was purified again by chromatography on silica gel (4 g cartridge, 0-10% MeOH in DCM) to afford the title compound (9.20 mg, 23 μmol, 14%). m/z 343.6 (M+H)⁺ (ES+); 341.0 (M−H)⁻ (ES−). ¹H NMR (500 MHz, DMSO) δ 12.14 (s, 1H), 10.86 (s, 1H), 7.13 (d, J=2.2 Hz, 1H), 7.05 (d, J=8.7 Hz, 1H), 6.93 (t, J=8.3 Hz, 1H), 5.87 (s, 1H), 3.93 (d, J=13.7 Hz, 1H), 3.81 (s, 3H), 3.15 (dd, J=13.7, 3.4 Hz, 1H), 2.86 (t, J=7.7 Hz, 1H), 2.66 (s, 1H), 2.57 (dd, J=13.8, 9.1 Hz, 1H), 2.16 (s, 4H), 1.75 (s, 1H), 1.70-1.59 (m, 1H), 1.60-1.43 (m, 3H). ¹⁹F NMR (471 MHz, DMSO) δ −147.49.

### Example 39: Synthesis of (R)-4-Fluoro-5-methoxy-3-((1-((1-methyl-1H-pyrazol-4-yl)methyl)pyrrolidin-2-yl)methyl)-1H-indole (1A)

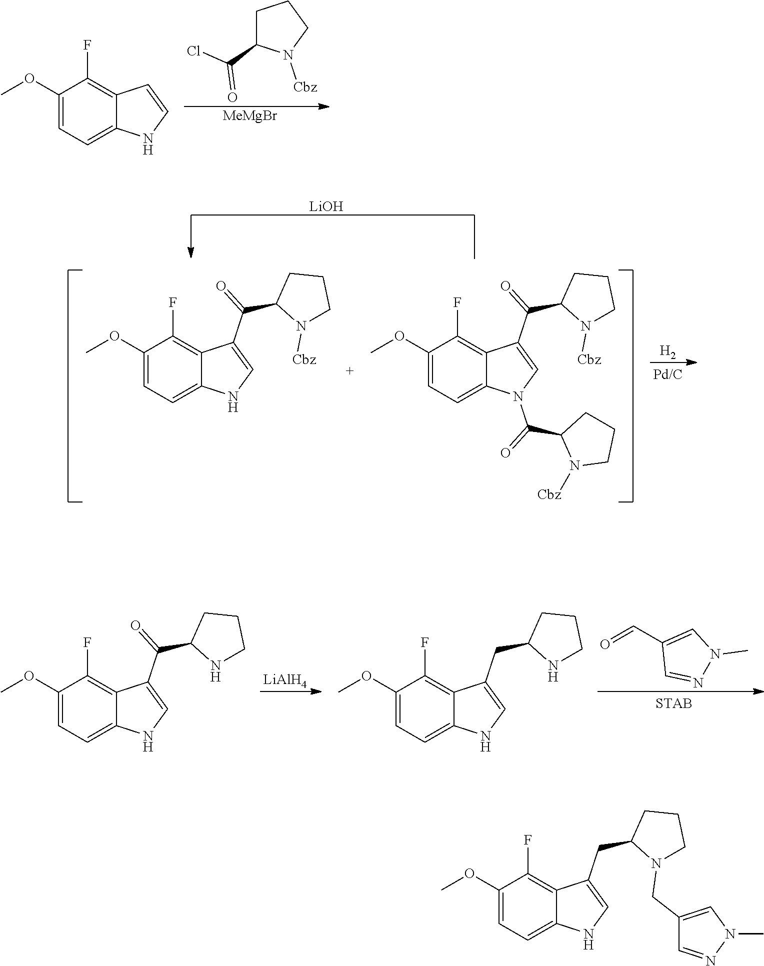

### Step 1: Benzyl (R)-2-(4-fluoro-5-methoxy-1H-indole-3-carbonyl)pyrrolidine-1-carboxylate

To a solution of 4-fluoro-5-methoxy-1H-indole (1.00 g, 1.25 Eq, 6.05 mmol) in toluene (20.00 mL) cooled to 0 °C was added drop-wise MeMgBr in Et₂O (753 mg, 2.10 mL, 3.00 molar, 1.3 Eq, 6.31 mmol). After 30 min the mixture was warmed to rt and stirred for a further 90 min. A solution of benzyl (R)-2-(chlorocarbonyl)pyrrolidine-1-carboxylate (1.30 g, 1 Eq, 4.86 mmol) in toluene (5.00 mL) was added drop-wise and stirring was continued for a further 1 h. The mixture was poured into sat. aq. NH₄Cl (100 mL) and extracted with EtOAc (3×50 mL). The combined organics were washed with water (50 mL), brine (50 mL), dried (MgSO₄), filtered and concentrated in vacuo to afford a brown gum. The crude product was purified by chromatography on silica gel (24 g cartridge, 0-100% EtOAc in isohexane, eluted at 80%) to afford the sub-title compound, contaminated with benzyl 2-(3-(((benzyloxy)carbonyl)-D-prolyl)-4-fluoro-5-methoxy-1H-indole-1-carbonyl)pyrrolidine-1-carboxylate (1.29 g, 3.25 mmol, 67.0%). This material was dissolved in THF (20.0 mL) and water (4.0 mL) to which was added LiOH (390 mg, 5 Eq, 16.3 mmol) and the mixture was stirred at rt for 5 h. The mixture was poured into sat. aq. NH₄Cl (200 mL) and extracted with EtOAc (3×25 mL). The combined organics were washed with water (50 mL), brine (50 mL), dried (MgSO₄), filtered and concentrated in vacuo. The crude product was purified by chromatography on silica gel (24 g cartridge, 0-100% EtOAc in isohexane, eluted at 80%) to afford the sub-title compound (576 mg, 1.2 mmol, 25%). Note: yield calculated over 2 steps. m/z 397.3 (M+H)⁺ (ES+).

### Step 2: (R)-4-Fluoro-5-methoxy-3-prolyl-1H-indole

A stirred suspension of the product from step 1 above (576 mg, 83% Wt, 1 Eq, 1.21 mmol) and Pd/C (87 L) (128 mg, 5% Wt, 0.05 Eq, 60.3 μmol) in EtOH (7.50 mL) under a nitrogen atmosphere at rt and the vessel was charged with H2 (2.44 mg, 1 Eq, 1.21 mmol). The reaction mixture was stirred at rt for 18 h. The reaction mixture was filtered through glass microfibres, rinsing with MeOH (250 mL). The resulting product was redissolved in MeOH (7.00 mL) and Pd/C (87 L) (128 mg, 5% Wt, 0.05 Eq, 60.3 μmol) was added. Under a nitrogen atmosphere at rt, the vessel was charged with H₂ (2.44 mg, 1 Eq, 1.21 mmol). The reaction mixture was stirred at rt for 6 h. The reaction mixture was filtered through glass microfibres, rinsing with MeOH (150 mL). The solvent was removed in vacuo to afford the sub-title compound (360 mg, 1.2 mmol, 100%). m/z 263.0 (M+H)+ (ES+); 261.2 (M−H)− (ES−). ¹H NMR (500 MHz, DMSO) δ 8.36 (s, 1H), 7.22 (d, J=8.8 Hz, 1H), 7.12 (dd, J=8.7, 7.3 Hz, 1H), 4.41 (dd, J=8.6, 6.0 Hz, 1H), 3.83 (s, 3H), 3.04 (dt, J=10.3, 6.1 Hz, 1H), 2.73 (dt, J=10.4, 6.6 Hz, 1H), 2.19-2.09 (m, 1H), 1.77-1.66 (m, 1H), 1.66-1.56 (m, 2H). 2 exchangeable protons not observed. ¹⁹F NMR (471 MHz, DMSO) δ −131.67.

### Step 3: (R)-4-Fluoro-5-methoxy-3-(pyrrolidin-2-ylmethyl)-1H-indol

To a stirred suspension of the product from step 2 above (360 mg, 90% Wt, 1 Eq, 1.24 mmol) in dry 2-Me-THF (10.0 mL) under a nitrogen atmosphere at 0 °C was added LiAlH₄ (2.4 M in THF) (188 mg, 2.06 mL, 2.40 molar, 4 Eq, 4.94 mmol). The reaction mixture was heated to 70 °C and stirred. The mixture was cooled to 0 °C and quenched with 4:1 THF/water mixture (10 mL). Na₂SO₄ (10 g) was added to the suspension and stirred at rt for 1 h. The suspension was filtered through celite, washing with EtOAc (~200 mL). The filtrate was filtered and concentrated in vacuo to afford the sub-title compound (383 mg, 0.77 mmol, 62%) as a crude brown oil. m/z 249.3 (M+H)⁺ (ES+).

### Step 4: (R)-4-Fluoro-5-methoxy-3-((1-((1-methyl-1H-pyrazol-4-yl)methyl)pyrrolidin-2-yl)methyl)-1H-indole

To a stirred solution of the product from step 3 above (80.0 mg, 50% Wt, 1 Eq, 161 μmol) in dry THF (2.00 mL) at rt was added 1-methyl-1H-pyrazole-4-carbaldehyde (21.3 mg, 1.2 Eq, 193 μmol). The reaction mixture was stirred for 30 min. NaBH(OAc)₃ (102 mg, 3 Eq, 483 μmol) was added and the reaction mixture left to stir for 18 h at rt. The reaction mixture was quenched with sat. aq. NaHCO₃ solution (3 mL) and left to stir for 20 min. The aq. suspension was extracted with EtOAc (3×5 mL). The combined organic layers were dried (Na₂SO₄), filtered and concentrated in vacuo. The crude product was purified by chromatography on silica gel (12 g cartridge, 0-10% (0.7 M NH₃/MeOH) in DCM) to afford the title compound (10.4 mg, 26 μmol, 16%). m/z 343.3 (M+H)⁺ (ES+); 341.1 (M−H)− (ES−). ¹H NMR (500 MHz, DMSO) δ 10.86 (s, 1H), 7.52 (s, 1H), 7.29-7.26 (m, 1H), 7.13 (d, J=2.2 Hz, 1H), 7.06 (d, J=8.7 Hz, 1H), 6.93 (t, J=8.3 Hz, 1H), 3.86 (d, J=13.4 Hz, 1H), 3.81 (s, 3H), 3.78 (s, 3H), 3.23 (d, J=13.5 Hz, 1H), 3.15 (dd, J=13.1, 3.0 Hz, 1H), 2.89-2.82 (m, 1H), 2.67-2.60 (m, 1H), 2.57 (dd, J=13.2, 9.1 Hz, 1H), 2.13 (q, J=8.3 Hz, 1H), 1.68-1.58 (m, 1H), 1.58-1.44 (m, 3H). ¹⁹F NMR (471 MHz, DMSO) δ −147.47.

### Example 40: Synthesis of 5-Methoxy-3-((1-methylazetidin-2-yl)methyl)-1H-indole (89)

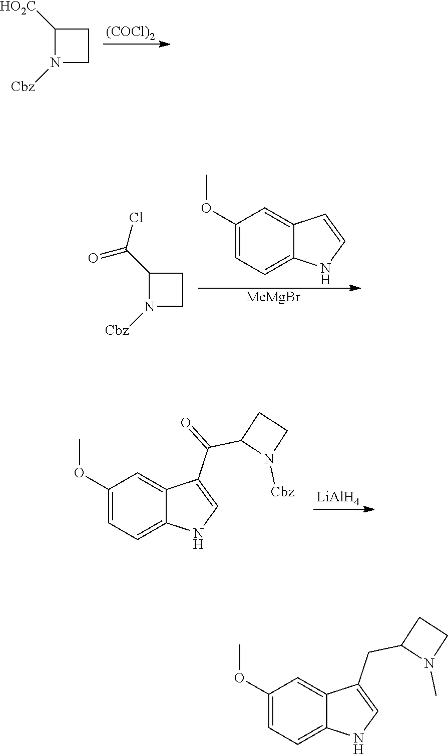

### Step 1: Benzyl 2-(chlorocarbonyl)azetidine-1-carboxylate

To a mixture of 1-((benzyloxy)carbonyl)azetidine-2-carboxylic acid (0.30 g, 1 Eq, 1.3 mmol) in DCM (4.00 mL) at 0 °C was added oxalyl chloride (0.49 g, 0.33 mL, 3 Eq, 3.8 mmol) followed by a few drops of DMF. The reaction mixture was stirred at the rt for 2 h and then the solvent was removed in vacuo to afford the sub-title compound (0.32 g, 1.3 mmol, 99%) as yellow oil. The product was used in the next without further purification.

### Step 2: Benzyl 2-(5-methoxy-1H-indole-3-carbonyl)azetidine-1-carboxylate

To a stirred solution of 5-methoxy-1H-indole (0.37 g, 2 Eq, 2.5 mmol) in dry toluene (10.0 mL) under a nitrogen atmosphere at 0 °C was added MeMgBr (0.30 g, 0.84 mL, 3.00 molar, 2 Eq, 2.5 mmol) dropwise over 10 min and the mixture was stirred at rt for 3 hours. The product from step 1 above (0.32 g, 1 Eq, 1.3 mmol) in toluene (1.00 mL) was added dropwise to the stirring solution of the deprotonated indole and the reaction mixture was stirred at rt for 16 h. The reaction mixture was quenched with sat. NaHCO₃ (150 mL) at 0 °C and left to stir for 1 h, warming to rt. The two phases were separated and the aq. phase was extracted with EtOAc (3×100 mL). The combined organic phases were washed with brine (50 mL), dried (MgSO₄), filtered and the solvent removed in vacuo. The crude product was purified by flash chromatography on silica gel (12 g column, 0-70% EtOAc in hexanes, load (silica)) to afford the sub-title compound (0.180 g, 0.43 mmol, 34%). m/z 320.8 (M-CO₂+H)⁺ (ES+); 363.3 (M−H)⁻ (ES−).

### Step 3: 5-Methoxy-3-((1-methylazetidin-2-yl)methyl)-1H-indole

To a mixture of the product from step 2 above (190 mg, 1 Eq, 521 μmol) in 2-Me-THF (5.00 mL) at 0 °C was added LiAlH₄ (98.9 mg, 1.09 mL, 2.40 molar, 5 Eq, 2.61 mmol) and the mixture was stirred at 66 °C for 3 h. The mixture was cooled to 0 °C, diluted with THF (10 mL), quenched with H₂O (1 mL), 2 M NaOH (0.25 mL) and Na₂SO₄ (ca 2 g) was added. The mixture was stirred for 10 min and the suspension filtered. The filtrate was concentrated in vacuo and the crude product purified by flash chromatography on silica gel (4 g column, 0-90% (10% (0.7 M NH₃ in MeOH) in DCM) in DCM, liquid load (dichloromethane)) to afford the title compound (38.0 mg, 0.16 mmol, 31%) as a brown oil. m/z 231.1 (M+H)⁺ (ES+). ¹H NMR (500 MHz, DMSO) δ 1.73-1.83 (m, 1H), 1.87-1.97 (m, 1H), 2.12 (s, 3H), 2.56-2.63 (m, 1H), 2.71 (dd, J=14.3, 7.8 Hz, 1H), 2.87 (dd, J=14.3, 5.5 Hz, 1H), 3.07-3.14 (m, 1H), 3.19-3.26 (m, 1H), 3.75 (s, 3H), 6.69 (dd, J=8.7, 2.4 Hz, 1H), 6.99 (d, J=2.4 Hz, 1H), 7.04 (d, J=2.2 Hz, 1H), 7.20 (d, J=8.7 Hz, 1H), 10.60 (s, 1H).

### Example 41: Synthesis of (R)-5-ethyl-3-((1-methylpyrrolidin-2-yl)methyl)-1H-indole (91A)

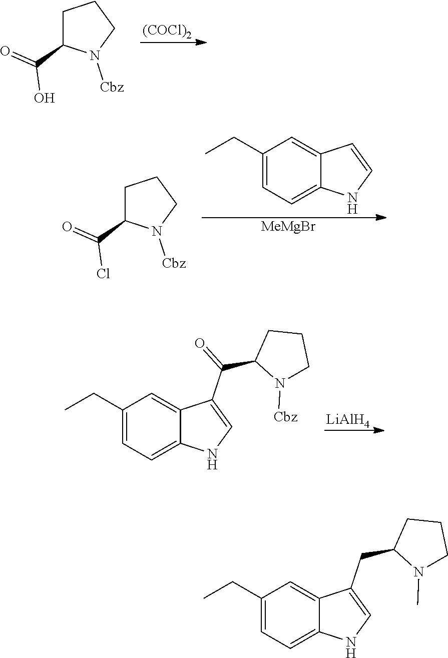

### Step 1: Benzyl (R)-2-(chlorocarbonyl)pyrrolidine-1-carboxylate

To a 0 °C solution of ((benzyloxy)carbonyl)-D-proline (3.00 g, 1 Eq, 12.0 mmol) in anhydrous DCM (75.0 mL) under an inert atmosphere of nitrogen was added oxalyl dichloride (4.58 g, 3.16 mL, 3 Eq, 36.1 mmol) and N,N-dimethylformamide (8.80 mg, 9.32 μL, 0.01 Eq, 120 μmol). The reaction mixture was warmed to ambient temperature and left to stir for 2.5 hours. The reaction mixture was concentrated in vacuo, re-suspended iso-hexane (3×30 mL) and concentrated in vacuo three times, yielding sub-title compound (3.22 g, 12 mmol, 99%, 99% Purity) as a yellow oil. m/z 264.00 (M−Cl+MeOH)⁺ (ES⁺).

### Step 2: Benzyl (R)-2-(5-ethyl-1H-indole-3-carbonyl)pyrrolidine-1-carboxylat

To a stirred solution of 1H-indole (1.00 g, 1.52 Eq, 8.54 mmol) in dry Toluene (20.0 mL) under a nitrogen atmosphere at 22 °C was added methylmagnesium bromide (1.54 g, 4.30 mL, 3.00 molar, 2.3 Eq, 12.9 mmol) dropwise slowly over 5 minutes. The reaction mixture was stirred for 1 hour, a solution of the product from step 1 above (1.50 g, 1 Eq, 5.60 mmol) in dry Toluene (20.0 mL) added dropwise, and the reaction mixture left to stir at room temperature for 18 hours. The reaction mixture was quenched with sat. sodium bicarbonate (80 mL) and left to vigorously stir at room temperature for 1 hour. The resulting suspension was extracted with EtOAc (3×80 mL) and the collected organic layers dried over MgSO₄ and concentrated in vacuo. The crude product was purified by chromatography on silica gel (40 g cartridge, 0-100% EtOAc/iso-hexane) to afford sub-title compound (1.01 g, 2.8 mmol, 50%, 97% Purity).

To a stirred solution of 5-ethyl-1H-indole (1.02 g, 1.5 Eq, 7.00 mmol) in dry Toluene (20.0 mL) under a nitrogen atmosphere at 22 °C was added methylmagnesium bromide (1.28 g, 3.58 mL, 3.00 molar, 2.3 Eq, 10.7 mmol) dropwise slowly over 5 minutes. The reaction mixture was stirred for 1 hour, a solution of the product from step 1 above (1.25 g, 1 Eq, 4.67 mmol) in dry Toluene (20.0 mL) was added dropwise and the reaction mixture was left to stir at room temperature for 18 hours. The reaction mixture was quenched with sat. sodium bicarbonate (80 mL) and left to vigorously stir at room temperature for 30 minutes. The resulting suspension was extracted with EtOAc (3×80 mL) and the collected organic layers were dried over MgSO₄ and concentrated in vacuo. The crude product was purified by chromatography on silica gel (40 g cartridge, 25-75% EtOAc/isohexane) to afford the product as a yellow oil. The product was then triturated with MTBE, and isolated via filtration to afford sub-title compound (0.7958 g, 2.11 mmol, 45.3%, 100% Purity). m/z 374.40 (M−H)⁻ (ES⁻). ¹H NMR (500 MHz, DMSO) δ 11.91 (s, 1H), 11.90-11.88 (m, 1H), 8.40 (d, J=3.1 Hz, 1H), 8.38 (d, J=3.2 Hz, 1H), 8.05 (s, 1H), 8.01 (s, 1H), 7.40-7.30 (m, 7H), 7.14-7.06 (m, 5H), 7.05-6.98 (m, 2H), 5.23 (dd, J=8.8, 3.5 Hz, 1H), 5.17 (dd, J=9.0, 3.5 Hz, 1H), 5.12-5.01 (m, 2H), 4.94 (q, J=13.3 Hz, 2H), 3.57-3.46 (m, 4H), 2.70 (p, J=7.9 Hz, 4H), 2.43-2.35 (m, 1H), 2.34-2.28 (m, 1H), 1.93-1.80 (m, 6H), 1.26-1.19 (m, 6H). Product is present as a ~1:1 mixture of tautomers with the spectrum possessing multiple regions of overlapping. Therefore, spectrum has been quoted as a mixture of both species.

### Step 3: (R)-5-ethyl-3-((1-methylpyrrolidin-2-yl)methyl)-1H-indole

To a solution of the product from step 2 above (150 mg, 1 Eq, 398 μmol) in dry 2Me-THF (4.00 mL) under a nitrogen atmosphere at 22 °C was added LiAlH4 (73 mg, 0.80 mL, 2.40 molar, 4.8 Eq, 1.9 mmol). The reaction mixture was heated to 66 °C and was stirred for 2 hours. The mixture was cooled to 0 °C, diluted with TBME (5 mL) then water (0.1 mL) was added followed by 2 M aq. NaOH (0.1 mL) and water (0.3 mL). The mixture was warmed to rt and stirred for 30 min then filtered washing with EtOAc (2×10 mL) and the filtrate was concentrated in vacuo. The crude product was purified by chromatography on silica gel (12 g cartridge, 0-100% (0.7 M Ammonia/MeOH)/DCM) to afford the sub-title compound (73.2 mg, 302 μmol, 75.8%, 100% Purity) as a clear oil. m/z 243.24 (M+H)⁺ (ES⁺). ¹H NMR (500 MHz, DMSO) δ 10.62 (s, 1H), 7.28 (s, 1H), 7.22 (d, J=8.3 Hz, 1H), 7.07 (s, 1H), 6.91 (d, J=8.3 Hz, 1H), 3.05-2.93 (m, 2H), 2.66 (q, J=7.6 Hz, 2H), 2.48-2.41 (m, 2H), 2.34 (s, 3H), 2.10 (q, J=8.8 Hz, 1H), 1.80-1.49 (m, 3H), 1.49-1.40 (m, 1H), 1.21 (t, J=7.5 Hz, 3H).

### Example 42: Synthesis of (R)-4-fluoro-3-((1-methylpyrrolidin-2-yl)methyl)-1H-indole (82A)

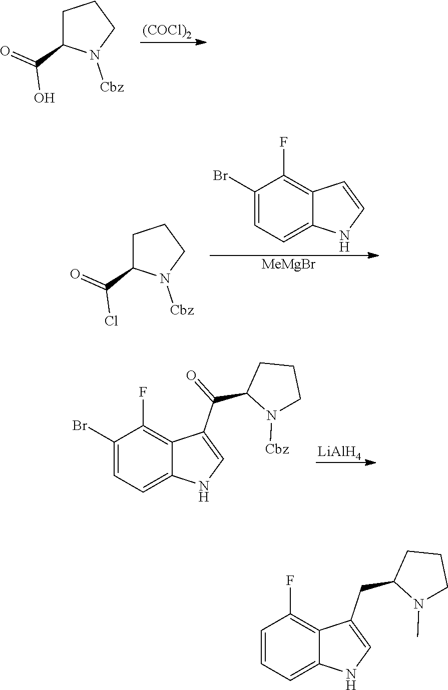

### Step 1: Benzyl (R)-2-(chlorocarbonyl)pyrrolidine-1-carboxylate

To a solution of ((benzyloxy)carbonyl)-D-proline (573 mg, 2.30 mmol) in DCM (10.0 mL) cooled to 0 °C was added drop-wise oxalyl dichloride (1.01 mL, 11.5 mmol) followed by DMF (17.8 μL, 230 μmol). After 30 min the mixture was warmed to RT and stirred for a further 2 h. The mixture was concentrated under reduced pressure to afford a yellow oil.

### Step 2: Benzyl (R)-2-(5-bromo-4-fluoro-1H-indole-3-carbonyl)pyrrolidine-1-carboxylate

To a solution of 5-bromo-4-fluoro-1H-indole (0.50 g, 2.3 mmol) in toluene (5.00 mL) cooled to 0 °C was added drop-wise methylmagnesium bromide (3.0 M in diethyl ether) (0.78 mL, 2.3 mmol). After 30 min the mixture was warmed to RT and stirred for a further 90 min.

A solution of the product of step 1 above in toluene (3.00 mL) was added drop-wise and stirring was continued for a further 18 h. The mixture was poured into sat. aq. NH₄Cl (100 mL) and extracted with EtOAc (3×20 mL). The combined organics were washed with water (50 mL), brine (50 mL), dried (MgSO₄), filtered and concentrated under reduced pressure to afford a brown gum. The crude product was purified by chromatography on silica gel (24 g cartridge, 0-100% EtOAc in isohexane) to afford a pale yellow foam.

The foam was dissolved in THF (20 mL), a solution of LiOH (0.17 g, 7.0 mmol) in water (2 mL) was added and the mixture was stirred at RT for 4 h. The mixture was poured into water (50 mL) and extracted with EtOAc (3×20 mL). The combined organics were washed with water (50 mL), brine (50 mL), dried (MgSO₄), filtered and concentrated under reduced pressure to afford benzyl (the sub-title compound (242 mg, 21%). m/z 445.3/447.3 (M+H)⁺ (ES⁺); 443.3/445.3 (M−H)⁻ (ES⁻).

### Step 3: (R)-4-fluoro-3-((1-methylpyrrolidin-2-yl)methyl)-1H-indole

To a solution of the product of step 2 above (120 mg, 269 μmol) in 2-methyltetrahydrofuran (5.00 mL) was added drop-wise aluminum(III) lithium hydride (2.0 M in THF) (808 μL, 1.62 mmol). After 10 min the mixture was heated to 80 °C and stirred for 2 h. After cooling to 0 °C the mixture was diluted with TBME (20 mL) then quenched by careful addition of water (170 μL), 2 M aq. NaOH (170 μL) and water (510 μL) and stirred rapidly for 30 min. The mixture was filtered washing with EtOAc (2×10 mL) and the filtrate concentrated under reduced pressure.

The crude product was purified by chromatography on RP Flash C18 (12 g cartridge, 5-20% (MeCN+0.1% formic acid) in (water+0.1% formic acid) to afford a gum which was partitioned between DCM (2 mL) and sat. aq. NaHCO₃ (2 mL), passed through a hydrophobic frit and concentrated under reduced pressure to afford the title compound (3.60 mg, 6%). m/z 233.5 (M+H)⁺ (ES⁺). ¹H NMR (500 MHz, DMSO) δ 11.06 (s, 1H), 7.14 (m, 2H), 6.99 (td, J=7.9, 5.1 Hz, 1H), 6.68 (dd, J=11.6, 7.7 Hz, 1H), 3.29 (s, 3H), 3.14 (dd, J=13.8, 3.6 Hz, 1H), 2.96 (app t, J=8.7 Hz, 1H), 2.09 (app q, J=8.8 Hz, 1H), 1.73-1.39 (m, 4H). Note 2 protons obscured by DMSO solvent signal.

### Example 43: Synthesis of (R)-3-((1-((1H-pyrazol-4-yl)methyl)pyrrolidin-2-yl)methyl)-4-fluoro-5-methoxy-1H-indole (5A)

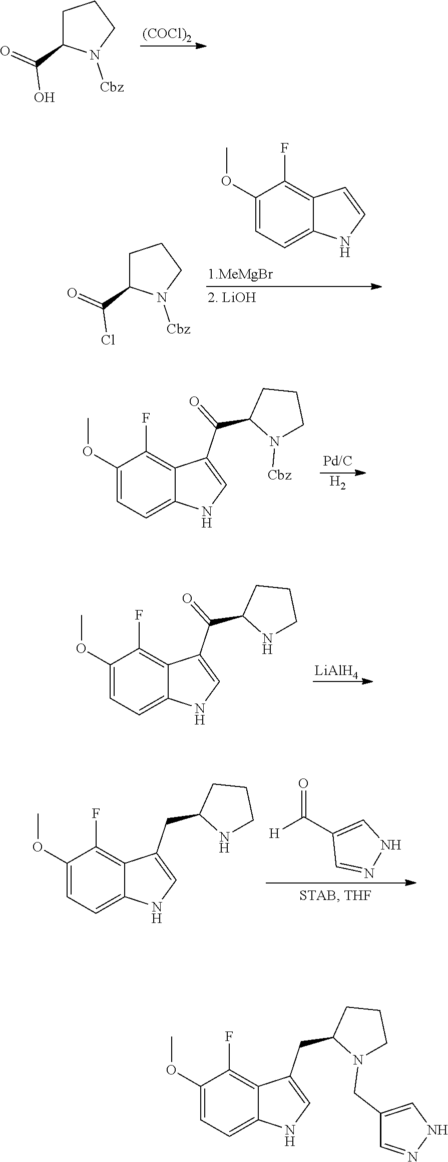

### Step 1: Benzyl (R)-2-(chlorocarbonyl)pyrrolidine-1-carboxylate

To a stirred solution of ((benzyloxy)carbonyl)-D-proline (4.50 g, 1 Eq, 18.1 mmol) in dry DCM (125 mL) under a nitrogen atmosphere at 0 °C was added oxalyl dichloride (6.87 g, 4.74 mL, 3 Eq, 54.2 mmol) and catalytic DMF. The reaction mixture was stirred at rt for 3 h. The reaction mixture was concentrated in vacuo and the resulting crude suspended in iso-hexane (3×30 mL), then concentrated in vacuo to afford sub-title compound (4.83 g, 18.0 mmol, 100%) as a pale-yellow oil. m/z 264.2 (M−Cl+MeOH)⁺ (ES⁺).

### Step 2: Benzyl (R)-2-(4-fluoro-5-methoxy-1H-indole-3-carbonyl)pyrrolidine-1-carboxylate

To a solution of 4-fluoro-5-methoxy-1H-indole (1.00 g, 1.25 Eq, 6.05 mmol) in Toluene (20.00 mL) cooled to 0 °C was added drop-wise methylmagnesium bromide in diethyl ether (753 mg, 2.10 mL, 3.00 molar, 1.3 Eq, 6.31 mmol). After 30 min the mixture was warmed to RT and stirred for a further 90 min. A solution of benzyl (R)-2-(chlorocarbonyl)pyrrolidine-1-carboxylate (1.30 g, 1 Eq, 4.86 mmol) in Toluene (5.00 mL) was added drop-wise and stirring was continued for a further hour.

The mixture was poured into sat. aq. NH₄Cl (100 mL) and extracted with EtOAc (3×50 mL). The combined organics were washed with water (50 mL), brine (50 mL), dried (MgSO₄), filtered and concentrated under reduced pressure to afford a brown gum.

The crude product was purified by chromatography on silica gel (24 g cartridge, 0-100% EtOAc in isohexane, eluted at 80%) to afford benzyl (R)-2-(4-fluoro-5-methoxy-1H-indole-3-carbonyl)pyrrolidine-1-carboxylate (1.29 g, 3.25 mmol, 67.0%).

To a solution of benzyl 2-(3-(((benzyloxy)carbonyl)-D-prolyl)-4-fluoro-5-methoxy-1H-indole-1-carbonyl)pyrrolidine-1-carboxylate (2.04 g, 1 Eq, 3.25 mmol) in THF (20.00 mL) and water (4.0 mL) was added LiOH (390 mg, 5 Eq, 16.3 mmol) and the mixture was stirred at RT for 5 h. The mixture was poured into sat. aq. NH₄Cl (200 mL) and extracted with EtOAc (3×25 mL). The combined organics were washed with water (50 mL), brine (50 mL), dried (MgSO₄), filtered and concentrated under reduced pressure.

The crude product was purified by chromatography on silica gel (24 g cartridge, 0-100% EtOAc in isohexane, eluted at 80%) to afford benzyl (R)-2-(4-fluoro-5-methoxy-1H-indole-3-carbonyl)pyrrolidine-1-carboxylate (576 mg, 1.2 mmol, 25%, 83% Purity). m/z 397.3 (M+H)+ (ES+).

### Step 3: (R)-4-fluoro-5-methoxy-3-prolyl-1H-indole

A stirred suspension of benzyl (R)-2-(4-fluoro-5-methoxy-1H-indole-3-carbonyl)pyrrolidine-1-carboxylate (576 mg, 83% Wt, 1 Eq, 1.21 mmol) and Pd/C (87 L) (128 mg, 5% Wt, 0.05 Eq, 60.3 μmol) in Ethanol (7.50 mL) under a nitrogen atmosphere at rt and the vessel was charged with H2 (2.44 mg, 1 Eq, 1.21 mmol). The reaction mixture was stirred at rt for 18 h. The reaction mixture was filtered through glass microfibres, rinsing with MeOH (250 mL).

The resulting product was redissolved in Methanol (7.00 mL) and Pd/C (87 L) (128 mg, 5% Wt, 0.05 Eq, 60.3 μmol) was added. Under a nitrogen atmosphere at rt, the vessel was charged with H2 (2.44 mg, 1 Eq, 1.21 mmol). The reaction mixture was stirred at rt for 6 h. The reaction mixture was filtered through glass microfibres, rinsing with MeOH (150 mL). The solvent was removed in vacuo to afford (R)-4-fluoro-5-methoxy-3-prolyl-1H-indole (360 mg, 1.2 mmol, 100%, 90% Purity). m/z 263.0 (M+H)+ (ES+); 261.2 (M−H)− (ES−). ¹H NMR in DMSO-d6 4235-133-1-NMR was consistent with product structure at 93% purity with MeOH (~7% wt) impurity. ¹H NMR (500 MHz, DMSO) δ 8.36 (s, 1H), 7.22 (d, J=8.8 Hz, 1H), 7.12 (dd, J=8.7, 7.3 Hz, 1H), 4.41 (dd, J=8.6, 6.0 Hz, 1H), 3.83 (s, 3H), 3.04 (dt, J=10.3, 6.1 Hz, 1H), 2.73 (dt, J=10.4, 6.6 Hz, 1H), 2.19-2.09 (m, 1H), 1.77-1.66 (m, 1H), 1.66-1.56 (m, 2H). 13/15 protons found—2 exchangeable protons not observed. 19F NMR (471 MHz, DMSO) δ −131.67.

### Step 4: (R)-4-fluoro-5-methoxy-3-(pyrrolidin-2-ylmethyl)-1H-indole

To a stirred suspension of (R)-4-fluoro-5-methoxy-3-prolyl-1H-indole (360 mg, 90% Wt, 1 Eq, 1.24 mmol) in dry 2-Methyltetrahydrofuran (10.0 mL) under a nitrogen atmosphere at 0 °C was added lithium aluminum hydride (2.4 M in THF) (188 mg, 2.06 mL, 2.40 molar, 4 Eq, 4.94 mmol). The reaction mixture was heated to 70 °C and stirred for X h.

The mixture was cooled to 0 °C and quenched with 4:1 THF/water mixture (10 mL). Na2SO4 (10 g) was added to the suspension and stirred at rt for 1 h. The suspension was filtered through celite, washing with EtOAc (~200 mL). The filtrate was filtered and concentrated in vacuo to afford (R)-4-fluoro-5-methoxy-3-(pyrrolidin-2-ylmethyl)-1H-indole (383 mg, 0.77 mmol, 62%, 50% Purity) as a crude brown oil. m/z 249.3 (M+H)+ (ES+).

### Step 5: (R)-3-((1-((1H-pyrazol-4-yl)methyl)pyrrolidin-2-yl)methyl)-4-fluoro-5-methoxy-1H-indole

A mixture of (R)-4-fluoro-5-methoxy-3-(pyrrolidin-2-ylmethyl)-1H-indole (130 mg, 1 Eq, 524 μmol) and 1H-pyrazole-4-carbaldehyde (60.4 mg, 1.2 Eq, 628 μmol) in THF (2.00 mL) was stirred at room temperature for 30 min and sodium triacetoxyborohydride (333 mg, 3 Eq, 1.57 mmol) was added and the mixture was stirred at room temperature for 16 hours. The reaction was quenched with 0.7 M NH₃ in MeOH (5 mL) and the solvent was removed under reduced pressure. The crude product was dissolved in 1.6 mL with DMSO, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130A, 5 μm, 30 mm×100 mm, flow rate 40 mL min⁻¹ eluting with a 0.3% Ammonia in water-MeCN gradient over 17.5 mins using UV across all wavelengths with PDA as well as a QDA and ELS detector. At-column dilution pump gives 2 mL min⁻¹ Methanol over the entire method, which is included in the following MeCN percentages. Gradient information: 0.0-0.5 min, 15% MeCN; 0.5-15.5 min, ramped from 15% MeCN to 45% MeCN; 15.5-15.6 min, ramped from 45% MeCN to 100% MeCN; 15.6-17.5 min, held at 100% MeCN. The clean fractions were evaporated in a Genevac to afford (R)-3-((1-((1H-pyrazol-4-yl)methyl)pyrrolidin-2-yl)methyl)-4-fluoro-5-methoxy-1H-indole (8.40 mg, 24 μmol, 4.6%, 94% Purity). 329.3 (M+H)+ (ES+). ¹H NMR (DMSO-d6) is in accordance with product structure at 94% purity. ¹H NMR (500 MHz, DMSO) δ 10.86 (s, 1H), 7.64-7.39 (m, 2H), 7.13 (s, 1H), 7.06 (d, J=8.6 Hz, 1H), 6.97-6.90 (m, 1H), 3.89 (d, J=13.3 Hz, 1H), 3.81 (d, J=1.1 Hz, 3H), 3.16 (d, J=13.7 Hz, 1H), 2.88-2.81 (m, 1H), 2.67-2.54 (m, 2H), 2.18-2.11 (m, 1H), 1.70-1.43 (m, 3H)—NH pyrazole missing and 2H under water residual peak. ¹⁹F NMR (471 MHz, DMSO) δ −147.47.

### Example 44: Synthesis of (R)-4-(2-((4-Fluoro-5-methoxy-1H-indol-3-yl)methyl)pyrrolidin-1-yl)tetrahydro-2H-thiopyran 1,1-dioxide (4A)

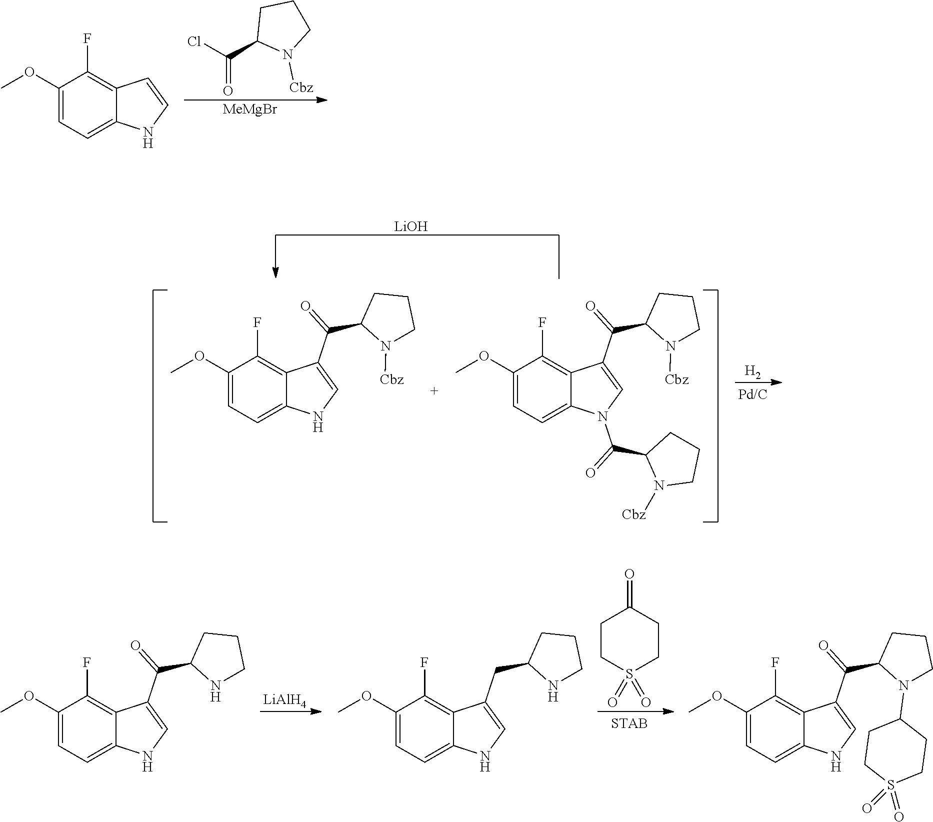

### Step 1: Benzyl (R)-2-(4-fluoro-5-methoxy-1H-indole-3-carbonyl)pyrrolidine-1-carboxylate

To a solution of 4-fluoro-5-methoxy-1H-indole (1.00 g, 1.25 Eq, 6.05 mmol) in toluene (20.00 mL) cooled to 0 °C was added drop-wise MeMgBr in Et₂O (753 mg, 2.10 mL, 3.00 molar, 1.3 Eq, 6.31 mmol). After 30 min the mixture was warmed to rt and stirred for a further 90 min. A solution of benzyl (R)-2-(chlorocarbonyl)pyrrolidine-1-carboxylate (1.30 g, 1 Eq, 4.86 mmol) in toluene (5.00 mL) was added drop-wise and stirring was continued for a further 1 h. The mixture was poured into sat. aq. NH₄Cl (100 mL) and extracted with EtOAc (3×50 mL). The combined organics were washed with water (50 mL), brine (50 mL), dried (MgSO₄), filtered and concentrated in vacuo to afford a brown gum. The crude product was purified by chromatography on silica gel (24 g cartridge, 0-100% EtOAc in isohexane, eluted at 80%) to afford the sub-title compound, contaminated with benzyl 2-(3-(((benzyloxy)carbonyl)-D-prolyl)-4-fluoro-5-methoxy-1H-indole-1-carbonyl)pyrrolidine-1-carboxylate (1.29 g, 3.25 mmol, 67.0%). This material was dissolved in THF (20.0 mL) and water (4.0 mL) to which was added LiOH (390 mg, 5 Eq, 16.3 mmol) and the mixture was stirred at rt for 5 h. The mixture was poured into sat. aq. NH₄Cl (200 mL) and extracted with EtOAc (3×25 mL). The combined organics were washed with water (50 mL), brine (50 mL), dried (MgSO₄), filtered and concentrated in vacuo. The crude product was purified by chromatography on silica gel (24 g cartridge, 0-100% EtOAc in isohexane, eluted at 80%) to afford the sub-title compound (576 mg, 1.2 mmol, 25%). Note: yield calculated over 2 steps. m/z 397.3 (M+H)⁺ (ES+).

### Step 2: (R)-4-Fluoro-5-methoxy-3-prolyl-1H-indole

A stirred suspension of the product from step 1 above (576 mg, 83% Wt, 1 Eq, 1.21 mmol) and Pd/C (87 L) (128 mg, 5% Wt, 0.05 Eq, 60.3 μmol) in EtOH (7.50 mL) under a nitrogen atmosphere at rt and the vessel was charged with H₂ (2.44 mg, 1 Eq, 1.21 mmol). The reaction mixture was stirred at rt for 18 h. The reaction mixture was filtered through glass microfibres, rinsing with MeOH (250 mL). The resulting product was redissolved in MeOH (7.00 mL) and Pd/C (87 L) (128 mg, 5% Wt, 0.05 Eq, 60.3 μmol) was added. Under a nitrogen atmosphere at rt, the vessel was charged with H₂ (2.44 mg, 1 Eq, 1.21 mmol). The reaction mixture was stirred at rt for 6 h. The reaction mixture was filtered through glass microfibres, rinsing with MeOH (150 mL). The solvent was removed in vacuo to afford the sub-title compound (360 mg, 1.2 mmol, 100%). m/z 263.0 (M+H)⁺ (ES+); 261.2 (M−H)⁻ (ES−). ¹H NMR (500 MHz, DMSO) δ 8.36 (s, 1H), 7.22 (d, J=8.8 Hz, 1H), 7.12 (dd, J=8.7, 7.3 Hz, 1H), 4.41 (dd, J=8.6, 6.0 Hz, 1H), 3.83 (s, 3H), 3.04 (dt, J=10.3, 6.1 Hz, 1H), 2.73 (dt, J=10.4, 6.6 Hz, 1H), 2.19-2.09 (m, 1H), 1.77-1.66 (m, 1H), 1.66-1.56 (m, 2H). 2 exchangeable protons not observed. ¹⁹F NMR (471 MHz, DMSO) δ −131.67.

### Step 3: (R)-4-Fluoro-5-methoxy-3-(pyrrolidin-2-ylmethyl)-1H-indol

To a stirred suspension of the product from step 2 above (360 mg, 90% Wt, 1 Eq, 1.24 mmol) in dry 2-Me-THF (10.0 mL) under a nitrogen atmosphere at 0 °C was added LiAlH₄ (2.4 M in THF) (188 mg, 2.06 mL, 2.40 molar, 4 Eq, 4.94 mmol). The reaction mixture was heated to 70 °C and stirred. The mixture was cooled to 0 °C and quenched with 4:1 THF/water mixture (10 mL). Na₂SO₄ (10 g) was added to the suspension and stirred at rt for 1 h. The suspension was filtered through celite, washing with EtOAc (~200 mL). The filtrate was filtered and concentrated in vacuo to afford the sub-title compound (383 mg, 0.77 mmol, 62%) as a crude brown oil. m/z 249.3 (M+H)⁺ (ES+).

### Step 4: (R)-4-(2-((4-Fluoro-5-methoxy-1H-indol-3-yl)methyl)pyrrolidin-1-yl)tetrahydro-2H-thiopyran 1,1-dioxide

To a stirred solution of the product from step 3 above (80.0 mg, 50% Wt, 1 Eq, 161 μmol) in dry THF (2.00 mL) at rt was added tetrahydro-4H-thiopyran-4-one 1,1-dioxide (35.8 mg, 1.5 Eq, 242 μmol). The reaction mixture was stirred for 1 h. NaBH(OAc)₃ (102 mg, 3 Eq, 483 μmol) was added and the reaction mixture left to stir for 18 h at rt. The reaction mixture was quenched with sat. aq. NaHCO₃ (5 mL) and left to stir. The aq. suspension was extracted with EtOAc (3×5 mL). The combined organic layers were dried (Na₂SO₄), filtered and concentrated in vacuo. The crude product was purified by chromatography on RP Flash C18 (12 g cartridge, 0-10% (MeCN+0.1% formic acid) in (water+0.1% formic acid)) to afford impure title compound (2.20 mg, 5.3 μmol, 3.3%) as a brown gum. The crude was purified again by chromatography on silica gel (4 g cartridge, 0-10% MeOH in DCM) to afford the title compound (2.20 mg, 5.3 μmol, 3.3%) as a gum. m/z 381.3 (M+H)⁺ (ES+); 379.4 (M−H)⁻ (ES−). ¹H NMR (500 MHz, DMSO) δ 10.89 (s, 1H), 7.13 (d, J=2.3 Hz, 1H), 7.06 (d, J=8.7 Hz, 1H), 6.93 (t, J=8.4 Hz, 1H), 3.80 (s, 3H), 3.23-3.15 (m, 1H), 3.13-3.00 (m, 5H), 2.99 (dd, J=13.9, 3.6 Hz, 1H), 2.81 (td, J=8.0, 3.0 Hz, 1H), 2.56 (dd, J=16.2, 8.3 Hz, 1H), 2.48-2.44 (m, 1H), 2.06 (d, J=4.8 Hz, 3H), 2.03-1.92 (m, 1H), 1.72-1.46 (m, 4H). ¹⁹F NMR (471 MHz, DMSO) δ −147.79.

### Example 45: Functional Assays at 5-HT_{2A}, 5-HT_{2C}, and 5-HT_{1A} Receptors

Agonist activity at 5-HT_{2A}, 5-HT_{2C}, and 5-HT_{1A} receptors was assessed using Revvity's HTRF assay kits (IP-One kit for 5-HT_{2A} and 5-HT_{2C}; cAMP kit for 5-HT_{1A}). Test compounds, dissolved in DMSO, were dispensed into 384-well plates using an ECHO acoustic dispenser to generate a ten-point half-log concentration-response curve. CHO-K1 cells stably expressing the respective serotonin receptors (ValiScreen GPCR Cell Lines ES-315-C and ES-313-C from PerkinElmer, or cAMP Hunter 95-0132C2 from DiscoverX) were added using a Multidrop dispenser and incubated at 37 °C with 5% CO2.

For assays using 5-HT_{1A}-expressing cells, test compounds were co-incubated with 2 μM forskolin to elevate basal cAMP levels and 10 nM SB 224289 to block endogenous 5-HT_{1B} receptor activity.

Following incubation, cells were lysed by adding the kit's lysis buffer supplemented with donor (cryptate-conjugated) and acceptor (d2-labeled) antibodies specific for IP₁ (for 5-HT_{2A} and 5-HT_{2C}) or cAMP (for 5-HT_{1A}). After room-temperature incubation to allow antibody binding and HTRF signal stabilization, fluorescence emissions at 620 nm and 665 nm were measured using a PHERAstar plate reader.

A standard curve for IP₁ or cAMP was prepared in parallel and treated with the same detection reagents. The HTRF signal (665/620 nm ratio) was used to interpolate analyte concentrations from the standard curve, enabling the calculation of compound potency (pEC₅₀). The results are shown in Table 1.

TABLE 1
Assay Results

| Compound | | | | |
|---|---|---|---|---|
| No. | Structure | 2A pEC50 | 1A pEC50 | 2C pEC50 |
| 2A | 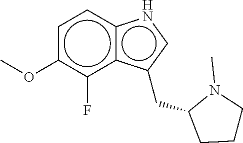 | 7.192 | 6.879 | 7.627 |
| 3A | 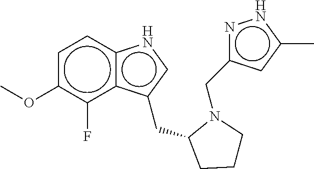 | 5.837 | — | — |
| 19A | 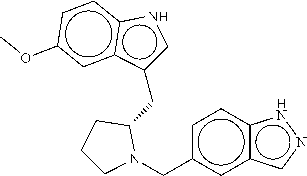 | 6.63 | 7.628 | 6.929 |
| 20A | 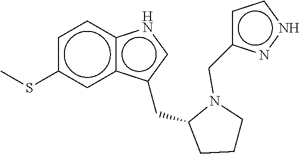 | 7.024 | 7.609 | 8.059 |
| 21A | 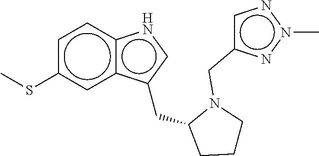 | 6.874 | 7.553 | 5 |
| 22B | 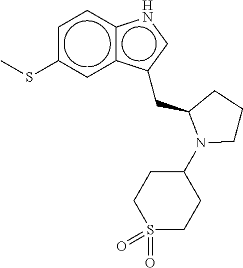 | — | — | 6.825 |
| 23A | 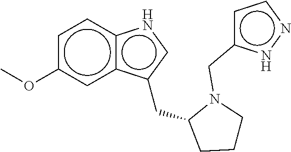 | 7.286 | 7.478 | 7.902 |
| 24A | 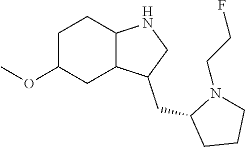 [SMILES: COC1CCC2NCC(C[C@H]3CCCN3CCF)C2C1] | 7.283 | 7.47 | 7.548 |
| 25A | 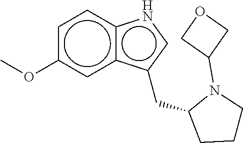 | 7.203 | 7.392 | 6.859 |
| 26A | 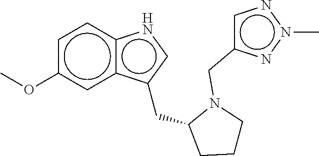 | 7.441 | 7.345 | 7.32 |
| 27A | 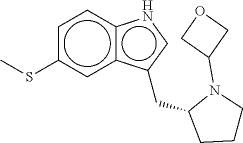 | 6.582 | 7.318 | 7.204 |
| 28B | 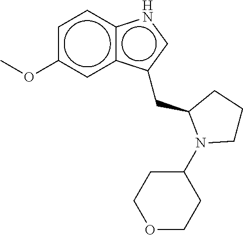 | 6.855 | 7.28 | 6.543 |
| 29B | 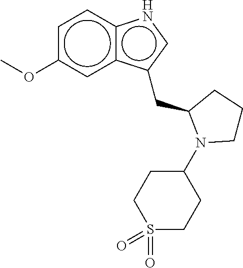 | 6.621 | 7.166 | 6.443 |
| 30B | 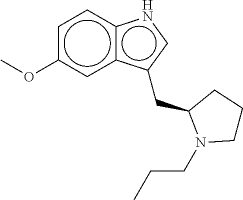 | 7.573 | 7.152 | 7.094 |
| 31A | 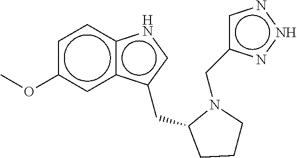 | 6.705 | 7.099 | 7.365 |
| 32A | 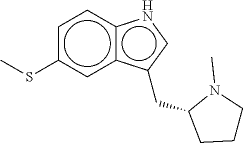 | 7.716 | 7.087 | 8.578 |
| 33A | 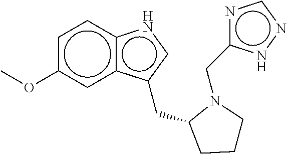 | 6.772 | 7.025 | 7.759 |
| 34A | 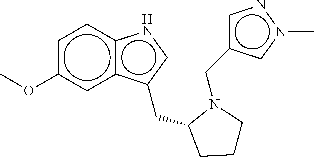 | 6.956 | 6.991 | 6.545 |
| 35A | 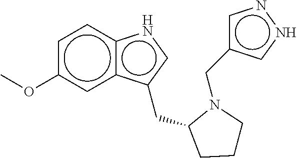 | 6.731 | 6.958 | 7.119 |
| 36A | 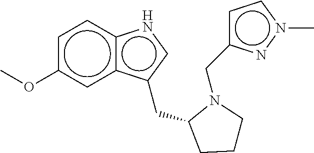 | 7.271 | 6.957 | 7.388 |
| 37A | 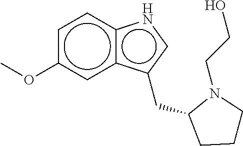 | 7.284 | 6.927 | 7.109 |
| 38A | 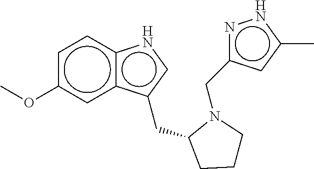 | 7.537 | 6.916 | 8.527 |
| 39B | 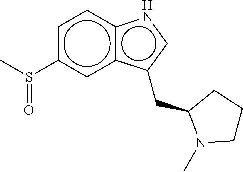 | 4.848 | 6.664 | 5.695 |
| 40A | 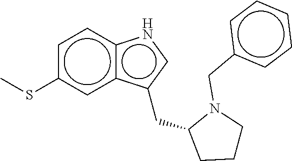 | 7.128 | 6.465 | 7.346 |
| 41B | 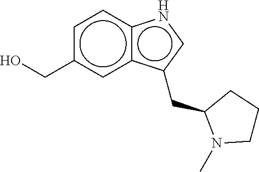 | 6.028 | 6.398 | 6.808 |
| 42A | 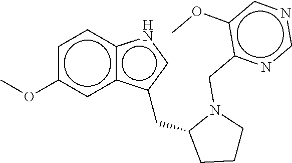 | 8.174 | 6.195 | 7.332 |
| 43B | 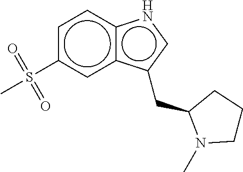 | .5 | 5.885 | 5.203 |
| 44A | 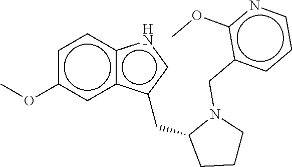 | 7.004 | 5.834 | 6.476 |
| 45A | 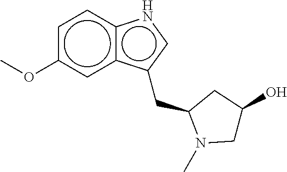 | 6.829 | 5.075 | 7.587 |
| 46A | 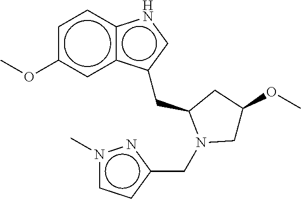 | — | — | 6.045 |
| 47A | 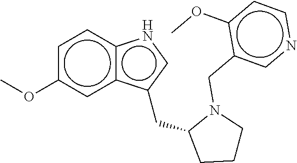 | 6.29 | — | 6.576 |
| 65A | 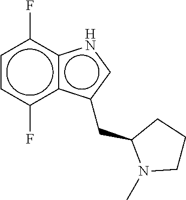 | 6.916 | 6.248 | 8.259 |
| 66A | 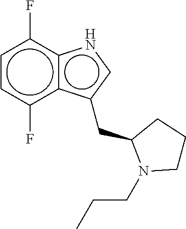 | 6.725 | — | 7.184 |
| 67A | 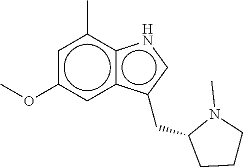 | 6.52 | 5.019 | 7.092 |
| 68A | 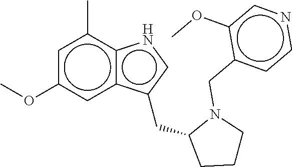 | 6.854 | 5 | 5.78 |
| 69B | 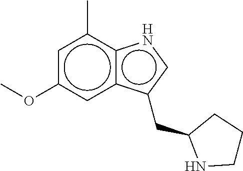 | 6.84 | 5 | 7.273 |
| 70A | 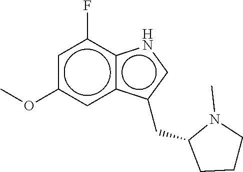 | 7.787 | 7.309 | 8.127 |
| 71A | 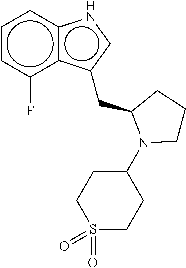 | 6.629 | 6.967 | 6.524 |
| 72A | 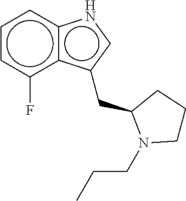 | 6.308 | 6.874 | 6.936 |
| 73A | 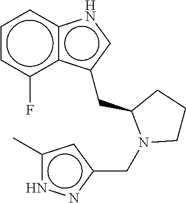 | 6.692 | 6.863 | 7.608 |
| 74A | 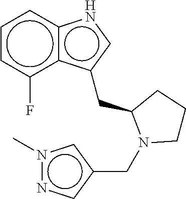 | 6.233 | 6.716 | 6.921 |
| 75A | 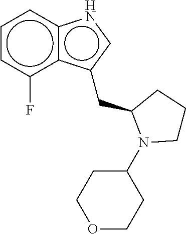 | 5.636 | 6.544 | 6.752 |
| 76A | 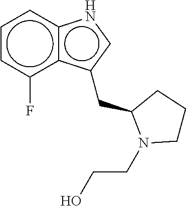 | 5.881 | 6.231 | 6.416 |
| 77A | 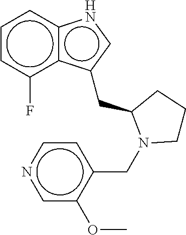 | 6.919 | 6.193 | 6.171 |
| 78A | 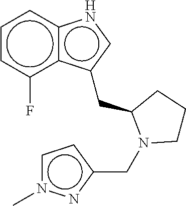 | 6.773 | 5.73 | 7.056 |
| 79A | 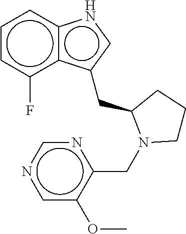 | 7.229 | 5.208 | 6.158 |
| 80A | 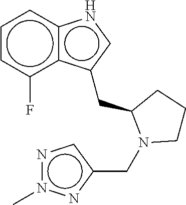 | 6.586 | 6.303 | 6.414 |
| 85A | 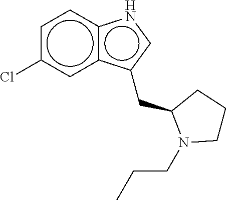 | 7.053 | 6.947 | 7.404 |
| 81B | 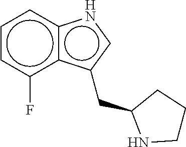 | 6.528 | 5.958 | 7.173 |
| 86B | 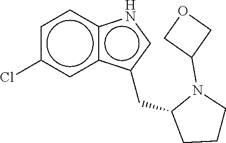 | 6.001 | 6.65 | 7.388 |
| 87A | 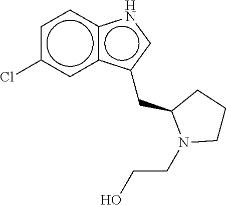 | 5.929 | 5.802 | 5 |
| 88A | 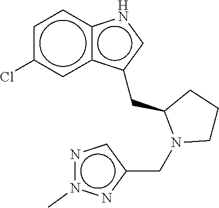 | 6.934 | — | 7.966 |

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A compound of Formula (I): 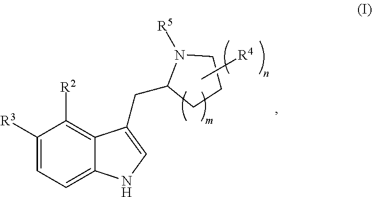
or a pharmaceutically acceptable salt, deuterated form or stereoisomer thereof
wherein:
R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl;
wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or —C₁₋₆ alkyl;
R² is -halo, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, —O—C₁₋₆ alkyl, —OH, —S—C₁₋₆ alkyl, —S(O)₂—C₁₋₆ alkyl, —SH, —NH—C₁₋₆ alkyl, —N(C₁₋₆ alkyl)₂, —NH₂, —CN, —C(C═O)—C₁₋₆ alkyl, —O(C═O)—C₁₋₆ alkyl, or —NH(C═O)—C₁₋₆ alkyl;
R³ is -halo, —O—C₁₋₆ alkyl, —OH, —S—C₁₋₆ alkyl, —S(O)₂—C₁₋₆ alkyl, —SH, —NH—C₁₋₆ alkyl, —N(C₁₋₆ alkyl)₂, —NH₂, —CN, —C(C═O)—C₁₋₆ alkyl, —O(C═O)—C₁₋₆ alkyl, or —NH(C═O)—C₁₋₆ alkyl;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —OH, —C₁₋₆ alkylene-O—C₁₋₆ alkyl, or —O—C₁₋₆ alkyl;
n is 0, 1, 2, or 3;
m is 0 or 1,
and wherein, R² and R³ are not both -halo.

2. The compound of claim 1, wherein R¹ is —C₁₋₆ alkyl, cycloalkyl, —C₁₋₆ alkylene-heteroaryl, or -heterocyclyl, wherein the heteroaryl is optionally substituted with —C₁₋₆ alkyl and the —C₁₋₆ alkyl is optionally substituted with -halo.

3. The compound of claim 2, wherein R¹ is methyl.

4. The compound of claim 2, wherein R¹ is —C₁₋₃ alkylene-5 to 6 membered heteroaryl, wherein the heteroaryl is optionally substituted with a methyl.

5. The compound of claim 2, wherein the —C₁₋₆ alkylene-heteroaryl is

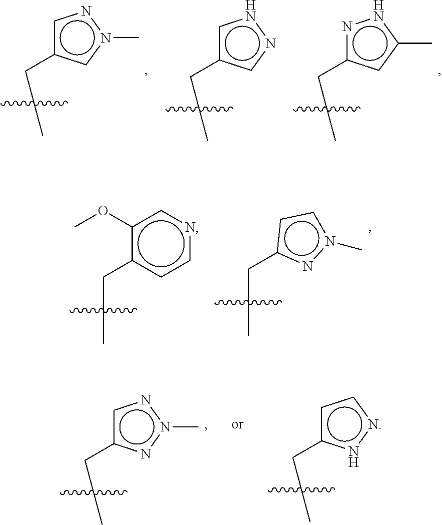

6. The compound of claim 2, wherein the -heterocyclyl is

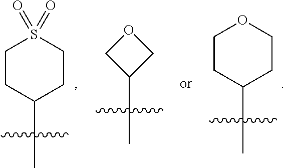

7. The compound of claim 2, wherein the cycloalkyl is

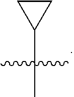 [SMILES: *C1CC1]

8. The compound of claim 1, wherein R² is -halo.

9. The compound of claim 8, wherein R² is —F.

10. The compound of claim 1, wherein R³ is —O—C₁₋₆ alkyl.

11. The compound of claim 8, wherein R³ is —O-Me.

12. The compound of claim 1, wherein n is 0.

13. The compound of claim 1, wherein m is 1.

14. The compound of claim 1, wherein
R¹ is —C₁₋₆ alkyl or —C₁₋₆ alkylene-heteroaryl, wherein the heteroaryl is optionally substituted with —C₁₋₆ alkyl;
R² is -halo; and
R³ is —O—C₁₋₆ alkyl.

15. The compound of claim 1, wherein
R¹ is —C₁₋₆ alkyl or —C₁₋₆ alkylene-heteroaryl, wherein the heteroaryl is optionally substituted with —C₁₋₆ alkyl;
R² is —F; and
R³ is —O-Me.

16. The compound of claim 1, wherein the compound is:

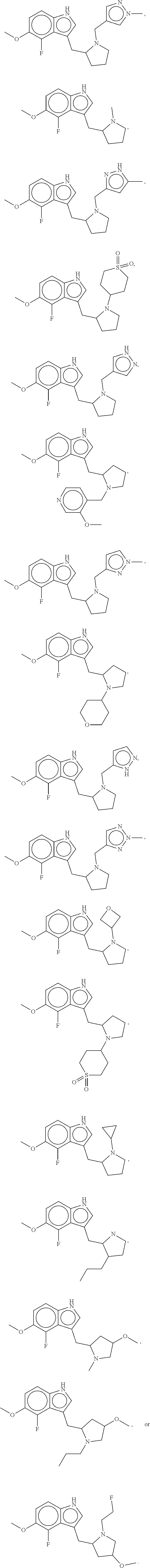

17. (canceled)

18. (canceled)

19. A compound of Formula (II): 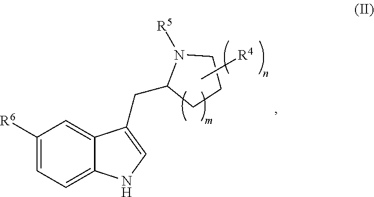
or a pharmaceutically acceptable salt, deuterated form or stereoisomer thereof
wherein:
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —OH, —C₁₋₆ alkylene-O—C₁₋₆ alkyl, or —O—C₁₋₆ alkyl;
R⁵ is —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl;
wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or —C₁₋₆ alkyl;
R⁶ is —O—C₁₋₆ alkyl, —S—C₁₋₆ alkyl, —S(O)₂—C₁₋₆ alkyl, —SH, —N(C₁₋₆ alkyl)₂, —CN, —C(C═O)—C₁₋₆ alkyl, —O(C═O)—C₁₋₆ alkyl, —C₁₋₆ alkylene-OH, or —S(O)—C₁₋₆ alkyl;
n is 0, 1, 2, or 3
m is 0 or 1,
and wherein, the compound is not:

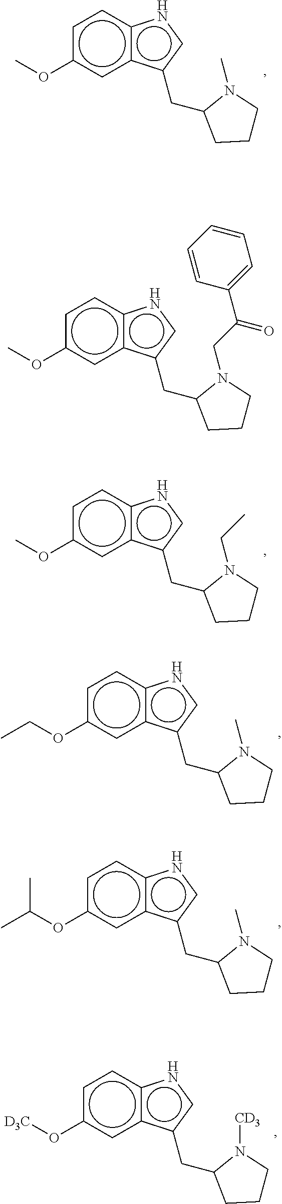

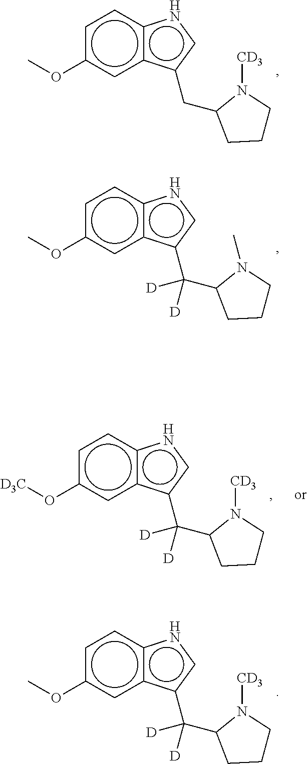

20. The compound of claim 19, wherein the compound is not deuterated.

21. The compound of claim 19, wherein R⁵ is —C₁₋₆ alkyl, -cycloalkyl, —C₁₋₆ alkylene-aryl, —C₁₋₆ alkylene-heteroaryl, or -heterocyclyl, each of which is optionally substituted with -halo or —OH.

22. The compound of claim 21, wherein R⁵ is —C₁₋₆ alkyl substituted with -halo or —OH.

23. The compound of claim 22, wherein R⁵ is —CH₂CH₂F or —CH₂CH₂OH.

24. The compound of claim 21, wherein R⁵ is —C₁₋₆ alkylene-aryl is

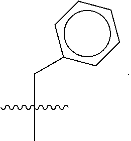

25. The compound of claim 21, wherein the —C₁₋₆ alkylene-heteroaryl is

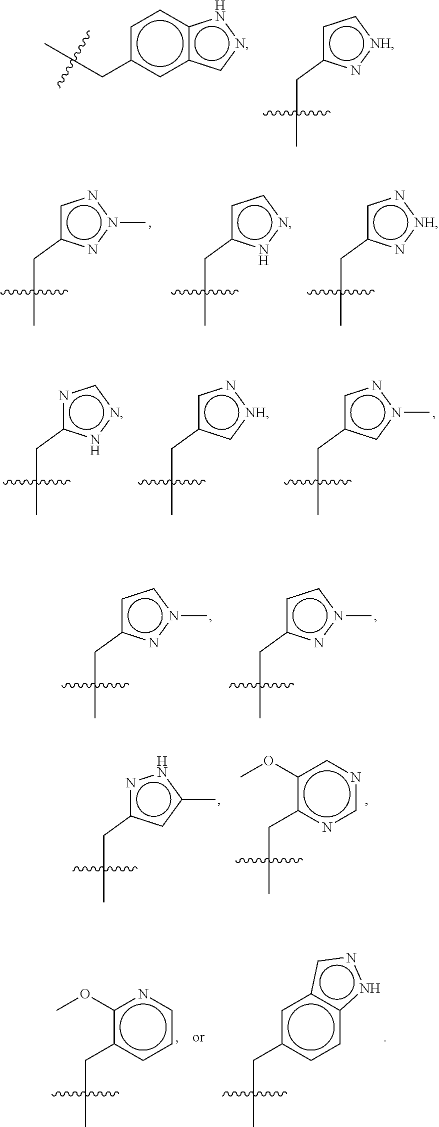

26. The compound of claim 21, wherein the -heterocyclyl is

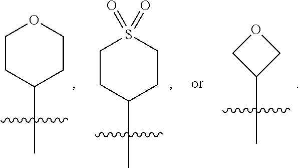

27. The compound of claim 21, wherein the -cycloalkyl is

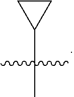 [SMILES: *C1CC1]

28. The compound of claim 19, wherein R⁶ is —O—C₁₋₆ alkyl, —S—C₁₋₆ alkyl, —S(O)—C₁₋₆ alkyl, —S(O)₂—C₁₋₆ alkyl, —C₁₋₆ alkylene-OH.

29. The compound of claim 28, wherein R⁶ is —O-Me, —S-Me, —CH₂—OH, —S(O)-Me, or —S(O)₂-Me.

30. The compound of claim 28, wherein R⁶ is —S-Me or —O-Me.

31. (canceled)

32. The compound of claim 19, wherein R⁴ is —O—C₁₋₆ alkyl or —OH.

33. The compound of claim 19, wherein n is 1.

34. The compound of claim 19, wherein n is 0.

35. The compound of claim 19, wherein m is 1.

36. The compound of claim 19, wherein the compound is:

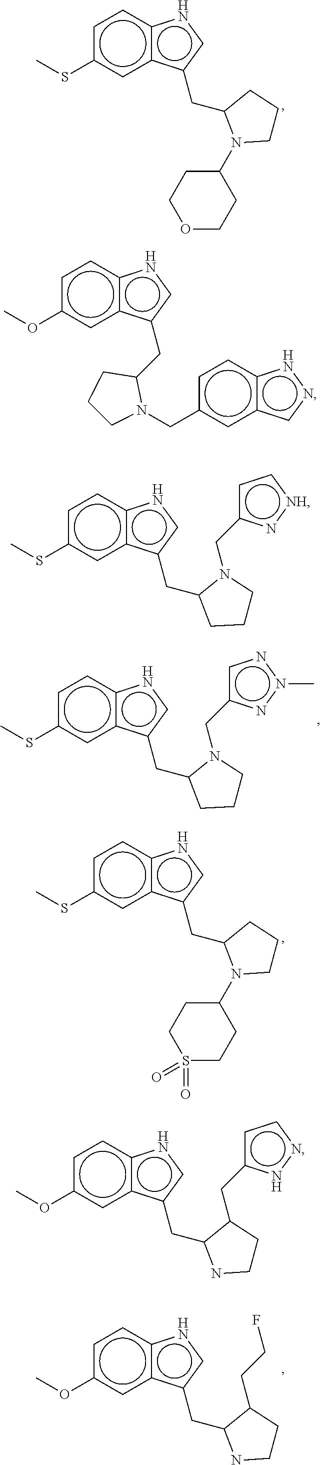

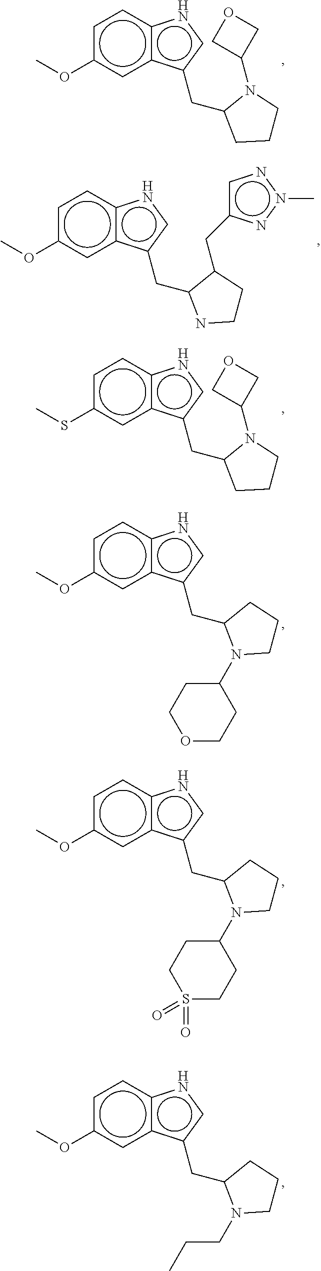

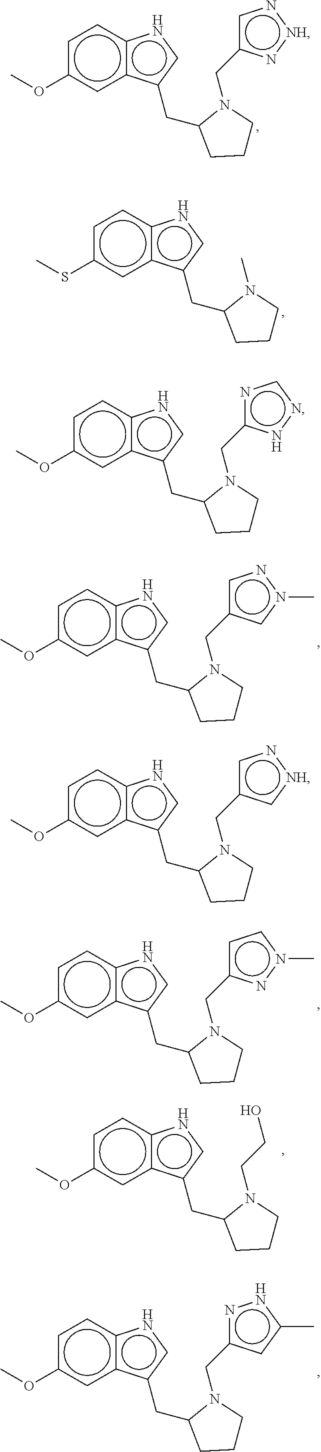

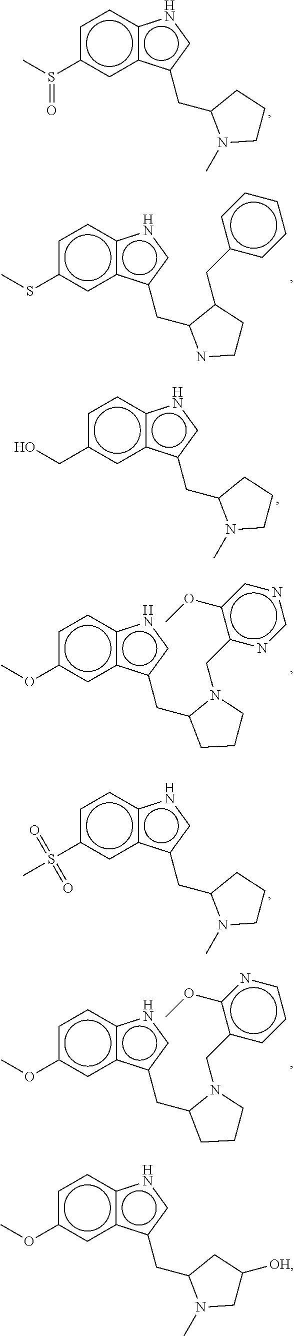

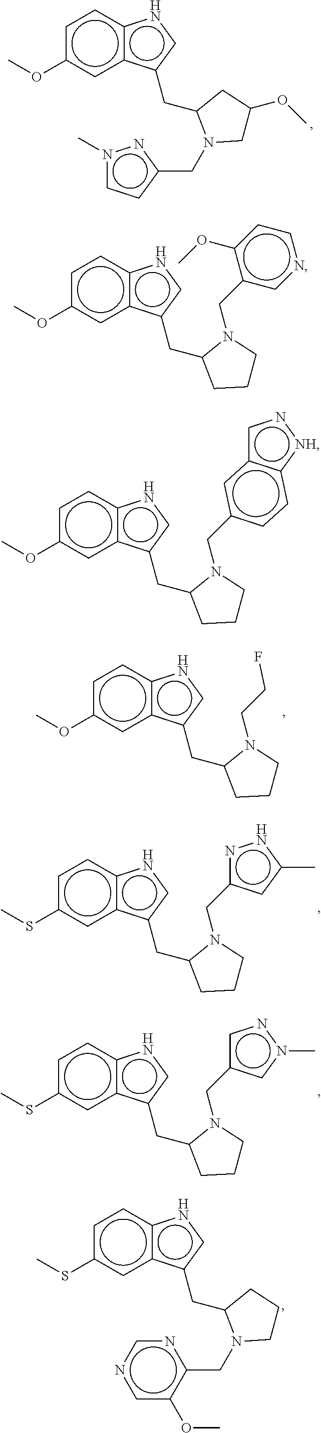

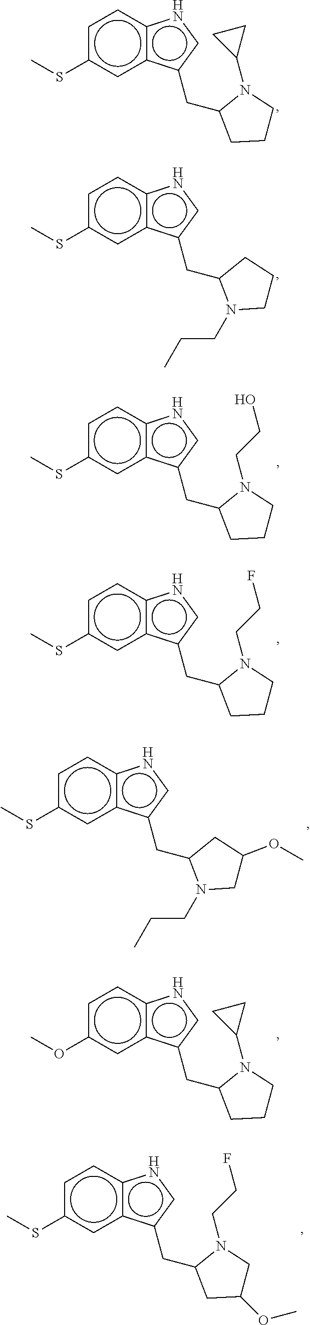

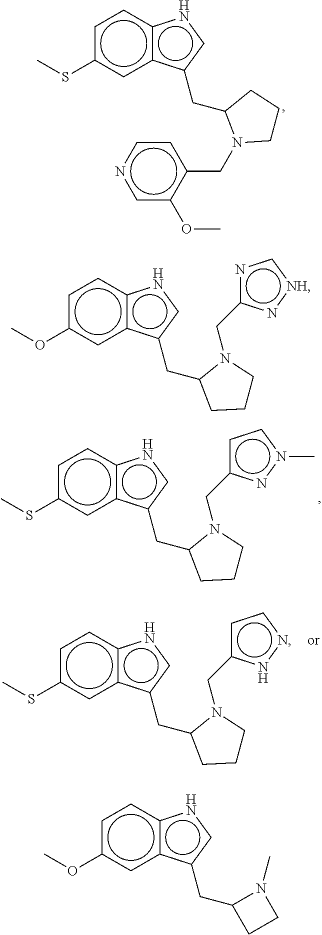

37. (canceled)

38. (canceled)

39. A compound of Formula (III): 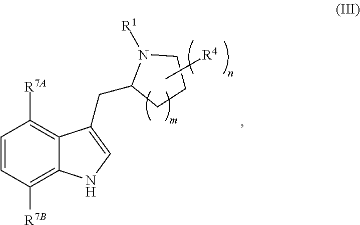
or a pharmaceutically acceptable salt, deuterated form or stereoisomer thereof
wherein:
R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl;
wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or —C₁₋₆ alkyl;
R^{7A} and R^{7B} are each independently -halo;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —OH, —C₁₋₆ alkylene-O—C₁₋₆ alkyl, or —O—C₁₋₆ alkyl;
n is 0, 1, 2, or 3; and
m is 0 or 1.

40. The compound of claim 39, wherein R¹ is —C₁₋₆ alkyl.

41. The compound of claim 39, wherein R¹ is methyl or butyl.

42. The compound of claim 39, wherein R^{7A} and R^{7B} are both —F.

43. The compound of claim 39, wherein n is 0.

44. The compound of claim 39, wherein m is 1.

45. The compound of claim 39, wherein the compound is:

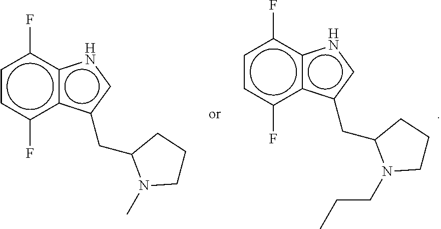

46. (canceled)

47. (canceled)

48. A compound of Formula (IV): 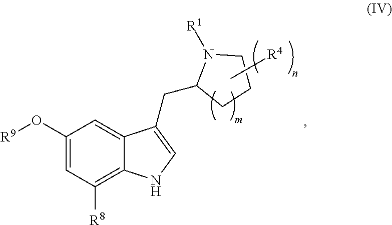
or a pharmaceutically acceptable salt, deuterated form or stereoisomer thereof
wherein:
R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl;
wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or —C₁₋₆ alkyl;
R⁸ is -halo or -methyl;
R⁹ is —C₁₋₆ alkyl;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —OH, —C₁₋₆ alkylene-O—C₁₋₆ alkyl, or —O—C₁₋₆ alkyl;
n is 0, 1, 2, or 3; and
m is 0 or 1.

49. The compound of claim 48, wherein R¹ is —H, —C₁₋₆ alkyl, or —C₁₋₆ alkylene-heteroaryl.

50. The compound of claim 49, wherein the —C₁₋₆ alkylene-heteroaryl is

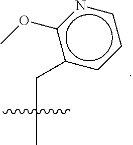

51. The compound of claim 48, wherein R⁸ is -halo.

52. The compound of claim 48, wherein R⁸ is -methyl.

53. The compound of claim 48, wherein R⁹ is -methyl.

54. The compound of claim 48, wherein n is 0.

55. The compound of claim 48, wherein m is 1.

56. The compound of claim 48, wherein the compound is:

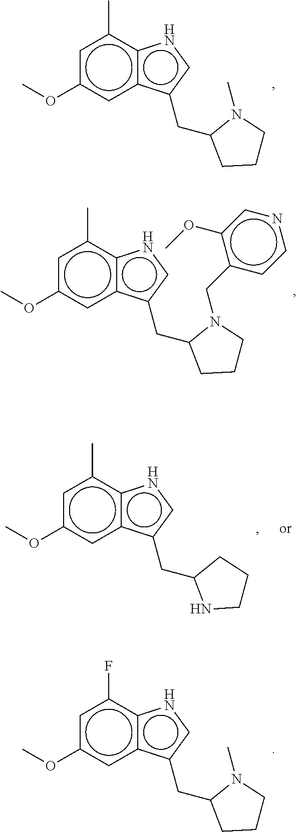

57. (canceled)

58. (canceled)

59. A compound of formula (V): 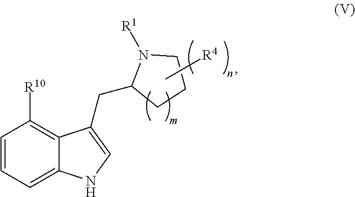
or a pharmaceutically acceptable salt, deuterated form or stereoisomer thereof
wherein:
R¹ is —H, —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl;
wherein the —C₁₋₆ alkyl, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, -cycloalkyl, —C₁₋₆ alkylene-O—C₁₋₆ alkyl, or —O—C₁₋₆ alkyl are optionally substituted with one or more -halo, oxo, —OH, —NH₂, —CN, or —C₁₋₆ alkyl;
R⁴ is —C₁₋₆ alkyl, -aryl, -heteroaryl, —C₁₋₆ alkylene-aryl, —C₁₋₆ alkylene-heteroaryl, —C₁₋₆ alkylene-OH, —OH, —C₁₋₆ alkylene-O—C₁₋₆ alkyl, or —O—C₁₋₆ alkyl;
R¹⁰ is -halo, —C₂₋₆ alkenyl, —C₂₋₆ alkynyl, -aryl, -heteroaryl, —C₁₋₆ alkylene-aryl, —C₁₋₆ alkylene-heteroaryl, -heterocyclyl, —C₁₋₆ alkylene-O—C₁₋₆ alkyl, —S—C₁₋₆ alkyl, —S(O)₂—C₁₋₆ alkyl, —SH, —NH—C₁₋₆ alkyl, —N(C₁₋₆ alkyl)₂, —NH₂, —CN, —C(C═O)—C₁₋₆ alkyl, —O(C═O)—C₁₋₆ alkyl, or —NH(C═O)—C₁₋₆ alkyl;
n is 0, 1, 2, or 3; and
m is 0 or 1.

60. The compound of claim 59, wherein R¹ is —H, -heterocyclyl, -cycloalkyl, —C₁₋₆ alkyl, or —C₁₋₆ alkylene-heteroaryl, wherein the —C₁₋₆ alkyl is optionally substituted with —OH or -halo.

61. The compound of claim 60, wherein the -heterocyclyl is:

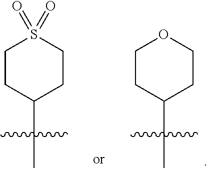 [SMILES: *C1CCS(=O)(=O)CC1 ; *C1CCOCC1]

62. The compound of claim 60, wherein the —C₁₋₆ alkylene-heteroaryl is:

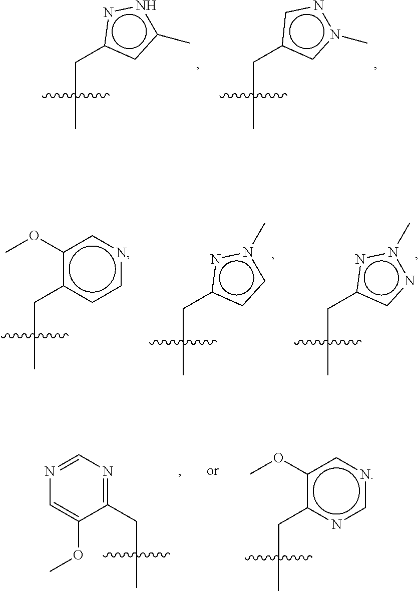

63. The compound of claim 60, wherein the -cycloalkyl is

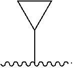 [SMILES: *C1CC1]

64. The compound of claim 60, wherein R¹ is —C₁₋₆ alkyl substituted with —OH.

65. The compound of claim 64, wherein R¹ is —CH₂CH₂OH.

66. The compound of claim 60, wherein R¹ is —CH₂CH₂CH₃.

67. The compound of claim 60, wherein R¹ is —C₁₋₆ alkyl substituted with -halo.

68. The compound of claim 67, wherein R¹ is —CH₂CH₂F.

69. The compound of claim 59, wherein R¹⁰ is -halo.

70. The compound of claim 69, wherein R¹⁰ is —F.

71. The compound of claim 59, wherein n is 0.

72. The compound of claim 59, wherein m is 1.

73. The compound of claim 59, wherein:
R¹ is -heterocyclyl, -cycloalkyl, —C₁₋₆ alkyl, or —C₁₋₆ alkylene-heteroaryl;
R¹⁰ is -halo; and
n is 0.

74. The compound of claim 59, wherein the compound is:

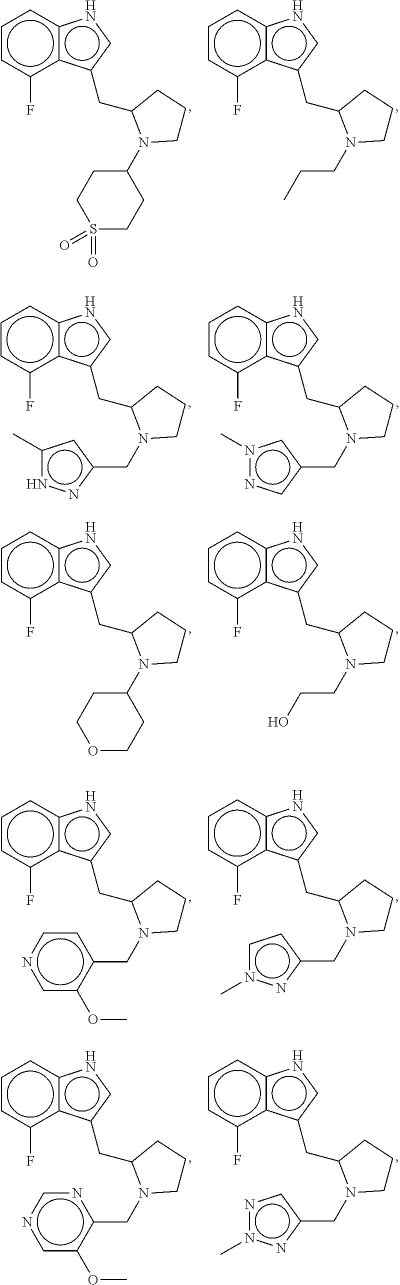

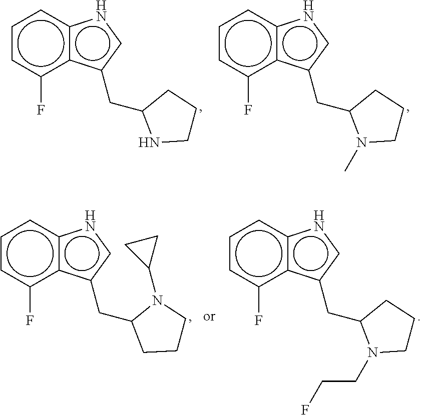

75. (canceled)

76. (canceled)

77. A compound selected from:

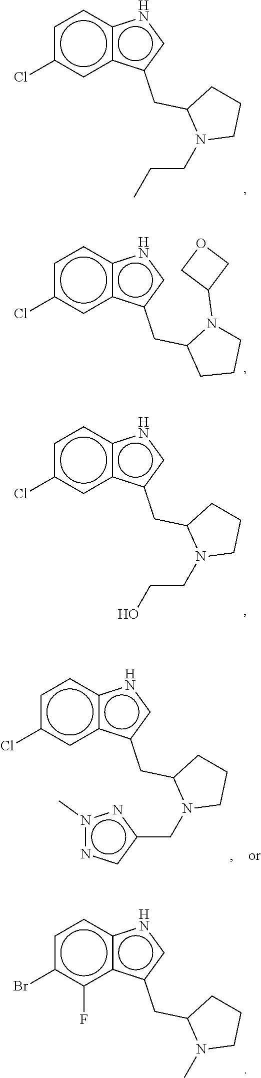

78. (canceled)

79. (canceled)

80. A pharmaceutical composition comprising a compound of claim 1, or a pharmaceutically acceptable salt or deuterated form thereof.

81. (canceled)

82. (canceled)

83. A method of treating 5-HT2A receptor associated disease or disorder, comprising administering a compound of claim 1, or a pharmaceutically acceptable salt or deuterated form thereof.

84-93. (canceled)
